SEQUENCE LISTING (104 sequences)

SEQ ID NO: 1 - protein, 11 aa, Artificial sequence
  sequence is synthesized
KASQXVGSXVA

SEQ ID NO: 2 - protein, 11 aa, Artificial sequence
  sequence is synthesized
RASQDIXXYLT

SEQ ID NO: 3 - protein, 11 aa, Artificial sequence
  sequence is synthesized
RASQDISXYLX

SEQ ID NO: 4 - protein, 11 aa, Artificial sequence
  sequence is synthesized
KASQXLRSKVA

SEQ ID NO: 5 - protein, 11 aa, Artificial sequence
  sequence is synthesized
KASQYVGTHVA

SEQ ID NO: 6 - protein, 11 aa, Artificial sequence
  sequence is synthesized
RASQSISSYLA

SEQ ID NO: 7 - protein, 7 aa, Artificial sequence
  sequence is synthesized
SASYRYS

SEQ ID NO: 8 - protein, 7 aa, Artificial sequence
  sequence is synthesized
YTSRLHS

SEQ ID NO: 9 - protein, 7 aa, Artificial sequence
  sequence is synthesized
SASYRKS

SEQ ID NO: 10 - protein, 7 aa, Artificial sequence
  sequence is synthesized
GASSRAS

SEQ ID NO: 11 - protein, 9 aa, Artificial sequence
  sequence is synthesized
HQYXXYPYT

SEQ ID NO: 12 - protein, 9 aa, Artificial sequence
  sequence is synthesized
QQGXTLPWT

SEQ ID NO: 13 - protein, 9 aa, Artificial sequence
  sequence is synthesized
QQYXXYPYT

SEQ ID NO: 14 - protein, 9 aa, Artificial sequence
  sequence is synthesized
QQRFSVPFT

SEQ ID NO: 15 - protein, 9 aa, Artificial sequence
  sequence is synthesized
QQYYSSPLT

SEQ ID NO: 16 - protein, 10 aa, Artificial sequence
  sequence is synthesized
GYTFTRFWIH

SEQ ID NO: 17 - protein, 10 aa, Artificial sequence
  sequence is synthesized
GYTLAXYGMX

SEQ ID NO: 18 - protein, 10 aa, Artificial sequence
  sequence is synthesized
GYXLAXYGLX

SEQ ID NO: 19 - protein, 10 aa, Artificial sequence
  sequence is synthesized
GFSFSSQGIS

SEQ ID NO: 20 - protein, 10 aa, Artificial sequence
  sequence is synthesized
GFTFSSYAMS

SEQ ID NO: 21 - protein, 18 aa, Artificial sequence
  sequence is synthesized
GEIXPSXGRTXYXEXFKX

SEQ ID NO: 22 - protein, 18 aa, Artificial sequence
  sequence is synthesized
GWIXTXTGKPTYSDEFKG

SEQ ID NO: 23 - protein, 18 aa, Artificial sequence
  sequence is synthesized
GWIXTXTGAPTYAEEFKG

SEQ ID NO: 24 - protein, 18 aa, Artificial sequence
  sequence is synthesized
SRISPSGGSTYYADSVKG

SEQ ID NO: 25 - protein, 17 aa, Artificial sequence
  sequence is synthesized
STISYDGSTYYADSVKG

SEQ ID NO: 26 - protein, 6 aa, Artificial sequence
  sequence is synthesized
GGRLDQ

SEQ ID NO: 27 - protein, 12 aa, Artificial sequence
  sequence is synthesized
SIYYYGSRYFXV

SEQ ID NO: 28 - protein, 12 aa, Artificial sequence
  sequence is synthesized
SIYYYASRYFXV

SEQ ID NO: 29 - protein, 12 aa, Artificial sequence
  sequence is synthesized
ESSYYEWGAMDV

SEQ ID NO: 30 - protein, 11 aa, Artificial sequence
  sequence is synthesized
EHYFHWGGMDV

SEQ ID NO: 31 - protein, 11 aa, Artificial sequence
  sequence is synthesized
EEYYYWGAMDV

SEQ ID NO: 32 - protein, 13 aa, Artificial sequence
  sequence is synthesized
QFMLWGKQFGMDV

SEQ ID NO: 33 - protein, 9 aa, Artificial sequence
  sequence is synthesized
QQYSNYPYT

SEQ ID NO: 34 - protein, 9 aa, Artificial sequence
  sequence is synthesized
QQYKHYPYT

SEQ ID NO: 35 - protein, 9 aa, Artificial sequence
  sequence is synthesized
QQYKKYPYT

SEQ ID NO: 36 - protein, 9 aa, Artificial sequence
  sequence is synthesized
QQYKNYPYT

SEQ ID NO: 37 - protein, 9 aa, Artificial sequence
  sequence is synthesized
QQYRIYPYT

SEQ ID NO: 38 - protein, 9 aa, Artificial sequence
  sequence is synthesized
QQYKRYPYT

SEQ ID NO: 39 - protein, 9 aa, Artificial sequence
  sequence is synthesized
QQYKSYPYT

SEQ ID NO: 40 - protein, 9 aa, Artificial sequence
  sequence is synthesized
QQYRSYPYT

SEQ ID NO: 41 - protein, 9 aa, Artificial sequence
  sequence is synthesized
QQYSKYPYT

SEQ ID NO: 42 - protein, 108 aa, Artificial sequence
  sequence is synthesized
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 43 - protein, 108 aa, Artificial sequence
  sequence is synthesized
DIQMTQTTSSLSASLGDKVTISCRASQDISNYLNWYQQKPDGTIKLLIYYTSRLHSGIPSRFSGSGSGTDYSLTISNLEQEDIATYFCQQGNTLPWTFGGGTKVEIKR

SEQ ID NO: 44 - protein, 108 aa, Artificial sequence
  sequence is synthesized
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPWTFGQGTKVEIKR

SEQ ID NO: 45 - protein, 108 aa, Artificial sequence
  sequence is synthesized
EVQLVESGGGLVQPGGSLRLSCAASGFTFSSYAMSWVRQAPGKGLEWVSVISGDGGSTYYADSVKGRFTISRDNSKNTLYLQMNSLRAEDTAVYYCARGFDYWGQGTL

SEQ ID NO: 46 - protein, 116 aa, Artificial sequence
  sequence is synthesized
QIQLVQSGPELKKPGETVKISCKASGYNLANYGLNWVKQAPGEGLKWMGWINTNTGAPTYAEEFKGRFVFFLETSASTAYLQINNLSDEDTATYFCARSIYYYASRYFNVWGAGTS

SEQ ID NO: 47 - protein, 116 aa, Artificial sequence
  sequence is synthesized
EVQLVESGGGLVQPGGSLRLSCAASGYNLANYGLNWVRQAPGKGLEWVGWINTNTGAPTYAEEFKGRFTISADTSKNTAYLQMNSLRAEDTAVYYCARSIYYYASRYFNVWGQGTL

SEQ ID NO: 48 - protein, 108 aa, Artificial sequence
  sequence is synthesized
DIQMTQSTSSLSASLGDRVTISCRASQDINNYLTWYQQKPDGTVKLLIYYTSRLHSGVPSRFSGSGSGTDYSLTITNLEQEDFATYFCQQGNTLPWTFGEGTKVEIKR

SEQ ID NO: 49 - protein, 108 aa, Artificial sequence
  sequence is synthesized
DIQMTQSPSSLSASVGDRVTITCRASQDINNYLTWYQQKPGKAPKLLIYYTSRLHSGVPSRFSGSGSGTDFTLTISSLQPEDFATYYCQQGNTLPWTFGQGTKVEIKR

SEQ ID NO: 50 - protein, 116 aa, Artificial sequence
  sequence is synthesized
QIQLVQSGPELKKPGETVKISCKASGYTLANYGMNWVKQAPGKGFKWMGWINTNTGKPTYSDEFKGRFVFSLETSASTAYLLINNLSNEDTATYFCARSIYYYGSRYFNVWGAGTT

SEQ ID NO: 51 - protein, 116 aa, Artificial sequence
  sequence is synthesized
EVQLVESGGGLVQPGGSLRLSCAASGYTLANYGMNWVRQAPGKGLEWVGWINTNTGKPTYSDEFKGRFTISADTSKNTAYLQMNSLRAEDTAVYYCARSIYYYGSRYFNVWGQGTL

SEQ ID NO: 52 - protein, 108 aa, Artificial sequence
  sequence is synthesized
DIVMTQSQKFMSTSLGDRVSVTCKASQNVGSNVAWYQQKPGQSPEALIYSASYRYSGVPDRFTGSGSGTDFTLTISNVQSEDLAEYFCHQYNNYPYTFGGGTKVEIKR

SEQ ID NO: 53 - protein, 108 aa, Artificial sequence
  sequence is synthesized
DIQMTQSPSSLSASVGDRVTITCKASQNVGSNVAWYQQKPGKAPKLLIYSASYRYSGVPSRFSGSGSGTDFTLTISSLQPEDFATYYCHQYNNYPYTFGQGTKVEIKR

SEQ ID NO: 54 - protein, 110 aa, Artificial sequence
  sequence is synthesized
QVQLQQPGAELVKPGASVTLSCKASGYTFTRFWIHWVKQRPGQGLEWIGEINPSNGRTNYNENFKNKATLTVDTSSSTAYMQLSSLTSEVSVVYYCARGGRLDQWGQGTT

SEQ ID NO: 55 - protein, 111 aa, Artificial sequence
  sequence is synthesized
EVQLVESGGGLVQPGGSLRLSCAASGYTFTRFWIHWVRQAPGKGLEWVGEINPSNGRTNYNENNFKNRFTISADTSKNTAYLQMNSLRAEDTAVYYCARGGRLDQWGQGTL

SEQ ID NO: 56 - protein, 27 aa, Artificial sequence
  sequence is synthesized
KASQNLRSKVASASYRYSQQYSNYPYT

SEQ ID NO: 57 - protein, 27 aa, Artificial sequence
  sequence is synthesized
KASQNVGSNVASASYRYSQQYKHYPYT

SEQ ID NO: 58 - protein, 27 aa, Artificial sequence
  sequence is synthesized
KASQNVGSNVASASYRKSQQYNNYPYT

SEQ ID NO: 59 - protein, 27 aa, Artificial sequence
  sequence is synthesized
KASQNVGSNVASASYRYSQQYKKYPYT

SEQ ID NO: 60 - protein, 27 aa, Artificial sequence
  sequence is synthesized
KASQNVGSNVASASYRYSQQYKNYPYT

SEQ ID NO: 61 - protein, 27 aa, Artificial sequence
  sequence is synthesized
KASQNVGSNVASASYRYSQQYRIYPYT

SEQ ID NO: 62 - protein, 27 aa, Artificial sequence
  sequence is synthesized
KASQNVGSNVASASYRYSQQYKRYPYT

SEQ ID NO: 63 - protein, 27 aa, Artificial sequence
  sequence is synthesized
KASQYVGTHVASASYRYSQQYKSYPYT

SEQ ID NO: 64 - protein, 27 aa, Artificial sequence
  sequence is synthesized
KASQNVGSNVASASYRYSQQYRSYPYT

SEQ ID NO: 65 - protein, 27 aa, Artificial sequence
  sequence is synthesized
KASQNVGSNVASASYRYSQQYSKYPYT

SEQ ID NO: 66 - protein, 108 aa, Artificial sequence
  sequence is synthesized
DIQMTQSPSSLSASVGDRVTITCRASQSISSYLAWYQQKPGKAPKLLIYGASSRASGVPSRFSGSGSGTDFTLTISSLQPEDFATYYCQQYYSSPLTFGQGTKVEIKR

SEQ ID NO: 67 - protein, 108 aa, Artificial sequence; identical to SEQ ID NO: 66
  sequence is synthesized

SEQ ID NO: 68 - protein, 108 aa, Artificial sequence
  sequence is synthesized
DIQMTQSPSSLSASVGDRVTITCRASQSISSYLAWYQQKPGKAPKLLIYGASSRASGVPSRFSGSGSGTDFTLTISSLQPEDFATYYCQQRFSVPFTFGQGTKVEIKR

SEQ ID NO: 69 - protein, 108 aa, Artificial sequence; identical to SEQ ID NO: 66
  sequence is synthesized

SEQ ID NO: 70 - protein, 115 aa, Artificial sequence
  sequence is synthesized
EVQLVESGGGLVQPGGSLRLSCAASGFTFSSYAMSWVRQAPGKGLEWVSRISPSGGSTYYADSVKGRFTISADTSKNTAYLQMNSLRAEDTAVYYCAREHYFHWGGMDVWGQGTL

SEQ ID NO: 71 - protein, 115 aa, Artificial sequence
  sequence is synthesized
EVQLVESGGGLVQPGGSLRLSCAASGFTFSSYAMSWVRQAPGKGLEWVSRISPSGGSTYYADSVKGRFTISADTSKNTAYLQMNSLRAEDTAVYYCAREEYYYWGAMDVWGQGTL

SEQ ID NO: 72 - protein, 116 aa, Artificial sequence
  sequence is synthesized
EVQLVESGGGLVQPGGSLRLSCAASGFSFSSQGISWVRQAPGKGLEWVSTISYDGSTYYADSVKGRFTISADTSKNTAYLQMNSLRAEDTAVYYCARQFMLWGKQFGMDVWGQGTL

SEQ ID NO: 73 - protein, 115 aa, Artificial sequence
  sequence is synthesized
EVQLVESGGGLVQPGGSLRLSCAASGFTFSSYAMSWVRQAPGKGLEWVSRISPSGGSTYYADSVKGRFTISADTSKNTAYLQMNSLRAEDTAVYYCARESYYEWGAMDVWGQGTL

SEQ ID NO: 74 - protein, 25 aa, Artificial sequence [sequence omitted]

SEQ ID NO: 75 - protein, 13 aa, Artificial sequence
  sequence is synthesized
WVRQAPGKGLEWV

SEQ ID NO: 76 - protein, 32 aa, Artificial sequence
  sequence is synthesized
RFTISRDXSKXTLYLQMXSLRAEDTAVYYCAR

SEQ ID NO: 77 - protein, 6 aa, Artificial sequence
  sequence is synthesized
WGQGTL

SEQ ID NO: 78 - protein, 23 aa, Artificial sequence
  sequence is synthesized
DIQMTQSPSSLSASVGDRVTITC

SEQ ID NO: 79 - protein, 15 aa, Artificial sequence
  sequence is synthesized
WYQQKPGKAPKLLIY

SEQ ID NO: 80 - protein, 32 aa, Artificial sequence
  sequence is synthesized
GVPSRFSGSGSGTDFTLTISSLQPEDFATYYC

SEQ ID NO: 81 - protein, 11 aa, Artificial sequence
  sequence is synthesized
FGQGTKVEIKR

SEQ ID NO: 82 - protein, 25 aa, Artificial sequence
  sequence is synthesized
XXQLXXXGXXLXXPGXXXXXSCXAS

SEQ ID NO: 83 - protein, 13 aa, Artificial sequence
  sequence is synthesized
WVXQXPGXGXXWX

SEQ ID NO: 84 - protein, 33 aa, Artificial sequence
  sequence is synthesized
XXXXXXXXSXXTXYXXXXXXLXXEDXXXYXCAR

SEQ ID NO: 85 - protein, 6 aa, Artificial sequence
  sequence is synthesized
WGXGTX

SEQ ID NO: 86 - protein, 23 aa, Artificial sequence
  sequence is synthesized
DIXMTQXXXXXSXSXGDXVXXXC

SEQ ID NO: 87 - protein, 15 aa, Artificial sequence
  sequence is synthesized
WYQQKPXXXXXXLIY

SEQ ID NO: 88 - protein, 32 aa, Artificial sequence
  sequence is synthesized
GXPXRFXGSGSGTDXXLTIXNXXXEDXAXYXC

SEQ ID NO: 89 - protein, 11 aa, Artificial sequence
  sequence is synthesized
FGXGTKVEIKR

SEQ ID NO: 90 - protein, 464 aa, Artificial sequence
  sequence is synthesized
MGWSCIILFLVATATGVHSEVQLVESGGGLVQPGGSLRLSCAASGYTFTRFWIHWVRQAPGKGLEWVGEINPSNGRTNYNENFKNRFTISADTSKNTAYLQMNSLRAEDTAVYYCARGGRLDQWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 233 aa, Artificial sequence
  sequence is synthesized
MGWSCIILFLVATATGVHSDIQMTQSPSSLSASVGDRVTITCKASQNVGSNVAWYQQKPGKAPKLLIYSASYRYSGVPSRFSGSGSGTDFTLTISSLQPEDFATYYCHQYNNYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 6 aa, Artificial sequence
  sequence is synthesized
SISSYL

SEQ ID NO: 93 - protein, 6 aa, Artificial sequence
  sequence is synthesized
GASSRA

SEQ ID NO: 94 - protein, 6 aa, Artificial sequence
  sequence is synthesized
YYSSPL

SEQ ID NO: 95 - protein, 9 aa, Artificial sequence
  sequence is synthesized
FTFSSYAMS

SEQ ID NO: 96 - protein, 10 aa, Artificial sequence
  sequence is synthesized
RISPSGGSTY

SEQ ID NO: 97 - protein, 11 aa, Artificial sequence
  sequence is synthesized
SRISPSGGSTY

SEQ ID NO: 98 - protein, 10 aa, Artificial sequence
  sequence is synthesized
REHYFHWGGM

SEQ ID NO: 99 - protein, 10 aa, Artificial sequence
  sequence is synthesized
REEYYYWGAM

SEQ ID NO: 100 - protein, 10 aa, Artificial sequence
  sequence is synthesized
RESYYEWGAM

SEQ ID NO: 101 - protein, 6 aa, Artificial sequence
  sequence is synthesized
RFSVPF

SEQ ID NO: 102 - protein, 9 aa, Artificial sequence
  sequence is synthesized
FSFSSQGIS

SEQ ID NO: 103 - protein, 10 aa, Artificial sequence
  sequence is synthesized
STISYDGSTY

SEQ ID NO: 104 - protein, 12 aa, Artificial sequence
  sequence is synthesized
RQFMLWGKQFGM

(19) United States
(12) Patent Application Publication
(10) Pub. No.: US 2009/0068110 A1
(43) Pub. Date: Mar. 12, 2009

(54) ANTIBODIES TO INSULIN-LIKE GROWTH FACTOR RECEPTOR

(75) Inventors: Yonglei Shang, Millbrae, CA (US); Jean-Philippe F. Stephan, San Carlos, CA (US); Yan Wu, Foster City, CA (US); Jiping Zha, Foster City, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/959,783

(22) Filed: Dec. 19, 2007

Related U.S. Application Data
(60) Provisional application No. 60/942,931, filed on Jun. 8, 2007.
(60) Provisional application No. 60/871,703, filed on Dec. 22, 2006.

(51) Int. Cl. A61K 39/395; C07K 16/28; C12P 21/02; A61K 49/00; C12N 5/00

(52) U.S. Cl. 424/9.2; 530/387.1; 530/387.3; 435/69.6; 435/375; 424/130.1

(57) ABSTRACT

The invention provides various antibodies that bind to insulin-like growth factor-I receptor (IGF-1R), methods for making such antibodies, compositions and articles incorporating such antibodies, and their uses in treating, for example, cancer or aging. The antibodies include murine, chimeric, and humanized antibodies.

### RELATED APPLICATIONS

This non-provisional application claims priority under U.S.C. § 119(e) to U.S. Provisional Application Ser. No. 60/871,703 filed on Dec. 22, 2006 and U.S. Provisional Application Ser. No. 60/942,931 filed on Jun. 8, 2007, both of which are incorporated herein by reference in their entirety.

### FIELD OF THE INVENTION

The present invention concerns antibodies and their uses. More particularly, the present invention concerns antibodies that bind to the insulin-like growth factor-I receptor (IGF-1R).

### BACKGROUND OF THE INVENTION

In several types of cancer, growth factors specifically bind to their receptors and then transmit growth, transformation, and/or survival signals to the tumoral cell. Over-expression of growth factor receptors at the tumoral cell surface is described, e.g., in Salomon et al., Crit. Rev. Oncol. Hematol., 19: 183 (1995); Burrow et al., J. Surg. Oncol., 69: 21 (1998); Hakam et al., Hum. Pathol., 30: 1128 (1999); Railo et al., Eur. J. Cancer, 30: 307 (1994); and Happerfield et al., J. Pathol., 183: 412 (1997). Targeting of such growth factor receptors (e.g., epidermal growth factor (EGF) receptor or Her2/neu) with humanized 4D5 (HERCEPTIN®) trastuzumab) or chimeric (C225) antibodies significantly inhibits tumoral growth in patients and increases efficacy of classical chemotherapy treatments (Carter, Nature Rev. Cancer, 1: 118 (2001); Hortobagyi, Semin. Oncol., 28: 43 (2001); Herbst et al., Semin. Oncol., 29: 27 (2002)).

Insulin-like growth factor-I (IGF-I; also called somatomedin-C) (Klapper et al., Endocrinol., 112: 2215 (1983); Rinderknecht et al., FEBS. Lett., 89: 283 (1978); U.S. Pat. No. 6,331,609; and U.S. Pat. No. 6,331,414) is a member of a family of related polypeptide hormones that also includes insulin, insulin-like growth factor-II (IGF-II) and more distantly nerve growth factor. Each of these growth factors has a cognate receptor to which it binds with high affinity, but some may also bind (albeit with lower affinity) to the other receptors as well (Rechler and Nissley, Ann. Rev. Physiol., 47: 425-42 (1985)). In the extracellular space, the IGF ligands potentially interact with four receptors and six binding proteins (Clemmons, Mol. Cell. Endocrinol., 140: 19-24 (1998)).

The IGFs exert mitogenic activity on various cell types, including tumor cells (Macaulay, Br. J. Cancer, 65:311 (1992); Ibrahim et al., Clin. Cancer Res., 11: 944s-50s (2005)), by binding to a common receptor named the insulin-like growth factor receptor-1 (IGF-1R) (Sepp-Lorenzino, Breast Cancer Research and Treatment, 47: 235 (1998)). IGF-1R (also known as EC 2.7.112, CD 221 antigen) belongs to the family of transmembrane protein tyrosine kinases (Ullrich et al., Cell, 61: 203-212, (1990), LeRoith et al., Endocrin. Rev., 16: 143-163 (1995); Traxler, Exp. Opin. Ther. Patents, 7: 571-588 (1997); Adams et al., Cell. Mol. Life. Sci., 57: 1050-1063 (2000)), and is involved in childhood growth ((Liu et al., Cell, 75: 59-72 (1993); Abuzzahab et al.; N Engl J Med, 349: 2211-2222 (2003)). Synthetic tyrosine kinase inhibitors (tyrphostins) have been described (Parrizas et al., Endocrinology, 138: 1427-1433 (1997)), including substrate-competitive inhibitors of IGF-1R kinase (Blum et al., Biochemistry, 39: 15705-15712 (2000)).

The cytoplasmic tyrosine kinase proteins are activated by the binding of the ligand to the extracellular domain of the receptor. After ligand binding, phosphorylated receptors recruit and phosphorylate docking proteins, including the insulin receptor substrate-1 protein family (IRS1), IRS-2, Shc, Grb 10, and Gabl (Avruch, Mol. Cell. Biochem., 182: 31-48 (1998); Tartare-Deckert et al., J. Biol. Chem., 270: 23456-23460 (1995); He et al., J. Biol. Chem. 271: 11641-11645 (1996); Dey et al., Mol. Endocrinol., 10: 631-641 (1996)); Peruzzi et al., J. Cancer Res. Clin. Oncol., 125:166-173 (1999); Dey et al., Mol. Endocrinol. 10: 631-641 (1996); Morrione et al., Cancer Res. 56: 3165-3167 (1996); Roth et al., Cold Spring Harbor Symp. Quant. Biol., 53: 537-543 (1988); White, Mol. Cell. Biochem., 182: 3-11 (1998); Laviola et al., J. Clin. Invest., 99: 830-837 (1997); Cheatham et al., Endocrin. Rev., 16: 117-142 (1995); Jackson et al., Oncogene, 20: 7318-7325 (2001); Nagle et al., Mol Cell Biol, 24: 9726-9735 (2004); Zhang et al., Breast Cancer Res. Treat., 83: 161-170 (2004)), leading to the activation of different intracellular mediators. IRS-1 is the predominant signaling molecule activated by IGF-I, insulin, and interleukin-4 in estrogen receptor-positive human breast cancer cells (Jackson et al., J. Biol. Chem. 273: 9994-10003 (1998); Pete et al., Endocrinology, 140: 5478-5487 (1999)). The phosphatase PTP1D (syp) binds to IGF-1R, insulin receptor, and others (Rocchi et al., Endocrinology, 137: 4944-4952 (1996)). mSH2-B and vav are also binders of the IGF-1R (Wang and Riedel, J. Biol. Chem., 273: 3136-3139 (1998)).

The availability of substrates can dictate the final biological effect connected with the activation of IGF-1R. When IRS-1 predominates, the cells tend to proliferate and transform. When Shc dominates, the cells tend to differentiate (Valentinis et al., J. Biol. Chem., 274: 12423-12430 (1999)). The route mainly involved in protection against apoptosis is via phosphatidyl-inositol 3-kinases (PI 3-kinases) (Prisco et al., Horm. Metab. Res., 31: 80-89 (1999)). IGF-1R and IRS-1 can influence cell-cell interactions by modulating interaction between components of adherens junctions, including cadherin and beta-catenin (Playford et al Proc Nat Acad Sci (USA), 97: 12103-12108 (2000); Reiss et al., Oncogene, 19: 2687-2694 (2000)). See also Blakesley et al., In: The IGF System. Humana Press., 143-163 (1999)). Garrett et al., Nature, 394: 395-399 (1998) discloses the crystal structure of the first three domains of IGF-1R.

IGFs activate IGF-1R by triggering autophosphorylation of the receptor on tyrosine residues (Butler et al., Comparative Biochemistry and Physiology, 121:19 (1998)). IGF-I and IGF-II function both as endocrine hormones in the blood, where they are predominantly present in complexes with IGF binding proteins, and as paracrine and autocrine growth factors that are produced locally (Humbel, Eur. J. Biochem., 190, 445-462 (1990); Cohick and Clemmons, Annu. Rev. Physiol. 55: 131-153 (1993)). The domains of IGF-1R critical for its mitogenic, transforming, and anti-apoptotic activities have been identified by mutational analysis. For example, the tyrosine 1251 residue of IGF-1R has been found critical for anti-apoptotic and transformation activities but not for mitogenic activity (O'Connor et al., Mol. Cell. Biol., 17: 427-435 (1997); Miura et al., J. Biol. Chem., 270: 22639-22644 (1995)).

IGF binding proteins (IGFBPs) exert growth-inhibiting effects by, e.g., competitively binding IGFs and preventing their association with IGF-1R. The interactions among IGF-I, IGF-II, IGF-1R, acid-labile subunit (ALS), and IGFBPs affect many physiological and pathological processes such as development, growth, and metabolic regulation. See, e.g., Grimberg et al., J. Cell. Physiol., 183: 1-9 (2000). Six IGF binding proteins (IGFBPs) with specific binding affinities for the IGFs have been identified in serum (Yu and Rohan, J. Natl. Cancer Inst., 92: 1472-89 (2000)). See also U.S. Pat. No. 5,328,891; U.S. Pat. No. 5,258,287; EP 406272B1; and WO 89/09268. Only about 1% of serum IGF-I is present as free ligand; the remainder is associated with IGFBPs (Yu and Rohan, J. Natl. Cancer Inst., 92:1472-89 (2000)). References regarding the actions of IGFBPs, their variants, receptors, and inhibitors, including treating cancer, include US 2004/072776; US 2004/072285; US 2001/0034433; U.S. Pat. No. 5,200,509; U.S. Pat. No. 5,681,818; WO 2000/69454; U.S. Pat. No. 5,840,673; WO 2004/07543; US 2004/0005294; WO 2001/05435; WO 2000/50067; WO 2006/0122141; U.S. Pat. No. 7,071,160; and WO 2000/23469.

IGF-1R is homologous to insulin receptor (1R), having a sequence similarity of 84% in the beta-chain tyrosine-kinase domain and of 48% in the alpha-chain extracellular cysteine-rich domain (Ullrich et al., EMBO, 5: 2503-2512 (1986); Fujita-Yamaguchi et al., J. Biol. Chem., 261: 16727-16731 (1986)). IR is also described, e.g., in Vinten et al., Proc. Natl. Acad. Sci. USA, 88: 249-252 (1991); Belfiore et al., J. Biol. Chem., 277: 39684-39695 (2002); and Dumesic et al., J. Endocrin. Metab., 89: 3561-3566 (2004).

Although IR and IGF-1R similarly activate major signaling pathways, differences exist in recruiting certain docking proteins and intracellular mediators between the receptors (Sasaoka et al., Endocrinology, 137: 4427-34 (1996); Nakae et al., Endocrin. Rev., 22: 818-35 (2001); DuPont and LeRoith, Horm. Res., 55, Suppl. 2, 22-26 (2001); Koval et al., Biochem. J., 330: 923-32 (1998)). Thus, IGF-1R mediates mitogenic, differentiation, and anti-apoptosis effects, while activation of IR mainly involves effects at the metabolic pathways level (Baserga et al., Biochim. Biophys. Acta, 1332: F105-126 (1997); Baserga, Exp. Cell. Res., 253: 1-6 (1999); De Meyts et al., Ann. N.Y. Acad. Sci., 766: 388-401 (1995); Prisco et al., Horm. Metab. Res., 31: 80-89 (1999); Kido et al., J. Clin. Endocrinol. Metab., 86: 972-79 (2001)). Insulin binds with high affinity to IR (100-fold higher than to IGF-1R), while IGFs bind to IGF-1R with 100-fold higher affinity than to IR.

Because of their homology, these receptors can form hybrids containing one IR dimer and one IGF-1R dimer (Pandini et al., Cliff Carte. Res., 5:1935-19 (1999); Soos et al., Biochem. J., 270, 383-390 (1990); Kasuya et al., Biochemistry, 32, 13531-13536 (1993); Seely et al., Endocrinology, 136: 1635-1641 (1995); Bailyes et al., Biochem. J., 327: 209-215 (1997); Federici et al., Mol. Cell. Endocrinol., 129: 121-126 (1997)). While both IR and IGF-1R were over-expressed in all breast cancer samples tested, hybrid receptor content consistently exceeded levels of both homo-receptors by approximately 3-fold (Pandini et al., Clin. Carc. Res. 5: 1935-44 (1999)). Although hybrid receptors are composed of IR and IGF-1R pairs, the hybrids bind selectively to IGFs, with affinity similar to that of IGF-1R, and only weakly bind insulin (Siddle and Soos, The IGF System. Humana Press, pp. 199-225 (1999)). Activation of IGF-1R mostly requires binding to ligand (Kozma and Weber, Mol. Cell. Biol., 10: 3626-3634 (1990)).

In liver, spleen, or placenta, hybrids are more represented than IGF-1R (Bailyes et al., supra). Breast tumoral cells specifically present on their surface IGF-1R, as well as IRs and many hybrids (Sciacca et al., Oncogene, 18: 2471-2479 (1999); Vella et al., Mol. Pathol., 54: 121-124 (2001)). Hybrids may also be overexpressed in thyroid and breast cancers (Belfiore et al., Biochimie (Paris) S1, 403-407 (1999)).

Two splice variants of IR have been reported. IR-B is the predominant IR isoform in normal adult tissues that are targets for the metabolic effects of insulin (Moller et al., Mol. Endocrinol., 3: 1263-1269 (1989); Mosthaf et al., EMBO J., 9: 2409-2413 (1990)). The IR isoform A variant is more often prevalent in cancer cells and fetal tissues (Frasca et al., Mol. Cell. Biol., 19: 3278-3288 (1999); DeChiara et al., Nature, 345: 78-80 (1990); Louvi et al., Dev. Biol., 189: 33-48 (1997); Pandini et al., J. Biol. Chem., 277: 39684-39695 (2002)).

The type II IGF receptor (IGF-IIR or mannose-6-phosphate (MOP) receptor) has high affinity for IGF-II, but lacks tyrosine kinase activity and does not apparently transmit an extracellular signal (Oases et al., Breast Cancer Res. Treat., 47: 269-281 (1998)). Because it results in the degradation of IGF-II, it is considered a sink for IGF-II, and its loss has been demonstrated in human cancer (MacDonald et al., Science, 239: 1134-1137 (1988)). Loss of IGF-IIR in tumor cells can enhance growth potential through release of its antagonistic effect on the binding of IGF-II with the IGF-1R (Byrd et al., J. Biol. Chem., 274: 24408-24416 (1999)).

Most normal tissues express IGF-1R (Werner et al., “The insulin-like growth factor receptor: molecular biology, heterogeneity, and regulation” In: Insulin-like Growth Factors: Molecular and Cellular Aspects, LeRoith (ed.) pp. 18-48 (1991)), which, e.g., promotes neuronal survival, maintains cardiac function, and stimulates bone formation and hematopoiesis (Zumkeller, Leuk. Lymphoma, 43: 487-491 (2002); Rosen, Best Pract Res Clin Endocrinol Metab, 18: 423-435 (2004); Leinninger and Feldman, Endocr Dev, 9: 135-159 (2005); Saetrum Opgaard and Wang, Growth Horm IGF Res, 15: 89-94 (2005); Wang et al., Mol Cancer Ther, 4: 1214-1221 (2005)). Also, disruption of IGF-1R affects survival of the pancreatic beta cells (Withers et al., Nat Genet, 23: 32-40 (1999)). See also LeRoith, Endocrinology, 141: 1287-1288 (2000) and LeRoith, New England J. Med., 336: 633-640 (1997).

IGF-1R has been considered to be quasi-obligatory for cell transformation (Adams et al., supra; Cohen et al., Clin. Cancer Res., 11: 2063-2073 (2005); Baserga, Oncogene, 19: 5574-5581 (2000)), and has been implicated in promoting growth, transformation, and survival of tumor cells (Blakesley et al., J. Endocr., 152: 339-344 (1997); Kaleko et al., Mol. Cell. Biol., 10: 464-473 (1990); Macaulay, supra; Baserga et al., Endocrine, 7: 99-102 (1997)). Several types of tumors are known to express higher than normal levels of IGF-1R (Khandwala et al., Endocrine Reviews, 21: 215-244 (2000); Werner and LeRoith, Adv. Cancer Res., 68: 183-223 (1996); Happerfield et al., J. Pathol., 183: 412-417 (1997); Frier et al., Gut, 44: 704-708 (1999); van Dam et al., J. Clin. Pathol., 47: 914-919 (1994); Xie et al., Cancer Res., 59: 3588-3591 (1999); Bergmann et al., Cancer Res., 55: 2007-2011 (1995)).

IGF-1R over-expression or elevated levels are shown, e.g., in human lung (Quinn et al., J. Biol. Chem., 271: 11477-11483 (1996); Kaiser et al., J. Cancer Res. Clin Oncol., 119: 665-668 (1993); Moody et al., Life Sciences, 52: 1161-1173 (1993); Macauley et al., Cancer Res., 50: 2511-2517 (1990)), ovary (Macaulay, Br. J. Cancer, 65: 311-320 (1990)), cervix (Steller et al., Cancer Res., 56: 1762 (1996)), breast (Ellis et al., Breast Cancer Res. Treat., 52:175 (1998); Cullen et al., Cancer Res., 50: 48-53 (1990); Gooch et al., Breast Cancer Res. Treat., 56:1-10 (1999); Webster et al., Cancer Res., 56: 2781 (1996); Pekonen et al., Cancer Res., 48: 1343 (1998); Peyrat and Bonneterre, Cancer Res., 22: 59-67 (1992); Lee and Yee, Biomed. Pharmacother., 49: 415-421 (1995); Turner et al., Cancer Research, 57: 3079-3083 (1997); Pollak et al., Cancer Lett., 38: 223-230 (1987); Pandini et al., Cancer Res., 5: 1935 (1999); Foekens et al., Cancer Res. 49: 7002-7009 (1989); Cullen et al., Cancer Res., 49: 7002-7009 (1990); Arteaga et al., J. Clin. Invest., 84: 1418-1423 (1989)), myeloma (Ge and Rudikoff, Blood, 96: 2856-2861 (2000)), sarcoma (van Valen et al., J. Cancer Res. Clin. Oncol., 118: 269-275 (1992); Xie et al., Cancer Res., 59: 3588 (1999); Scotlandi et al., Cancer Res., 56: 4570-4574 (1996)), prostate (Nickerson et al., Cancer Res., 61: 6276-6280 (2001); Chan et al., Science, 279:563 (1998); Hellawell et al., Cancer Res., 62: 2942-2950 (2002)), melanoma ((Hellawell et al., Cancer Res., 62: 2942-2950 (2002); All-Ericsson et al., Invest. Opthalmol. Vis. Sci., 43: 1-8 (2002)), and colon and colorectum (Hassan and Macaulay, Ann. Oncol., 13: 349-356 (2002); Weber et al., Cancer, 95: 2086-2095 (2002); Remaole-Bennet et al., J. Clin. Endocrinol. Metab., 75: 609-616 (1992); Guo et al., Gastroenterol., 102: 1101-1108 1992)). See also Goldring et al., Eukar. Gene Express., 1: 319-326 (1991).

Overexpression of human IGF-1R resulted in ligand-dependent anchorage-independent growth of NIH 3T3 or Rat-1 fibroblasts, and inoculation of these cells caused a rapid tumor formation in nude mice (Kaleko et al., Mol. Cell. Biol., 10: 464-473 (1990)). Soluble IGF-1R has been used to induce apoptosis in tumor cells in vivo and inhibit tumorigenesis in an experimental animal system (D'Ambrosio et al., Cancer Res. 56: 4013-4020 (1996)). See also Navarro and Baserga, Endocrinology, 142, 1073-1081 (2001).

Several reviews describe reasons for targeting the IGF system in cancer. See, for example, Pollak et al., Nat Rev Cancer, 4: 505-518 (2004); Yee, British J. Cancer, 94: 465-468 (2006); Bohula et al., Anti-Cancer Drugs, 14: 669-682 (2003); Surmacz, Oncogene, 22: 6589-97 (2003); Bahr and Groner, Growth Hormone and IGF Research 14: 287-295 (2004); Guillemard and Saragovi, Current Cancer Drug Targets, 4: 313-326 (2004); Jerome et al., Seminars in Oncology 31/1 Suppl. 3 (54-63) (2004); Zhang and Yee, Breast Disease, 17: 115-124 (2003); Samani and Brodt, Surgical Oncology Clinics of North America, 10: 289-312 (2001); Nahta et al., Oncologist, 8: 5-17 (2003); Dancey and Chen, Nature Reviews, 5: 649-659 (2006); Jones et al., Endocr. Relat. Cancer, 11:793-814 (2004); Schedin, Nature Reviews, 6: 281-290 (2006); Thome and Lee, Breast Disease, 17: 105-114 (2003); Minchinton and Tannock, Nature Reviews, 6: 583-592 (2006); and Kurmasheva and Houghton, Biochim. Biophys. Acta, 1766: 1-22 (2006).

Epidemiological studies show a correlation of elevated plasma level of IGF-I with increased risk for prostate cancer, colon cancer, lung cancer, and breast cancer, including in humans (Chan et al., Science, 279: 563-566 (1998); Wolk et al., J. Natl. Cancer Inst., 90: 911-915 (1998); Ma et al., J. Natl. Cancer Inst., 91: 620-625 (1999); Yu et al., J. Natl. Cancer Inst., 91: 151-156 (1999); Pollak, Eur. J. Cancer 36:1224-1228 (2000); Wu et al., Cancer Res. 62: 1030-1035 (2002); Wu et al., Clin. Cancer Res., 11: 3065-3074 (2005); Renehan et al., Lancet, 363(9418): 1346-1353 (2004); Hankinson et al., Lancet, 351: 1393-1396 (1998)). Constitutive expression of IGF-I in epidermal basal cells of transgenic mice promotes spontaneous tumor formation (DiGiovanni et al., Cancer Res., 60: 1561-1570 (2000); Bol et al., Oncogene, 14: 1725-1734 (1997)). See also Pravtcheva and Wise, J Exp Zool, 281(I): 43-57 (1998) regarding studies showing that the IGF system can drive tumorigenesis in animal models. IGF-I and IGF-II have been shown in vitro to be potent mitogens for several human tumor cell lines such as lung cancer, breast cancer, colon cancer, osteosarcoma and cervical cancer (Ankrapp and Bevan, Cancer Res., 53: 3399-3404 (1993); Hermanto et al., Cell Growth& Differentiation, 11: 655-664 (2000); Guo et al., J. Am. Coll. Surg., 181: 145-154 (1995); Kappel et al., Cancer Res., 54: 2803-2807 (1994); Steller et al., Cancer Res., 56: 1761-1765 (1996)). Strategies are reported to prevent cancer by lowering plasma IGF-I levels or inhibiting IGF-1R function (e.g., Wu et al., Cancer Res., 62: 1030-1035 (2002); Grimberg and Cohen, J. Cell. Physiol., 183: 1-9 (2000)).

Over-expression of IGF-II in cell lines and tumors occurs with high frequency and may result from loss of genomic imprinting of the IGF-II gene (Yaginuma et al., Oncology, 54: 502-507 (1997)). Epigenetic changes (such as loss of imprinting at the IGF-II locus) frequently occurs in colon and ovarian cancers as well as in several pediatric malignancies (Feinberg, Semin Cancer Biol, 14: 427-432 (2004)). WO 2004/10850 discloses identifying loss of imprinting of the IGF-II gene in a subject by analyzing a biological sample for hypomethylation of a differentially methylated region (DMR) of the H19 gene and/or IGF-II gene.

In addition, metastatic cancer cells possess higher expression of IGF-II and IGF-1R than tumor cells less likely to metastasize (Guerra et al., Int. J. Cancer, 65: 812-820 (1996)). IGF-1R knockout-derived mouse embryo fibroblasts grow at significantly reduced rates in culture medium containing 10% serum and fail to be transformed by many oncogenes (Sell et al., Proc. Natl. Acad. Sci., USA, 90: 11217-11221 (1993); Sell et al., Mol. Cell. Biol., 14: 3604-3612 (1994); Morrione, Virol., 69: 5300-5303 (1995); Coppola et al., Mol. Cell. Biol., 14: 4588-4595 (1994); DeAngelis et al., J. Cell. Physiol., 164: 214-221 (1995)). Resistance to the HER-2 antibody HERCEPTIN® (trastuzumab) in some forms of breast cancer may be caused by activation of IGF-1R signaling (Nahta et al., Cancer Res, 65: 11118-11128 (2005); Lu et al., J. Natl. Cancer Inst. 93: 1852-1857 (2001)).

For reviews of how IGF-M/IGF-1R interaction mediates cell proliferation and plays a role in the growth of a variety of human tumors, see, e.g., Goldring et al., Eukar. Gene Express., 1:31-326 (1991) and Werner and LeRoith, Adv. Cancer Res. 68: 183-223 (1996). IGF-1R mechanisms and signaling are described, for example, in Datta et al., Genes and Development, 13: 2905-2927 (1999); Kulik et al., Mol. Cell. Biol. 17: 1595-1606 (1997); Dufourny et al., J. Biol. Chem., 272: 31163-31171 (1997); and Parrizas et al., J. Biol. Chem., 272: 154-161 (1997). See also Baserga, Expert Opin Ther Targets, 9: 753-768 (2005)).

Enhanced tyrosine phosphorylation of IGF-1R has been detected in human medulloblastoma (Del Valle et al., Clin. Cancer Res., 8: 1822-1830 (2002)) and in human breast cancer (Resnik et al., Cancer Res., 58: 1159-1164 (1998)). Deregulated expression of IGF-I in prostate epithelium leads to neoplasia in transgenic mice (DiGiovanni et al., Proc. Natl. Acad. Sci. USA, 97: 3455-3460 (2000)). Also, IGF-I appears to be an autocrine stimulator of human gliomas (Sandberg-Nordqvist et al., Cancer Res., 53: 2475-2478 (1993)), while IGF-I stimulated the growth of fibrosarcomas that overexpressed IGF-1R (Butler et al., Cancer Res., 58: 3021-3027 (1998)). Individuals with “high-normal” levels of IGF-I have an increased risk of common cancers compared to individuals with IGF-I levels in the “low-normal” range (Rosen et al., Trends Endocrinol. Metab., 10: 136-41 (1999)). Many of these tumor cell types respond to IGF-I with a proliferative signal in culture (Nakanishi et al., J. Clin. Invest., 82: 354-359 (1988); Freed et al., J. Mol. Endocrinol., 3: 509-514 (1989)), and autocrine or paracrine loops for proliferation in vivo have been suggested (Yee et al., Mol. Endocrinol., 3: 509-514 (1989); Yu and Rohan, J. Natl. Cancer Inst., 92: 1472-1489 (2000)).

IGF-1R activation can retard programmed cell death (Harrington et al., EMBO J., 13: 3286-3295 (1994); Sell et al., Cancer Res., 55: 303-305 (1995); Rodriguez-Tarduchy et al., J. Immunol., 149: 535-540 (1992); Singleton et al., Cancer Res., 56: 4522-4529 (1996)). Activated IGF-1R signals PI3K and downstream phosphorylation of Akt, or protein kinase B. Akt can block via phosphorylation molecules such as BAD that are essential for initiating programmed cell death and inhibit initiation of apoptosis (Datta et al., Cell, 91: 231-241 (1997)). The anti-apoptotic effect induced by the IGF-I/IGF-1R system correlates to chemo-resistance induction in various tumors (Grothey et al., J. Cancer Res. Clin. Oncol., 125: 166-173 (1999)).

Activation of IGF signaling can promote the formation of spontaneous tumors in a mouse transgenic model (DiGiovanni et al., Cancer Res., 60: 1561-1570 (2000)). IGF over-expression can rescue cells from chemotherapy-induced cell death and may be important in tumor cell drug resistance (Gooch et al., Breast Cancer Res. Treat., 56: 1-10 (1999)). Hence, modulation of the IGF signaling pathway has increased tumor cell sensitivity to chemotherapeutic agents (Benin et al., Clinical Cancer Res., 7: 1790-1797 (2001)).

A decrease in the level of IGF-1R below wild-type levels was also shown to cause massive apoptosis of tumor cells in vivo, using, e.g., anti-sense inhibition (Resnicoff et al., Cancer Res., 54: 2218-2222 (1994); Resnicoff et al., Cancer Res., 54: 4848-4850 (1994); Liu et al., Cancer Res., 58, 5432-5438 (1998); Chemicky et al., Cancer Gene Therapy, 7: 384-395 (2000), Sun et al., Cell research (China), 11: 107-115 (2001); Resnicoff et al., Cancer Res., 55: 2463-2469 (1995); Lee et al., Cancer Res., 56: 3038-3041 (1996); Muller et al., Int. J. Cancer, 77: 567-571 (1998); Shapiro et al., J. Clin. Invest., 94: 1235-1242 (1994); Resnicoff et al., Cancer Res., 55: 3739-3741 (1995); Trojan et al., Science, 259: 94-97 (1993); Kalebic et al., Cancer Res., 54: 5531-5534 (1994); Prager et al., Proc. Natl. Acad, Sci. USA, 91: 2181-2185 (1994); Burfeind et al., Proc. Natl. Acad. Sci. USA, 93: 7263-7268 (1996); Wraight et al., Nat. Biotech., 18: 521-526 (2000); Baserga, Cancer Res., 55: 249-252 (1995); and U.S. Pat. No. 6,340,674). Using the yeast two-hybrid system it was shown that p85, the regulatory domain of phosphatidyl inositol 3 kinase (PI3K), interacts with IGF-1R (Lamothe et al., FEBS Lett., 373: 51-55 (1995); Tartare-Decker et al., Endocrinology, 137: 1019-1024 (1996)). Another binding partner of IGF-1R, SHC, binds to other tyrosine kinases such as Trk, Met, EGF-R, and IR (Tartare-Deckert et al., J. Biol. Chem., 270: 23456-23460 (1995)). Downregulation of IGF-1R in mouse melanoma cells led to enhancement of radiosensitivity, reduced radiation-induced p53 accumulation and serine phosphorylation, and radioresistant DNA synthesis (Macaulay et al., Oncogene, 20: 4029-4040 (2001)). See also Wraight et al. (Nature Biotechnology, 18: 521-526 (2000)), showing reversal of epidermal hyperplasia in a mouse model of psoriasis using IGF-1R anti-sense oligonucleotides. Transgenic mice overexpressing IGF-II specifically in the mammary gland develop mammary adenocarcinoma (Bates et al., Br. J. Cancer, 72: 1189-1193 (1995)), and transgenic mice overexpressing IGF-II under the control of a more general promoter develop more tumor types (Rogler et al., J. Biol. Chem., 269: 13779-13784 (1994)). At physiologic concentrations of insulin, breast cancer cells are stimulated to proliferate in vitro (Osborne et al., Proc Natl Acad Sci USA, 73: 4536-4540 (1976)). Activation of IR-A by IGF-II has been shown in breast cancer cell lines (Sciacca et al., supra). Hence, inhibition of both IGF-1R and IR may be required for optimal suppression of IGF signaling pathways.

Clinical trials testing monoclonal antibodies directed against IGF-1R are ongoing to determine the therapeutic window for long- or short-term inhibition of IGF-1R.

Activation of the IGF system has been implicated in several pathologies besides cancer, including acromegaly and gigantism (Drange and Melmed. In: The IGF System. Humana Press., 699-720 (1999); Barkan, Cleveland Clin. J. Med., 65:343:347-349 (1998); Ben-Schlomo et al., Endocrin. Metab. Clin. North. Am., 30: 565-583 (2001)), atherosclerosis and smooth muscle restenosis of blood vessels following angioplasty (Bayes-Genis et al., Circ. Res., 86: 125-130 (2000)), diabetes or complications thereof, such as microvascular proliferation and retinal neovascularization (Smith et al., Nature Med., 12: 1390-95 (1999)), and psoriasis (Wraight et al., Nature Biotech., 18: 521-526 (2000)). Decreased IGF-I levels are associated with, e.g., small stature (Laron, Paediatr. Drugs, 1: 155-159 (1999)), neuropathy, decrease in muscle mass, and osteoporosis (Rosen et al., Trends Endocrinol. Metab., 10: 136-141 (1999)).

Calorie restriction has been reported to increase life span in a number of animal species, including mammals, and is additionally the most potent broadly acting cancer-prevention regimen in experimental carcinogenesis models. A key biological mechanism underlying many of its beneficial effects is the IGF-I pathway (Hursting et al., Annu. Rev. Med., 54:131-152 (2003). US 2006/0078533 discloses a method for prevention and treatment of aging and age-related disorders, including atherosclerosis, peripheral vascular disease, coronary artery disease, osteoporosis, type 2 diabetes, dementia, and some forms of arthritis and cancer in a subject using an effective dosage of, e.g., tyrosine kinase inhibitors/antibodies. EP 1808070 (Institute Pasteur) discloses a non-human animal as an experimental model for neurodegenerative diseases with an alteration in the biological activity of the IGF-1R found in the epithelial cells in the choroid plexus of the cerebral ventricles.

Using anti-sense and nucleic acids to antagonize IGF-1R is described, e.g., in Wraight et al., Nat. Biotech., 18: 521-526 (2000); U.S. Pat. No. 5,643,788; U.S. Pat. No. 6,340,674; US 2003/0031658; U.S. Pat. No. 6,340,674; U.S. Pat. No. 5,456,612; U.S. Pat. No. 5,643,788; U.S. Pat. No. 6,071,891; WO 2002/101002; CN 1237582A; CN 1117097B; WO 1999/23259; WO 2003/100059; US 2004/127446; US 2004/142895; US 2004/110296; US 2004/006035; US 2003/206887; US 2003/190635; US 2003/170891; US 2003/096769; U.S. Pat. No. 5,929,040; U.S. Pat. No. 6,284,741; US 2006/0234239; and U.S. Pat. No. 5,872,241.

Further, US 2005/0255493 discloses reducing IGF-1R expression by RNA interference using short double-stranded RNA.

In addition, inhibitory peptides targeting IGF-1R have been generated that possess anti-proliferative activity in vitro and in vivo (Pietrzkowski et al., Cancer Res., 52:6447-6451 (1992); Haylor et al., J. Am. Soc. Nephrol., 11:2027-2035 (2000)). Growth can also be inhibited using peptide analogues of IGF-I (Pietrzkowski et al., Cell Growth &Diff., 3: 199-205 (1992); Pietrzkowski et al., Mol. Cell. Biol., 12: 3883-3889 (1992)). In addition, dominant-negative mutants of IGF-1R (Li et al., J. Biol. Chem., 269: 32558-32564 (1994); Jiang et al., Oncogene, 18: 6071-6077 (1999); Scotlandi et al., Int. J. Cancer, 101: 11-16 (2002); Seely et al., BMC Cancer, 2: 15 (2002)) can reverse the transformed phenotype, inhibit tumorigenesis, and induce loss of the metastatic phenotype. A C-terminal peptide of IGF-1R has been shown to induce apoptosis and significantly inhibit tumor growth (Reiss et al., J. Cell. Phys., 181:124-135 (1999)). Also, a soluble form of IGF-1R inhibits tumor growth in vivo (D'Ambrosio et al., Cancer Res., 56: 4013-4020 (1996)).

Additional peptides that antagonize IGF-1R or treat cancer involving IGF-I include those described by U.S. Pat. No. 6,084,085; U.S. Pat. No. 5,942,489; WO 2001/72771; WO 2001/72119; US 2004/0086863; U.S. Pat. No. 5,633,263; and US 2003/0092631. See also U.S. Pat. No. 7,173,005 on peptide sequences capable of binding to insulin and/or IGF receptors with either agonist or antagonist activity. Moreover, the company Allostera is developing IGF-1R-directed peptides (Bioworld Today published May 19, 2006 (Vol. 17, page 1).

U.S. Pat. No. 7,020,563 discloses a method of designing agonists and antagonists to IGF-1R, by identifying compounds that modulate binding of a ligand to IGF-1R. This method comprises designing or screening for a compound that binds to the structure formed by amino acids having certain atomic coordinates, where binding of the compound to the structure is favored energetically, and testing the compound designed or screened for its ability to modulate binding of the ligand to IGF-1R in vivo or in vitro. U.S. Pat. No. 7,020,563 and EP 1,034,188 disclose identifying agonist and antagonist candidates to IGF-1R using its molecular structure. Selection of anti-cancer candidate compounds involving IGF-I or IGF-1R is described, e.g., in US 2004/0142381; US 2004/0121407; US 2003/0182668; U.S. Pat. No. 6,699,658 and U.S. Pat. No. 6,331,391.

Modified IGF-1R or IGF molecules are described, e.g., in WO 2003/80101; US 2004/0116335; U.S. Pat. No. 6,358,916; U.S. Pat. No. 6,610,302; U.S. Pat. No. 6,084,085; U.S. Pat. No. 5,942,412; U.S. Pat. No. 5,470,829; WO 2000/20023; U.S. Pat. No. 6,015,786; U.S. Pat. No. 6,025,332; U.S. Pat. No. 6,025,368; U.S. Pat. No. 6,514,937; U.S. Pat. No. 6,518,238; WO 2000/53219; and JP 5199878. Further, US 2006/0040358 and U.S. Pat. No. 6,913,883 report IGF-1R-interacting proteins.

Combination therapies involving IGF-1R inhibitors or IGF-I are described, e.g., in US 2004/0072760; US 2004/209930; WO 2004/030627; US 2004/0106605; WO 1993/21939; U.S. Pat. No. 5,731,325; US 2005/043233; US 2005/075358; WO 2005/041865; and U.S. Pat. No. 6,140,346. US 2006/0258569 discloses a method of treating cancer involving administering an IGF-1R agonist and a chemotherapeutic agent, as well as compounds for treating cancer comprising an IGF-1R ligand or IR ligand coupled to a chemotherapeutic agent. Additionally, EP 1,671,647 discloses a medicament for treating cancer in which a cancer therapeutic effect is synergistically increased using a substance inhibiting activities of IGF-I and IGF-II. IGF-1R inhibitors are useful to treat cancer (e.g., US 2004/0044203), as either single agents or with other anti-cancer agents (Burtrum et al., Cancer Research, 63: 8912-8921 (2003)). Also, US 2006/0193772 describes inhibitors of IGF-I and IGF-II to treat cancer.

Cancer vaccines involving IGF-I are described, e.g., in U.S. Pat. No. 5,919,459; EP 702563B1; WO 1994/27635; EP 1284144A1; WO 2003/015813; U.S. Pat. No. 6,420,172; EP 637201A4; and WO 1993/20691.

Small-molecule inhibitors to IGF-1R are described, e.g., in Garcia-Echeverria et al., Cancer Cell, 5: 231-239 (2004); Mitsiades et al., Cancer Cell, 5: 221-230 (2004); and Carboni et al., Cancer Res, 65: 3781-3787 (2005). Further, compounds have been developed that disrupt receptor activation, such as, for example, Vasilcanu et al., Oncogene, 23: 7854-7862 (2004), which describes a cyclolignan, picropodophyllin, which appears to be specific for IGF-1R (Gimita et al., Cancer Res, 64: 236-242 (2004); Stromberg et al., Blood, 107: 669-678 (2006)). Nordihydroguaiaretic acid (NDGA) also disrupts IGF-1R function (Youngren et al., Breast Cancer Res Treat, 94: 37-46 (2005)). Further examples of disclosures on such small-molecule inhibitors include WO 2002/102804; WO 2002/102805; WO 2004/55022; U.S. Pat. No. 6,037,332; WO 2003/48133; US 2004/053931; US 2003/125370; U.S. Pat. No. 6,599,902; U.S. Pat. No. 6,117,880; WO 2003/35619; WO 2003/35614; WO 2003/35616; WO 2003/35615; WO 1998/48831; U.S. Pat. No. 6,337,338; US 2003/0064482; U.S. Pat. No. 6,475,486; U.S. Pat. No. 6,610,299; U.S. Pat. No. 5,561,119; WO 2006/080450; WO 2006/094600; and WO 2004/093781 See also WO 2007/099171 (bicyclo-pyrazole inhibitors) and WO 2007/099166 (pyrazolo-pyridine derivative inhibitors). See also (Hubbard et al., AACR-NCI-EORTC Int Conf Mol Targets Cancer Ther (October 22-26, San Francisco) 2007, Abst A227) on Abbott Corporation's molecule A-928605.

Diagnostics involving IGF or IGF-1R are described in, e.g., US 2003/0044860; U.S. Pat. No. 6,410,335; US 2001/0018190 U.S. Pat. No. 6,645,770; U.S. Pat. No. 6,410,335; U.S. Pat. No. 6,448,086; WO 2001/53837; WO 2004/65583; WO 2001/25790; and WO 2002/31500. WO 2006/060419 and US 2006/0140960 disclose certain biomarkers for pre-selection of patients for anti-IGF-1R therapy. US 2007/190583 reports use of various biomarkers for cancer (including TGF-α, pS6, and IGF-1R) to assess a subject's suitability for treatment with an EGFR/ErbB2 kinase inhibitor such as lapatinib. U.S. Pat. No. 5,442,043 describes detecting IGF-1R on tumors.

WO 2002/17951 describes treatment of brain cancer with an IGF-I structural analog such as des-IGF; US 2003/0017146; U.S. Pat. No. 5,851,985; and U.S. Pat. No. 6,261,557 describe treatment of amino-acid deprived cancer patients with IGF-I optionally with arginine-decomposing enzyme; WO 1993/09816 describes a conjugate of IGF-I and radionucleotide to treat cancer; and WO 200413177 discloses use of mannose-6-phosphate/insulin-like growth factor-2 receptor (CD222) as regulator of urokinase plasminogen activator functions, useful for treating arteriosclerosis, restenosis, autoimmunity, inflammation and cancer.

Several antibodies, small molecules, and anti-sense molecules against IGF-1R have shown promise in mouse tumor models with little or no toxicity (Garber et al., J. Natl. Cancer Inst., 97: 790-92 (2005). Gualberto et al., “Inhibition of the insulin like growth factor 1 receptor by a specific monoclonal antibody in multiple myeloma”, J. Clin. Oncology, 41st Annual Meeting of the American-Society-of-Clinical-Oncology (May 13-17, 2005, Orlando, Fla. (published Jun. 1, 2005, vol. 23 (16): 1 Supp 203S, states that a biomarker assay was generated to support the clinical development of the anti-IGF-1R antibody CP-751,871. Flow cytometry of granulocytes was found to be a reliable biomarker of the activity of this antibody, and may contribute to define a therapeutic dose and regimen. Further, this antibody was found to effectively downregulate IGF-1R expression on peripheral blood leucocytes (PBLs).

Because small-molecule inhibitors of the IGF-1R kinase, however, often cross-inhibit the insulin receptor, antibody-based approaches afford better selectivity toward IGF-1R. In addition, unlike small-molecule agents, antibodies are not likely to cross the blood-brain barrier (Rubenstein et al., Blood, 101(2): 466-268 (2003)), reducing the risk of possible interference with the central nervous system. This is particularly relevant to cognitive function, because IGF-I has been suggested to be required for optimal performance of memory and learning throughout life (Sonntag et al., Ageing Res Rev, 4: 195-212 (2005)).

Antibodies to various growth-factor receptors and their ligands are known. For example, antibodies to EGF receptor are reported, e.g., in U.S. Pat. No. 5,891,996 and U.S. Pat. No. 7,060,808. Antibodies to IGF are described, e.g., in EP 1,505,075; EP 656,908B1; US 2006/0240015; WO 1994/04569; US 2006/0165695; EP 1,676,862; and EP 1,671,647. See also Feng et al., “Novel human monoclonal antibodies to insulin-like growth factor (IGF)-II that potently inhibit the IGF receptor type I signal transduction function,” Mol Cancer Ther., 5 (1): 114-120 (2006) and US 2007196376 on antibodies to IGF-II.

Antibodies to IGF-1R, e.g., a mouse IgG1 monoclonal antibody designated αIR3 (Kull et al., J. Biol. Chem., 258:6561-6566 (1983); Arteaga and Osborne, Cancer Research, 49:6237-6241 (1989)), inhibit proliferation of many tumor cell lines (Arteaga et al., Breast Cancer Res. Treat., 22:101-106 (1992); Rohlik et al., Biochem. Biophys. Res. Commun., 149: 276-281 (1987); Arteaga et al., J. Clin. Invest., 84:1418-1423 (1989)). αIR3 is commonly used for IGF-1R studies in vitro, and exhibits agonistic activity in transfected 3T3 and CHO cells expressing human IGF-1R (Kato et al., J. Biol. Chem., 268:2655-2661 (1993); Steele-Perkins and Roth, Biochem. Biophys. Res. Commun., 171:1244-1251 (1990)). The binding epitope of αIR3 is inferred from chimeric insulin-IGF-I receptor constructs to be the 223-274 region of IGF-1R (Gustafson and Rutter, J. Biol. Chem., 265:18663-18667 (1990)). In MCF-7 human breast cancer cells (Dufourny et al., J. Biol. Chem., 272:31163-31171 (1997)), αIR3 incompletely blocks the stimulatory effect of exogenously added IGF-I and IGF-II in serum-free conditions by approximately 80%. Also, αIR3 does not significantly inhibit (less than 25%) the growth of MCF-7 cells in 10% serum (Cullen et al., Cancer Res., 50:48-53 (1990)).

Additional mouse monoclonal antibodies that inhibit IGF-1R activity include 1H7 (Li et al., Biochem. Biophys. Res. Comm., 196: 92-98 (1993); Xiong et al., Proc. Natl. Acad. Sci., U.S.A., 89: 5356-5360 (1992)) and MAB391 (R&D Systems; Minneapolis, Minn.). See also Zia et al., J. Cell. Biol., 24:269-275 (1996) regarding mouse monoclonal antibodies. Further, single-chain antibodies against IGF-1R have been shown to inhibit growth of MCF-7 cells in xenografts models (Li et al., Cancer Immunol. Immunother., 49: 243-252 (2000)) and to lead to down-regulation of cell-surface receptors (Sachdev et al., Cancer Res, 63: 627-635 (2003)).

Antibodies directed against human IGF-1R have also been shown to inhibit tumor-cell proliferation in vitro and tumorigenesis in vivo including cell lines derived from Ewing's osteosarcoma (Scotlandi et al., Cancer Res., 58:4127-4131 (1998)) and melanoma (Furlanetto et al., Cancer Res., 53:2522-2526 (1993)). See also Park and Smolen. In: Advances in Protein Chemistry. Academic Press. pp:360-421 (2001); Thompson et al., Pediat. Res., 32: 455-459 (1988); Tappy et al., Diabetes, 37: 1708-1714 (1988); Weightman et al., Autoimmunity, 16:251-257 (1993); and Drexhage et al., Nether. J. of Med., 45:285-293 (1994).

Other publications on IGF-1R antibodies and their anti-tumor effects include, e.g., Benini et al., Clin. Cancer Res., 7: 1790-1797 (2001); Scotlandi et al., Cancer Gene Ther., 9: 296-307 (2002); Scotlandi et al., Int. J. Cancer, 101: 11-16 (2002); Brunetti et al., Biochem. Biophys. Res. Commun., 165: 212-218 (1989); Prigent et al., J. Biol. Chem., 265: 9970-9977 (1990); Pessino et al., Biochem. Biophys. Res. Commun., 162: 1236-1243 (1989); Surinya et al., J. Biol. Chem., 277: 16718-16725 (2002); Soos et al., J. Biol. Chem., 267: 12955-12963 (1992); Soos et al., Proc. Natl. Acad. Sci. USA, 86: 5217-5221 (1989); O'Brien et al., EMBO J., 6: 4003-4010 (1987); Taylor et al., Biochem. J., 242: 123-129 (1987); Soos et al., Biochem. J., 235: 199-208 (1986); Li et al., Biochem. Biophys. Res. Commun., 196: 92-98 (1993); Delafontaine et al., J. Mol. Cell. Cardiol., 26: 1659-1673 (1994); Morgan and Roth, Biochemistry, 25: 1364-1371 (1986); Forsayeth et al., Proc. Natl. Acad. Sci. USA, 84: 3448-3451 (1987); Schaefer et al., J. Biol. Chem., 265: 13248-13253 (1990); Hoyne et al., FEBS Lett., 469: 57-60 (2000); Tulloch et al., J. Struct. Biol., 125: 11-18 (1999); Dricu et al., Glycobiology, 9: 571-579 (1999); Kanter-Lewensohn et al., Melanoma Res., 8: 389-397 (1998); Hailey et al., Mol. Cancer. Ther., 1: 1349-1353 (2002); Maloney et al., Cancer Res, 63: 5073-5083 (2003); Goetsch et al., Int J Cancer, 113: 316-328 (2005); and Wang et al., supra). The monoclonal antibody binding sometimes results in endosomal degradation of the receptor (Sachdev et al., supra; Wang et al., supra).

Antibodies, nanobodies, and antibody-like molecules targeting growth factor receptors and receptor protein tyrosine kinases, including IGF-1R, and their various uses, including treating cancer, are described also in, e.g., US 2001/0005747; U.S. Pat. No. 5,833,985; EP 749325B1; WO 1995/24220; WO 2002/053596; WO 2004/083248; WO 2005/005635; US 2003/0165502; US 2002/0009739; US 2003/0158109; WO 2000/022130; WO 2007/000328; US 2003/0235582; US 2004/0265307; US 2005/186203; WO 2005/061541; US 2006/0233810; WO 2006/113483; US 2005/0249728; US 2004/0018191; US 2007/0059241; US 2007/0059305 U.S. Pat. No. 7,037,498; US 2005/244408; US 2005/281812; US 2004/0116330; US 2004/0202651; US 2004/0202655; US 2004/0228859; US 2005/0008642; US 2005/0069539; WO 2005/016967; US 2005/0084906; U.S. Pat. No. 7,241,444; WO 2007/092453; WO 2007/115814; WO 2007/115813; US 2007/0248600; US 2007/0243194; US 2005/0249730; WO 2003/59951; WO 2005/058967; WO 2002/05359; WO 2003/100008; WO 2003/106621; WO 2006/013472; US 2005/0136063; US 2005/048050; WO 2002/102973; WO 2002/102972; WO 2002/102854; WO 2004/87756; WO 2005/016967; U.S. Pat. No. 7,217,796; WO 2005/016970; WO 2005/082415; US 2006/0018910; US 2005/0281814; WO 2006/069202; WO 2007/00328; WO 2007/042289; WO 2007/093008; U.S. Pat. No. 6,524,832; WO 2007/012614; and US 2007/0099847. US 2004/0213792 discloses inhibiting cellular activation by IGF-I by administering an antagonist inhibiting binding of IAP to SHPS-1). WO 2007/095337 discloses an antibody-buffer formulation, including antibodies to receptors, and WO 2007/110339 discloses a formulation of IGF-1R monoclonal antibodies. There is a continuing need in the art to produce antibodies having improved function that bind and block IGF-1R, since it is associated with various types of cancer and other human diseases. There is also a special need for antibodies that will recognize IGF-1R specifically and with great affinity and minimal or no interaction with IR.

### SUMMARY OF THE INVENTION

The invention is in part based on the identification of a variety of antagonists of the IGF-1R biological pathway, which is a biological/cellular process presenting as an important therapeutic target. The invention provides compositions and methods based on interfering with IGF-1R activation, including but not limited to interfering with IGFs binding to IGF-1R.

Accordingly, the invention is as claimed. In one aspect, the invention provides an isolated anti-IGF-1R antibody (preferably human anti-IGF-1R antibody) comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid (10H5.vX or 9F2.vX or 2B4.vX or 10H5.v2 or 10H5.v48 or YW95.6, respectively);
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10) (10H5.vX or 9F2.vX; 2B4.vX, or 10H5.v10 or YW95.6, respectively);
(c) a HVR-L3 sequence comprising amino acids C1-C9, wherein C1-C9 is HQYNNYPYT (SEQ ID NO: 11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid (10H5.vX or 9F2.vX/2B4.vX or 10H5.v10 or YW95.81 or YW95.6, respectively);
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid (10H5.vX or 9F2.vX or 2B4.vX or YW95.6 or YW95.87, respectively);
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25) (10H5.vX or 9F2.vX or 2B4.vX or YW95.6 or YW95.81, respectively); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32) (10H5.vX or 9F2.vX or 2B4.vX or YW95.87 or YW95.3 or YW95.6 or YW95.81, respectively).

In a preferred embodiment, SEQ ID NO: 13 is QQYSNYPYT (SEQ ID NO:33), QQYKHYPYT (SEQ ID NO:34), QQYKKYPYT (SEQ ID NO:35), QQYKNYPYT (SEQ ID NO:36), QQYRIYPYT (SEQ ID NO:37), QQYKRYPYT (SEQ ID NO:38), QQYKSYPYT (SEQ ID NO:39), QQYRSYPYT (SEQ ID NO:40), or QQYSKYPYT (SEQ ID NO:41) (10H5.v2, 10H5.v9, 10H5.v16, 10H5.v32, 10H5.v39, 10H5.v46, 10H5.v48, 10H5.v96A, or 10H5.v96B, respectively).

In another preferred embodiment, the HVR-H3 is SEQ ID NO:26.

In another preferred aspect, the antibody comprises either (i) all of the HVR-L1 to HVR-L3 amino acid sequences of SEQ ID NOS:1, 7, and 11, or of SEQ ID NOS:2, 8, and 12, or of SEQ ID NOS:3, 8, and 12 or of SEQ ID NOS:6, 10, and 15, or of SEQ ID NOS:4, 7, and 33, or of SEQ ID NOS:1, 7, and 34, or of SEQ ID NOS:1, 9, and 13, or of SEQ ID NOS:1, 7, and 35, or of SEQ ID NOS:1, 7, and 36, or of SEQ ID NOS:1, 7, and 37, or of SEQ ID NOS:1, 7, and 38, or of SEQ ID NOS:5, 7, and 39, or of SEQ ID NOS:1, 7, and 40, or of SEQ ID NOS:1, 7, and 41; or (ii) all of the HVR-H1 to HVR-H3 amino acid sequences of SEQ ID NOS:16, 21, and 26 or of SEQ ID NOS:17, 22, and 27, or of SEQ ID NOS:18, 23, and 28, or of SEQ ID NOS:19, 24, and 31.

In a more preferred aspect, the antibody comprises all of SEQ ID NOS:1, 7, and 11 or all of SEQ ID NOS:16, 21, and 26.

In other still more preferred embodiments, the antibody comprises (i) all of the HVR-L1 to HVR-L3 amino acid sequences of SEQ ID NOS:1, 7, and 11, or of SEQ ID NOS:2, 8, and 12, or of SEQ ID NOS:3, 8, and 12 or of SEQ ID NOS:6, 10, and 15, or of SEQ ID NOS:4, 7, and 33, or of SEQ ID NOS:1, 7, and 34, or of SEQ ID NOS:1, 9, and 13, or of SEQ ID NOS:1, 7, and 35, or of SEQ ID NOS:1, 7, and 36, or of SEQ ID NOS:1, 7, and 37, or of SEQ ID NOS:1, 7, and 38, or of SEQ ID NOS:5, 7, and 39, or of SEQ ID NOS:1, 7, and 40, or of SEQ ID NOS:1, 7, and 41; and (ii) all of the HVR-H1 to HVR-H3 amino acid sequences of SEQ ID NOS:16, 21, and 26 or of SEQ ID NOS:17, 22, and 27, or of SEQ ID NOS:18, 23, and 28, or of SEQ ID NOS:19, 24, and 31.

Still more preferably, the antibody comprises (i) all of SEQ ID NOS:1, 7, and 11, or all of SEQ ID NOS:4, 7, and 33, or all of SEQ ID NOS:1, 7, and 34, or all of SEQ ID NOS:1, 9, and 13, or all of SEQ ID NOS:1, 7, and 35, or all of SEQ ID NOS:1, 7, and 36, or all of SEQ ID NOS:1, 7, and 37, or all of SEQ ID NOS:1, 7, and 38, or all of SEQ ID NOS:5, 7, and 39, or all of SEQ ID NOS:1, 7, and 40, or all of SEQ ID NOS:1, 7, and 41, and (ii) all of SEQ ID NOS:16, 21, and 26.

Most preferably, the antibody comprises all of SEQ ID NOS:1, 7, and 11 and all of SEQ ID NOS:16, 21, and 26.

In addition, the antibodies herein are preferably chimeric or humanized, most preferably humanized. A humanized antibody of the invention may comprise one or more suitable human and/or human consensus non-HVR (e.g., framework) sequences in its heavy- and/or light-chain variable domains, provided the antibody exhibits the desired biological characteristics (e.g., a desired binding affinity). Preferably, at least a portion of such humanized antibody framework sequence is a human consensus framework sequence.

In some embodiments, one or more additional modifications are present within the human and/or human consensus non-HVR sequences. In one embodiment, the heavy-chain variable domain of an antibody of the invention comprises at least a portion of (preferably all of) a human consensus framework sequence, which in one embodiment is the subgroup III consensus framework sequence. In one embodiment, an antibody of the invention comprises at least a portion of (preferably all of) a variant subgroup III consensus framework sequence modified at least one amino acid position. For example, in one embodiment, a variant subgroup III consensus framework sequence may comprise a substitution at one or more of positions 71, 73, or 78. In one embodiment, said substitution is R71A, N73T, or N78A, in any combination thereof, preferably all three. In another embodiment, these antibodies comprise or further comprise at least a portion of (preferably all of) a human κ subgroup I light-chain consensus framework sequence. In a preferred embodiment, an antibody of the invention comprises at least a portion of (preferably all of) a human κ subgroup I framework consensus sequence.

The amino acid position/boundary delineating a HVR of an antibody can vary, depending on the context and the various definitions known in the art (as described below). Some positions within a variable domain may be viewed as hybrid hypervariable positions in that these positions can be deemed to be within a HVR under one set of criteria while being deemed to be outside a HVR under a different set of criteria. One or more of these positions can also be found in extended HVRs (as further defined below). The invention provides antibodies comprising modifications in these hybrid hypervariable positions. In one embodiment, these hybrid hypervariable positions include one or more of positions 26-30, 33-35B, 47-49, 57-65, 93, 94, and 102 in a heavy-chain variable domain. In one embodiment, these hybrid hypervariable positions include one or more of positions 24-29, 35-36, 46-49, 56, and 97 in a light-chain variable domain. In one embodiment, an antibody of the invention comprises a variant human subgroup consensus framework sequence modified at one or more hybrid hypervariable positions.

In another preferred aspect, the antibody specifically binds to human IGF-1R and blocks the interaction of an insulin-like growth factor (IGF) with IGF-1R, wherein said antibody is an antagonist of human IGF-1R and has an Fc region. Preferably, the IGF is IGF-I. Also, in a preferred embodiment, the antibody does not bind specifically to (or does not cross-react with) the human insulin receptor.

In other aspects, the anti-IGF-1R antibody substantially neutralizes at least one activity of IGF-1R, that is, it blocks at least one biological activity of IGF-1R by at least about 50%, more preferably by at least about 70%, more preferably still by at least about 80%, even more preferably by at least about 90%, and most preferably by at least about 95%.

In another preferred aspect, the antibody is an antibody fragment.

In still further embodiments, the sequence of the light-chain variable region of the antibody herein has about 1-10 amino acid insertions, deletions, or substitutions from SEQ ID NO:53. Preferably, the sequence of its light-chain variable region comprises no more than about eight amino acid changes from SEQ ID NO:53.

In other aspects, the sequence of the heavy-chain variable region of the antibody herein has about 1-10 amino acid insertions, deletions, or substitutions from SEQ ID NO:55. Preferably, the sequence of its heavy-chain variable region comprises no more than about eight amino acid changes from SEQ ID NO:55.

The preferred antibody binds IGF-1R with an affinity of at least about 10⁻¹² M (picomolar levels), and more preferably at least about 10⁻¹³ M. Also preferred is an IgG antibody, more preferably human IgG. Human IgG encompasses any of the human IgG isotypes of IgG1, IgG2, IgG3, and IgG4. Murine IgG encompasses the isotypes of IgG1, 2a, 2b, and 3. More preferably, the murine IgG is IgG2a and the human IgG is IgG1. In other preferred embodiments of the human IgG, the VH and VL sequences provided are joined to human IgG1 constant region.

In another embodiment, the invention provides an anti-IGF-1R antibody having a light-chain variable domain comprising SEQ ID NO:44, 49, 53, 57, 64, 65, 66, 67, 68, 69, 70, 71, 72, or 73, or a heavy-chain variable domain comprising SEQ ID NO:47, 51, 55, or 61, or having light-chain and heavy-chain variable domains comprising both SEQ ID NOS:44 and 47, or both SEQ ID NOS:49 and 51, or both SEQ ID NOS:53 and 55, or both SEQ ID NOS:57 and 61, or both SEQ ID NOS: 64, 65, 66, 67, 68, 69, 70, 71, 72, or 73 and 55.

In a still further embodiment, the invention provides an IGF-1R antibody having a light-chain variable domain comprising SEQ ID NO:53 or a heavy-chain variable domain comprising SEQ ID NO:55, or having light-chain and heavy-chain variable domains comprising both SEQ ID NO:53 and 55.

In further aspects, the invention provides an antibody having the full-length heavy-chain sequence of SEQ ID NO:90 and the full-length light-chain sequence of SEQ ID NO:91.

In other preferred embodiments, the antibody has an Fc region. In one aspect, such Fc region of the antibody is a wild-type (or native-sequence) Fc region. In another embodiment, the antibody further comprises one or more amino acid substitutions in its Fc region that result in the polypeptide exhibiting at least one of the following properties: increased FcγR binding, increased antibody-dependent cell-mediated cytotoxicity (ADCC), increased complement-dependent cytotoxicity (CDC), decreased CDC, increased ADCC and CDC function, increased ADCC but decreased CDC function, increased FcRn binding, and increased serum half life, as compared to an antibody having a native-sequence Fc region.

In a particularly preferred embodiment, the antibody has amino acid substitutions in its Fc region at any one or any combination of positions that are 268D, or 298A, or 326D, or 333A, or 334A, or 298A together with 333A, or 298A together with 334A, or 239D together with 332E, or 239D together with 298A and 332E, or 239D together with 268D and 298A and 332E, or 239D together with 268D and 298A and 326A and 332A, or 239D together with 268D and 298A and 326A and 332E, or 239D together with 268D and 283L and 298A and 332E, or 239D together with 268D and 283L and 298A and 326A and 332E, or 239D together with 330L and 332E and 272Y and 254T and 256E, or 250Q together with 428L, or 265A, or 297A, wherein the 265A substitution is in the absence of 297A and the 297A substitution is in the absence of 265A. In one particular embodiment, the Fc region has from one to three such amino acid substitutions, for example, substitutions at positions 298, 333, and 334, and more preferably the combination of 298A, 333A, and 334A. The letter after the number in each of these designations represents the changed amino acid at that position. Such anti-IGF-1R antibodies effect varying degrees of disruption of the IGF-1R signaling pathway. For example, in one embodiment, the invention provides an anti-IGF-1R antibody (preferably humanized) wherein the monovalent affinity of the antibody to human IGF-1R (e.g., affinity of the antibody as a Fab fragment to human IGF-1R) is about the same as or greater than that of a murine antibody (e.g., affinity of the murine antibody as a Fab fragment to human IGF-1R) produced by a hybridoma cell line deposited on Sep. 20, 2005 under American Type Culture Collection Accession Number PTA-7007, PTA-7008, PTA-7009, PTA-7010, PTA-7011, PTA-7012, PTA-7013, PTA-7014, PTA-7015, PTA-7016, PTA-7017, PTA-7018, or PTA-7019, which are identified below. The monovalent affinity is preferably expressed as a Kd value and/or is measured by optical biosensor that uses surface plasmon resonance (SPR) (BIACORE® technology) or radioimmunoassay.

Further antibodies herein include those with any of the properties above having reduced fusose relative to the amount of fucose on the same antibody produced in a wild-type Chinese hamster ovary cell. More preferred are those antibodies having no fucose.

In another embodiment, the invention provides an antibody composition comprising the antibodies described herein having an Fc region, wherein about 20-100% of the antibodies in the composition comprise a mature core carbohydrate structure in the Fc region that lacks a fucose. Preferably, such composition comprises antibodies having an Fc region that has been altered to change one or more of the ADCC, CDC, or pharmacokinetic properties of the antibody compared to a wild-type IgG Fc sequence by substituting an amino acid selected from the group consisting of A, D, E, L, Q, T, and Y at any one or any combination of positions of the Fc region selected from the group consisting of: 238, 239, 246, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 309, 312, 314, 315, 320, 322, 324, 326, 327, 329, 330, 331, 332, 333, 334, 335, 337, 338, 340, 360, 373, 376, 378, 382, 388, 389, 398, 414, 416, 419, 428, 430, 434, 435, 437, 438 and 439.

The above-described antibody composition is more preferably one wherein the antibody further comprises an Fc substitution that is 268D or 326D or 333A together with 334A, or 298A together with 333A, or 298A together with 334A, or 239D together with 332E, or 239D together with 298A and 332E, or 239D together with 268D and 298A and 332E, or 239D together with 268D and 298A and 326A and 332A, or 239D together with 268D and 298A and 326A and 332E, or 239D together with 268D and 283L and 298A and 332E, or 239D together with 268D and 283L and 298A and 326A and 332E, or 239D together with 330L and 332E, wherein the letter after the number in each of these designations represents the changed amino acid at that position.

The above-described antibody composition is additionally preferably one wherein the antibody binds an FcγRIII. The composition is preferably one wherein the antibody has ADCC activity in the presence of human effector cells or has increased ADCC activity in the presence of human effector cells compared to the otherwise same antibody comprising a human wild-type IgG1Fc. The composition is also preferably one wherein the antibody binds the FcγRIII with better affinity, or mediates ADCC more effectively, than a glycoprotein with a mature core carbohydrate structure including fucose attached to the Fc region of the glycoprotein. In addition, the composition is preferably one wherein the antibody has been produced by a Chinese hamster ovary (CHO) cell, preferably a Lec13 cell. The composition is also preferably one wherein the antibody has been produced by a mammalian cell lacking a fucosyltransferase gene, more preferably the FUT8 gene.

In one embodiment, the above-described composition is one wherein the antibody is free of bisecting N-acetylglucosamine (GlcNAc) attached to the mature core carbohydrate structure. In an alternative embodiment, the composition is one wherein the antibody has bisecting GlcNAc attached to the mature core carbohydrate structure.

In another aspect, the above-described composition is one wherein the antibody has one or more galactose residues attached to the mature core carbohydrate structure. In an alternative embodiment, the composition is one wherein the antibody is free of one or more galactose residues attached to the mature core carbohydrate structure.

In a further aspect, the above-described composition is one wherein the antibody has one or more sialic acid residues attached to the mature core carbohydrate structure. In an alternative aspect, the composition is one wherein the antibody is free of one or more sialic acid residues attached to the mature core carbohydrate structure.

The above-described composition preferably comprises at least about 2% afucosylated antibodies, more preferably at least about 4% afucosylated antibodies, still more preferably at least about 10% afucosylated antibodies, even more preferably at least about 19% afucosylated antibodies, and most preferably about 100% afucosylated antibodies.

Also included herein is an anti-idiotype antibody that specifically binds any of the antibodies herein.

An antibody for use in a host subject preferably elicits little to no immunogenic response against the agent in said subject. In one embodiment, the invention provides a chimeric or humanized antibody that elicits and/or is expected to elicit a human anti-mouse antibody response (HAMA) at a substantially reduced level compared to a murine antibody in a host subject. In another example, the invention provides a chimeric or humanized antibody that elicits and/or is expected to elicit minimal or no human anti-mouse antibody response (HAMA). In one example, an antibody of the invention elicits an anti-mouse antibody response that is at or below a clinically acceptable maximum level.

The invention further provides an anti-idiotype antibody that specifically binds an antibody herein.

In another aspect, the invention supplies a composition comprising one or more of the antibodies herein and a carrier. This composition may further comprise a second medicament, wherein the antibody(ies) is a first medicament.

For cancer treatment, this second medicament may be, for example, another antibody, chemotherapeutic agent, cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, or growth-inhibitory agent. More preferably, this second medicament for cancer treatment is tamoxifen, an aromatase inhibitor, cetuximab, an antagonist to vascular endothelial growth factor (VEGF) or to ErbB or the Erb receptor or to Her-1 or Her-2, an Apo2L/TRAIL DR5 agonist (such as apomab, a DR-5-targeted dual proapoptotic receptor agonist), a poly(ADP-ribose) polymerase 1 (PARP) inhibitor, a heat-shock protein 90 (Hsp90) inhibitor, a medicament conjugated to a cytotoxin, a c-met inhibitor, a MAP-erk kinase (MEK) inhibitor, a phosphatidylinositol 3-kinase (P13K) inhibitor, a AKT inhibitor, or a pan-HER tyrosine kinase inhibitor (TKI). Still more preferably, this second medicament is tamoxifen, apomab, letrozole, irinotecan, cetuximab, fulvestrant, vinorelbine, erlotinib, bevacizumab, vincristine, lapatinib, docetaxel, gefitinib, trastuzumab, trastuzumab conjugated to a maytansinoid, or a monoclonal antibody to c-met. Most preferably, this second medicament is erlotinib, apomab, bevacizumab, or trastuzumab.

For treatment of aging, this second medicament may be, for example, a statin, bisphosphonate, cholesterol-lowering agent, hypertension-treating agent, interleukin-6 inhibitor, interleukin-6 receptor inhibitor, interleukin-6 anti-sense oligonucleotide, gp130 protein inhibitor, growth hormone, growth-hormone-releasing hormone, growth-hormone secretagogue, or insulin-resistance-treating agent.

For treating autoimmune disorders, this second medicament may be, for example, an antagonist binding to a B-cell surface marker, a BAFF antagonist, a TNF antagonist, a chemotherapeutic agent, an immunosuppressive agent, a cytotoxic agent, an integrin antagonist, a cytokine, a cytokine antagonist, a hormone, a disease-modifying anti-rheumatic drug (DMARD), a non-steroidal anti-inflammatory drug (NSAID), an anti-rheumatic agent, a muscle relaxant, a narcotic, or a combination thereof.

In another embodiment, the invention provides a murine hybridoma deposited at the American Type Culture Collection (ATCC) on Sep. 20, 2005 under Deposit No. PTA-7007, PTA-7008, PTA-7009, PTA-7010, PTA-7011, PTA-7012, PTA-7013, PTA-7014, PTA-7015, PTA-7016, PTA-7017, PTA-7018, or PTA-7019. Further provided is an antibody secreted by such a hybridoma.

Another aspect of the invention is an isolated nucleic acid encoding an antibody of any one of the preceding embodiments. Expression vectors comprising such nucleic acid, and those encoding the antibodies of the invention, are also provided. Also provided is a host cell comprising a nucleic acid encoding an antibody of the invention. Any of a variety of host cells can be used. In one embodiment, the host cell is a prokaryotic cell, for example, E. coli. In another embodiment, the host cell is a eukaryotic cell, for example, a yeast cell or mammalian cell such as a CHO cell.

In another aspect, the invention provides methods for making an antibody of the invention. For example, the invention provides a method of making or producing an IGF-1R antibody herein, said method comprising (i) culturing a suitable host cell comprising a nucleic acid encoding an antibody of the invention (preferably comprising a recombinant vector of the invention encoding said antibody (or fragment thereof)), under conditions to produce the antibody, and (ii) recovering said antibody. The antibody may be recovered from the host cell or host cell culture. In a preferred embodiment, the antibody is a naked antibody. In another preferred embodiment, the antibody is conjugated with another molecule, the other molecule preferably being a cytotoxic agent.

In another aspect, the invention provides a method of inhibiting IGF-1R-activated cell proliferation, said method comprising contacting a cell or tissue with an effective amount of an antibody of the invention. The cell proliferation to be inhibited is preferably acromegaly, retinal neovascularization, psoriasis, or cancer. In another aspect, the invention involves the use of an antibody of the invention in the manufacture of a pharmaceutical composition for inhibiting IGF-1R-activated cell proliferation.

In a still further aspect, the invention provides a method for inhibiting growth of a cancer cell comprising contacting the cell with an antibody of the invention. In another aspect, the invention involves the use of an antibody of the invention in the manufacture of a pharmaceutical composition for inhibiting growth of a cancer cell.

In a further aspect, the invention provides a method of treating a cancer in a subject comprising administering to the subject an effective amount of an antibody of the invention.

In another aspect, the invention involves the use of an antibody of the invention in the manufacture of a pharmaceutical composition for treating a cancer in a subject.

Preferably, the cancer is selected from the group consisting of multiple myeloma, breast cancer, colon cancer, ovarian cancer, osteosarcoma, cervical cancer, prostate cancer, lung cancer, kidney cancer, liver cancer, synovial carcinoma, and pancreatic cancer. More preferably, the cancer is multiple myeloma, breast cancer, ovarian cancer, colorectal cancer, lung cancer, and prostate cancer. Still more preferably, the cancer is lung cancer, colorectal cancer, or breast cancer. Even more preferably, the cancer is non-small lung cell cancer (NSCLC), including adenocarcinoma and squamous cell carcinoma. Most preferably, the subject with NSCLC has been previously treated with another medicament (that is, the patient is treated for “second-line” NSCLC in that the patients who have been treated with another drug failed on that drug, whether the cancer had progressed or the patient's cancer did not respond to the drug). The medicament with which the subject has been previously treated is most preferably a chemotherapeutic agent or bevacizumab or both.

In another embodiment of this method for treating cancer, a second medicament is administered to the subject in an effective amount, wherein the antibody is a first medicament. In one aspect, this second medicament is more than one medicament, and is preferably another antibody, chemotherapeutic agent, cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, or growth-inhibitory agent.

More specific such second medicaments for treating cancer include, for example, irinotecan (CAMPTOSAR®), cetuximab (ERBITUX®), fulvestrant (FASLODEX®)), vinorelbine (NAVELBINE®), EGF-receptor antagonists such as erlotinib (TARCEVA®), VEGF antagonists such as bevacizumab (AVASTIN®), vincristine (ONCOVIN®), an Apo2L/TRAIL DR5 agonist (such as apomab, which is a DR-5-targeted dual proapoptotic receptor agonist), inhibitors of mTor (a serine/threonine protein kinase) such as rapamycin and CCI-779, and anti-HER1, HER2, ErbB, and/or EGFR antagonists such as trastuzumab (HERCEPTIN®), pertuzumab (OMNITARG™), or lapatinib, and other cytotoxic agents including chemotherapeutic agents. The second medicament is preferably an anti-estrogen drug such as tamoxifen, fulvestrant, or an aromatase inhibitor, an antagonist to vascular endothelial growth factor (VEGF), an Apo2L/TRAIL DR5 agonist, an antagonist to ErbB or the Erb receptor, or an antagonist to Her-1 or Her-2. More preferably, the second medicament is tamoxifen, apomab, letrozole, exemestane, anastrozole, irinotecan, cetuximab, fulvestrant, vinorelbine, erlotinib, bevacizumab, vincristine, lapatinib, or trastuzumab, and still more preferably, the second medicament is apomab (a DR-5-targeted dual proapoptotic receptor agonist), erlotinib, bevacizumab, or trastuzumab.

More preferably, the cancer is prostate cancer, lung cancer, especially non-small-cell lung cancer, ovarian cancer, pancreatic cancer, colorectal cancer, or breast cancer. Still more preferred is that the cancer is prostate cancer and the second medicament is a taxane or bevacizumab. Alternatively preferred is that the cancer is colorectal cancer and the second medicament is apomab, erlotinib, cetuximab, bevacizumab, and/or irinotecan, more preferably apomab, erlotinib, bevacizumab, or irinotecan. Still alternatively preferred is that the cancer is breast cancer, especially estrogen-receptor-positive breast cancer, HER-2-positive cancer, breast cancer that requires doxorubicin treatment, or breast cancer that does not require doxorubicin treatment, and the second medicament is an anti-estrogen drug such as fulvestrant, apomab, tamoxifen, or an aromatase inhibitor such as letrozole, exemestane, or anastrozole, or is bevacizumab, trastuzumab, lapatinib, or a combination thereof. Also alternatively preferred is that the cancer is lung cancer and the second medicament is apomab, erlotinib or bevacizumab.

Still more preferably, the cancer is colorectal cancer and the second medicament is bevacizumab, apomab, cetuximab, or erlotinib; or the cancer is breast cancer and the second medicament is bevacizumab, apomab, fulvestrant, tamoxifen, letrozole, or trastuzumab; or the cancer is non-small-cell lung cancer and the second medicament is bevacizumab, apomab, or erlotinib.

Preferred combinations are wherein the second medicament for treating cancer is tamoxifen, an aromatase inhibitor, cetuximab, an antagonist to vascular endothelial growth factor (VEGF), an Apo2L/TRAIL DR5 agonist (such as apomab, a DR-5-targeted dual proapoptotic receptor agonist), an antagonist to ErbB or the Erb receptor, an antagonist to Her-1 or Her-2, a poly(ADP-ribose) polymerase 1 (PARP) inhibitor, a heat-shock protein 90 (Hsp90) inhibitor, a medicament conjugated to a cytotoxin, a c-met inhibitor, a MAP-erk kinase (MEK) inhibitor, a phosphatidylinositol 3-kinase (P13K) inhibitor, a AKT inhibitor, or a pan-HER tyrosine kinase inhibitor (TKI). In another preferred embodiment, the combination is wherein the second medicament is tamoxifen, apomab, letrozole, irinotecan, cetuximab, fulvestrant, vinorelbine, erlotinib, bevacizumab, vincristine, lapatinib, docetaxel, gefitinib, trastuzumab, trastuzumab conjugated to a maytansinoid, or a monoclonal antibody to c-met. Most preferably, the second medicament is erlotinib, apomab, bevacizumab, or trastuzumab.

In another embodiment, the invention supplies a method for treating aging in a subject comprising administering to the subject an effective amount of an antibody of this invention. In a preferred embodiment, a second medicament is administered in an effective amount, wherein the antibody is a first medicament. Examples of suitable second medicaments include a statin, bisphosphonate, cholesterol-lowering agent, hypertension-treating agent, interleukin-6 inhibitor, interleukin-6 receptor inhibitor, interleukin-6 anti-sense oligonucleotide, gp130 protein inhibitor, growth hormone, growth-hormone-releasing hormone, growth-hormone secretagogue, or insulin-resistance-treating agent. In another aspect, the invention involves the use of an antibody of the invention in the manufacture of a pharmaceutical composition for treating aging in a subject.

In a further aspect, the invention provides a method of treating an autoimmune disorder in a subject comprising administering to the subject an effective amount of an antibody of this invention. Examples of such disorders include rheumatoid arthritis, lupus, Wegener's disease, inflammatory bowel disease (IBD), idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjögren's syndrome, glomerulonephritis, Hashimoto's thyroiditis, Graves' disease, helicobacter-pylori gastritis, and chronic hepatitis C. Preferred such disorders are rheumatoid arthritis, multiple sclerosis, Sjögren's syndrome, systemic lupus erythematosus (SLE), lupus nephritis, myasthenia gravis, or IBD. In one embodiment, a second medicament is administered in an effective amount to treat the autoimmune disorder, wherein the antibody is a first medicament. Examples of such second medicaments include an antagonist binding to a B-cell surface marker, a BAFF antagonist, a TNF antagonist, a chemotherapeutic agent, an immunosuppressive agent, a cytotoxic agent, an integrin antagonist, a cytokine, a cytokine antagonist, a hormone, a disease-modifying anti-rheumatic drug (DMARD), a non-steroidal anti-inflammatory drug (NSAID), an anti-rheumatic agent, a muscle relaxant, a narcotic, or a combination thereof. In another aspect, the invention involves the use of an antibody of the invention in the manufacture of a pharmaceutical composition for treating an autoimmune disorder in a subject.

In one preferred aspect of these treatment methods, the subject has never been previously administered a medicament for the cancer or aging, or for any autoimmune disorder.

In another aspect of these treatment methods, the subject has been previously administered at least one medicament for the cancer or aging, or for any autoimmune disorder. In a further embodiment, the subject was not responsive to at least one medicament that was previously administered, with exemplary such previously administered medicament or medicaments for cancer being selected from the group consisting of a chemotherapeutic agent, cytotoxic agent, anti-angiogenic agent, immunosuppressive agent, pro-drug, cytokine, cytokine antagonist, cytotoxic radiotherapy, corticosteroid, anti-emetic, cancer vaccine, analgesic, anti-vascular agent, and growth-inhibitory agent. More preferably, the subject was not responsive to at least one chemotherapeutic agent, cytotoxic agent, anti-angiogenic agent, or immunosuppressive agent. In an alternative preferable embodiment, the subject was not responsive to at least one antagonist to IGF-1R, preferably an antagonist that is not the antibody of this invention (such as small-molecule inhibitors of IGF-1R, or anti-sense oligonucleotides, antagonistic peptides, or antibodies to IGF-1R that are not the antibodies of this invention). In a still alternative preferable embodiment, the subject was not responsive to an epidermal growth factor receptor (EGFR) inhibitor such as erlotinib, more preferably erlotinib.

In a further part of this aspect, the invention relates to a method of reducing the risk of a negative side effect (e.g., selected from the group consisting of an infection, cancer, heart failure, and demyelination) of a medicament that was administered to a subject for treatment (e.g., of a disorder) comprising administering to the previously treated subject an effective amount of an antibody herein, preferably wherein the subject was and is being treated for cancer, and especially the preferred cancer types listed above.

In another aspect of the treatment methods, the antibody is a naked antibody. Alternatively, the antibody is conjugated with another molecule, such as, for example, a polyethylene glycol that extends half-life. The antibody may be administered, for example, intravenously or subcutaneously. The subject is preferably human.

Still another aspect of the invention is an article of manufacture comprising a container and a composition contained therein, wherein the composition comprises an antibody of any of the preceding embodiments and a package insert indicating that the composition can be used to treat the indication the antibody as intended for, such as cancer or aging, or alternatively, an autoimmune disorder. This article may further comprise a container comprising a second medicament, wherein the antibody is a first medicament, and further comprising instructions on the package insert for treating the subject with the second medicament.

Specifically, in one embodiment, the invention provides an article of manufacture comprising a container and a composition contained therein, wherein the composition comprises an antibody of any one of the preceding aspects, and a package insert indicating that the composition can be used to treat a cancer. Preferably, the article further comprises a container comprising a second medicament, wherein the antibody is a first medicament, and further comprises instructions on the package insert for treating the subject with the second medicament. Preferably, the second medicament is another antibody, chemotherapeutic agent, cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, or growth-inhibitory agent. In another embodiment, the second medicament is tamoxifen, letrozole, irinotecan, cetuximab, fulvestrant, apomab, vinorelbine, erlotinib, bevacizumab, vincristine, lapatinib, or trastuzumab. In still another aspect, the second medicament is erlotinib, apomab, bevacizumab, or trastuzumab.

In another specific embodiment, the invention provides an article of manufacture comprising a container and a composition contained therein, wherein the composition comprises any of the antibodies herein, and a package insert indicating that the composition can be used to treat aging. In a preferred aspect, the article further comprises a container comprising a second medicament, wherein the antibody is a first medicament, and further comprising instructions on the package insert for treating the subject with the second medicament. The preferred second medicament is a statin, bisphosphonate, cholesterol-lowering agent, hypertension-treating agent, interleukin-6 inhibitor, interleukin-6 receptor inhibitor, interleukin-6 anti-sense oligonucleotide, gp130 protein inhibitor, growth hormone, growth-hormone-releasing hormone, growth-hormone secretagogue, or insulin-resistance treating agent.

In another aspect, the invention provides a method for assessing activity of an anti-IGF-1R antibody in tumor tissue comprising subjecting tissue from tumors treated with the antibody to positron emission tomography with 2-fluoro-2-deoxy-D-glucose (FDG-PET) imaging and determining if the antibody inhibits FDG uptake into the tissue, with inhibition of FDG uptake correlating with delayed tumor growth. Preferably, the tumor tissue is breast cancer or neuroblastoma tissue.

In a preferred embodiment, the antibody comprises at least one HVR sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid;
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10);
(c) a HVR-L3 sequence comprising amino acids C1-C9, wherein C1-C9 is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid;
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid;
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32).

In another embodiment, the invention comprises a method for packaging a pharmaceutical composition of an antibody herein comprising combining the pharmaceutical composition of the antibody with a package insert instructing a user to treat a patient in need of said antibody with a dose of about 200 mg of the antibody.

In another aspect, the invention provides a method for advertising use of an antibody herein comprising communicating, for example, promoting, to a target audience, use of the antibody for treating a patient or patient population in need of said antibody.

In a further aspect, the invention supplies a method for promoting therapeutic treatment of a human patient with a minimal effective dose of a an antibody herein comprising including in a commercial package of a composition of the antibody for use in such therapy a package insert directing a user to employ a dose of about 100 to 500 mg of the antibody in the composition to treat said patient.

In a still further aspect, the invention provides a method for minimizing side-effects associated with combination therapy of disorders characterized by expression or overexpression of IGF-1R, comprising including in a commercial package of a therapeutic anti-IGF-1R antibody composition for use in such therapy, a package insert with instructions to avoid the use of an anthracycline-type chemotherapeutic in combination with said composition. Preferably, the method is such wherein the anthracycline-type chemotherapeutic is doxorubicin or epirubicin. Also, preferably, the anti-IGF-1R antibody is an antibody herein. Also preferred is that the therapy is for a human.

In a further embodiment, the invention supplies a method for minimizing side-effects associated with combination therapy of disorders characterized by expression or overexpression of IGF-1R comprising:
(a) obtaining a commercial package comprising a therapeutic anti-IGF-1R antibody composition and a package insert containing instructions for use of said composition in combination therapy, said instructions having information that directs a user to not use an anthracycline-type chemotherapeutic in said combination therapy; and
(b) utilizing the package insert to select a chemotherapeutic agent other than an anthracycline-type chemotherapeutic for use in combination with the therapeutic anti-IGF-1R antibody composition. In a preferred embodiment, the chemotherapeutic agent other than an anthracycline-type chemotherapeutic is a taxoid. Also, preferably, the anti-IGF-1R antibody is an antibody herein. Also preferred is that the therapy is for a human.

### BRIEF DESCRIPTION OF THE DRAWINGS

FIG. 1 depicts alignment of sequences of the light-chain variable domain for the following with respect to the anti-IGF-1R clone 2B4: human light kappa subgroup consensus sequence (SEQ ID NO:42), murine 2B4 anti-IGF-1R clone (m2B4) (SEQ ID NO:43), and humanized 2B4.vX antibody based on murine 2B4 (h2B4.vX) (SEQ ID NO:44). The L1, L2, and L3 HVR portions of the sequences are boxed with bold lines, with the Kabat, Chothia, and contact complementarity-determining regions (CDRs) shown in smaller boxes above each of the respective L1, L2, and L3 boxed sequences.

FIG. 2 depicts alignment of sequences of the heavy-chain variable domain for the following with respect to the anti-IGF-1R clone 2B4: human heavy subgroup III consensus sequence (SEQ ID NO:45), murine 2B4 anti-IGF-1R clone (m2B4) (SEQ ID NO:46), and humanized 2B4.vX antibody based on, murine 2B4 (h2B4.vX) (SEQ ID NO:47). The H1, H2, and H3 HVR portions of the sequences are boxed with bold lines, with the Kabat, Chothia, and contact CDRs shown in smaller boxes above each of the respective H1, H2, and H3 boxed sequences. The H2 HVR is an extended HVR as defined herein. The boxes for residues 71, 73, and 78 show changes in these positions as compared to the human heavy subgroup III consensus sequence.

FIG. 3 depicts alignment of sequences of the light-chain variable domain for the following with respect to the anti-IGF-1R clone 9F2: human light kappa subgroup I consensus sequence (SEQ ID NO:42), murine 9F2 anti-IGF-1R clone (m9F2) (SEQ ID NO:48), and humanized 9F2.vX antibody based on murine 9F2 (h9F2.vX) (SEQ ID NO:49). The L1, L2, and L3 HVR portions of the sequences are boxed with bold lines, with the Kabat, Chothia, and contact CDRs shown in smaller boxes above each of the respective L1, L2, and L3 boxed sequences.

FIG. 4 depicts alignment of sequences of the heavy-chain variable domain for the following with respect to the anti-IGF-1R clone 9F2: human heavy subgroup III consensus sequence (SEQ ID NO:45), murine 9F2 anti-IGF-1R clone (m9F2) (SEQ ID NO:50), and humanized 9F2.vX antibody based on, murine 9F2 (h9F2.vX) (SEQ ID NO:51). The H1, H2, and H3 HVR portions of the sequences are boxed with bold lines, with the Kabat, Chothia, and contact CDRs shown in smaller boxes above each of the respective H1, H2, and H3 boxed sequences. The H2 HVR is an extended HVR as defined herein. The boxes for residues 71, 73, and 78 show changes in these positions as compared to the human heavy subgroup III consensus sequence.

FIG. 5 depicts alignment of sequences of the light-chain variable domain for the following with respect to the anti-IGF-1R clone 10H5: human light kappa subgroup I consensus sequence (SEQ ID NO:42), murine 10H5 anti-IGF-1R clone (m10H5) (SEQ ID NO:52), and humanized 10H5.vX antibody (also called herein h10H5) based on murine 10H5 (h10H5.vX) (SEQ ID NO:53). The L1, L2, and L3 HVR portions of the sequences are boxed with bold lines, with the Kabat, Chothia, and contact CDRs shown in smaller boxes above each of the respective L1, L2, and L3 boxed sequences.

FIG. 6 depicts alignment of sequences of the heavy-chain variable domain for the following with respect to the anti-IGF-1R clone 10H5: human heavy subgroup III consensus sequence (SEQ ID NO:45), murine 10H5 anti-IGF-1R clone (m10H5) (SEQ ID NO:54), and humanized 10H5.vX antibody based on, murine 10H5 (h10H5.vX) (SEQ ID NO:55). The H1, H2, and H3 HVR portions of the sequences are boxed with bold lines, with the Kabat, Chothia, and contact CDRs shown in smaller boxes above each of the respective H1, H2, and H3 boxed sequences. The H2 HVR is an extended HVR as defined herein. The boxes for residues 71, 73, and 78 show changes in these positions as compared to the human heavy subgroup III consensus sequence.

FIG. 7 depicts how to design HVR randomization for 10H5.vX affinity maturation. As used herein h10H5 is the same as 10H5.vX. Most of the HVR residues were randomized using the strategy of soft randomization with a bias toward wild-type residues as 50%. To achieve that, the codon for an individual amino acid was synthesized according to the following rule: 5 equals to 70% of adenosine (A) and 10% of the other three nucleotides; 6 equals to 70% of guanosine (G) and 10% of the other three nucleotides; 7 equals to 70% of cytidine (C) and 10% of the other three nucleotides; and 8 equals to 70% of thymidine (T) and 10% of the other three nucleotides. Several residues were limited in sequence diversity using degenerate codons, for example, ARA encoded K and R position 24 in HVR-L1. The H2 HVR is a Kabat CDR as set forth herein.

FIG. 8 depicts how four combinational HVR libraries were generated to improve affinity of the humanized 10H5.vX clone. Four combinatorial HVR libraries were generated using different HVR stop library templates. For example, the HVR-L1/L2/L3 library with the HVR-L3 stop library template ended up with four different combinations: HVR-L3, HVR-L1/L3, HVR-L2/L3, and HVR-L1/L2/L3. In the figure, CDR is equivalent to HVR.

FIG. 9 depicts the stringent panning process to isolate affinity-improved clones. All four combinatorial HVR libraries were subject to human IGF-1R selection on a plate-supported format for round 1, and four subsequent rounds were selected against human IGF-1R in a solution phase with increasing stringency. Significant enrichment of phage selection at round 4 of solution phase panning was observed in two sets of libraries, HVR-L1/L2/L3 and HVR-L3/H1/H2. Plate-supported panning with 5 μg/ml human IGF-1R antigen was used for the round 1 selection at 37° C. for 2 hours. In the figure, CDR is equivalent to HVR.

FIGS. 10A and 10B depict the single-spot competition ELISA to identify affinity-improved clones. FIG. 10A depicts the results of screening 96 clones from the round-4 selection of the HVR-L1, L2, and L3 soft-randomized library, which were screened against 1 nM human IGF-1R in the single-spot phage competition ELISA. Six clones of interest were identified. FIG. 10B depicts the results of screening 96 clones from the round-4 selection of the HVR-L3, H1, and H2 soft-randomized library, which were screened against 1 nM human IGF-1R in the single-spot phage competition ELISA. Four clones of interest were identified. In the figures, CDR is equivalent to HVR.

FIG. 11 depicts a purified phage-competition ELISA to determine the affinity (IC50) of the improved clones binding to human IGF-1R. Six phage clones, h10H5.v2, h10H5.v9, h10H5.v10, h10H5.v39, h10H5.v48, and h10H5.v96A, derived from the HVR-L1/L2/L3 library, and four phage clones, h10H5.v16, h10H5.v32, h10H5.v46, and h10H5.v96B, derived from the HVR-L3/H1/H2 library, were purified and phage binding affinities (IC50) determined using phage-competition ELISA. Relative fold of improvement was calculated with parental clone h10H5.vX.

FIG. 12 depicts the sequences of the light-chain HVRs of the affinity-matured clones derived from h10H5.vX (SEQ ID NO:53), including phage IC50 to human IGF-1R: h10H5.v2 (SEQ ID NO:56), h10H5.v9 (SEQ ID NO:57), h10H5.v10 (SEQ ID NO:58), h10H5.v16 (SEQ ID NO:59), h10H5.v32 (SEQ ID NO:60), h10H5.v39 (SEQ ID NO:61), h10H5.v46 (SEQ ID NO:62), h10H5.v48 (SEQ ID NO:63), h10H5.v96A (SEQ ID NO:64), and h10H5.v96B (SEQ ID NO:65).

FIG. 13 depicts a phage-competition ELISA to determine phage IC50 against human IGF-1R for the YW95 phage-derived clones displayed on the phage as a bivalent Fab-Zip format and for the humanized hybridoma-derived clones displayed on the phage as a monovalent Fab format.

FIG. 14 depicts the results of a BIACORE® instrument analysis of clones YW95.6, murine 2B4, murine 9F2, and murine 10H5Fab against human and murine IGF-1R ligands. IGF-1R ligands were immobilized on the CM5 sensor chip of 500 RU (Response Unit), and 2-fold serial diluted clones YW95.6, murine 2B4, murine 9F2, and murine 10H5Fab from 500 nM to 3.1 nM, respectively, were injected through the sensor chip to determine binding affinities and kinetics at 25° C. The apparent affinity (Kd), including Kₒₙ and K_{off} rates, was derived from a one-to-one Langmuir binding model.

FIG. 15 depicts the results of a BIACORE® instrument analysis of chimeric 9F2-IgG, humanized 9F2.vX-IgG, chimeric 10H5-IgG, and humanized 10H5.vX-IgG against human and cynomolgus-monkey (cyno) IGF-1R ligands. IGF-1R ligands were immobilized on the CM5 sensor chip of 500 RU, and 2-fold serial diluted chimeric 9F2-IgG, humanized 9F2.vX-IgG, chimeric 10H5-IgG, and humanized 10H5.vX-IgG from 250 nM to 0.78 nM, respectively, were injected through the sensor chip to determine binding affinities and kinetics at 25° C. The apparent affinity (Kd), including Kₒₙ and K_{off} rates, was derived from a one-to-one Langmuir binding model.

FIG. 16 depicts alignment of sequences of the light-chain variable domain for the following with respect to the anti-IGF-1R YW95 phage-display clones: human light kappa subgroup I consensus sequence (SEQ ID NO:42), anti-IGF-1R clone YW95.3 (SEQ ID NO:66), anti-IGF-1R clone YW95.6 (SEQ ID NO:67), anti-IGF-1R clone YW95.81 (SEQ ID NO:68), and anti-IGF-1R clone YW95.87 (SEQ ID NO:69). The L1, L2, and L3 HVR portions of the sequences are boxed with bold lines, with the Kabat, Chothia, and contact CDRs shown in smaller boxes above each of the respective L1, L2, and L3 boxed sequences.

FIG. 17 depicts alignment of sequences of the heavy-chain variable domain for the following with respect to the anti-IGF-1R YW95 phage-display clones: human heavy subgroup III consensus sequence (SEQ ID NO:45), anti-IGF-1R clone YW95.3 (SEQ ID NO:70), anti-IGF-1R clone YW95.6 (SEQ ID NO:71), anti-IGF-1R clone YW95.81 (SEQ ID NO:72), and anti-IGF-1R clone YW95.87 (SEQ ID NO:73). The H1, H2, and H3 HVR portions of the sequences are boxed with bold lines, with the Kabat, Chothia, and contact CDRs shown in smaller boxes above each of the respective H1, H2, and H3 boxed sequences. The H2 HVR is an extended HVR as defined herein. The boxes for residues 71, 73, and 78 show changes in these positions as compared to the human heavy subgroup III consensus sequence.

FIGS. 18A and 18B depict the crystal structures of h4D5 (HERCEPTIN® trastuzumab) and YW95.6, respectively.

FIGS. 19A-19D show how various anti-IGF-1R antibodies block IGF-I and IGF-11 binding to IGF-1R. IGF-1R-ECD was coated on a 96-well plate. Anti-IGF-1R antibodies of a serial dilution were pre-incubated with IGF-1R-ECD. Biotinylated IGF-I (FIGS. 19A and 19C) or IGF-II (FIGS. 19B and 19D) was then added, and the remaining IGF-1R-IGF-I or IGF-1R-IGF-11 complexes after multiple washes were detected by binding of streptavidin-horseradish peroxidase (HRP) and subsequent color substrate development. In FIGS. 19A and 19B, hybridoma and phage antibodies are distinguished in the figures by using different symbols. For IGF-1, anti-IR3 antibody was used as a control. For FIGS. 19C and 19D, the solid-phase binding assays were performed by incubating serially diluted antibodies h10H5 (triangles), anti-IR3 (squares), and anti-gp120 (circles) with pre-coated IGF-1R-ECD on the plate, and the error bars indicate the standard deviations of mean results (n=3).

FIGS. 20A and 20B show how various anti-IGF-1R antibodies block IGF-1- or IGF-1′-mediated IGF-1R activation. MCF7 cells were pre-incubated with medium control or various concentrations of anti-IGF-1R antibodies, and stimulated with IGF-I at 10 ng/ml (1.4 nM) (FIG. 20A) or IGF-II at 50 ng/ml (7 nM) (FIG. 20B). The degree of IGF-1R tyrosine phosphorylation was determined by the KIRA assay, which captures IGF-1R to a solid phase through 3B7, an anti-IGF-1R monoclonal antibody, and utilizes an anti-phospho-tyrosine antibody, 4G10, to measure IGF-1R phosphorylation. The IC50 values for various hybridoma clones are listed in the table.

FIGS. 21A and 21B show how various anti-IGF-1R antibodies inhibit IGF-1- and IGF-II-dependent IGF-1R phosphorylation and downstream signaling, as compared to anti-IR3 antibody as control. MCF7 cells were serum-starved overnight, and incubated briefly with medium alone or anti-IGF-1R antibodies, followed by IGF-I (FIG. 21A) or IGF-II (FIG. 21B) stimulation. The cell lysates were prepared directly using SDS sample buffer, separated by SDS-PAGE, and analyzed by Western blotting using the antibodies against IGF-1R, phospho-IGF-1R (pIGF-1R), MAPK1/2, phospho-MAPK1/2 (pMAPK1/2), AKT, and phospho-AKT (pAKT).

FIG. 22 shows how various anti-IGF-1R antibodies performed in a test for inducing IGF-1R down-regulation. MCF7 cells were treated with 2B4, 9F2, 10H5, or YW95.6 for 1, 4, 8, or 24 hours in the presence of 10% serum-containing medium, and cell lysates were harvested, separated by SDS-PAGE, and analyzed by Western blotting using an anti-IGF-1R beta-chain or anti-beta-actin antibody. This figure shows that IGF-1R depletion was induced by antibody treatment.

FIGS. 23A-23E show the effect on inhibition of MCF7 cell proliferation of a selected panel of anti-IGF-1R antibodies. MCF7 cells were incubated with various concentrations of anti-IGF-1R antibodies in the presence of 1% serum for 7 days. The cell viability (FIG. 23A) was measured by CELLTITER-GLO® kit assays, and cell morphology (FIG. 23B) was recorded at day 7 post-treatment. The conditions for the viability assay remained the same for the data shown in FIGS. 23C-E. FIG. 23C shows that mouse monoclonal antibodies 10H5 and 9F2 had a similar inhibitory effect on MCF7 proliferation. FIG. 23D shows that human chimeric antibodies 10H5, 9F2, and 2B4 had a similar inhibitory effect on MCF7 proliferation. FIG. 23E shows that humanized 10H5 (h10H5) was potent in inhibiting MCF7 proliferation. The IC50 was 40 ng/ml.

FIG. 24 shows the dose-dependent efficacy of wild-type antibody h10H5 in a SK-N-AS (10 million cells/mouse) neuroblastoma xenograft model in athymic nude mice. Three different weekly dosings of h10H5 were delivered via intraperitoneal injection to treat SK-N-AS xenografts. The loading doses were twice as much as subsequent doses. Tumor growth is represented as mean tumor volumes over time. The standard error of the mean (SEM) is indicated by the error bars.

FIG. 25 shows that h10H5 mediated IGF-1R down-regulation and inhibited AKT activation in SK-N-AS xenograft tumors. SK-N-AS tumors were grown to 400-600 mm3, treated by two different doses of h10H5 (5 or 20 mg/kg), and collected at 6, 24 or 48 hours post-treatment. Western blotting analysis of tumor lysates was performed using the anti-IGF-1R beta chain, beta-actin, AKT, and phospho-AKT (pAKT) antibodies.

FIG. 26 shows the efficacy of h10H5 as a single agent in a SW527 breast-cancer cell line xenograft model in SCID beige mice (5 million cells/mouse). Two different weekly dosings of h10H5 were delivered via intraperitoneal injection to treat the SW527 xenografts. The loading doses were twice as much as subsequent doses. Tumor growth is represented as mean tumor volumes over time. Standard error of the mean (SEM) is indicated by the error bars.

FIGS. 27A and 27B show the efficacy of h10H5 vs. Colo205 (5 million cells/mouse) tumors in athymic nude mice. FIG. 27A shows the single-agent activity of h10H5 in the Colo205 colorectal cancer xenograft model. FIG. 27B shows that h10H5 enhances 5-FU's inhibitory effect on Colo205-e215 xenograft tumors when used in combination with 5-FU. Weekly dosings of h10H5 were delivered via intraperitoneal injection throughout the study, while 5-FU (100 mg/kg) was given weekly for the first three weeks. The loading doses were twice as much as subsequent ones. Tumor growth is represented as mean tumor volumes over time. Standard error of the mean (SEM) is indicated by the error bars.

FIG. 28 shows the efficacy of h10H5 alone and in combination with vinorelbine vs. A549 (5 million cells/mouse) tumors in athymic nude mice. Antibody h10H5 is shown to enhance vinorelbine's inhibitory effect on A549 xenograft tumors when used in combination. Antibody h10H5 was delivered via intraperitoneal injection two times per week throughout the study (arrows), while vinorelbine (9 mg/kg) was given weekly for the first three weeks (arrow-heads). The loading doses were twice as much as subsequent ones. Tumor growth is represented as mean tumor volumes over time. Standard error of the mean (SEM) is indicated by the error bars.

FIG. 29A-H show the time course of internalization of IGF-1R by h10H5 in MCF7 cells. MCF7 cells were incubated with 5 μg/mL of h10H5 in the presence of lysosomal protease inhibitors for 5 minutes (A-B), 20 minutes (C-D), 1 hour (E-F), or 4 hours (G-H), then fixed, permeabilized, and stained with Cy3-anti-human (A, C, E, G). The right panels show either co-internalized ALEXA488™-transferrin (B, D) or mouse anti-LAMP1 co-staining (F, H). Scale bar=20 μM in main panels. Insets show the boxed regions at 3× magnification.

FIGS. 30A and B show that h10H5-induced IGF-1R down-regulation is mediated by proteasome and lysosome pathways. SK-N-AS cells were pre-treated with a combination of 5 μM of pepstatin A and 10 μg/ml of leupeptin (FIG. 30A) or 30 μM of the proteasome inhibitor VELCADE® bortezomib (FIG. 30B) for one hour, and subsequently exposed to h10H5 treatment for the indicated times in the presence of lysosomal protease inhibitors. Cell lysates were analyzed for IGF-1R α subunit and β-subunit cytoplasmic region by Western blotting. β-actin was used as a loading control.

FIG. 31 shows that h10H5 effectively cooperates with docetaxel and anti-VEGF antibody to inhibit the growth of SW527 breast cancer xenograft tumors. Solid arrows (for vehicle, h10H5, and B20-4.1) and open arrows (docetaxel) indicate the days on which test materials were administered through intraperitoneal (IP) and intravenous (IV) injections, respectively. Tumor volume changes were monitored for 14 days. Error bars indicate the standard errors of mean results. Slash marks indicate animals that were euthanized due to large tumor size, ulcerated tumor, or more than 20% weight loss. Various treatment regimens are indicated in the inset.

FIG. 32A-C show that h10H5 treatment results in decreased tumor FDG uptake. For FIG. 32A, SK-N-AS cells were incubated with serially diluted h10H5 for 48 hrs in either serum-free or 0.1% serum-containing media, distinguished by different symbols in the inset. [³H]-FDG was added during the last 24 hr of the incubation, and radioactivity incorporated (CPM) was measured by a scintillation counter. FIG. 32B shows inhibition of SK-N-AS xenograft tumor growth by h10H5. Arrows indicate the intravenous injection of 10 mg/kg of 10H5 or vehicle control on Day 0. Tumor volume changes were monitored for 14 days. Error bars indicate the standard errors of mean results. Slash marks indicate animals that were euthanized due to large tumor. Treatment regimens are indicated in the inset. For FIG. 32C parallel FDG-PET measurements of SK-N-AS tumors were taken to evaluate the changes in the FDG uptake rate constant by h10H5 treatment over time. Error bars indicate standard errors of mean results.

FIG. 33A-B shows that h10H5 does not mediate significant ADCC. For FIG. 33A, in vitro ADCC assays were performed by incubating SK-N-AS and BT474 cells with serially diluted 10H5 and HERCEPTIN® (trastuzumab), respectively. PBMCs were used as effector cells. Percentages of cytotoxicity were measured by released lactate dehydrogenase activity. Various antibody/target cell combinations are indicated in the inset. FIG. 33B shows that wild-type (WT) 10H5 (h10H5) and a Fcγ-binding defective mutant of h10H5 (D265A (Kabat numbering)) exhibited similar anti-tumor activity. SK-N-AS xenograft tumors were treated with WT or mutant h10H5 at 0.2 and 5 mg/kg. Error bars indicate the standard errors of mean results. Slash marks indicate animals that were euthanized due to large tumor size.

### DETAILED DESCRIPTION OF THE PREFERRED EMBODIMENTS

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

### DEFINITIONS

“Insulin-like growth factor-I receptor” or “IGF-1R” is defined herein as a mammalian biologically active polypeptide, which, if human, has the amino acid sequence of SEQ ID NO:67 of U.S. Pat. No. 6,468,790. Preferably, the IGF-1R herein referred to is human.

“IGF” or “insulin-like growth factor” refers to IGF-I and IGF-II, which bind to IGF-1R and are well known in the literature, e.g., U.S. Pat. No. 6,331,609 and U.S. Pat. No. 6,331,414. They are normally mammalian as used herein, and most preferably human.

“Blocking the interaction of an insulin-like growth factor (IGF) with IGF-1R” refers to interfering with the binding of an IGF to IGF-1R, whether complete or partial interfering or inhibiting.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

An “isolated” antibody is one that has been identified and separated and/or recovered from a component of its natural environment. Contaminant components of its natural environment are materials that would interfere with research, diagnostic or therapeutic uses for the antibody, and may include enzymes, hormones, and other proteinaceous or non-proteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or non-reducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)).

The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al., Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody-hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med., 9:129-134 (2003); and Hollinger et al., Proc. Natl. Acad. Sci. USA, 90: 6444-6448 (1993). Triabodies and tetrabodies are also described in Hudson et al., Nat. Med., 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique-clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol., 222: 581-597 (1992); Sidhu et al., J. Mol. Biol., 338(2): 299-310 (2004); Lee et al., J. Mol. Biol., 340(5): 1073-1093 (2004); Fellouse, Proc. Natl. Acad. Sci. USA, 101(34): 12467-12472 (2004); and Lee et al., J. Immunol. Methods, 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90: 2551 (1993); Jakobovits et al., Nature, 362: 255-258 (1993); Bruggemann et al., Year in Immunol., 7:33 (1993); U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnol., 14: 845-851 (1996); Neuberger, Nature Biotechnol., 14: 826 (1996); and Lonberg and Huszar, Intern. Rev. Immunol., 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1:105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 227:381 (1991); Marks et al., J. Mol. Biol., 222:581 (1991)). Also available for the preparation of human monoclonal antibodies are methods described in Cole et al., Monoclonal Antibodies and Cancer Therapy, Alan R. Liss, p. 77 (1985) and Boerner et al., J. Immunol., 147(1):86-95 (1991). See also van Dijk and van de Winkel, Curr. Opin. Pharmacol., 5: 368-374 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein, refers to the regions of an antibody-variable domain that are hypervariable in sequence and/or form structurally defined loops. Generally, antibodies comprise six HVRs, three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity, 13:37-45 (2000); Johnson and Wu in Methods in Molecular Biology, 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature, 363:446-448 (1993) and Sheriff et al., Nature Struct. Biol., 3:733-736 (1996).

A number of HVR delineations are in use and are encompassed herein. The HVRs that are Kabat CDRs are based on sequence variability and are the most commonly used (Kabat et al., supra). Chothia refers instead to the location of the structural loops (Chothia and Lesk, J. Mol. Biol., 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat |
| | | | | | Number- |
| | | | | | ing) |
| H1 | H31-H35 | H26-H35 | H26-H32 | H30-H35 | (Chothia |
| | | | | | Number- |
| | | | | | ing) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc., according to Kabat) after heavy-chain FR residue 82. The Kabat numbering of residues may be determined for a given antibody by alignment at regions of homology of the sequence of the antibody with a “standard” Kabat-numbered sequence.

An “affinity-matured” antibody is an antibody with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

A “blocking” antibody or an “antagonist” antibody is one that inhibits or reduces biological activity of the antigen it binds. Certain blocking antibodies or antagonist antibodies substantially or completely inhibit the biological activity of the antigen.

Antibodies that “induce apoptosis” are those that induce programmed cell death as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

An “agonist antibody,” as used herein, is an antibody that partially or fully mimics at least one of the functional activities of a polypeptide of interest.

Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding, CDC, Fc-receptor binding, ADCC, phagocytosis, down-regulation of cell-surface receptors (e.g., B-cell receptor), and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Functional fragments” of the antibodies of the invention comprise a portion of an intact antibody, generally including the antigen-binding or variable region of the intact antibody or the Fc region of an antibody that retains FcR binding capability. Examples of antibody fragments include linear antibodies, single-chain antibody molecules, and multispecific antibodies formed from antibody fragments.

A “functional Fc region” possesses an “effector function” of a native-sequence Fc region. Exemplary “effector functions” include C1q binding, CDC, Fc-receptor binding, ADCC, phagocytosis, down-regulation of cell-surface receptors (e.g., B-cell receptor; BCR), etc. Such effector functions generally require the Fc region to be combined with a binding domain (e.g., an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes), native-sequence human IgG2 Fc region, native-sequence human IgG3 Fc region, and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and more preferably at least about 90% homology therewith, and most preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

A polypeptide with a variant Fc region having “altered” FcR binding affinity or ADCC activity is one that has either enhanced or diminished FcR binding activity (e.g., FcγR or FcRn) and/or ADCC activity compared to a parent polypeptide or to a polypeptide comprising a native-sequence Fc region. The polypeptide with a variant Fc region that “exhibits increased binding” to an FcR binds at least one FcR with better affinity than the parent polypeptide. The improvement in binding compared to a parent polypeptide may be about three-fold, preferably about 5-, 10-, 25-, 50-, 60-, 100-, 150-, 200-, and up to 500-fold, or about 25% to 1000% improvement in binding. The polypeptide with a variant Fc region that “exhibits decreased binding” to an FcR binds at least one FcR with less affinity than a parent polypeptide. The decrease in binding compared to a parent polypeptide may be about 40% or more decrease in binding. Such polypeptides with variant Fc regions that display decreased binding to an FcR may possess little or no appreciable binding to an FcR, e.g., about 0-20% binding to the FcR compared to a native-sequence Fc region.

The polypeptide having a variant Fc region that binds an FcR with “better affinity” or “higher affinity” than a polypeptide or parent polypeptide having a wild-type or native-sequence Fc region is one that binds any one or more of the FcRs as defined herein with substantially better binding affinity than the parent polypeptide with a native-sequence Fc region, when the amounts of polypeptide with variant Fc region and parent polypeptide in the binding assay are essentially the same. For example, the polypeptide with a variant Fc region having improved FcR binding affinity may display, e.g., from about two-fold to about 300-fold, more preferably, from about three-fold to about 170-fold, improvement in FcR binding affinity compared to the parent polypeptide or polypeptide with a variant Fc region, where FcR-binding affinity is determined as known in the art and/or as described herein.

The polypeptide having a variant Fc region that “exhibits increased ADCC” or mediates ADCC in the presence of human effector cells more effectively than a polypeptide having a wild-type Fc region is one that in vitro or in vivo is substantially more effective at mediating ADCC, when the amounts of polypeptide with a variant Fc region and the polypeptide with a wild-type Fc region used in the assay are essentially the same. Generally, such variants will be identified using the in vitro ADCC assay as herein disclosed, but other assays or methods for determining ADCC activity, e.g., in an animal model, etc., are contemplated. The preferred polypeptide with a variant Fc region is from about five-fold to about 100-fold, more preferably, from about 25- to about 50-fold, more effective at mediating ADCC than the parent polypeptide or polypeptide with a wild-type Fc region.

“Fc receptor” or “FcR” describes a receptor that binds to the Fc region of an antibody. In some embodiments, an FcR is a native-human FcR. In some embodiments, an FcR is one that binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor-tyrosine-based activation motif (ITAM) in its cytoplasmic domain. Inhibiting receptor FcγRIIB contains an immunoreceptor-tyrosine-based inhibition motif (ITIM) in its cytoplasmic domain. (See, e.g., Daëron, Annu. Rev. Immunol. 15:203-234 (1997).) FcRs are reviewed, for example, in Ravetch and Kinet, Annu. Rev. Immunol., 9:457-492 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol., 117:587 (1976) and Kim et al., J. Immunol., 24:249 (1994)) and regulation of homeostasis of immunoglobulins. Methods of measuring binding to FcRn are known (see, e.g., Ghetie and Ward, Immunology Today, 18 (12):592-8 (1997); Ghetie et al., Nature Biotechnology, 15 (7):637-40 (1997); Hinton et al., J. Biol. Chem., 279(8):6213-6 (2004); WO 2004/92219 (Hinton et al.)).

Binding to human FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2000/42072 (Presta) describes antibody variants with improved or diminished binding to FcRs. See, also, for example, Shields et al., J. Biol. Chem., 9(2): 6591-6604 (2001).

“Human effector cells” are leukocytes that express one or more FcRs and perform effector functions. In certain embodiments, the cells express at least FcγRIII and perform ADCC effector function(s). Examples of human leukocytes that mediate ADCC include PBMC, natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto FcRs present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, supra. To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA), 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods, 202:163 (1996), may be performed. Polypeptide variants with altered Fc-region amino acid sequences (polypeptides with a variant Fc region) and increased or decreased C1q binding capability are described, e.g., in U.S. Pat. No. 6,194,551 and WO 1999/51642. See, also, e.g., Idusogie et al., J. Immunol., 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol., 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res., 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive-binding assays.

According to another embodiment, the Kd or Kd value is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 RU of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol., 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H, and 78H; for instance, the amino acid residues at those positions may be 71A, 73T, and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable-domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). In one embodiment, for the VL, the subgroup is subgroup kappa I as in Kabat et al., supra. In one embodiment, for the VH, the subgroup is subgroup III as in Kabat et al., supra.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences: EVQLVESGGGLVQPGGSLRLSCAAS (SEQ ID NO:74)-H1-WVRQAPGKGLEWV (SEQ ID NO:75)-H2-RFTISRDNSKNTLYLQMNSLRAEDTAVYYCAR (SEQ ID NO:76)-H3-WGQGTL (SEQ ID NO:77), where N is any amino acid.

A “VL subgroup I consensus framework” comprises the consensus sequence obtained from the amino acid sequences in variable light kappa subgroup I of Kabat et al., supra. In one embodiment, the VL subgroup I consensus framework amino acid sequence comprises at least a portion or all of each of the following sequences: DIQMTQSPSSLSASVGDRVTITC (SEQ ID NO:78)-L1-WYQQKPGKAPKLLIY (SEQ ID NO:79)-L2-GVPSRFSGSGSGTDFTLTISSLQPEDFATYYC (SEQ ID NO:80)-L3-FGQGTKVEIKR (SEQ ID NO:81), where N is any amino acid.

An “amino-acid modification” at a specified position, e.g., of the Fc region, refers to the substitution or deletion of the specified residue, or the insertion of at least one amino acid residue adjacent to the specified residue. By insertion “adjacent to” a specified residue is meant insertion within one to two residues thereof. The insertion may be N-terminal or C-terminal to the specified residue. The preferred amino acid modification herein is a substitution.

As used herein, the term “immunoadhesin” designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity that is other than the antigen recognition and binding site of an antibody (i.e., is “heterologous”), and an immunoglobulin constant-domain sequence. The adhesin part of an immunoadhesin molecule typically is a contiguous amino acid sequence comprising at least the binding site of a receptor or a ligand. The immunoglobulin constant-domain sequence in the immunoadhesin can be obtained from any immunoglobulin, such as IgG-1, IgG-2, IgG-3, or IgG-4 subtypes, IgA (including IgA-1 and IgA-2), IgE, IgD, or IgM. For example, useful immunoadhesins as second medicaments herein include polypeptides that comprise the BLyS-binding portions of a BLyS receptor without the transmembrane or cytoplasmic sequences of the BLyS receptor. In one embodiment, the extracellular domain of BR3, TACI, or BCMA is fused to a constant domain of an immunoglobulin sequence.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

“Percent (%) amino acid sequence identity” and “homology” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN™ (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, authored by Genentech, Inc. The source code of ALIGN-2 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described in the immediately preceding paragraph using the ALIGN-2 computer program.

A “disorder” is any condition that would benefit from treatment with an antibody or method of the invention, regardless of mechanism, but including inhibiting tyrosine phosphorylation of IGF-1R. This condition includes, but is not limited to, a medical condition mediated by elevated expression or activity of IGF-1R, and/or an illness related to an over-expression and/or an abnormal activation of IGF-1R and/or EGFR, and/or related to a hyperactivation of the transduction pathway of the signal mediated by the interaction of IGF-I or IGF-II with IGF-1R and/or of EGF with EGFR. This includes chronic and acute disorders such as those pathological conditions that predispose the mammal to the disorder in question.

Non-limiting examples of disorders to be treated herein include malignant and benign tumors, autoimmune disorders, non-leukemias and lymphoid malignancies, neuronal, glial, astrocytal, hypothalamic and other glandular, macrophagal, epithelial, stromal and blastocoelic disorders, diarrhea associated with metastatic carcinoid, vasoactive intestinal peptide-secreting tumors, gigantism, atherosclerosis, smooth muscle restenosis of blood vessels, inappropriate microvascular proliferation, bone and cartilage-related disorders such as skeletal disorders or a craniosynostosis disorder, and inflammatory, immunologic and angiogenesis-related disorders. Skeletal disorders include herein, e.g., a skeletal dysplasia, thanatophoric dysplasia (TD), hypochondroplasia, severe achondroplasia with developmental delay, and acanthuses nigricans (SADDAN) dysplasia, preferably achondroplasia. Craniosynostosis disorders include, e.g., primary Muenke coronal craniosynostosis or Crouzon syndrome with acanthuses nigricans.

Examples of IGF-1R-dependent disorders, one of the conditions treatable herein, include benign and malignant neoplasms, the latter including carcinomas such as breast cancer and prostate cancer, leukemia, malignant melanoma, sarcomas such as Ewing's sarcoma, neuroectodermal tumors, gliomas, myeloproliferative and lymphoproliferative diseases, acromegaly, arteriosclerosis, psoriasis, restenosis following coronary angioplasty, restenosis of the coronary arteries after vascular surgery, certain endocrine disorders such as acromegaly, metabolic disorders such as syndrome X, and also virus-infected cells and self-reactive lymphocytes (T-cells), when these cells are dependent on IGF-1R for their survival.

Also included herein are disorders associated with ligand-dependent activation of a receptor protein tyrosine kinase (RPTK), or with constitutive activation of a RPTK, such as one involving a malignant-cell proliferative disease associated with abnormal RPTK, e.g., a hematopoietic malignancy, including multiple myeloma, as well as treatment of solid tumors, such as mammary, colon, cervical, bladder, colorectal, chondrosarcoma, or osteosarcoma.

Specific examples of disorders to be treated herein include cancer, a thymus disorder, a T-cell-mediated autoimmune disease, an endocrinological disorder, ischemia, and a neurodegenerative disorder. A preferred thymus disorder is thymoma or thyroiditis; a preferred T-cell-mediated autoimmune disease is multiple sclerosis, rheumatoid arthritis, systemic lupus erythematosus (SLE), Grave's disease, Hashimoto's thyroiditis, psoriasis, myasthenia gravis, auto-immune thyroiditis, or Bechet's disease; a preferred endocrinological disorder is Type II diabetes, hyperthyroidism, hypothyroidism, thyroiditis, hyperadrenocorticism, or hypoadrenocorticism; a preferred ischemia is post-cardiac ischemia; and a preferred neurodegenerative disorder is Alzheimer's disease.

The preferred cancers to be treated herein include prostate cancer such as hormone-resistant prostate cancer, osteosarcoma, breast cancer, endometrial cancer, lung cancer such as non-small cell lung carcinoma, ovarian cancer, colorectal cancer, pediatric cancer, pancreatic cancer, bone cancer, bone or soft tissue sarcoma or myeloma, bladder cancer, primary peritoneal carcinoma, fallopian tube carcinoma, Wilm's cancer, benign prostatic hyperplasia, cervical cancer, squamous cell carcinoma, head and neck cancer, synovial sarcoma, liquid tumors, multiple myeloma, cervical cancer, kidney cancer, liver cancer, synovial carcinoma, and pancreatic cancer. Preferred liquid tumors herein are acute lymphocytic leukemia (ALL) or chronic milogenic leukemia (CML); a preferred liver cancer is hepatoma, hepatocellular carcinoma, cholangiocarcinoma, angiosarcoma, hemangiosarcoma, or hepatoblastoma. More preferred cancers to be treated are multiple myeloma, breast cancer, colon cancer, ovarian cancer, osteosarcoma, cervical cancer, prostate cancer, lung cancer, kidney cancer, liver cancer, synovial carcinoma, and pancreatic cancer. Still more preferred are multiple myeloma, breast cancer, ovarian cancer, colorectal cancer, lung cancer, and prostate cancer.

The terms “cell-proliferative disorder” and “proliferative disorder” refer to disorders that are associated with some degree of abnormal cell proliferation. In one embodiment, the cell-proliferative disorder is cancer, acromegaly, retinal neovascularization, or psoriasis.

“IGF-1R-activated cell proliferation” refers to a proliferative disorder activated by IGF-1R. Preferably, such cell proliferation is acromegaly, retinal neovascularization, psoriasis, or cancer.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “aging” refers to the accumulation of diverse adverse changes that increase the risk of death as a subject gets increasingly old, after maturation. These changes can be attributed to development, genetic defects, the environment, disease, and the inborn aging process. The chance of death at a given age serves as a measure of the number of accumulated changes, that is, of physiologic age, and the rate of change of this measure is the rate of aging. Harman, Ann. N.Y. Acad. Sci., 854:1-7 (1998). Aging is manifested, for example, by such conditions as atherosclerosis, peripheral vascular disease, coronary artery disease, osteoporosis, type 2 diabetes, dementia, arthritis, stroke, a cardiovascular disease, high blood pressure, Alzheimer's disease, senescence, and cancer. Aging as defined herein may be by any mechanism, including, for example, aging based on inhibition of interleukin-6 inflammation through regulation of cholesterol metabolism, isoprenoid depletion, and/or inhibition of the signal-transduction pathway. Aging is accompanied by a progressive decrease in physiological capacity, but the rate of physiological decline varies from organ to organ and from individual to individual. The physiological decline results in a reduced ability to respond adaptively to environmental stimuli, and increased susceptibility and vulnerability to disease. Preferably, the antibodies herein inhibit aging for at least a subpopulation of mature (post-puberty) adult subjects.

The most widely accepted method of measuring the rate of aging is by reference to the average or the maximum lifespan. If a drug treatment achieves a statistically significant improvement in average or maximum lifespan in the treatment group over the control group, then it is inferred that the rate of aging was retarded in the treatment group. Similarly, one can compare long-term survival between the two groups. The term average (median) “lifespan” is the chronological age to which 50% of a given population survive. The maximum lifespan potential is the maximum age achievable by a member of the population. As a practical matter, it is estimated as the age reached by the longest lived member (or former member) of the population. The (average) life expectancy is the number of remaining years that an individual of a given age can expect to live, based on the average remaining life spans of a group of matched individuals.

Preferably, the antibodies of the present invention have the effect of increasing the average lifespan and/or the maximum lifespan for at least a subpopulation of mature (post-puberty) adult subjects. This subpopulation may be defined by sex and/or age. If defined in part by age, then it may be defined by a minimum age (e.g., at least 30, at least 40, at least 50, at least 55, at least 60, at least 65, at least 70, at least 75, at least 80, at least 90, etc.) or by a maximum age (not more than 40, not more than 50, not more than 55, not more than 60, not more than 65, not more than 70, not more than 75, not more than 80, not more than 90, not more than 100, etc.), or by a rational combination of a minimum age and a maximum age so as to define a preferred close-ended age range, e.g., 55-75. The subpopulation may additionally be defined by race, e.g., Caucasian, African, or Asian, and/or by ethnic group, and/or by place of residence (e.g., North America, Europe). The subpopulation may additionally be defined by non-age risk factors for age-associated diseases, e.g., by blood pressure, body mass index, etc. Preferably, the subpopulation in which an antibody of the present invention is reasonably expected to be effective is large, e.g., in the United States, preferably at least 100,000 individuals, more preferably at least 1,000,000 individuals, still more preferably at least 10,000,000, even more preferably at least 20,000,000, most preferably at least 40,000,000.

These expectancies can be calculated for the entire age cohort, or broken down by sex, race, country of residence, etc. Individuals who live longer than expected can be said, after the fact, to have biologically aged more slowly than their peers. Since lifespan studies are extremely time-consuming, scientists have sought to identify biological markers (biomarkers) of biological aging, that is, characteristics that can be measured while the subjects are still alive, which correlate to lifespan. These biological markers can be used to calculate a “biological age” (or “physiological age”); it is the chronological age at which an average member of the population (or relevant subpopulation) would have the same value of a biomarker of biological aging (or the same value of a composite measure of biomarkers of biological aging) as does the subject. This is the definition that is used herein.

The effect of aging varies from system to system, organ to organ, etc. For example, between ages 30 and 70 years, nerve conduction velocity decreases by only about 10%, but renal function decreases on average by nearly 40%. Thus, there is not just one biological age for a subject. By a suitable choice of biomarker, one may obtain a whole organism, or a system-, organ- or tissue-specific measure of biological aging, e.g., one can say that a person has the nervous system of a 30-year-old but the renal system of a 60-year old. Biomarkers may measure changes at the molecular, cellular, tissue, organ, system, or whole organism levels.

Generally speaking, in the absence of some form of intervention (drugs, diet, exercise, etc.), biological ages will increase with time. The antibodies of the present invention preferably reduce the time rate of change of a biological age of the subject. The term “a biological age” could refer to the overall biological age of the subject, to the biological age of a particular system, organ, or tissue of that subject, or to some combination of the foregoing. More preferably, the antibodies of the present invention cannot only reduce the rate of increase of a biological age of the subject, but can actually reduce a biological age of the subject.

A simple biologic marker (biomarker) is a single biochemical, cellular, structural, or functional indicator of an event in a biologic system or sample. A composite biomarker is a mathematical combination of two or more simple biomarkers. (Chronological age may be one of the components of a composite biomarker.) A plausible biomarker of biological age would be a biomarker that shows a cross-sectional and/or longitudinal correlation with chronological age. Nakamura suggests that it is desirable that a biomarker show (a) significant cross-sectional correlation with chronological age, (b) significant longitudinal change in the same direction as the cross-sectional correlation, (c) significant stability of individual differences, and (d) rate of age-related change proportional to differences in life span among related species. See Nakamura, Exp Gerontol., 2 9(2):151-77 (1994), using desiderata (a)-(c). A superior biomarker of biological age would be a better predictor of lifespan than is chronological age (preferably for a chronological age at which 90% of the population is still alive).

The biomarker preferably also satisfies one or more of the following desiderata: a statistically significant age-related change is apparent in humans after a period of at most a few years; not affected dramatically by physical conditioning (e.g., exercise), diet, and drug therapy (unless it is possible to discount these confounding influences, e.g., by reference to a second marker that measures them); can be tested repeatedly without harming the subject; works in lab animals as well as humans; simple and inexpensive to use; does not alter the result of subsequent tests for other biomarkers if it is to be used in conjunction with them; and monitors a basic mode of action that underlies the aging process, not the effects of disease.

A biomarker of aging may be used to predict, instead of lifespan, the “Healthy Active Life Expectancy” (HALE) or the “Quality Adjusted Life Years” (QALY), or a similar measure that takes into account the quality of life before death as well as the time of death itself. For HALE, see Jagger, in Outcomes Assessment for Healthcare in Elderly People, 67-76 (Farrand Press: 1997). For QALY, see Rosser R M. A health index and output measure, in Stewart S R and Rosser R M (eds.) Quality of Life: Assessment and Application (Lancaster: MTP, 1988). A biomarker of aging may be used to predict, instead of lifespan, the timing and/or severity of a change in one or more age-related phenotypes. A biomarker of aging may be used to estimate, rather than overall biological age for a subject, a biological age for a specific body system or organ. The determination of the biological age of the liver or kidney, and the inhibition of biological aging of the liver or kidney, are of particular interest, in one embodiment.

Body systems include the nervous system (including the brain, the sensory organs, and the sense receptors of the skin), the cardiovascular system (including the heart, the red blood cells, and the reticuloendothelial system), the respiratory system, the gastrointestinal system, the endocrine system (pituitary, thyroid, parathyroid and adrenal glands, gonads, pancreas, and parganglia), the musculoskeletal system, the urinary system (kidneys, bladder, ureters, and urethra), the reproductive system, and the immune system (bone marrow, thymus, lymph nodes, spleen, lymphoid tissue, white blood cells, and immunoglobulins). A biomarker may be useful in estimating the biological age of a system because the biomarker is a chemical produced by that system, because it is a chemical whose activity is primarily exerted within that system, because it is indicative of the morphological character or functional activity of that system, etc. A given biomarker may be thus associated with more than one system. In a like manner, a biomarker may be associated with the biological age, and hence the state, of a particular organ or tissue.

The prediction of lifespan, or of duration of system or organ function at or above a particular desired level, may require knowledge of the value of at least one biomarker of aging at two or more times, adequately spaced, rather than of the value at a single time. See McClearn, Exp. Gerontol., 32:87-94 (1997). See also WO 2005/005668 for further disclosure.

An “autoimmune disorder” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disease, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways may participate to different degrees in the pathology of autoimmune diseases.

“Autoimmune disorder” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, systemic lupus erythematosus (SLE), rheumatoid arthritis (RA), polymyositis, etc.). Preferred such diseases include autoimmune rheumatologic disorders (such as, for example, RA, Sjögren's syndrome, scleroderma, lupus such as SLE and lupus nephritis, polymyositis-dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, ulcerative colitis, ANCA-associated vasculitis, lupus, multiple sclerosis, Sjögren's syndrome, Graves' disease, IDDM, pernicious anemia, thyroiditis, and glomerulonephritis.

Specific examples of other autoimmune disorders as defined herein, which in some cases encompass those listed above, include, but are not limited to, arthritis (acute and chronic, RA including juvenile-onset RA and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, guttate psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN (RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, agranulocytosis, vasculitides (including large-vessel vasculitis such as polymyalgia rheumatica and giant-cell (Takayasu's) arteritis, medium-vessel vasculitis such as Kawasaki's disease and polyarteritis nodosa/periarteritis nodosa, immunovasculitis, CNS vasculitis, cutaneous vasculitis, hypersensitivity vasculitis, necrotizing vasculitis such as fibrinoid necrotizing vasculitis and systemic necrotizing vasculitis, ANCA-negative vasculitis, and ANCA-associated vasculitis such as Churg-Strauss syndrome (CSS), Wegener's granulomatosis, and microscopic polyangiitis), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behçet's disease/syndrome, Castleman's syndrome, Goodpasture's syndrome, Reynaud's syndrome, Sjögren's syndrome, Stevens-Johnson syndrome, pemphigoid or pemphigus such as pemphigoid bullous, cicatricial (mucous membrane) pemphigoid, skin pemphigoid, pemphigus vulgaris, paraneoplastic pemphigus, pemphigus foliaceus, pemphigus mucus-membrane pemphigoid, and pemphigus erythematosus, epidermolysis bullosa acquisita, ocular inflammation, preferably allergic ocular inflammation such as allergic conjunctivis, linear IgA bullous disease, autoimmune-induced conjunctival inflammation, autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, Grave's eye disease (opthalmopathy or thyroid-associated opthalmopathy), polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs. NSIP, Guillain-Barré syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, keratitis such as Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, trypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic fasciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis obiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermatogenesis, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica (sympathetic ophthalmitis), neonatal ophthalmitis, optic neuritis, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

As used herein, “treatment” refers to clinical intervention designed to alter the natural course of the individual or cell being treated, and can be performed either for prophylaxis or during the course of clinical pathology. Desirable effects of treatment include prevention of occurrence or recurrence of disease, alleviation of symptoms, diminishing of any direct or indirect pathological consequences of the disease, prevention of metastasis, decreasing of the rate of disease progression, amelioration or palliation of the disease state, and remission or improved prognosis. In some embodiments, the antibodies of the invention are used to delay development of a disease or disorder. A subject is successfully “treated,” for example, for cancer, aging, or an autoimmune disorder if, after receiving a therapeutic amount of an antibody of the invention according to the methods herein, the subject shows observable and/or measurable reduction in or absence of one or more signs and symptoms of the particular disease. In the context of cancer, the term “treating” herein includes treating or inhibiting tumor formation, primary tumors, tumor progression, or tumor metastasis. Tumor progression includes the progression of transitional cell carcinoma, osteo- or chondrosarcoma, or multiple myeloma.

In one embodiment of successful treatment, the antibody induces a major clinical response in a subject with RA. For purposes herein, a “major clinical response” is defined as achieving an American College of Rheumatology 70 response (ACR 70) for six consecutive months. ACR response scores are categorized as ACR 20, ACR 50 and ACR 70, with ACR 70 being the highest level of sign and symptom control in this evaluation system. ACR response scores measure improvement in RA disease activity, including joint swelling and tenderness, pain, level of disability, and overall patient and physician assessment. An example of a different type of antibody that induces a major clinical response as recognized by the FDA and as defined herein is etanercept (ENBREL®).

An “effective amount” refers to an amount effective, at dosages and for periods of time necessary, to achieve the desired therapeutic or prophylactic result.

A “therapeutically effective amount” of a medicament herein may vary according to factors such as the disease state, age, sex, and weight of the individual, and the ability of the medicament, e.g., antibody, to elicit a desired response in the individual. A therapeutically effective amount is also one in which any toxic or detrimental effects of the drug in question, e.g., antibody, are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “cytotoxic agent” as used herein refers to a substance that inhibits or prevents the function of cells and/or causes destruction of cells. The term includes radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), and toxins such as small-molecule toxins or enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omegall (see, e.g., Nicolaou et al., Angew. Chem. Intl. Ed. Engl., 33: 183-186 (1994)); dynemicin, including dynemicin A; an esperamicin; as well as neocarzinostatin chromophore and related chromoprotein enediyne antibiotic chromophores), other antibiotics such as aclacinomycin, actinomycin, authramycin, azaserine, bleomycin, cactinomycin, carabicin, caminomycin, carzinophilin, chromomycin, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), and deoxydoxorubicin), epirubicin, esorubicin, idarubicin, marcellomycin, mitomycins such as mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids, or derivatives of any of the above.

A “growth-inhibitory agent” refers to a compound or composition that inhibits growth of a cell, which growth depends on receptor activation either in vitro or in vivo. Thus, the growth-inhibitory agent includes one that significantly reduces the percentage of receptor-dependent cells in S phase. Examples of growth-inhibitory agents include agents that block cell-cycle progression (at a place other than S phase), such as agents that induce G1 arrest and M-phase arrest. Classical M-phase blockers include the vincas and vinca alkaloids (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA-alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anti-cancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb).

The term “cytokine” is a generic term for proteins released by one cell population that act on another cell as intercellular mediators. Examples of such cytokines are lymphokines, monokines, interleukins (ILs) such as IL-1, IL-1α, IL-2, IL-3, IL-4, IL-5, IL-6, IL-7, IL-8, IL-9, IL-11, IL-12, and IL-15, including PROLEUKIN® rIL-2, a tumor-necrosis factor such as TNF-α or TNF-β, and other polypeptide factors including leukocyte-inhibitory factor (LIF) and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

“Tumor-necrosis factor-alpha”, TNF-alpha”, or “TNF-α” refers to a human TNF-α molecule comprising the amino acid sequence of Pennica et al., Nature, 312:721 (1984) or Aggarwal et al., JBC, 260:2345 (1985).

A “TNF antagonist” or “TNF inhibitor” is defined herein as a molecule that decreases, blocks, inhibits, abrogates, or otherwise interferes with TNF-α activity in vitro, in situ, and/or preferably in vivo. Such an agent inhibits, to some extent, a biological function of TNF-α, generally through binding to TNF-α and neutralizing its activity. A suitable TNF antagonist can also decrease block, abrogate, interfere, prevent, and/or inhibit TNF RNA, DNA, or protein synthesis, TNF-α release, TNF-α receptor signaling, membrane TNF-α cleavage, TNF-α activity, and TNF-α production, and/or synthesis. Such TNF antagonists include, but are not limited to, anti-TNF-α antibodies, antigen-binding fragments thereof, specified mutants or domains thereof that bind specifically to TNF-α that, upon binding to TNF-α, destroy or deplete cells expressing the TNF-α in a mammal and/or interfere with one or more functions of those cells, a soluble TNF receptor (e.g., p55, p70 or p85) or fragment, fusion polypeptides thereof, a small-molecule TNF antagonist, e.g., TNF binding protein I or II (TBP-I or TBP-II), nerelimonmab, CDP-571, infliximab (REMICADE®), etanercept (ENBREL™), adalimulab (HUMIRA™), CDP-571, CDP-870, afelimomab, lenercept, and the like), antigen-binding fragments thereof, and receptor molecules that bind specifically to TNF-α; compounds that prevent and/or inhibit TNF-α synthesis, TNF-α release, or its action on target cells, such as thalidomide, tenidap, phosphodiesterase inhibitors (e.g, pentoxifylline and rolipram), A2b adenosine receptor agonists, and A2b adenosine receptor enhancers; compounds that prevent and/or inhibit TNFα receptor signaling, such as mitogen-activated protein (MAP) kinase inhibitors; compounds that block and/or inhibit membrane TNF-α cleavage, such as metalloproteinase inhibitors; compounds that block and/or inhibit TNF-α activity, such as angiotensin-converting enzyme (ACE) inhibitors (e.g., captopril); and compounds that block and/or inhibit TNF-α production and/or synthesis, such as MAP kinase inhibitors. The preferred antagonist comprises an antibody or an immunoadhesin.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF), granulocyte-macrophage-CSF (GM-CSF), and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells, and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1, etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

The term “immunosuppressive agent” refers to a substance that acts to suppress or mask the immune system of the subject being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); trocade (Ro32-355); a peripheral sigma receptor antagonist such as ISR-31747; alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, rimexolone, and dexamethasone; dihydrofolate reductase inhibitors such as methotrexate (oral or subcutaneous); anti-malarial agents such as chloroquine and hydroxychloroquine; sulfasalazine; leflunomide; cytokine release inhibitors such as SB-210396 and SB-217969 monoclonal antibodies and a MHC II antagonist such as ZD2315; a PG1 receptor antagonist such as ZD4953; a VLA4 adhesion blocker such as ZD7349; anti-cytokine or anti-cytokine receptor antibodies including anti-interferon-alpha, -beta, or -gamma antibodies, anti-TNF-α antibodies (infliximab (REMICADE®) or adalimumab), anti-TNF-α immunoadhesin (etanercept), anti-TNF-beta antibodies, interleukin-1 (IL-1) blockers such as recombinant HuIL-1Ra and IL-1B inhibitor, anti-interleukin-2 (IL-2) antibodies and anti-IL-2 receptor antibodies; IL-2 fusion toxin; anti-L3T4 antibodies; leflunomide; heterologous anti-lymphocyte globulin; OPC-14597; NISV (immune response modifier); an essential fatty acid such as gammalinolenic acid or eicosapentaenoic acid; CD-4 blockers and pan-T antibodies, preferably anti-CD3 or anti-CD4/CD4a antibodies; co-stimulatory modifier (e.g., CTLA4-Fc fusion, also known as ABATACEPT™; anti-interleukin-6 (IL-6) receptor antibodies and antagonists; anti-LFA-1 antibodies, including anti-CD11a and anti-CD18 antibodies; soluble peptide containing a LFA-3-binding domain (WO 1990/08187); streptokinase; IL-10; transforming growth factor-beta (TGF-beta); streptodomase; RNA or DNA from the host; FK506; RS-61443; enlimomab; CDP-855; PNP inhibitor; CH-3298; GW353430; 4162W94, chlorambucil; deoxyspergualin; rapamycin; T-cell receptor (U.S. Pat. No. 5,114,721); T-cell receptor fragments (Offner et al., Science, 251: 430-2 (1991); WO 1990/11294; Janeway, Nature, 341: 482-483 (1989); and WO 1991/01133); BAFF antagonists such as BAFF antibodies and BR3 antibodies; zTNF4 antagonists (Mackay and Mackay, Trends Immunol., 23:113-5 (2002)); biologic agents that interfere with T-cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate (MTX).

“Disease-modifying anti-rheumatic drugs” or “DMARDs” include, e.g., chloroquine, hydroxycloroquine, myocrisin, auranofin, sulfasalazine, methotrexate, leflunomide, etanercept, infliximab (and oral and subcutaneous MTX), azathioprine, D-penicilamine, gold salts (oral), gold salts (intramuscular), minocycline, cyclosporine, e.g., cyclosporine A and topical cyclosporine, staphylococcal protein A (Goodyear and Silverman, J. Exp. Med., 197:1125-39 (2003)), including salts and derivatives thereof, etc.

“Non-steroidal anti-inflammatory drugs” or “NSAIDs” include, e.g., aspirin, acetylsalicylic acid, ibuprofen, ibuprofen retard, fenoprofen, piroxicam, flurbiprofen, naproxen, ketoprofen, naproxen, tenoxicam, benorylate, diclofenac, naproxen, nabumetone, indomethacin, ketoprofen, mefenamic acid, diclofenac, fenbufen, azapropazone, acemetacin, tiaprofenic acid, indomethacin, sulindac, tolmetin, phenylbutazone, diclofenac, diclofenac retard, cyclooxygenase (COX)-2 inhibitors such as GR 253035, MK966, celecoxib (CELEBREX®; 4-(5-(4-methylphenyl)-3-(trifluoromethyl)-1H-pyrazol-1-yl)benzenesulfonamide), valdecoxib (BEXTRA®), and meloxicam (MOBIC®), including salts and derivatives thereof, etc. Preferred are aspirin, naproxen, ibuprofen, indomethacin, or tolmetin. NSAIDs are optionally used with an analgesic, e.g., codenine, tramadol, and/or dihydrocodinine, or narcotic, e.g., morphine.

A “B cell” is a lymphocyte that matures within the bone marrow, and includes a naïve B cell, memory B cell, or effector B cell (plasma cells). The B cell herein may be normal or non-malignant.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85, and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2×5, HLA-DOB, CXCR5, FCER2, BR3, Btig, NAG14, SLGC16270, FcRH1, IRTA2, ATWD578, FcRH3, IRTA1, FcRH6, BCMA, and 239287. The preferred B-cell surface marker is preferentially expressed on B cells compared to other non-B-cell tissues of a mammal and may be expressed on both precursor and mature B cells. The most preferred such markers are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35.” The CD20 antigen is described in Clark et al., Proc. Natl. Acad. Sci. (USA), 82:1766 (1985), for example.

The “CD22” antigen, or “CD22,” also known as BL-CAM or Lyb8, is a type-1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al., J. Exp. Med., 173:137 (1991) and Wilson et al., J. Immunol., 150:5013 (1993).

An “antibody that binds to a B-cell surface marker” is a molecule that, upon binding to a B-cell surface marker, destroys or depletes B cells in a mammal and/or interferes with one or more B-cell functions, e.g., by reducing or preventing a humoral response elicited by the B cell. The antibody preferably is able to deplete B cells (i.e., reduce circulating B-cell levels) in a mammal treated therewith. Such depletion may be achieved via various mechanisms such as ADCC and/or CDC, inhibition of B-cell proliferation, and/or induction of B-cell death (e.g., via apoptosis).

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al., Blood, 69(2):584-591 (1987)) and variants thereof including “framework-patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HUMAX-CD20™, a fully human, high-affinity antibody targeted at the CD20 molecule in the cell membrane of B-cells (Genmab, Denmark; see, for example, Glennie and van de Winkel, Drug Discovery Today, 8: 503-510 (2003) and Cragg et al., Blood, 101: 1045-1052 (2003)); the human monoclonal antibodies set forth in WO 2004/035607 (Teeling et al.); AME-133™ antibodies (Applied Molecular Evolution); GA101 (GlycArt; US 2005/0123546); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), and HUMAX-CD20 human CD20 antibody (Genmab).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20.

Purely for the purposes herein and unless indicated otherwise, a “humanized 2H7” refers to a humanized CD20 antibody, or an antigen-binding fragment thereof, wherein the antibody is effective to deplete primate B cells in vivo. The antibody includes those set forth in US 2006/0062787 and the figures thereof, and including version 114, the sequences of which are provided in US 2006/0188495. See also US 2006/0034835 and US 2006/0024300. In a summary of various preferred embodiments of the invention, the V region of variants based on 2H7 version 16 as disclosed in US 2006/0062787 will have the amino acid sequences of v16 except at the positions of amino acid substitutions that are indicated in the table below. Unless otherwise indicated, the 2H7 variants will have the same L chain as that of v16.

| 2H7 | Heavy chain | Light chain | |
|---|---|---|---|
| version | (V_{H}) changes | (V_{L}) changes | Fc changes |
| 16 | | | — |
| A | — | — | S298A, E333A, K334A |
| B | N100A | M32L | |
| C | N100A | M32L | S298A, E333A, K334A |
| D | D56A, | S92A | |
| | N100A | | |
| E | D56A, | M32L, S92A | S298A, E333A, K334A |
| | N100A | | |
| F | D56A, | M32L, S92A | S298A, E333A, K334A, |
| | N100A | | E356D, M358L |
| G | D56A, | M32L, S92A | S298A, K334A, K322A |
| | N100A | | |
| H | D56A, | M32L, S92A | S298A, E333A, K334A, |
| | N100A | | K326A |
| I | D56A, | M32L, S92A | S298A, E333A, K334A, |
| | N100A | | K326A, N434W |
| J | — | — | K334L |

One preferred humanized 2H7 is an intact antibody or antibody fragment having the sequence of version 16. Another preferred humanized 2H7 is any of the other versions shown above, including version E.

“BAFF antagonists” are any molecules that block the activity of BAFF or BR3. They include immunoadhesins comprising a portion of BR3, TACI, or BCMA that binds BAFF, or variants thereof that bind BAFF. In other aspects, the BAFF antagonist is a BAFF antibody. A “BAFF antibody” is an antibody that binds BAFF, and preferably binds BAFF within a region of human BAFF comprising residues 162-275 of human BAFF. In another aspect, the BAFF antagonist is a BR3 antibody. A “BR3 antibody” is an antibody that binds BR3, and preferably binds BR3 within a region of human BR3 comprising residues 23-38 of human BR3. The sequences of human BAFF and human BR3 are found, e.g., in US 2006/0062787. Other examples of BAFF-binding polypeptides or BAFF antibodies can be found in, e.g., WO 2002/092620, WO 2003/014294, Gordon et al., Biochemistry, 42(20):5977-83 (2003), Kelley et al., J. Biol. Chem., 279:16727-35 (2004), WO 1998/18921, WO 2001/12812, WO 2000/68378 and WO 2000/40716.

An “insulin-resistance-treating agent” or “hypoglycemic agent” (used interchangeably herein) is an agent that is used to treat an insulin-resistant disorder, such as, e.g., insulin (one or more different types of insulin), insulin mimetics, such as a small-molecule insulin, e.g., L-783,281, insulin analogs (e.g., LYSPRO™ (Eli Lilly Co.), Lys^{B28}insulin, Pro^{B29}insulin, or Asp^{B28}insulin or those described in, for example, U.S. Pat. Nos. 5,149,777 and 5,514,646), or physiologically active fragments thereof, insulin-related peptides (C-peptide, GLP-1, IGF-1, or IGF-1/IGFBP-3 complex), or analogs or fragments thereof, ergoset, pramlintide, leptin, BAY-27-9955, T-1095, a dickkopf protein such as dickkopf-5 (dkk-5), antagonists to insulin receptor tyrosine kinase inhibitor, TNF-α antagonists, a growth-hormone-releasing agent, amylin or antibodies to amylin, an insulin sensitizer, such as compounds of the glitazone family, including those described in U.S. Pat. No. 5,753,681, such as troglitazone, pioglitazone, englitazone, and related compounds, LINALOL™ alone or with Vitamin E (U.S. Pat. No. 6,187,333), and insulin-secretion enhancers, such as nateglinide (AY-4166), calcium (2S)-2-benzyl-3-(cis-hexahydro-2-isoindolinylcarbonyl)propionate dihydrate (mitiglinide, KAD-1229), repaglinide, and sulfonylurea drugs, for example, acetohexamide, chlorpropamide, tolazamide, tolbutamide, glyclopyramide, and its ammonium salt, glibenclamide, glibornuride, gliclazide, 1-butyl-3-metanilylurea, carbutamide, glipizide, gliquidone, glisoxepid, glybuthiazole, glibuzole, glyhexamide, glymidine, glypinamide, phenbutamide, tolcyclamide, glimepiride, etc., as well as biguanides (such as phenformin, metformin, buformin, etc.), and α-glucosidase inhibitors (such as acarbose, voglibose, miglitol, emiglitate, etc.), and such non-typical treatments as pancreatic transplant or autoimmune reagents.

As used herein, “insulin” refers to any and all substances having an insulin action, and exemplified by, for example, animal insulin extracted from bovine or porcine pancreas, semi-synthesized human insulin that is enzymatically synthesized from insulin extracted from porcine pancreas, and human insulin synthesized by genetic engineering techniques typically using E. coli or yeasts, etc. Further, insulin can include insulin-zinc complex containing about 0.45 to 0.9 (w/w) % of zinc, protamine-insulin-zinc produced from zinc chloride, protamine sulfate and insulin, etc. Insulin may be in the form of its fragments or derivatives, e.g., INS-1. Insulin may also include insulin-like substances, such as L83281 and insulin agonists. While insulin is available in a variety of types, such as super immediate-acting, immediate-acting, bimodal-acting, intermediate-acting, long-acting, etc., these types can be appropriately selected according to the patient's condition.

“Chronic” administration refers to administration of the medicament(s) in a continuous as opposed to acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers that are non-toxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; anti-oxidants including ascorbic acid; low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Mammal” refers to any animal classified as a mammal, including humans, domestic and farm animals, zoo, sports, or pet animals, such as dogs, horses, cats, cows, etc. The preferred mammal is human.

A “package insert” refers to instructions customarily included in commercial packages of medicaments that contain information about the indications, usage, dosage, administration, contraindications, other medicaments to be combined with the packaged product, and/or warnings concerning the use of such medicaments, etc.

A “medicament” is an active drug to treat the disorder in question or its symptoms or side effects.

The expression “not responsive to,” as it relates to the reaction of subjects or patients to one or more of the medicaments that were previously administered to them, describes those subjects or patients who, upon administration of such medicament(s), did not exhibit any or adequate signs of treatment of the disorder for which they were being treated, or they exhibited a clinically unacceptably high degree of toxicity to the medicament(s), or they did not maintain the signs of treatment after first being administered such medicament(s), with the word treatment being used in this context as defined herein. The phrase “not responsive” includes a description of those subjects who are resistant and/or refractory to the previously administered medication(s), and includes the situations in which a subject or patient has progressed while receiving the medicament(s) that he or she is being given, and in which a subject or patient has progressed within 12 months (for example, within six months) after completing a regimen involving the medicament(s) to which he or she is no longer responsive. The non-responsiveness to one or more medicaments thus includes subjects who continue to have active disease following previous or current treatment therewith. For instance, a patient may have active disease activity after about one to three months of therapy with the medicament(s) to which they are non-responsive. Such responsiveness may be assessed by a clinician skilled in treating the disorder in question.

For purposes of non-response to medicament(s), a subject who experiences “a clinically unacceptably high level of toxicity” from previous or current treatment with one or more medicaments experiences one or more negative side-effects or adverse events associated therewith that are considered by an experienced clinician to be significant, such as, for example, serious infections, congestive heart failure, demyelination (leading to multiple sclerosis), significant hypersensitivity, neuropathological events, high degrees of autoimmunity, a cancer such as endometrial cancer, non-Hodgkin's lymphoma, breast cancer, prostate cancer, lung cancer, ovarian cancer, or melanoma, tuberculosis (TB), etc.

By “reducing the risk of a negative side effect” is meant reducing the risk of a side effect resulting from therapy with the antibodies herein to a lower extent than the risk observed resulting from therapy to the same patient or another patient with a previously administered medicament. Such side effects include those set forth above regarding toxicity, and are preferably infection, cancer, heart failure, or demyelination.

“Overall survival” refers to the situation wherein a patient remains alive for a defined period of time, such as one year, five years, etc., e.g., from the time of diagnosis or treatment.

“Progression-free survival” refers to the situation wherein a patient remains alive, without the cancer getting worse.

An “objective response” refers to a measurable clinical response, including complete response (CR) or partial response (PR).

By “complete response” or “complete remission” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

A “sterile” formulation is aseptic or free from all living microorganisms and their spores.

The term “about” as used herein refers to the usual error range for the respective value readily known to the skilled person in this technical field.

### MODES FOR CARRYING OUT THE INVENTION

### Invention Aspects

Antibodies of the invention can be used to modulate one or more aspects of IGF-1R-associated effects, including but not limited to IGF-1R activation, downstream molecular signaling, cell proliferation, cell migration, cell survival, cell morphogenesis, and angiogenesis. Without being limited to any one theory, these effects can be modulated by any biologically relevant mechanism, including disruption of ligand (e.g., IGF-1), binding to IGF-1R, or receptor phosphorylation, and/or receptor multimerization.

In one embodiment, the invention contemplates an isolated anti-IGF-1R antibody comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid (10H5.vX or 9F2.vX or 2B4.vX or 10H5.v2 or 10H5.v48 or YW95.6, respectively);
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10) (10H5.vX or 9F2.vX; 2B4.vX, or 10H5.v10 or YW95.6, respectively);
(c) a HVR-L3 sequence comprising amino acids C1-C9, wherein C1-C9 is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid (10H5.vX or 9F2.vX/2B4.vX or 10H5.v10, or YW95.81 or YW95.6, respectively);
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid (10H5.vX or 9F2.vX or 2B4.vX or YW95.6 or YW95.87, respectively);
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25) (10H5.vX or 9F2.vX or 2B4.vX or YW95.6 or YW95.81, respectively); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32) (10H5.vX or 9F2.vX or 2B4.vX or YW95.87 or YW95.3 or YW95.6 or YW95.81, respectively).

In a preferred embodiment, SEQ ID NO:13 is QQYSNYPYT (SEQ ID NO:33), QQYKHYPYT (SEQ ID NO:34), QQYKKYPYT (SEQ ID NO:35), QQYKNYPYT (SEQ ID NO:36), QQYRIYPYT (SEQ ID NO:37), QQYKRYPYT (SEQ ID NO:38), QQYKSYPYT (SEQ ID NO:39), QQYRSYPYT (SEQ ID NO:40), or QQYSKYPYT (SEQ ID NO:41) (10H5.v2, 10H5.v9, 10H5.v16, 10H5.v32, 10H5.v39, 10H5.v46, 10H5.v48, 10H5.v96A, or 10H5.v96B, respectively).

In another preferred embodiment, the HVR-H3 is SEQ ID NO:26.

In another preferred aspect, the antibody comprises either:

(i) all of the HVR-L1 to HVR-L3 amino acid sequences of:

(a) SEQ ID NOS:1, 7, and 11, or

(b) SEQ ID NOS:2, 8, and 12, or

(c) SEQ ID NOS:3, 8, and 12, or

(d) SEQ ID NOS:6, 10, and 15, or

(e) SEQ ID NOS:4, 7, and 33, or

(f) SEQ ID NOS:1, 7, and 34, or

(g) SEQ ID NOS:1, 9, and 13, or

(h) SEQ ID NOS:1, 7, and 35, or

(i) SEQ ID NOS:1, 7, and 36, or

(j) SEQ ID NOS:1, 7, and 37, or

(k) SEQ ID NOS:1, 7, and 38, or

(l) SEQ ID NOS:5, 7, and 39, or

(m) SEQ ID NOS:1, 7, and 40, or

(n) SEQ ID NOS:1, 7, and 41; or

(ii) all of the HVR-H1 to HVR-H3 amino acid sequences of:

(a) SEQ ID NOS:16, 21, and 26, or

(b) SEQ ID NOS:17, 22, and 27, or

(c) SEQ ID NOS:18, 23, and 28, or

(d) SEQ ID NOS:19, 24, and 31

In a more preferred aspect, the antibody comprises all of SEQ ID NOS:1, 7, and 11, or all of SEQ ID NOS:16, 21, and 26.

In other still more preferred embodiments, the antibody comprises:

(i) all of the HVR-L1 to HVR-L3 amino acid sequences of:

(a) SEQ ID NOS:1, 7, and 11, or

(b) SEQ ID NOS:2, 8, and 12, or

(c) SEQ ID NOS:3, 8, and 12, or

(d) SEQ ID NOS:6, 10, and 15, or

(e) SEQ ID NOS:4, 7, and 33, or

(f) SEQ ID NOS:1, 7, and 34, or

(g) SEQ ID NOS:1, 9, and 13, or

(h) SEQ ID NOS:1, 7, and 35, or

(i) SEQ ID NOS:1, 7, and 36, or

(j) SEQ ID NOS:1, 7, and 37, or

(k) SEQ ID NOS:1, 7, and 38, or

(l) SEQ ID NOS:5, 7, and 39, or

(m) SEQ ID NOS:1, 7, and 40, or

(n) SEQ ID NOS:1, 7, and 41; and

(ii) all of the HVR-H1 to HVR-H3 amino acid sequences of:

(a) SEQ ID NOS:16, 21, and 26, or

(b) SEQ ID NOS:17, 22, and 27, or

(c) SEQ ID NOS:18, 23, and 28, or

(d) SEQ ID NOS:19, 24, and 31.

Still more preferably, the antibody comprises:

(i) all of the HVR-L1 to HVR-L3 amino acid sequences of:

(a) SEQ ID NOS:1, 7, and 11, or

(b) SEQ ID NOS:4, 7, and 33, or

(c) SEQ ID NOS:1, 7, and 34, or

(d) SEQ ID NOS:1, 9, and 13, or

(e) SEQ ID NOS:1, 7, and 35, or

(f) SEQ ID NOS:1, 7, and 36, or

(g) SEQ ID NOS:1, 7, and 37, or

(h) SEQ ID NOS:1, 7, and 38, or

(i) SEQ ID NOS:5, 7, and 39, or

(j) SEQ ID NOS:1, 7, and 40, or

(k) SEQ ID NOS:1, 7, and 41; and

(ii) all of the HVR-H1 to HVR-H3 amino acid sequences of SEQ ID NOS:16, 21, and 26.

Most preferably, the antibody comprises all of SEQ ID NOS:1, 7, and 11 and all of SEQ ID NOS:16, 21, and 26.

Antibodies in other embodiments comprise a human κ subgroup 1 consensus framework sequence, and/or they comprise a heavy-chain human subgroup III consensus framework sequence, wherein the framework sequence preferably comprises a substitution at position 71, 73, and/or 78. Such substitutions are preferably R71A, N73T, or N78A, or any combination thereof.

The antibodies of this invention preferably specifically bind to human IGF-1R and block the interaction of an IGF with IGF-1R, wherein said antibody is an antagonist of human IGF-1R and has an Fc region. The Fc region may be wild-type or an Fc variant as defined above, including those set forth in, for example, WO 2006/105338. Preferably, the IGF is IGF-I. Also, preferably the antibody does not bind specifically to (or cross-react with) the human insulin receptor. Also, preferably, the antibodies herein are humanized, affinity-matured anti-IGF-1R monoclonal antibodies of the IgG1 isotype. More preferably, such antibodies react with human and cynomolgus-monkey IGF-1R but not with rodent IGF-1R. Still more preferably, the antibodies herein block ligand binding (IGF to IGF-1R) and activation of IGF-1R. Still more preferably, the antibody herein down-regulates IGF-1R. Yet more preferably, the antibodies herein inhibit proliferation of multiple tumor cell lines in vitro. Still more preferred, such antibodies inhibit tumor growth in multiple xenografts.

Further, in one embodiment, the sequence of the light-chain variable region of the antibody has about 1-10 amino acid insertions, deletions, or substitutions from SEQ ID NO:53. More preferably, the sequence of its light-chain variable region comprises no more than about eight amino acid changes from SEQ ID NO:53. In another aspect, the sequence of the heavy-chain variable region of the antibody has about 1-10 amino acid insertions, deletions, or substitutions from SEQ ID NO:55. More preferably, the sequence of its heavy-chain variable region comprises no more than about eight amino acid changes from SEQ ID NO:55. In another aspect, the antibody has the above substitutions in both the light-chain and heavy-chain variable regions.

In another embodiment, the invention provides an anti-IGF-1R antibody having a light-chain variable domain comprising SEQ ID NO:44, 49, 53, 57, 64, 65, 66, 67, 68, 69, 70, 71, 72, or 73, or a heavy-chain variable domain comprising SEQ ID NO:47, 51, 55, or 61, or comprising both SEQ ID NOS:44 and 47, or both SEQ ID NOS:49 and 51, or both SEQ ID NOS:53 and 55, or both SEQ ID NOS:57 and 61, or both SEQ ID NOS: 64, 65, 66, 67, 68, 69, 70, 71, 72, or 73 and 55.

In a still further embodiment, the invention provides an IGF-1R antibody having a light-chain variable domain comprising SEQ ID NO:53 or a heavy-chain variable domain comprising SEQ ID NO:55 or having light-chain and heavy-chain variable domains comprising both SEQ ID NO:53 and SEQ ID NO:55.

Most preferred is an antibody having the full-length heavy-chain sequence of SEQ ID NO:90 and the full-length light-chain sequence of SEQ ID NO:91.

Further preferred antibodies bind IGF-1R with an affinity of at least about 10⁻¹² M, more preferably at least about 10⁻¹³ M. The antibodies also preferably are of the IgG isotype, such as IgG1, IgG2a, IgG2b, or IgG3, more preferably human IgG, and most preferably IgG1 or IgG2a (most preferably human IgG1 or IgG2a).

Another preferred antibody has a monovalent affinity to human IGF-1R that is about the same as or greater than the monovalent affinity to human IGF-1R of a murine antibody produced by a hybridoma cell line deposited under ATCC No. PTA-7007, PTA-7008, PTA-7009, PTA-7010, PTA-7011, PTA-7012, PTA-7013, PTA-7014, PTA-7015, PTA-7016, PTA-7017, PTA-7018, or PTA-7019 (murine hybridoma; Lymph nodes: IGFIR: 4373 (10H5.3.4), murine hybridoma; Lymph nodes: IGFIR: 4376 (1C2.8.1), murine hybridoma; Lymph nodes: IGFIR: 4364 (2B2.2.8), murine hybridoma; Lymph nodes: IGFIR: 4362 (2A7.5.1), murine hybridoma; Lymph nodes: IGFIR: 4363 (2B7.4.1), murine hybridoma; Lymph nodes: IGFIR: 4365 (3B9.4.1), murine hybridoma; Lymph nodes: IGFIR: 4366 (4D3.6.2), murine hybridoma; Lymph nodes: IGFIR: 4369 (6F10.1.1), murine hybridoma; Lymph nodes: IGFIR: 4367 (5e3.1.1), murine hybridoma; Lymph nodes: IGFIR: 4368 (6D2.6.1), murine hybridoma; Lymph nodes: IGFIR: 4375 (4D7.1.4), murine hybridoma; Lymph nodes: IGFIR: 4372 (9F2.6.2), or murine hybridoma; Lymph nodes: IGFIR: 4371 (9A11.3.1)), respectively.

As is well established in the art, binding affinity of a ligand to its receptor can be determined using any of a variety of assays, and expressed in terms of a variety of quantitative values. Accordingly, in one embodiment, the binding affinity is expressed as Kd values and reflects intrinsic binding affinity (e.g., with minimized avidity effects). Generally and preferably, binding affinity is measured in vitro, whether in a cell-free or cell-associated setting. Fold difference in binding affinity can be quantified in terms of the ratio of the monovalent binding affinity value of a humanized antibody (e.g., in Fab form) and the monovalent binding affinity value of a reference/comparator antibody (e.g., in Fab form) (e.g., a murine antibody having donor HVR sequences), wherein the binding affinity values are determined under similar assay conditions.

Thus, in one embodiment, the fold difference in binding affinity is determined as the ratio of the Kd values of the humanized antibody in Fab form and said reference/comparator Fab antibody. For example, in one embodiment, if an antibody of the invention (A) has an affinity that is “three-fold lower” than the affinity of a reference antibody (M), then if the Kd value for A is 3×, the Kd value of M would be 1×, and the ratio of Kd of A to Kd of M would be 3:1. Conversely, in one embodiment, if an antibody of the invention (C) has an affinity that is “three-fold greater” than the affinity of a reference antibody (R), then if the Kd value for C is 1×, the Kd value of R would be 3×, and the ratio of Kd of C to Kd of R would be 1:3. Any assays known in the art, including those described herein, can be used to obtain binding affinity measurements, including, for example, an optical biosensor that uses SPR (BIACORE® instrument technology), RIA, and ELISA. Preferably, the measurement is by optical biosensor or radioimmunoassay.

The antibodies herein are preferably chimeric or humanized, more preferably humanized, and still more preferably antibodies wherein at least a portion of their framework sequence is a human consensus framework sequence.

The antibodies of the present invention preferably have the native-sequence Fc region. However, they may further comprise other amino acid substitutions that, e.g., improve or reduce other Fc function or further improve the same Fc function, increase antigen-binding affinity, increase stability, alter glycosylation, or include allotypic variants. The antibodies may further comprise one or more amino acid substitutions in the Fc region that result in the antibody exhibiting one or more of the properties selected from increased FcγR binding, increased ADCC, increased CDC, decreased CDC, increased ADCC and CDC function, increased ADCC but decreased CDC function (e.g., to minimize infusion reaction), increased FcRn binding, and increased serum half life, as compared to the polypeptide and antibodies that have wild-type Fc. These activities can be measured by the methods described herein.

For additional amino acid alterations that improve Fc function, see, e.g., U.S. Pat. No. 6,737,056. Any of the antibodies of the present invention may further comprise at least one amino acid substitution in the Fc region that decreases CDC activity, for example, comprising at least the substitution K322A (see, e.g., U.S. Pat. No. 6,528,624). Mutations that improve ADCC and CDC include S298A/E333A/K334A also referred to herein as the triple Ala mutant. K334L increases binding to CD16. K322A results in reduced CDC activity. K326A or K326W enhances CDC activity. D265A results in reduced ADCC activity. Glycosylation variants that increase ADCC function are described, e.g., in WO 2003/035835. Stability variants are variants that show improved stability with respect to e.g., oxidation and deamidation. See also WO 2006/105338 for additional Fc variants.

Another embodiment herein is an anti-idiotype antibody that specifically binds the antibody herein.

### Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives, e.g., X63-Ag8-653 cells available from the ATCC, Manassas, Va. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as RIA or ELISA.

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal, e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-SEPHAROSE™ medium) or ion-exchange chromatography, hydroxylapatite chromatography, gel electrophoresis, dialysis, etc.

Certain murine hybridomas of this invention were deposited at the ATCC on Sep. 20, 2005 under the Deposit No. PTA-7007, PTA-7008, PTA-7009, PTA-7010, PTA-7011, PTA-7012, PTA-7013, PTA-7014, PTA-7015, PTA-7016, PTA-7017, PTA-7018, or PTA-7019. The invention also covers antibodies secreted by such hybridomas.

For production of recombinant monoclonal antibodies, DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, CHO cells, or myeloma cells that do not otherwise produce antibody protein, to synthesize monoclonal antibodies in the recombinant host cells. Review articles on recombinant expression in bacteria of DNA encoding the antibody include Skerra et al., Curr. Opinion in Immunol., 5:256-62 (1993) and Plückthun, Immunol. Revs., 130:151-88 (1992).

In a further embodiment, monoclonal antibodies or antibody fragments can be isolated from antibody phage libraries generated using the techniques described in McCafferty et al., Nature, 348:552-54 (1990). Clackson et al., Nature, 352:624-28 (1991) and Marks et al., J. Mol. Biol., 222:581-97 (1991) describe the isolation of murine and human antibodies, respectively, using phage libraries. High-affinity (nM range) human antibodies may be produced by chain shuffling (Marks et al., Bio/Technology, 10:779-83 (1992)), and by combinatorial infection and in vivo recombination as a strategy for constructing very large phage libraries (Waterhouse et al., Nuc. Acids. Res., 21:2265-66 (1993)). Thus, these techniques are viable alternatives to traditional monoclonal antibody hybridoma techniques for isolation of monoclonal antibodies.

The DNA encoding the antibody may be modified to produce chimeric or fusion antibody polypeptides, e.g., by substituting human heavy-chain and light-chain constant-domain (C_{H} and C_{L}) sequences for the homologous murine sequences (U.S. Pat. No. 4,816,567 and Morrison et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Humanized Antibodies

The present invention encompasses humanized antibodies. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting HVR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. Humanized antibodies are typically human antibodies in which some HVR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework for the humanized antibody (Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993).

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the HVR residues are directly and most substantially involved in influencing antigen binding.

The humanized antibody may be an antibody fragment, such as a Fab, that is optionally conjugated with another molecule to generate an immunoconjugate. Alternatively, the humanized antibody may be a full-length antibody, such as a full-length IgG1 antibody.

### Human Antibodies and Phage-Display Methodology

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-58 (1993); Bruggemann et al., Year in Immuno., 7:33 (1993); U.S. Pat. No. 5,545,806; U.S. Pat. No. 5,569,825; U.S. Pat. No. 5,591,669; U.S. Pat. No. 5,545,807; and WO 1997/17852.

Alternatively, phage-display technology (McCafferty et al., Nature, 348:552-553 (1990)) can be used to produce human antibodies and antibody fragments in vitro, from immunoglobulin variable (V) domain gene repertoires from unimmunized donors. In this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the IGF-1R-expressing cell. Phage display can be performed in a variety of formats, reviewed in, e.g., Johnson and Chiswell, Current Opinion in Structural Biology, 3:564-71 (1993). Several sources of V-gene segments can be used for phage display. Clackson et al., Nature, 352:624-28 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) isolated essentially following the techniques described by Marks et al., J. Mol. Biol., 222:581-97 (1991), or Griffith et al., EMBO J., 12:725-34 (1993). See also U.S. Pat. No. 5,565,332 and U.S. Pat. No. 5,573,905.

Human antibodies may also be generated by in vitro-activated B cells (see, e.g., U.S. Pat. No. 5,567,610 and U.S. Pat. No. 5,229,275).

### Antibody Fragments

In certain instances, using antibody fragments rather than whole antibodies is advantageous. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992) and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology, 10: 163-67 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See, e.g., WO 1993/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. The antibody fragment may also be a “linear antibody,” e.g., as described in U.S. Pat. No. 5,641,870 for example. Such linear antibody fragments may be monospecific or bispecific.

### Bispecific Antibodies

Bispecific antibodies are antibodies that have binding specificities for at least two different epitopes. Exemplary bispecific antibodies may bind to two different epitopes of the IGF-1R protein. Other such antibodies may combine a IGF-1R-binding site with a binding site for another protein. Alternatively, an anti-IGF-1R arm may be combined with an arm that binds to a triggering molecule on a leukocyte such as a T-cell receptor molecule (e.g., CD3), or Fc receptors for IgG (FcγR), such as FcγRI (CD64), FcγRII (CD32), and FcγRIII (CD16), or NKG2D or other NK-cell-activating ligand, so as to focus and localize cellular defense mechanisms to the IGF-1R-expressing cell. Bispecific antibodies may also be used to localize cytotoxic agents to cells that express IGF-1R. These antibodies possess a IGF-1R-binding arm and an arm that binds the cytotoxic agent (e.g. saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies).

WO 1996/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO 1998/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy-chain/light chain pairs, where the two chains have different specificities (Millstein et al., Nature, 305:537-39 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 1993/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy-chain constant domain, comprising at least part of the hinge, C_{H}2, and C_{H}3 regions. It is preferred to have the first heavy-chain constant region (C_{H}1) containing the site necessary for light-chain bonding, present in at least one of the fusions. DNAs encoding the immunoglobulin heavy-chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a preferred aspect of this approach, the bispecific antibodies are composed of a hybrid immunoglobulin heavy chain with a first binding specificity in one arm, and a hybrid immunoglobulin heavy-chain/light-chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 1994/04690. For further details of generating bispecific antibodies see, for example, Suresh et al., Meth. Enzymol., 121:210 (1986).

In another approach (U.S. Pat. No. 5,731,168), the interface between a pair of antibody molecules can be engineered to maximize the percentage of heterodimers that are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 1991/00360, WO 1992/20373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describes a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can be directly recovered from E. coli, and then chemically coupled to form bispecific antibodies. Shalaby et al., J. Exp. Med., 175: 217-25 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture are also known. For example, bispecific antibodies have been produced using leucine zippers. Kostelny et al., J. Immunol., 148(5):1547-53 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be used to produce antibody homodimers. The “diabody” technology of Holliger et al., Proc. Natl. Acad. Sci. USA, 90:6444-48 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another method to make bispecific antibody fragments using single-chain Fv (sFv) dimers is reported in Gruber et al., J. Immunol., 152:5368 (1994).

Antibodies with more than two valencies are contemplated. For example, trispecific antibodies can be prepared. Tutt et al., J. Immunol., 147: 60 (1991).

### Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen-binding sites (e.g., tetravalent antibodies), which can be readily produced by recombinant expression of nucleic acid encoding the polypeptide chains of the antibody. The multivalent antibody can comprise a dimerization domain and three or more antigen-binding sites. The preferred dimerization domain comprises (or consists of) an Fc region or a hinge region. In this scenario, the antibody will comprise an Fc region and three or more antigen-binding sites amino-terminal to the Fc region. The preferred multivalent antibody herein comprises (or consists of) three to about eight, but preferably four, antigen-binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light-chain variable-domain polypeptides. The multivalent antibody herein may, e.g., comprise from about two to about eight light-chain variable-domain polypeptides. Such polypeptides herein generally comprise a light-chain variable domain and, optionally, further comprise a CL domain.

### Vectors, Host Cells and Recombinant Methods

Selection and Transformation of Host Cells

Suitable host cells for cloning or expressing the recombinant monoclonal antibodies, immunoadhesins and other polypeptide antagonists described herein are prokaryote, yeast, or higher eukaryotic cells. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coliW3110 (ATCC 27,325) are suitable.

Full-length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction and safety. Full-length antibodies have greater half-life in circulation. Production in E. coli is faster and more cost efficient. For expression of antibody fragments and polypeptides in bacteria, see, e.g., U.S. Pat. No. 5,648,237; U.S. Pat. No. 5,789,199; and U.S. Pat. No. 5,840,523, describing translation-initiation region (TIR) and signal sequences for optimizing expression and secretion. After expression, the antibody can be isolated from the E. coli cell paste in a soluble fraction and purified through, e.g., a protein A or G column depending on the isotype. Final purification may mimic the process for purifying antibody expressed, e.g, in CHO cells. For general monoclonal antibody production, see U.S. Pat. No. 7,011,974.

In addition to prokaryotes, eukaryotic microbes such as filamentous fungi or yeast are suitable cloning or expression hosts for antibody-encoding, such as IGF-1R antibody-encoding, vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of, e.g., glycosylated IGF-1R-binding antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco are useful hosts.

Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); CHO cells, including those that are −DHFR (Urlaub et al., Proc. Natl. Acad. Sci. USA, 77:4216 (1980), and also including, but not limited to, CHO KI, CHO pro3-, CHO DG44, CHO DUXB11, Lec13, B-Ly1, and CHO DP12 cells, preferably a CHO DUX (DHFR-) or subclone thereof (herein called “CHO DUX”); C127 cells, mouse L cells; Ltk⁻ cells; mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse myeloma cells; NS0; hybridoma cells such as mouse hybridoma cells; COS cells; mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2).

Host cells are transformed with expression or cloning vectors for production of the IGF-1R-binding antibody herein, and cultured in conventional nutrient media modified as appropriate for inducing promoters, selecting transformants, or amplifying the genes encoding the desired sequences.

Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz., 58:44 (1979), Barnes et al., Anal. Biochem., 102:255 (1980), U.S. Pat. No. 4,767,704; 4,657,866; 4,927,762; 4,560,655; or 5,122,469; WO 1990/03430; WO 1987/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In one particular aspect, a suitable medium contains a basal medium component such as a DMEM/HAM F-12 based formulation (for composition of DMEM and HAM F12 media and especially serum-free media, see culture media formulations in the American Type Culture Collection Catalogue of Cell Lines and Hybridomas, Sixth Edition, 1988, pages 346-349) (the formulations of medium as described in U.S. Pat. No. 5,122,469 may be appropriate) with suitably modified, if necessary, concentrations of some components such as amino acids, salts, sugar, and vitamins, and optionally containing glycine, hypoxanthine, and thymidine; recombinant human insulin, hydrolyzed peptone, such as PROTEASE PEPTONE 2 and 3™, PRIMATONE HS™ or PRIMATONE RL™ (Difco, USA; Sheffield, England), or the equivalent; a cell-protective agent, such as PLURONIC F68™ or the equivalent PLURONIC™ polyol; GENTAMYCIN™ antibiotic; and trace elements. Preferably the cell culture media is serum free.

In one aspect herein, antibody production is conducted in large quantity by a fermentation process. Various large-scale fed-batch fermentation procedures are available for production of recombinant proteins. Large-scale fermentations have at least about 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to operating in a fermentor having a volumetric capacity of no more than about 100 liters, generally ranging from about one to 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art. Cells may be grown for shorter periods prior to induction. Cells are usually induced for about 12-50 hours, although longer or shorter induction time may be used.

The production yield and quality of the polypeptides of the invention can be improved by modifying various fermentation conditions. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al., J. Bio. Chem., 274:19601-19605 (1999); U.S. Pat. No. 6,083,715; U.S. Pat. No. 6,027,888; Bothmann and Pluckthun, J. Biol. Chem., 275:17100-17105 (2000); Ramm and Pluckthun, J. Biol. Chem., 275:17106-17113 (2000); and Arie et al., Mol. Microbiol., 39:199-210 (2001).

Proteolysis of expressed heterologous proteins (especially those that are proteolytically sensitive) can be minimized by using certain host strains deficient in proteolytic enzymes. For example, host cell strains may be modified to effect genetic mutation(s) in the genes encoding known bacterial proteases such as Protease III, OmpT, DegP, Tsp, Protease I, Protease Mi, Protease V, Protease VI, and combinations thereof. Some E. coli protease-deficient strains are available and described in, e.g., U.S. Pat. No. 5,264,365; U.S. Pat. No. 5,508,192; and Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system herein.

In one preferred embodiment for manufacturing the antibody 10H5, a cell-culture process is used involving a CHO cell line, preferably DP12-based, which is cultured using standard conditions as described herein and as known in the art.

Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an AMICON™ or MILLIPORE PELLICON™ ultrafiltration unit. A protease inhibitor such as phenylmethylsulphonylfluoride (PMSF) may be included in any of the foregoing steps to inhibit proteolysis, and antibiotics may be included to prevent the growth of adventitious contaminants.

In one embodiment, the antibody produced herein is further purified to obtain preparations that are substantially homogeneous for further assays and uses. Standard protein purification methods known in the art can be employed. The following procedures are exemplary of suitable purification procedures: hydroxylapatite chromatography, SP SEPHAROSE FAST-FLOW™ (SPSFF) chromatography, chromatography on heparin SEPHAROSE™, gel electrophoresis, dialysis, fractionation on immunoaffinity columns, ethanol precipitation, reverse-phase HPLC, chromatography on silica, chromatography on an anion- or cation-exchange resin (such as DEAE or a polyaspartic acid column), chromatofocusing, SDS-PAGE, ammonium sulfate precipitation, and gel filtration using, for example, SEPHADEX G-75™ medium. Affinity chromatography is one preferred purification technique.

For analytical-scale purification, smaller volumes are passed through columns and used; for preparative- or commercial-scale purification to produce quantities of antibody useful in therapeutic applications, larger volumes are employed. The skilled artisan will understand which scale should be used for which application. Preferably, preparative scale is employed for this invention.

The suitability of protein A as an affinity ligand depends on the species and isotype of any immunoglobulin Fc domain present in the antibody. Protein A can be used to purify antibodies that are based on human γ1, γ2, or γ4 heavy chains (Lindmark et al., J. Immunol. Meth., 62:1-13 (1983)). Protein G is recommended for all mouse isotypes and for human γ3 (Guss et al., EMBO J., 5:1567-1575 (1986)). The matrix to which the affinity ligand is attached is most often agarose, but other matrices are available. Mechanically stable matrices such as controlled-pore glass or poly(styrenedivinyl)benzene allow for faster flow rates and shorter processing times than can be achieved with agarose. Where the antibody comprises a C_{H}3 domain, the BAKERBOND ABX™ resin (J. T. Baker, Phillipsburg, N.J.) is useful for purification.

For Protein A chromatography, the solid phase to which Protein A is immobilized is preferably a column comprising a glass or silica surface, more preferably a controlled pore glass column or a silicic acid column. In some applications, the column is coated with a reagent, such as glycerol, to prevent nonspecific adherence of contaminants. The solid phase is then washed to remove contaminants non-specifically bound to the solid phase. Finally the antibody of interest is recovered from the solid phase by elution.

Following any preliminary purification step(s), the mixture comprising the antibody herein and contaminants may be subjected to low pH hydrophobic-interaction chromatography using an elution buffer at a pH of about 2.5-4.5, preferably performed at low salt concentrations (e.g., from about 0 to 0.25 M salt).

The antibodies herein are preferably recovered using, for example, multiple, preferably three, chromatography steps that may use various resins and may include a protein-recapture step. Suitable resin types for chromatography columns include, but are not limited to, affinity resins, anion-exchange resins, cation-exchange resins, and the like. Preferably, one of the chromatography steps uses an SPSFF resin.

### Generating Variant Antibodies Exhibiting Reduced or Absence of HAMA Response

Reduction or elimination of a HAMA response is a significant aspect of clinical development of suitable therapeutic agents. See, e.g., Khaxzaeli et al., J. Natl. Cancer Inst., 80:937 (1988); Jaffers et al., Transplantation, 41:572 (1986); Shawler et al., J. Immunol., 135:1530 (1985); Sears et al., J. Biol. Response Mod, 3:138 (1984); Miller et al., Blood, 62:988 (1983); Hakimi et al., J. Immunol., 147:1352 (1991); Reichmann et al., Nature, 332:323 (1988); and Junghans et al., Cancer Res, 50:1495 (1990). In some aspects herein, the invention provides antibodies that are humanized such that HAMA response is reduced or eliminated. Variants of these antibodies can further be obtained using routine methods known in the art.

For example, an amino acid sequence from an antibody herein can serve as a starting (parent) sequence for diversification of the framework and/or HVR sequence(s). A selected framework sequence to which a starting HVR sequence is linked is referred to as an acceptor human framework. While the acceptor human frameworks may be from, or derived from, a human immunoglobulin (the VL and/or VH regions thereof), preferably the acceptor human frameworks are from, or derived from, a human consensus framework sequence, as such frameworks are shown to have minimal, or no, immunogenicity in humans.

Where the acceptor is derived from a human immunoglobulin, one may optionally select a human framework sequence based on its homology to the donor framework sequence by aligning the donor framework sequence with various human framework sequences in a collection of human framework sequences and selecting the most homologous framework sequence as the acceptor.

In one embodiment, human consensus frameworks herein are from, or derived from, VH subgroup III and/or VL kappa subgroup I consensus framework sequences.

Thus, the VH acceptor human framework may comprise one, two, three, or all of the following framework sequences:

FR1 comprising Z1X1QLX2Z2X3GX4Z3LX5Z4PGX6X7Z5X8X9SCX10AS (SEQ ID NO:82), wherein Z1 is E or Q, X1 is V or I, X2 is Q or V, Z2 is E or Q, X3 is P or S, X4 is G, A, or P, Z3 is G or E, X5 is V or K, Z4 is K or Q, X6 is A, G, or E, X7 is S or T, Z5 is L or V, X8 is T, R, or K, X9 is L or I, and X10 is K or A;

FR2 comprising WVZ1QX1PGX2GX3X4WX5 (SEQ ID NO:83), wherein Z1 is R or K, X1 is R or A, X2 is Q, K, or E, X3 is L or F, X4 is E or K, and X5 is V, I, or M;

FR3 comprising X1X2X3X4X5X6X7Z1SX8Z2TZ3YX9X10X11X12X13X14LX15X16EDX17X18X19YX20CAR (SEQ ID NO:84), wherein X1 is K or R, X2 is A or F, X3 is T or V, X4 is I, L or F, X5 is T, S, or F, X6 is R, A, V or L, X7 is D or E, Z1 is N or T, X8 is K, S or A, Z2 is N or S, Z3 is L or A, X9 is M or L, X10 is Q or L, X1 is M, L or I, X12 is S or N, X13 is S or N, X14 is L, T or S, X15 is R, S, N, or D, X16 is A, V or D, X17 is S or T, X18 is V or A, X19 is V or T, and X20 is Y or F, where N is any amino acid; and

FR4 comprising WGX1GTX2 (SEQ ID NO:85), wherein X1 is Q or A and X2 is L, T, or S.

The VL acceptor human framework may comprise one, two, three, or all of the following framework sequences:

FR1 comprising DIX1MTQX2X3X4X5X6SX7SZ1GDX8VX9X10X11C (SEQ ID NO:86), wherein X1 is V or Q, X2 is S or T, X3 is P, Q or T, X4 is K or S, X5 is F or S, X6 is M or L, X7 is T or A, Z1 is L or V, X8 is R or K, X9 is S or T, X10 is V or I, and X11 is T or S,

FR2 comprising WYQQKPX1X2X3X4X5X6LIY (SEQ ID NO:87), wherein X1 is G or D, X2 is K, Q or G, X3 is A, S or T, X4 is P, V, or I, X5 is E or K, and X6 is A or L,

FR3 comprising GX1PX2RFX3GSGSGTDX4X5LTIX6NX7X8X9EDX10AX11YX12C (SEQ ID NO:88), wherein X1 is V or I, X2 is D or S, X3 is T or S, X4 is F or Y, X5 is T or S, X6 is S or T, X7 is V or L, X8 is Q or E, X9 is P, S or Q, X10 is L, F, or I, X11 is E or T, and X12 is Y or F, wherein N is any amino acid; and

FR4 comprising FGX1GTKVEIKR (SEQ ID NO:89), where X1 is Q, G or E.

While the acceptor may be identical in sequence to the human framework sequence selected, whether that be from a human immunoglobulin or a human consensus framework, according to this invention the acceptor sequence may comprise pre-existing amino acid substitutions relative to the human immunoglobulin sequence or human consensus framework sequence. These pre-existing substitutions are preferably minimal, usually only four, three, two, or one amino acid differences only relative to the human immunoglobulin sequence or consensus framework sequence.

HVR residues of the non-human antibody are incorporated into the VL and/or VH acceptor human frameworks. For example, one may incorporate residues corresponding to the Kabat CDR residues, the Chothia hypervariable loop residues, the AbM residues, the extended HVR residues, and/or contact residues. Optionally, the extended HVR residues as follows are incorporated: 24-34 (L1), 50-56 (L2) and 89-97 (L3), 26-35 (H1), 50-65 or 49-65 (H2), and 93-102, 94-102, or 95-102 (H3).

“Incorporation” of HVR residues can be achieved in various ways, e.g., nucleic acid encoding the desired amino acid sequence can be generated by mutating nucleic acid encoding the mouse variable domain sequence so that the framework residues thereof are changed to acceptor human framework residues, or by mutating nucleic acid encoding the human variable domain sequence so that the HVR residues are changed to non-human residues, or by synthesizing nucleic acid encoding the desired sequence, etc.

HVR-grafted variants may be generated by Kunkel mutagenesis of nucleic acid encoding the human acceptor sequences, using a separate oligonucleotide for each HVR. Kunkel et al., Methods Enzymol., 154:367-382 (1987). Appropriate changes can be introduced within the framework and/or HVR, using routine techniques, to correct and re-establish proper HVR-antigen interactions.

Phage(mid) display (also referred to herein as phage display in some contexts) can be used as a convenient and fast method for generating and screening many different potential variant antibodies in a library generated by sequence randomization. However, other methods for making and screening altered antibodies are available to the skilled person.

Phage(mid)-display technology has provided a powerful tool for generating and selecting novel proteins that bind to a ligand, such as an antigen. Using the techniques of phage(mid) display allows the generation of large libraries of protein variants that can be rapidly sorted for those sequences that bind to a target molecule with high affinity. Nucleic acids encoding variant polypeptides are generally fused to a nucleic acid sequence encoding a viral coat protein, such as the gene III protein or the gene VIII protein. Monovalent phagemid display systems where the nucleic acid sequence encoding the protein or polypeptide is fused to a nucleic acid sequence encoding a portion of the gene III protein have been developed. (Bass, Proteins, 8:309 (1990); Lowman and Wells, Methods: A Companion to Methods in Enzymology, 3:205 (1991)). In a monovalent phagemid display system, the gene fusion is expressed at low levels and wild type gene III proteins are also expressed so that infectivity of the particles is retained. Methods of generating peptide libraries and screening those libraries have been disclosed in many patents (e.g., U.S. Pat. No. 5,723,286; U.S. Pat. No. 5,432,018; U.S. Pat. No. 5,580,717; U.S. Pat. No. 5,427,908; and U.S. Pat. No. 5,498,530).

Libraries of antibodies have been prepared in a number of ways including by altering a single gene by inserting random DNA sequences or cloning a family of related genes. Methods for displaying antibodies or antigen binding fragments using phage(mid) display are described in U.S. Pat. No. 5,750,373; U.S. Pat. No. 5,733,743; U.S. Pat. No. 5,837,242; U.S. Pat. No. 5,969,108; U.S. Pat. No. 6,172,197; U.S. Pat. No. 5,580,717; and U.S. Pat. No. 5,658,727. The library is then screened for expression of antibodies or antigen-binding proteins with the desired characteristics.

The sequence of oligonucleotides includes one or more of the designed codon sets for the HVR residues to be altered. A codon set is a set of different nucleotide triplet sequences used to encode desired variant amino acids. Codon sets can be represented using symbols to designate particular nucleotides or equimolar mixtures of nucleotides as shown below according to the IUB code.

IUB Codes

G Guanine

A Adenine

T Thymine

C Cytosine

R (A or G)

Y (C or T)

M (A or C)

K (G or T)

S(C or G)

W (A or T)

H (A or C or T)

B (C or G or T)

V (A or C or G)

D (A or G or T) H

N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid-phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used herein, have sequences that allow for hybridization to a variable-domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al., Nucleic Acids Res., 10:6487-6504 (1987). Briefly, nucleic acid sequences encoding variant amino acids are created by hybridizing an oligonucleotide set encoding the desired codon sets to a DNA template, where the template is the single-stranded form of the plasmid containing a variable-region nucleic acid template sequence. After hybridization, DNA polymerase is used to synthesize an entire second complementary strand of the template that will thus incorporate the oligonucleotide primer, and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that in Crea et al., Proc. Nat'l. Acad. Sci. USA, 75:5765 (1978).

The DNA template is generated by those vectors that are derived from bacteriophage M13 vectors (the commercially available M13mp18 and M13mp19 vectors are suitable), or that contain a single-stranded phage origin of replication as described by Viera et al., Meth. Enzymol., 153:3 (1987). Thus, the DNA to be mutated can be inserted into one of these vectors to generate single-stranded template. Production of the single-stranded template is described in sections 4.21-4.41 of Sambrook et al., supra.

To alter the native DNA sequence, the oligonucleotide is hybridized to the single stranded template under suitable hybridization conditions. A DNA-polymerizing enzyme, for example, T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabeled with a ³²-P (phosphate) to identify the bacterial colonies that contain the mutated DNA.

The method described immediately above may be modified such that a homoduplex molecule is created wherein both strands of the plasmid contain the mutation(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously, the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al., Meth. Enzymol., 153:3 (1987). Thus, the DNA that is to be mutated must be inserted into one of these vectors in order to generate single-stranded template. Production of the single-stranded template is described in sections 4.21-4.41 of Sambrook et al., supra.

In another method, a library can be generated by providing upstream and downstream oligonucleotide sets, each set having a plurality of oligonucleotides with different sequences. These sequences are established by the codon sets provided within the sequence of the oligonucleotides. The upstream and downstream oligonucleotide sets, along with a variable-domain template nucleic acid sequence, can be used in a polymerase chain reaction (PCR) to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

The sequence of the PCR primers includes one or more of the designed codon sets for the solvent-accessible and highly diverse positions in a HVR. As described above, a codon set is a set of different nucleotide triplet sequences used to encode desired variant amino acids.

Antibody selectants that meet the desired criteria, as selected through appropriate screening/selection steps, can be isolated and cloned using standard recombinant techniques.

### Antibody Variants

In some embodiments, amino-acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that the sequence is made.

A useful method for identification of certain residues or regions of the antibody that are preferred locations for mutagenesis is called “alanine-scanning mutagenesis” as described by Cunningham and Wells, Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino-acid-sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide that increases the serum half-life of the antibody.

Another type of variant is an amino acid substitution variant. These variants have at least one amino acid residue in the antibody molecule replaced by a different residue. The sites of greatest interest for substitutional mutagenesis include the HVRs, but FR alterations are also contemplated. Conservative substitutions are shown in the table below under the heading of “preferred substitutions.” If such substitutions result in a change in biological activity, then more substantial changes, denominated “exemplary substitutions” in the table below, or as further described below in reference to amino acid classes, may be introduced and the products screened.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining; (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (as described, for example, in A. L. Lehninger, Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (O)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

One type of substitutional variant involves substituting one or more HVR residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several HVR sites (e.g., 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g., binding affinity) as herein disclosed. For location of candidate HVR sites for modification, alanine-scanning mutagenesis can be performed to identify HVR residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. Further, a phage display method for rapidly selecting framework mutations that improve the binding of humanized antibodies to their cognate antigens by random mutagenesis of important framework residues is described in Baca et al., J. Biol. Chem., 272: 10678-84 (1997). This technique is useful herein to prepare affinity matured antibodies and other improved variants as an alternative to framework optimization based on cycles of site-directed mutagenesis.

It may be desirable to introduce one or more amino acid modifications in an Fc region of the immunoglobulin polypeptides of the invention, thereby generating a Fc-region variant. The Fc-region variant may comprise a human Fc region sequence (e.g., a human IgG1, IgG2, IgG3 or IgG4 Fc region) comprising an amino acid modification (e.g., a substitution) at one or more amino acid positions including that of a hinge cysteine. These antibodies would nonetheless retain substantially the same characteristics required for therapeutic utility as compared to their wild-type counterpart. For example, it is thought that certain alterations can be made in the Fc region that would result in altered (i.e., either improved or diminished) C1q binding and/or CDC, e.g., as described in WO 1999/51642. See also Duncan and Winter Nature 322:738-40 (1988); U.S. Pat. No. 5,648,260; U.S. Pat. No. 5,624,821; and WO 1994/29351 concerning other examples of Fc-region variants.

For additional amino acid alterations that improve Fc function, see, e.g., U.S. Pat. No. 6,737,056. Any of the antibodies of the present invention may further comprise at least one amino acid substitution in the Fc region that decreases CDC activity, for example, comprising at least the substitution K322A. See U.S. Pat. No. 6,528,624 (Idusogie et al.).

In another preferred embodiment, the antibody has amino acid substitutions at any one or any combination of positions that are 268D, or 298A, or 326D, or 333A, or 334A, or 298A together with 333A, or 298A together with 334A, or 239D together with 332E, or 239D together with 298A and 332E, or 239D together with 268D and 298A and 332E, or 239D together with 268D and 298A and 326A and 332A, or 239D together with 268D and 298A and 326A and 332E, or 239D together with 268D and 283L and 298A and 332E, or 239D together with 268D and 283L and 298A and 326A and 332E, or 239D together with 330L and 332E and 272Y and 254T and 256E, or 250Q together with 428L, or 265A, or 297A, wherein the 265A substitution is in the absence of 297A and the 297A substitution is in the absence of 265A. The letter after the number in each of these designations represents the changed amino acid at that position.

Mutations that improve ADCC and CDC include substitutions at one to three positions of the Fc region, including positions 298, 333, and/or 334 of the Fc region (Eu numbering of residues), especially S298A together with E333A and K334A (S298A/E333A/K334A, or synonymously a combination of 298A, 333A, and 334A), also referred to herein as the triple Ala mutant. K334L increases binding to CD16. K322A results in reduced CDC activity; K326A or K326W enhances CDC activity. D265A results in reduced ADCC activity.

Stability variants are variants that show improved stability with respect to, e.g., oxidation and deamidation.

A further type of amino acid variant of the antibody alters the original glycosylation pattern of the antibody. Such altering includes deleting one or more carbohydrate moieties found in the antibody, and/or adding one or more glycosylation sites that are not present in the antibody. Glycosylation variants that increase ADCC function are described, e.g., in WO 2003/035835. See also US 2006/0067930.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US 2003/0157108 (Presta). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in, e.g., WO 2003/011878, Jean-Mairet et al. and U.S. Pat. No. 6,602,684 (Umana et al.). Antibodies with at least one galactose residue in the oligosaccharide attached to an Fc region of the antibody are reported, for example, in WO 1997/30087 (Patel et al.). See, also, WO 1998/58964 (Raju) and WO 1999/22764 (Raju) concerning antibodies with altered carbohydrate attached to the Fc region thereof. See also US 2005/0123546 (Umana et al.); US 2004/0072290 (Umana et al.); US 2003/0175884 (Umana et al.); WO 2005/044859 (Umana et al.); and US 2007/0111281 (Sondermann et al.) on antigen-binding molecules with modified glycosylation, including antibodies with an Fc region containing N-linked oligosaccharides; and US 2007/0010009 (Kanda et al.)

One preferred glycosylation antibody variant herein comprises an Fc region wherein a carbohydrate structure attached to the Fc region has reduced fucose or lacks fucose, which may improve ADCC function. Specifically, antibodies are contemplated herein that have reduced fusose relative to the amount of fucose on the same antibody produced in a wild-type CHO cell. That is, they are characterized by having a lower amount of fucose than they would otherwise have if produced by native CHO cells. Preferably the antibody is one wherein less than about 10% of the N-linked glycans thereon comprise fucose, more preferably wherein less than about 5% of the N-linked glycans thereon comprise fucose, and most preferably, wherein none of the N-linked glycans thereon comprise fucose, i.e., wherein the antibody is completely without fucose, or has no fucose.

Such “defucosylated” or “fucose-deficient” antibodies may be produced, for example, by culturing the antibodies in a cell line such as that disclosed in, for example, US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; US 2006/0063254; US 2006/0064781; US 2006/0078990; US 2006/0078991; Okazaki et al. J. Mol. Biol. 336:1239-1249 (2004); and Yamane-Ohnuki et al., Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US 2003/0157108 A1 (Presta) and WO 2004/056312 A1 (Adams et al., especially at Example 11) and knockout cell lines, such as alpha-1,6-fucosyltransferase gene, FUT8-knockout CHO cells (Yamane-Ohnuki et al., Biotech. Bioeng. 87: 614 (2004)). See also Kanda et al., Biotechnol. Bioeng., 94: 680-8 (2006). US 2007/0048300 (Biogen-IDEC) discloses a method of producing aglycosylated Fc-containing polypeptides, such as antibodies, having desired effector function, as well as aglycosylated antibodies produced according to the method and methods of using such antibodies as therapeutics, all being applicable herein. Additionally, U.S. Pat. No. 7,262,039 relates to a polypeptide having an alpha-1,3-fucosyltransferase activity, including a method for producing a fucose-containing sugar chain using the polypeptide.

See also US 2006/024304 (Germgross et al.); U.S. Pat. No. 7,029,872 (Gerngross); US 2004/018590 (Gerngross et al.); US 2006/034828 (Gerngross et al.); US 2006/034830 (Gerngross et al.); US 2006/029604 (Gerngross et al.); WO 2006/014679 (Gerngross et al.); WO 2006/014683 (Gerngross et al.); WO 2006/014685 (Gerngross et al.); WO 2006/014725 (Gerngross et al.); WO 2006/014726 (Gerngross et al.); and US 2007/0248600/WO 2007/115813 (Hansen et al.) on recombinant glycoproteins and glycosylation variants that are applicable herein.

In another embodiment, the invention provides an antibody composition comprising the antibodies described herein having an Fc region, wherein about 20-100% of the antibodies in the composition comprise a mature core carbohydrate structure in the Fc region that lacks a fucose. Preferably, such composition comprises antibodies having an Fc region that has been altered to change one or more of the antibody-dependent cell-mediated cytotoxicity (ADCC), complement-dependent cytotoxicity (CDC), or pharmacokinetic properties of the antibody compared to a wild-type IgG Fc sequence by substituting an amino acid selected from the group consisting of A, D, E, L, Q, T, and Y at any one or any combination of positions of the Fc region selected from the group consisting of: 238, 239, 246, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 309, 312, 314, 315, 320, 322, 324, 326, 327, 329, 330, 331, 332, 333, 334, 335, 337, 338, 340, 360, 373, 376, 378, 382, 388, 389, 398, 414, 416, 419, 428, 430, 434, 435, 437, 438, and 439.

The composition is more preferably one wherein the antibody further comprises an Fc substitution that is 268D or 326D or 333A together with 334A, or 298A together with 333A, or 298A together with 334A, or 239D together with 332E, or 239D together with 298A and 332E, or 239D together with 268D and 298A and 332E, or 239D together with 268D and 298A and 326A and 332A, or 239D together with 268D and 298A and 326A and 332E, or 239D together with 268D and 283L and 298A and 332E, or 239D together with 268D and 283L and 298A and 326A and 332E, or 239D together with 330L and 332E, wherein the letter after the number in each of these designations represents the changed amino acid at that position.

The composition is additionally preferably one wherein the antibody binds an FcγRIII. The composition further is preferably such that the antibody has ADCC activity in the presence of human effector cells or has increased ADCC activity in the presence of human effector cells compared to the otherwise same antibody comprising a human wild-type IgG1Fc region.

The composition is also preferably one wherein the antibody binds the FcγRIII with better affinity, or mediates ADCC more effectively, than a glycoprotein with a mature core carbohydrate structure including fucose attached to the Fc region of the glycoprotein. In addition, the composition is preferably one wherein the antibody has been produced by a CHO cell, preferably a Lec13 cell. The composition is also preferably one wherein the antibody has been produced by a mammalian cell lacking a fucosyltransferase gene, more preferably the FUT8 gene.

In one aspect, the composition is one wherein the antibody is free of bisecting N-acetylglucosamine (GlcNAc) attached to the mature core carbohydrate structure. Alternatively, the composition is such that the antibody has bisecting GlcNAc attached to the mature core carbohydrate structure.

In another aspect, the composition is one wherein the antibody has one or more galactose residues attached to the mature core carbohydrate structure. Alternatively, the composition is such that the antibody is free of one or more galactose residues attached to the mature core carbohydrate structure.

In a further aspect, the composition is one wherein the antibody has one or more sialic acid residues attached to the mature core carbohydrate structure. Alternatively, the composition is such that the antibody is free of one or more sialic acid residues attached to the mature core carbohydrate structure.

This composition preferably comprises at least about 2% afucosylated antibodies. The composition more preferably comprises at least about 4% afucosylated antibodies. The composition still more preferably comprises at least about 10% afucosylated antibodies. The composition even more preferably comprises at least about 19% afucosylated antibodies. The composition most preferably comprises about 100% afucosylated antibodies.

### Immunoconjugates

The invention also pertains to immunoconjugates, or antibody-drug conjugates (ADC), comprising an antibody conjugated to a cytotoxic agent such as a chemotherapeutic agent, a drug, a growth-inhibitory agent, a toxin (e.g., an enzymatically active toxin of bacterial, fungal, plant, or animal origin, or fragments thereof), or a radioactive isotope (i.e., a radioconjugate). Such ADC must show a safety profile that is acceptable.

The use of ADCs for the local delivery of cytotoxic or cytostatic agents, e.g., drugs to kill or inhibit tumor cells in the treatment of cancer (Syrigos and Epenetos Anticancer Research, 19:605-614 (1999); Niculescu-Duvaz and Springer, Adv. Drug Del. Rev., 26:151-172 (1997); U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., Lancet, 603-605 (1986); Thorpe, “Antibody Carriers Of Cytotoxic Agents In Cancer Therapy: A Review,” in Monoclonal Antibodies '84: Biological And Clinical Applications, A. Pinchera et al. (eds), pp. 475-506 (1985)). Maximal efficacy with minimal toxicity is sought thereby. Both polyclonal antibodies and monoclonal antibodies have been reported as useful in these strategies (Rowland et al., Cancer Immunol. Immunother., 21:183-187 (1986)). Drugs used in these methods include daunomycin, doxorubicin, methotrexate, and vindesine. Toxins used in antibody-toxin conjugates include bacterial toxins such as diphtheria toxin, plant toxins such as ricin, small molecule toxins such as geldanamycin (Mandler et al., J. Nat. Cancer Inst., 92(19):1573-1581 (2000); Mandler et al., Bioorganic & Med. Chem. Letters, 10: 1025-1028 (2000); and Mandler et al., Bioconjugate Chem., 13: 786-791 (2002)), maytansinoids (EP 1391213 and Liu et al., Proc. Natl. Acad. Sci. USA, 93: 8618-8623 (1996)), and calicheamicin (Lode et al., Cancer Res., 58:2928 (1998) and Hinman et al., Cancer Res. 53:3336-3342 (1993)). Without being limited to any one theory, the toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, for example, WO 1994/11026.

Conjugates of an antibody and at least one small-molecule toxin, e.g., a calicheamicin, maytansinoid, trichothecene, or CC1065, or derivatives of these toxins with toxin activity, are also included.

In the ADCs of the invention, an antibody (Ab) is conjugated to one or more drug moieties (D), e.g., about one to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody.

Ab−(L−D)ₚ Formula I

Nucleophilic groups on antibodies include, but are not limited to: (i) N-terminal amine groups, (ii) side-chain amine groups, e.g., lysine, (iii) side-chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleiimide groups. Certain antibodies have reducible interchain disulfides, i.e., cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

ADCs of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups that may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Domen et al., J. Chromatog., 510: 293-302 (1990)). In another embodiment, proteins containing N-terminal serine or threonine residues can react with sodium meta-periodate, resulting in production of an aldehyde in place of the first amino acid (Geoghegan and Stroh, Bioconjugate Chem., 3:138-46 (1992) and U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide that does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

The ADCs herein are optionally prepared with cross-linker reagents such as, for example, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), which are commercially available (e.g., Pierce Biotechnology, Inc., Rockford, Ill.).

### Antibody Derivatives

The antibodies of the present invention can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Pharmaceutical Formulations

Therapeutic formulations of the antibodies herein are prepared for storage by mixing an antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as, e.g., TWEEN™, PLURONICS™, or polyethylene glycol (PEG).

A further formulation and delivery method herein involves that described, for example, in WO 2004/078140, including the ENHANZE™ drug delivery technology (Halozyme Inc.). This technology is based on a recombinant human hyaluronidase (rHuPH20). rHuPH20 is a recombinant form of the naturally occurring human enzyme approved by the FDA that temporarily clears space in the matrix of tissues such as skin. That is, the enzyme has the ability to break down hyaluronic acid (HA), the space-filling “gel”-like substance that is a major component of tissues throughout the body. This clearing activity is expected to allow rHuPH20 to improve drug delivery by enhancing the entry of therapeutic molecules through the subcutaneous space. Hence, when combined or co-formulated with certain injectable drugs, this technology can act as a “molecular machete” to facilitate the penetration and dispersion of these drugs by temporarily opening flow channels under the skin. Molecules as large as 200 nanometers may pass freely through the perforated extracellular matrix, which recovers its normal density within approximately 24 hours, leading to a drug delivery platform that does not permanently alter the architecture of the skin.

Hence, the present invention includes a method of delivering an antibody herein to a tissue containing excess amounts of glycosaminoglycan, comprising administering a hyaluronidase glycoprotein (sHASEGP) (this protein comprising a neutral active soluble hyaluronidase polypeptide and at least one N-linked sugar moiety, wherein the N-linked sugar moiety is covalently attached to an asparagine residue of the polypeptide) to the tissue in an amount sufficient to degrade glycosaminoglycans sufficiently to open channels less than about 500 nanometers in diameter; and administering the antibody to the tissue comprising the degraded glycosaminoglycans.

In another embodiment, the invention includes a method for increasing the diffusion of an antibody herein that is administered to a subject comprising administering to the subject a sHASEGP polypeptide in an amount sufficient to open or to form channels smaller than the diameter of the antibody and administering the antibody, whereby the diffusion of the therapeutic substance is increased. The sHASEGP and antibody may be administered separately or simultaneously in one formulation, and consecutively in either order or at the same time.

Exemplary anti-IGF-1R antibody formulations may be made generally as set forth in WO 1998/56418, which include a liquid multidose formulation comprising an antibody at 40 mg/mL, 25 mM acetate, 150 mM trehalose, 0.9% benzyl alcohol, 0.02% POLYSORBATE 20 surfactant at pH 5.0 that has a minimum shelf life of two years storage at 2-8° C. Another suitable anti-IGF-1R formulation comprises 10 mg/mL antibody in 9.0 mg/mL sodium chloride, 7.35 mg/mL sodium citrate dihydrate, 0.7 mg/mL POLYSORBATE™ 80 surfactant, and Sterile Water for Injection, pH 6.5.

The antibody herein may also be formulated, for example, as described in WO 1997/04801, which teaches a stable lyophilized protein formulation that can be reconstituted with a suitable diluent to generate a high-protein concentration reconstituted formulation suitable for subcutaneous administration. Preferably, however, the antibody herein is formulated as described in U.S. Pat. No. 6,171,586. This patent teaches a stable aqueous pharmaceutical formulation comprising a therapeutically effective amount of an antibody not subjected to prior lyophilization, an acetate buffer from about pH 4.8 to about 5.5, a surfactant, and a polyol, wherein the formulation lacks a tonicifying amount of sodium chloride. The polyol is preferably a nonreducing sugar, more preferably trehalose or sucrose, most preferably trehalose, preferably at an amount of about 2-10% w/v. The antibody concentration in the formulation is preferably from about 0.1 to about 50 mg/mL, and the surfactant is preferably a POLYSORBATE™ surfactant, preferably an amount of about 0.01-0.1% v/v. The acetate is preferably present in an amount of about 5-30 mM, more preferably about 10-30 mM. The formulation optionally further contains a preservative, which is preferably benzyl alcohol.

One especially preferred formulation herein is about 20 to 50 mg/mL antibody, sodium acetate in an amount of about 10-30 mM, pH about 4.8 to about 5.5, trehalose, and a POLYSORBATE™ surfactant. One particularly preferred formulation herein is one in which the bulk concentration of the antibody is about 20 mg/mL and the formulation also contains about 20 mM sodium acetate, pH 5.3±0.3, about 200-300 mM trehalose, more preferably about 240 mM trehalose, and about 0.02% POLYSORBATE™ 20 surfactant.

Lyophilized formulations adapted for subcutaneous administration are described in U.S. Pat. No. 6,267,958. Such lyophilized formulations may be reconstituted with a suitable diluent to a high protein concentration and the reconstituted formulation may be administered subcutaneously to the subject to be treated herein.

Crystallized forms of the antibody are also contemplated. See, for example, US 2002/0136719.

The formulation herein may also contain more than one active compound (a second medicament as noted herein) as necessary for the particular indication being treated, preferably those with complementary activities that do not adversely affect each other. For example, it may be desirable to further provide a cytotoxic agent, chemotherapeutic agent, cytokine antagonist, integrin antagonist, or immunosuppressive agent (e.g., one that acts on T cells, such as cyclosporin or an antibody that binds T cells, e.g., one that binds LFA-1). The type and effective amounts of such second medicaments depend, for example, on the amount of antibody present in the formulation, the type of disease or disorder or treatment, the clinical parameters of the subjects, and other factors discussed above. These are generally used in the same dosages and with administration routes as described herein or about from about 1 to 99% of the heretofore employed dosages.

The active ingredients may also be entrapped in microcapsules prepared, e.g., by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nano-capsules) or in macroemulsions. Such techniques are disclosed, for example, in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in-vivo administration must be sterile. This is readily accomplished by filtration through sterile filtration membranes.

### Uses

An antibody of the present invention may be used in, for example, in vitro, ex vivo, and in vivo therapeutic methods. Antibodies of the invention can be used as an antagonist to partially or fully block the specific IGF-1R activity in vitro, ex vivo, and/or in vivo. Moreover, at least some of the antibodies of the invention can neutralize antigen activity from other species. Accordingly, the antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus, rhesus, pig, or mouse). In one embodiment, the antibody of the invention can be used for inhibiting antigen activities by contacting the antibody with the antigen such that antigen activity is inhibited. Preferably, the antigen is a human protein molecule.

In one aspect, an antibody of the invention can be used in a method for inhibiting an antigen in a subject suffering from a disorder in which the antigen activity is detrimental, comprising administering to the subject the antibody such that the antigen activity in the subject is inhibited. Preferably, the antigen is a human protein and the subject is a human subject. Alternatively, the subject can be a mammal expressing the antigen to which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). The antibodies of the invention can be used to treat, inhibit, delay progression of, prevent/delay recurrence of, ameliorate, or prevent diseases, disorders or conditions associated with abnormal expression and/or activity of one or more antigen molecules, including but not limited to malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders.

In one aspect, a blocking antibody of the invention is specific to a ligand antigen, and inhibits the antigen activity by blocking or interfering with the ligand-receptor interaction involving the ligand antigen, thereby inhibiting the corresponding signal pathway and other molecular or cellular events. The invention also features IGF-1R-specific antibodies that do not necessarily prevent ligand binding but interfere with receptor activation, thereby inhibiting any responses that would normally be initiated by the ligand binding. The invention also encompasses antibodies that either preferably or exclusively bind to ligand-receptor complexes. An antibody of the invention can also act as an agonist of a particular antigen receptor, thereby potentiating, enhancing or activating either all or partial activities of the ligand-mediated receptor activation.

The antibody may be a naked antibody or alternatively is conjugated with another molecule, e.g. a cytotoxic agent if the resulting immunoconjugate has an acceptable safety profile. In certain aspects, the immunoconjugate and/or antigen to which it is bound is/are internalized by the cell, resulting in increased therapeutic efficacy of the immunoconjugate in killing the target cell to which it binds. In one aspect, the cytotoxic agent targets or interferes with nucleic acid in the target cell. Examples of such cytotoxic agents include any chemotherapeutic agents noted herein (e.g., a maytansinoid or a calicheamicin), a radioactive isotope, a ribonuclease, or a DNA endonuclease. Preferably, the antibodies herein are conjugated to a cell toxin and/or a radioelement.

In one embodiment, the subject has never been previously administered any drug(s), such as immunosuppressive agent(s), to treat the disorder. In a still further aspect, the subject or patient is not responsive to therapy for the disorder. In another embodiment, the subject or patient is responsive to therapy for the disorder.

In another embodiment, the subject or patient has been previously administered one or more drug(s) to treat the disorder. In a further embodiment, the subject or patient was not responsive to one or more of the medicaments that had been previously administered. Such drugs to which the subject may be non-responsive include, for example, chemotherapeutic agents, cytotoxic agents, anti-angiogenic agents, immunosuppressive agents, pro-drugs, cytokines, cytokine antagonists, cytotoxic radiotherapies, corticosteroids, anti-emetics, cancer vaccines, analgesics, anti-vascular agents, growth-inhibitory agents, epidermal growth factor receptor (EGFR) inhibitors such as erlotinib, an Apo2L/TRAIL DR5 agonist (such as apomab, a DR-5-targeted dual proapoptotic receptor agonist), or antagonists to IGF-1R (e.g., a molecule that inhibits or reduces a biological activity of IGF-1R, such as one that substantially or completely inhibits, blocks, or neutralizes one or more biological activities of IGF-1R). More particularly, the drugs to which the subject may be non-responsive include chemotherapeutic agents, cytotoxic agents, anti-angiogenic agents, immunosuppressive agents, EGFR inhibitors such as erlotinib, apomab, or antagonists to IGF-1R. Preferably, such IGF-1R antagonists do not include an antibody of this invention (such IGF-1R antagonists include, for example, small-molecule inhibitors of IGF-1R, or anti-sense oligonucleotides, antagonistic peptides, or antibodies to IGF-1R that are not the antibodies of this invention, as noted, for example, in the background section above). In a further aspect, such IGF-1R antagonists include an antibody of this invention, such that re-treatment is contemplated with one or more antibodies of this invention.

In a further aspect, the invention involves a method of reducing the risk of a negative side effect in a subject (e.g., selected from the group consisting of an infection, cancer, heart failure and demyelination) comprising administering to the subject an effective amount of the antibody herein. Preferably, such subject has cancer.

In yet another embodiment, the antibody herein is the only medicament administered to the subject to treat the disorder. In a further aspect, the antibody herein is one of the medicaments used to treat the disorder. Preferably, the subject being treated herein is human.

The antibodies herein are especially useful in treating cancer and inhibiting tumor growth. Examples of types of cancers treatable herein are provided hereinabove, including preferred cancers, such as particularly breast, lung (such as small-cell lung cancer), prostate (such as hormone-resistant prostate), ovarian, and colorectal cancers.

The antibodies herein are also useful to treat aging in a subject when used in an effective amount. Aging is defined herein.

As to autoimmune disorders, while the antibodies may be used to treat such disorder as noted above, in one aspect, the subject only has RA as an autoimmune disorder. In a still further aspect, the subject only has MS as an autoimmune disorder. In a yet further embodiment, the subject only has lupus or ANCA-associated vasculitis or Sjögren's syndrome as an autoimmune disorder as defined above. In another aspect, the subject has RA and the antibody induces a major clinical response in the subject.

In a still further embodiment, the subject has an abnormal level of one or more regulatory cytokines, anti-nuclear antibodies (ANA), anti-rheumatoid factor (RF) antibodies, creatinine, blood urea nitrogen, anti-endothelial antibodies, anti-neutrophil cytoplasmic antibodies (ANCA), infiltrating CD20 cells, anti-double stranded DNA (dsDNA) antibodies, anti-Sm antibodies, anti-nuclear ribonucleoprotein antibodies, anti-phospholipid antibodies, anti-ribosomal P antibodies, anti-Ro/SS-A antibodies, anti-Ro antibodies, anti-La antibodies, antibodies directed against Sjögren's-associated antigen A or B (SS-A or SS-B), antibodies directed against centromere protein B (CENP B) or centromere protein C (CENP C), autoantibodies to ICA69, anti-Smith antigen (Sm) antibodies, anti-nuclear ribonucleoprotein antibodies, anti-ribosomal P antibodies, autoantibodies staining the nuclear or perinuclear zone of neutrophils (pANCA), anti-Saccharomyces cerevisiae antibodies, cross-reactive antibodies to GM1 ganglioside or GQ1b ganglioside, anti-acetylcholine receptor (AchR), anti-AchR subtype, or anti-muscle specific tyrosine kinase (MuSK) antibodies, serum anti-endothelial cell antibodies, IgG or anti-desmoglein (Dsg) antibodies, anti-centromere, anti-topoisomerase-1 (Scl-70), anti-RNA polymerase or anti-U3-ribonucleoprotein (U3-RNP) antibodies, anti-glomerular basement membrane (GBM) antibodies, anti-glomerular basement membrane (GBM) antibodies, anti-mitochondrial (AMA) or anti-mitochondrial M2 antibodies, anti-thyroid peroxidase (TPO), anti-thyroglobin (TG) or anti-thyroid stimulating hormone receptor (TSHR) antibodies, anti-nucleic (AN), anti-actin (AA) or anti-smooth muscle antigen (ASM) antibodies, IgA anti-endomysial, IgA anti-tissue transglutaminase, IgA anti-gliadin or IgG anti-gliadin antibodies, anti-CYP21A2, anti-CYP11A1 or anti-CYP17 antibodies, anti-ribonucleoprotein (RNP), or myosytis-specific antibodies, anti-myelin associated glycoprotein (MAG) antibodies, anti-hepatitis C virus (HCV) antibodies, anti-GM1 ganglioside, anti-sulfate-3-glycuronyl paragloboside (SGPG), or IgM anti-glycoconjugate antibodies, IgM anti-ganglioside antibody, anti-thyroid peroxidase (TPO), anti-thyroglobin (TG) or anti-thyroid stimulating hormone receptor (TSHR) antibodies, anti-myelin basic protein or anti-myelin oligodendrocytic glycoprotein antibodies, IgM rheumatoid factor antibodies directed against the Fc portion of IgG, anti-Factor VIII antibodies, or a combination thereof.

The parameters for assessing efficacy or success of treatment of a disorder will be known to the physician of skill in the appropriate disease. Generally, the physician of skill will look for reduction in the signs and symptoms of the specific disease. The following are by way of examples.

For cancer, the physician would look for reduction in tumor size, elimination of the tumor, lack of recurrence or spread of the tumor, and other symptoms.

For RA, the antibodies can be used as first-line therapy in patients with early RA (i.e., methotrexate (MTX) naive), or in combination with, e.g., MTX or cyclophosphamide. Alternatively, the antibodies can be used in treatment as second-line therapy for patients who were DMARD and/or MTX refractory, in combination with, e.g., MTX. The IGF-1R-binding antibodies can also be administered in combination with B-cell mobilizing agents such as integrin antibodies that mobilize B cells into the bloodstream for more effective killing. The IGF-1R-binding antibodies are useful to prevent and control joint damage, delay structural damage, decrease pain associated with inflammation in RA, and generally reduce the signs and symptoms in moderate-to-severe RA. The RA patient can be treated with the IGF-1R antibody prior to, after, or together with treatment with other drugs used in treating RA (see combination therapy described herein). Patients who had previously failed DMARDs and/or had an inadequate response to MTX alone are, in one embodiment, treated with a IGF-1R-binding antibody. In another embodiment, such patients are administered humanized IGF-1R-binding antibody plus cyclophosphamide or IGF-1R-binding antibody plus MTX.

One method of evaluating treatment efficacy in RA is based on American College of Rheumatology (ACR) criteria, which are used to measure the percentage of improvement in tender and swollen joints, among other things. The RA patient can be scored at for example, ACR 20 (20 percent improvement) compared with no antibody treatment (e.g., baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire (HAQ) score, AIMS score, or SF-36 at time periods during or after treatment. The ACR 20 criteria may include 20% improvement in both tender (painful) joint count and swollen joint count plus a 20% improvement in at least three of five additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.

The ACR 50 and 70 are defined analogously. Preferably, the patient is administered an amount of an IGF-1R-binding antibody herein effective to achieve at least a score of ACR 20, preferably at least ACR 30, more preferably at least ACR 50, even more preferably at least ACR 70, and most preferably at least ACR 75.

Psoriatic arthritis has unique and distinct radiographic features. For psoriatic arthritis, joint erosion and joint space narrowing can be evaluated by the Sharp score as well. The antibodies disclosed herein can be used to prevent the joint damage as well as reduce disease signs and symptoms of the disorder.

Yet another aspect of the invention is a method of treating lupus, including SLE and lupus nephritis, by administering to the subject having such disease an effective amount of an antibody of the invention. For example, SLEDAI scores provide a numerical quantitation of disease activity. The SLEDAI is a weighted index of 24 clinical and laboratory parameters known to correlate with disease activity, with a numerical range of 0-103. See Gescuk and Davis, Current Opinion in Rheumatology, 14: 515-521 (2002). Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, psoriatic arthritis, and Crohn's disease. Treatment success can be determined by validated patient and physician global assessment measuring tools.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. Treatment efficacy for psoriasis is assessed by monitoring changes in clinical signs and symptoms of the disease, including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual Analog Scale (VAS) used to indicate the degree of itching experienced at specific time points.

### Assays

### Activity Assays

The antibodies of the present invention can be characterized for their physical/chemical properties and biological functions by various assays known in the art.

The purified immunoglobulins can be further characterized by a series of assays including, but not limited to, N-terminal sequencing, amino-acid analysis, non-denaturing size exclusion high pressure liquid chromatography (HPLC), mass spectrometry, ion-exchange chromatography and papain digestion.

In some embodiments, the antibodies of the present invention are tested for their antigen-binding activity. The antigen-binding assays that are known in the art and can be used herein include without limitation any direct or competitive-binding assays using techniques such as western blots, radioimmunoassays, ELISA (enzyme-linked immunosorbent assay), direct or indirect “sandwich” immunoassays, immunoprecipitation assays, fluorescent immunoassays, and protein A immunoassays.

In one embodiment, the present invention provides an altered antibody that possesses some but not all effector functions, which make it a desired candidate for many applications in which the half-life of the antibody in vivo is important, yet certain effector functions (such as complement and ADCC) are unnecessary or deleterious. In certain embodiments, the Fc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, supra.

Cell-based assays and animal models are useful to understand the interaction of the ligands with IGF-1R and the development and pathogenesis of the conditions and diseases referred to herein.

An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Natl. Acad. Sci. (USA), 95:652-656 (1998). C1q binding assays may also be carried out to confirm that the antibody is unable to bind C1q and hence lacks CDC activity. To assess complement activation, a CDC assay, e.g., as described in Gazzano-Santoro et al., J. Immunol. Methods, 202:163 (1996), may be performed. FcRn binding and in vivo clearance/half life determinations can also be performed using methods known in the art.

In one approach, mammalian cells may be transfected with the ligands or receptors described herein, and the ability of the antibody herein to inhibit binding or activity is analyzed. Suitable cells can be transfected with the desired gene, and monitored for activity. Such transfected cell lines can then be used to test the ability of antibody, for example, to modulate IGF-1R expression of the cells.

In addition, primary cultures derived from transgenic animals can be used in the cell-based assays. Techniques to derive continuous cell lines from transgenic animals are well known in the art. See, e.g., Small et al., Mol. Cell. Biol., 5:642-8 (1985).

One suitable cell-based assay is the addition of epitope-tagged ligand (e.g., IGF-1R) to cells that have or express the respective receptor, and the analysis of binding (in presence or absence of prospective antibodies) by FACS staining with anti-tag antibody. In another assay, the ability of the antibody herein to inhibit the expression of IGF-1R on cells expressing it is assayed. For example, suitable expressing cell lines are cultured in the presence or absence of prospective antibodies and the modulation of IGF-1R expression can be measured by ³H-thymidine incorporation or cell number.

The results of the cell-based in vitro assays can be further verified using in vivo animal models. Many animal models can be used to test the efficacy of the antibodies identified herein in relation to, for instance, cancer or immune-related disease. The in vivo nature of such models makes them particularly predictive of responses in human patients. Animal models of cancer and immune-related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, and implantation under the renal capsule. A mouse dose can be converted to a human dose by dividing the mouse dose by 12, and the rat dose to human dose by dividing the rat dose by 6, assuming a 60-kg human. Thus, e.g., 20 mg/kg in a mouse is equivalent to about 1.7 mg/kg in a human.

Effective mouse tumor models are described, for example, in Garber et al., J. Natl. Cancer Inst., 97: 790-792 (2005). Further, Gualberto et al., supra, specifies one suitable biomarker assay for use herein to define a possible therapeutic dose and regimen, namely, flow cytometry of granulocytes.

Rodent models for assaying effects of the antibodies on aging are described in the below examples.

Graft-versus-host disease is an example of a disease for which an animal model has been designed. Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing T-cell reactivity against MHC antigens and minor transplant antigens. A suitable procedure is described in detail in Current Protocols in Immunology, Eds. Cologan et al., (John Wiley & Sons, Inc., 1994), unit 4.3.

An animal model for skin allograft rejection tests the ability of T cells to mediate in vivo tissue destruction to measure of their role in anti-viral immunity, and is described, for example, in Current Protocols in Immunology, Collogan et al., ed, supra, unit 4.4. Other transplant rejection models useful to test the antibodies herein include the allogeneic heart transplant models described by Tanabe et al., Transplantation, 58:23 (1994) and Tinubu et al., J. Immunol., 4330-4338 (1994).

Animal models for delayed-type hypersensitivity provide an assay of cell-mediated immune function. Delayed type hypersensitivity reactions are a T-cell-mediated in vivo immune response characterized by inflammation that does not reach a peak until after a period of time has elapsed after challenge with an antigen. These reactions also occur in tissue specific autoimmune diseases such as MS and experimental autoimmune encephalomyelitis (EAE, a model for MS). A suitable model is described in detail in Current Protocols in Immunology, supra, Collogan et al., ed., unit 4.5.

The collagen-induced arthritis (CIA) model is considered a suitable model for studying potential drugs or biologics active in human arthritis because of the many immunological and pathological similarities to human RA, the involvement of localized major histocompatibility, complete class-II-restricted T helper lymphocyte activation, and the similarity of histological lesions. Features of this CIA model that are similar to that found in RA patients include: erosion of cartilage and bone at joint margins (as can be seen in radiographs), proliferative synovitis, and symmetrical involvement of small and medium-sized peripheral joints in the appendicular, but not the axial, skeleton. Jamieson et al., Invest. Radiol., 20:324-329 (1985). Furthermore, IL-1 and TN-α appear to be involved in CIA as in RA. Joosten et al., J. Immunol., 163:5049-5055 (1999). TNF-neutralizing antibodies and separately, TNFR.Fc reduced the symptoms of RA in this model (Williams et al., Proc. Natl. Acad. Sci. USA, 89:9784-9788 (1992); Wooley et al., J. Immunol., 151: 6602-6607 (1993)).

In this model for RA, type II collagen is purified from bovine articular cartilage (Miller, Biochemistry 11:4903 (1972)) and used to immunized mice (Williams et al, Proc. Natl. Acad. Sci. USA, 91:2762 (1994)). Symptoms of arthritis include erythema and/or swelling of the limbs as well as erosions or defects in cartilage and bone as determined by histology. This widely used model is also described, for example, by Holmdahl et al., APMIS, 97:575 (1989), and in Current Protocols in Immunology, supra, Collogan et al., ed., units 15.5, and in Issekutz et al., Immunology, 88:569 (1996).

A model of asthma has been described in which antigen-induced airway hyper-reactivity, pulmonary eosinophilia and inflammation are induced by sensitizing an animal with ovalbumin and challenging the animal with the same protein delivered by aerosol. Animal models such as rodent and non-human primate models exhibit symptoms similar to atopic asthma in humans upon challenge with aerosol antigens. Suitable procedures to test the antibodies herein for suitability in treating asthma include those described by Wolyniec et al., Am. J. Respir. Cell Mol. Biol., 18:777 (1998).

Additionally, the antibodies herein can be tested in the SCID/SCID mouse model for immune disorders. For example, as described by Schon et al., Nat. Med., 3:183 (1997), the mice demonstrate histopathologic skin lesions resembling psoriasis. Another suitable model is the human skin/SCID mouse chimera prepared as described by Nickoloff et al., Am. J. Path., 146:580 (1995).

### Dosage

For the prevention or treatment of disease, the appropriate dosage of an antibody of the invention (when used alone or in combination with a second medicament as noted below) will depend, for example, on the type of disease to be treated, the type of antibody, the severity and course of the disease, whether the antibody is administered for preventive or therapeutic purposes, previous therapy, the patient's clinical history and response to the antibody, and the discretion of the attending physician. The dosage is preferably efficacious for the treatment of that indication while minimizing toxicity and side effects.

The antibody is suitably administered to the patient at one time or over a series of treatments. Depending on the type and severity of the disease, about 1 μg/kg to 500 mg/kg (preferably about 0.1 mg/kg to 400 mg/kg) of antibody is an initial candidate dosage for administration to the patient, whether, for example, by one or more separate administrations, or by continuous infusion. One typical daily dosage might range from about 1 μg/kg to 500 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 400 mg/kg. Thus, one or more doses of about 0.5 mg/kg, 2.0 mg/kg, 4.0 mg/kg or 10 mg/kg or 50 mg/kg or 100 mg/kg or 300 mg/kg or 400 mg/kg (or any combination thereof) may be administered to the patient. Such doses may be administered intermittently, e.g., every week or every three weeks (e.g., such that the patient receives from about two to about twenty, e.g., about six doses of the antibody). An initial higher loading dose, followed by one or more lower doses, may be administered. An exemplary dosing regimen comprises administering an initial loading dose of about 4 to 500 mg/kg, followed by a weekly maintenance dose of about 2 to 400 mg/kg of the antibody. However, other dosage regimens may be useful. The progress of this therapy is easily monitored by conventional techniques and assays.

For the treatment of cancer, aging, or an autoimmune disorder, the therapeutically effective dosage will typically be in the range of about 50 mg/m² to about 3000 mg/m², preferably about 50 to 1500 mg/m², more preferably about 50-1000 mg/m². In one embodiment, the dosage range is about 125-700 mg/m². For treating RA, in one embodiment, the dosage range for the humanized antibody is about 50 mg/m² or 125 mg/m² (equivalent to about 200 mg/dose) to about 1000 mg/m², given in two doses, e.g., the first dose of about 200 mg is administered on day one followed by a second dose of about 200 mg on day 15. In different embodiments, the dosage is about any one of 50 mg/dose, 80 mg/dose, 100 mg/dose, 125 mg/dose, 150 mg/dose, 200 mg/dose, 250 mg/dose, 275 mg/dose, 300 mg/dose, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose, or 600 mg/dose, or 700 mg/dose, or 800 mg/dose, or 900 mg/dose, or 1000 mg/dose, or 1500 mg/dose.

In treating disease, the IGF-1R-binding antibodies of the invention can be administered to the patient chronically or intermittently, as determined by the physician of skill in the disease.

A patient administered a drug by intravenous infusion or subcutaneously may experience adverse events such as fever, chills, burning sensation, asthenia, and headache. To alleviate or minimize such adverse events, the patient may receive an initial conditioning dose(s) of the antibody followed by a therapeutic dose. The conditioning dose(s) will be lower than the therapeutic dose to condition the patient to tolerate higher dosages.

The antibodies herein may be administered at a frequency that is within the skill and judgment of the practicing physician, depending on various factors noted above, for example, the dosing amount. This frequency includes twice a week, three times a week, once a week, bi-weekly, or once a month, In a preferred aspect of this method, the antibody is administered no more than about once every other week, more preferably about once a month.

### Route of Administration

The antibodies used in the methods of the invention (as well as any second medicaments) are administered to a subject or patient, including a human patient, in accord with suitable methods, such as those known to medical practitioners, depending on many factors, including whether the dosing is acute or chronic. These routes include, for example, parenteral, intravenous administration, e.g., as a bolus or by continuous infusion over a period of time, by subcutaneous, intramuscular, intra-arterial, intraperitoneal, intrapulmonary, intracerebrospinal, intra-articular, intrasynovial, intrathecal, intralesional, or inhalation routes (e.g., intranasal). Parenteral infusions include intramuscular, intravenous, intraarterial, intraperitoneal, or subcutaneous administration. In addition, the antibody is suitably administered by pulse infusion, particularly with declining doses of the antibody. Preferred routes herein are intravenous or subcutaneous administration.

More preferably, the antibody is administered intravenously, still more preferably about every 21 days, still more preferably over about 30 to 90 minutes. In another embodiment, such iv-infused or treated subjects have cancer, preferably advanced or metastatic solid tumors, more preferably non-small-cell lung cancer. Additionally, such treated subjects preferably have progressed on prior therapy (such as, for example, chemotherapy) and/or preferably have not been previously treated with EGFR inhibitors such as erlotinib or apomab, or are those for whom there is no effective therapy. More preferably, such subjects are treated with a combination of the antibody herein and erlotinib or apomab.

In one embodiment, the antibody herein is administered by intravenous infusion, and more preferably with about 0.9 to 20% sodium chloride solution as an infusion vehicle.

### Combination Therapy

In any of the methods herein, one may administer to the subject or patient along with the antibody herein an effective amount of a second medicament (where the antibody herein is a first medicament), which is another active agent that can treat the condition in the subject that requires treatment. For instance, an antibody of the invention may be co-administered with another antibody, chemotherapeutic agent(s) (including cocktails of chemotherapeutic agents), cytotoxic agent(s), anti-angiogenic agent(s), immunosuppressive agent(s), cytokine(s), cytokine antagonist(s), and/or growth-inhibitory agent(s). The type of such second medicament depends on various factors, including the type of disorder, such as cancer or an autoimmune disorder, the severity of the disease, the condition and age of the patient, the type and dose of first medicament employed, etc.

Where an antibody of the invention inhibits tumor growth, for example, it may be particularly desirable to combine it with one or more other therapeutic agents that also inhibit tumor growth. For instance, an antibody of the invention may be combined with an anti-VEGF antibody (e.g., AVASTIN®), an Apo2L/TRAIL DR5 agonist (such as apomab, a DR-5-targeted dual proapoptotic receptor agonist), and/or anti-ErbB antibodies (e.g. HERCEPTIN® trastuzumab anti-HER2 antibody or an anti-HER2 antibody that binds to Domain II of HER2, such as OMNITARG™ pertuzumab anti-HER2 antibody or erlotinib (TARCEVA™)) in a treatment scheme, e.g., in treating any of the diseases described herein, including lung cancer such as non-small-cell lung cancer, colorectal cancer, metastatic breast cancer and kidney cancer. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies. In addition, combining an antibody of this invention with a relatively non-cytotoxic agent such as another biologic molecule, e.g., another antibody, is expected to reduce cytotoxicity versus combining the antibody with a chemotherapeutic agent or other agent that is highly toxic to cells.

Treatment with a combination of the antibody herein with one or more second medicaments preferably results in an improvement in the signs or symptoms of cancer. For instance, such therapy may result in an improvement in survival (overall survival and/or progression-free survival) relative to a patient treated with the second medicament only (e.g., a chemotherapeutic agent only), and/or may result in an objective response (partial or complete, preferably complete). Moreover, treatment with the combination of an antibody herein and one or more second medicament(s) preferably results in an additive, and more preferably synergistic (or greater than additive), therapeutic benefit to the patient. Preferably, in this combination method the timing between at least one administration of the second medicament and at least one administration of the antibody herein is about one month or less, more preferably, about two weeks or less.

For treatment of cancers, the second medicament is preferably another antibody, chemotherapeutic agent (including cocktails of chemotherapeutic agents), cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, and/or growth-inhibitory agent. The cytotoxic agent includes a small-molecule inhibitor to IGF-1R as well as other peptides and anti-sense oligonucleotides and other molecules used to target IGF-1R, such as, e.g., BMS-536924, BMS-55447, BMS-636924, AG-1024, OSIP Compound 2/OSI005, NVP-ADW-742 or NVP-AEW541 (see AACR annual meeting abstracts, Apr. 1-6, 2006), bicyclo-pyrazole inhibitors such as those described in WO 2007/099171, pyrazolo-pyridine derivative inhibitors such as those described in WO 2007/099166, or another IGF-1R antibody that those claimed herein, such as those set forth above, an agent interacting with DNA, the anti-metabolites, the topoisomerase I or II inhibitors, a hyaluronidase glycoprotein as an active delivery vehicle as set forth in, for example, WO 2004/078140, or the spindle inhibitor or stabilizer agents (e.g., preferably vinca alkaloid, more preferably selected from vinblastine, deoxyvinblastine, vincristine, vindesine, vinorelbine, vinepidine, vinfosiltine, vinzolidine and vinfunine), or any agent used in chemotherapy such as 5-FU, a taxane, doxorubicin, or dexamethasone.

In another embodiment, the second medicament is another antibody used to treat cancer such as those directed against the extracellular domain of the HER2/neu receptor, e.g., trastuzumab, or one of its functional fragments, pan-HER inhibitor, a Src inhibitor, a MEK inhibitor, or an EGFR inhibitor (e.g., an anti-EGFR antibody (such as one inhibiting the tyrosine kinase activity of the EGFR), which is preferably the mouse monoclonal antibody 225, its mouse-man chimeric derivative C225, or a humanized antibody derived from this antibody 225 or derived natural agents, dianilinophthalimides, pyrazolo- or pyrrolopyridopyrimidines, quinazilines, gefitinib (IRESSA®), Apo2 ligand or tumor necrosis factor-related apoptosis-inducing ligand (Apo2L/TRAIL), a dual pro-apoptotic receptor agonist designed to activate both pro-apoptotic receptors DR4 and DR5 (including the polypeptides disclosed in WO 1997/01633, WO 1997/25428, and WO 2001/00832, where Apo2L/TRAIL is a soluble fragment of the extracellular domain of Apo2 ligand, corresponding to amino acid residues 114-281, available from Genentech, Inc./Amgen/Immunex), an Apo2L/TRAIL DR5 agonist (e.g. apomab that is a fully human monoclonal antibody that is a DR5-targeted pro-apoptotic receptor agonist, as described, for example, in US 2007/0031414 and US 2006/0088523, available from Genentech, Inc.), systemic hedgehog antagonist, erlotinib (TARCEVA™), cetuximab, ABX-EGF, canertinib, EKB-569 and PKI-166), or dual-EGFR/HER-2 inhibitor such as lapatanib. Additional second medicaments include alemtuzumab (CAMPATH™), FavID (IDKLH), CD20 antibodies with altered glycosylation, such as GA 101/GLYCART™, oblimersen (GENASENSE™), thalidomide and analogs thereof, such as lenalidomide (REVLIMID™), ofatumumab (HUMAX-CD20™), anti-CD40 antibody, e.g., SGN-40, and anti-CD80 antibody, e.g. galiximab.

Additional molecules that can be used in combination with the anti-IGF-1R antibodies herein for treatment of cancer include pan-HER tyrosine kinase inhibitors (TKI)TKI that irreversibly inhibit all HER receptors. Examples include such molecules as CI-1033 (also known as PD183805; Pfizer), GW572016 and GW2016 (GlaxoSmithKIine) and BMS-599626 (Bristol-Meyers-Squibb).

Additionally included is an inhibitor of apoptosis protein (IAP) antagonist such as, for example, Jafrac2, Diablo/Smac, and other inhibitors described, for example, in Vucic et al., Biochem. J. 385:11-20 (2005).

Also included as second medicaments for cancer treatment are c-Met inhibitors such as, for example, a monoclonal antibody to c-Met such as METMAB™ (a recombinant, humanized, monovalent monoclonal antibody directed against c-Met produced by Genentech, Inc., the variable region sequence of which is described in US 2006/0134104), as well as one-armed formats of METMAB™ antibody such as that described in US 2005/0227324, anti-HGF monoclonal antibodies, truncated variants of c-Met that act as decoys for HGF, and protein kinase inhibitors that block c-Met induced pathways (e.g., ARQ197, XL880, SGX523, MP470, PHA665752, and PF2341066).

Additional such second medicaments for cancer treatment include poly(ADP-ribose) polymerase 1 (PARP) inhibitors such as, for example, KU-59436 (KuDOS Pharma), 3-aminobenzamide (Trevigen, Inc.), INO-1001 (Inotek Pharmaceuticals and Genentech), AG014699 (Pfizer, Inc.), BS-201 and BS-401 (BiPar Sciences), ABT-888 (Abbott), AZD2281 (AstraZeneca), as described, for example, in Nature, 434: 913-917 (2005) and Nature, 434: 917-921 (2005) on the role for PARP inhibition in the development of targeted cancer therapy.

Also included are MAP-erk kinase (MEK) inhibitors such as, for example, U0124 and U0126 (Promega), ARRY-886 (AZD6244) (Array Biopharma), PD 0325901, CI-1040 (Pfizer), PD98059 (Cell Signaling Technology), and SL 327.

Further included are phosphatidylinositol 3-kinase (P13K) inhibitors such as described, for example, in WO 2007/030360, such as LY294002 and wortmannin. Further examples include analogs of 17-hydroxywortmannin (see, e.g., US 2006/0128793), azolidinone-vinyl benzene derivatives, which are described, for example, in WO 2004/007491, and 2-imino-azolinone-vinyl fused-benzene derivatives, which are described, for example, in WO 2005/011686.

Also included are, for example, AKT (protein kinase B) inhibitors such as, for example, SR13668 (SRI International), AG 1296, A-443654, KP372-1, perifosine (also known as KRX-0401; Keryx Biopharmaceuticals), and others such as those described in WO 2006/113837 (for example, imidazo[4,5-c]pyridine analogs with Akt (PKB) kinase antagonist activity containing a 4-amino-1,2,5-oxadiazole substituent at the 2-position of the ring system with an alkyne substituent at the 4-position, and diverse functionality at the 6-position.), 1L-6-hydroxymethyl-chiro-inositol 2(R)-2-O-methyl-3-O-octadecylcarbonate, PI (phosphatidylinositol) analogs, a peptide derived from the proto-oncogene TCL1, which binds to the same region on the PH domain as PIP₃, compounds that inhibit by preventing the activation of Akt via inhibition of upstream effectors such as Akt Inhibitor IV, Akt Inhibitor V, and TRICIRIBINE™ (6-amino-4-methyl-8-(β-D-ribofuranosyl).

An alternative approach to blocking PI3K/Akt signaling is the use of small molecules that inactivate the kinase mammalian target of rapamycin (mTOR), which functions downstream of Akt. Three mTOR inhibitors being tested in clinical trials for patients with breast cancer and other solid tumors are CCI-779 (otherwise known as temsirolimus; Wyeth, Madison, N.J.), RAD001 (also known as everolimus; Novartis, New York, N.Y.), and AP23573 (Ariad, Cambridge, Mass.)

Further included are inhibitors of heat-shock protein 90 (HSP90), a chaperone protein that in its activated form controls the folding of many key signal transduction client proteins including HER2, for example, for patients with HER2-overexpressing breast cancer. Examples of HSP90 inhibitors include SNX-5422 (Serenex), geldanamycin and its derivatives such as 17-allylamino-17-demethoxygeldanamycin (17-AAG), pyrazole HSP90 inhibitor CCT0180159 (The Institute of Cancer Research), and tanespimycin (KOS-953) (Kosan Biosciences).

Additional compounds include trastuzumab (HERCEPTIN®) combined with a toxin such as the fungal toxin maytansinoid (DM-1), also called T-DM1 or Herceptin DM1.

See, e.g., Am. J. Clin Pathol., 122(4):598-609 (2004) for other possible combination agents.

In addition to targeting the HER2 protein, strategies that prevent the synthesis of the HER2 transcript are useful herein, such as the one based on the finding that the HER2 gene can be repressed by the adenovirus E1A gene. Delivery of E1A expression constructs into human tumor cell lines using liposomes inhibited HER2 expression and tumorigenicity. A phase I clinical trial of E1A therapy showed that intracavitary injection of the EIA gene complexed with DC-Chol cationic liposome (DCC-E1A; Targeted Genetics Corp., Seattle, Wash.) was feasible in patients with breast cancer. See, for example, Nahta et al., Nat Clin Pract Oncol. 3(5):269-280 (2006).

Additionally, HER2 vaccines might be useful as adjuvant therapies to prevent relapse by establishing an effective memory response or as treatments for patients whose disease has progressed during treatment with trastuzumab.

The anti-emetic agent is preferably ondansetron hydrochloride, granisetron hydrochloride, metroclopramide, domperidone, haloperidol, cyclizine, lorazepam, prochlorperazine, dexamethasone, levomepromazine, or tropisetron. The vaccine is preferably GM-CSF DNA and cell-based vaccines, dendritic cell vaccines, recombinant viral vaccines, heat shock protein (HSP) vaccines, allogeneic or autologous tumor vaccines. The analgesic agent preferably is ibuprofen, naproxen, choline magnesium trisalicylate, or oxycodone hydrochloride. The anti-vascular agent preferably is bevacizumab, or rhuMAb-VEGF. Further second medicaments include anti-proliferative agents such as farnesyl protein transferase inhibitors, anti-VEGF inhibitors, p53 inhibitors, or PDGFR inhibitors. The second medicament herein includes also biologic-targeted therapy such as treatment with antibodies as well as small-molecule-targeted therapy, for example, against certain receptors.

Further second medicaments include agents that lower IGF-I concentrations such as growth-hormone releasing hormone (GHRH) antagonists (Letsch et al., Proc Natl Acad Sci USA, 100: 1250-1255 (2003)), and a PEGylated GH receptor antagonist (pegvisomant) useful to disrupt GH signaling in patients with acromegaly and cancer (McCutcheon et al., J. Neurosurg., 94: 487-492 (2001)). IGF-I neutralizing monoclonal antibodies and IGFBPs are also useful second medicaments in breast cancer (Van den Berg et al., Eur J Cancer, 33: 1108-1113 (1997)) and prostrate cancer (Goya et al., Cancer Res, 64: 6252-6258 (2004)).

Preferred chemotherapeutic agents useful herein include a taxane (e.g., paclitaxel and docetaxel), a topoisomerase inhibitor (e.g., etoposide, topotecan, camptothecin and irinotecan), a signal-transduction inhibitor, a cell-cycle inhibitor, an IGF/IGF-1R system modulator, a dual EGFR/HER-2 kinase inhibitor (e.g., lapatinib), a HER-2 downregulator or client protein of Hsp90 downregulator such as 17AAG (geldanamycin derivative that is a heat shock protein (Hsp) 90 poison), an anti-estrogen such as fulvestrant, a Kit inhibitor such as imatinib or EXEL-0862, a tyrosine kinase inhibitor (see AACR annual meeting abstracts, Apr. 1-5, 2006), a farnesyl protein transferase (FPT) inhibitor (e.g., lonafarnib and tipifamib (R155777)), a HER2 inhibitor (e.g., trastuzumab, 2C4, HKI-272, CP-724714 or TAK-165), a vascular epidermal growth factor (VEGF) receptor inhibitor, a mitogen-activated protein (MAP) kinase inhibitor, a MEK inhibitor, an AKT inhibitor, a mTOR inhibitor, a pl3 kinase inhibitor, a Raf inhibitor, a cyclin-dependent kinase (CDK) inhibitor, a microtubule stabilizer, a microtubule inhibitor (e.g., vincristine, vinblastine, a podophyllotoxin, epothilone B, BMS-247550, BMS-310705, allocolchicine, Halichondrin B, colchicine, colchicine derivatives, dolastatin 10, maytansine, rhizoxin, paclitaxel, a paclitaxel derivative, etoposide, thiocolchicine, trityl cysteine, vinblastine sulfate, vincristine sulfate, epothilone A, epothilone B, discodermolide, estramustine, nocodazole, or MAP4), 5-FU, a platinum coordination complex such as cisplatin or carboplatin, a natural product (such as a vinca alkaloid, an antitumor antibiotic, an enzyme, lymphokine, epipodophyllotoxin, vinblastine, vincristine, vindesine, bleomycin, dactinomycin, daunorubicin, doxorubicin, epirubicin, idarubicin, ara-C, mithramycin, deoxyco-formycin, mitomycin-C, L-asparaginase, an interferon, etoposide, or teniposide), a selective estrogen-receptor modulator (SERM)/antiestrogen (such as tamoxifen, raloxifene, fulvestrant, acolbifene, pipendoxifene, arzoxifene, toremifene, lasofoxifene, bazedoxifene (TSE-424), idoxifene, HMR-3339 or ZK-186619), an aromatase inhibitor, an anthracycline (e.g., doxorubicin, liposomal doxorubicin, daunorubicin, or epirubicin), an mTOR inhibitor, an agent that inhibits IGF production, an anti-sense inhibitor of IGF-1R, IGF-I or IGF-II, or radiation, including iodizing radiation.

Exemplary second medicaments include an alkylating agent, a folate antagonist, a pyrimidine antagonist, a cytotoxic antibiotic, a platinum compound or platinum-based compound, a taxane, a vinca alkaloid, an Apo2L/TRAIL DR5 agonist (such as apomab, a DR-5-targeted dual proapoptotic receptor agonist), a c-Kit inhibitor, a topoisomerase inhibitor, an anti-angiogenesis inhibitor such as an anti-VEGF inhibitor, a HER-2 inhibitor, an EGFR inhibitor or dual EGFR/HER-2 kinase inhibitor, an anti-estrogen such as fulvestrant, and a hormonal therapy agent, such as carboplatin, cisplatin, gemcitabine, capecitabine, epirubicin, tamoxifen, an aromatase inhibitor, and prednisone. Most preferably, the cancer is colorectal cancer and the second medicament is an EGFR inhibitor such as erlotinib, apomab, an anti-VEGF inhibitor such as bevacizumab, or is cetuximab, arinotecan, irinotecan, or FOLFOX, or the cancer is breast cancer and the second medicament is an anti-estrogen modulator such as fulvestrant, tamoxifen, apomab, or an aromatase inhibitor such as letrozole, exemestane, or anastrozole, or is a VEGF inhibitor such as bevacizumab, or is a chemotherapeutic agent such as doxorubicin, and/or a taxane such as paclitaxel, or is an anti-HER-2 inhibitor such as trastuzumab, or a dual EGFR/HER-2 kinase inhibitor such as lapatinib or a HER-2 downregulator such as 17AAG (geldanamycin derivative that is a heat shock protein [Hsp] 90 poison) (for example, for breast cancers that have progressed on trastuzumab). In other embodiments, the cancer is lung cancer, such as small-cell lung cancer, and the second medicament is a VEGF inhibitor such as bevacizumab, apomab, or an EGFR inhibitor such as, e.g., erlotinib or a c-Kit inhibitor such as, e.g., imatinib. Further, a preferred chemotherapeutic agent for front-line therapy of cancer is docetaxel (TAXOTERE®), alone or in combination with other second medicaments. Most preferably, if chemotherapy is administered, it is given first, followed by the antibodies herein.

In a preferred combination embodiment for cancer, the antibodies herein are given with another biological agent such as an antibody or another non-chemotherapeutic agent such as an anti-estrogen inhibitor or other targeted inhibitor, more preferably a biological agent or anti-estrogen inhibitor. It is expected that an anti-estrogen inhibitor in combination with an antibody herein may show additive or even synergistic effects in treating breast cancer, particular ER-positive breast cancer.

Examples of suitable second medicaments for use in an effective amount to treat aging include one or more of the following, given separately, in sequence, or simultaneously: statins, bisphosphonates, cholesterol-lowering agents or techniques, interleukin-6 inhibitor/antibody, interleukin-6 receptor inhibitor/antibody, interleukin-6 anti-sense oligonucleotide (ASON), gp130 protein inhibitor/antibody, diabetes treatment, tyrosine kinases inhibitors/antibodies other than the antibodies herein, a hyaluronidase glycoprotein (as an active delivery vehicle), serine/threonine kinases inhibitors/antibodies, mitogen-activated protein (MAP) kinase inhibitors/antibodies, an insulin-resistance-treating agent (e.g., insulin (one or more different types of insulin), insulin mimetics, such as a small-molecule insulin, e.g., L-783,281, insulin analogs (e.g., LYSPRO™ (Eli Lilly Co.), Lys^{B28}insulin, Pro^{B29}insulin, or Asp^{B28}insulin or those described in, for example, U.S. Pat. Nos. 5,149,777 and 5,514,646) or physiologically active fragments thereof, and insulin-related peptides (C-peptide, GLP-1, IGF-1, or IGF-1/IGFBP-3 complex) or analogs or fragments thereof), phosphatidyl inositol 3-kinase (PI3K) inhibitors/antibodies, Nuclear factor κB (NF-κB) inhibitors/antibodies, IκB kinase (IKK) inhibitors/antibodies, activator protein-1 (AP-1) inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof. In preferred aspects, they include a statin, bisphosphonate, cholesterol-lowering agent, hypertension-treating agent, interleukin-6 inhibitor, interleukin-6 receptor inhibitor, interleukin-6 anti-sense oligonucleotide, gp130 protein inhibitor, growth hormone, growth-hormone-releasing hormone, growth-hormone secregatogue, or insulin-resistance-treating agent.

Such second medicaments may be administered within 48 hours after the antibodies herein are administered, or within 24 hours, or within 12 hours, or within 3-12 hours after said agent, or may be administered over a preselected period of time, which is preferably about 1 to 2 days. Further, the dose of such agent may be sub-therapeutic.

Where an antibody of the invention inhibits inflammation or autoimmune reactions, the second medicament is one designed for treating such condition. For instance, an antibody of the invention may be combined with methotrexate in a treatment scheme, e.g. in treating any of the diseases described herein, including rheumatoid arthritis or lupus. Such combined therapies noted above include combined administration (where the two or more agents are included in the same or separate formulations), and separate administration, in which case, administration of the antibody of the invention can occur prior to, and/or following, administration of the adjunct therapy or therapies. Such second medicament includes, for example, a chemotherapeutic agent, an immunosuppressive agent, an antagonist (such as an antibody) that binds a B-cell surface marker such as rituximab or humanized 2H7, a BAFF antagonist such as BR3-Fc, a disease-modifying anti-rheumatic drug (DMARD), a cytotoxic agent, an integrin antagonist, a non-steroidal anti-inflammatory drug (NSAID), a cytokine antagonist, a hormone, a TNF antagonist, an anti-rheumatic agent, a muscle relaxant, a narcotic, an analgesic, an anesthetic, a sedative, a local anesthetic, a neuromuscular blocker, an antimicrobial, an anti-psoriatic drug, a corticosteriod, an anabolic steroid, an erythropoietin, an immunization, an immunoglobulin, a radiopharmaceutical, an antidepressant, an anti-psychotic drug, a stimulant, an asthma medication, a beta agonist, a hyaluronidase glycoprotein (as an active delivery vehicle), an inhaled steroid, an epinephrine, a cytokine, cells for repressing B-cell autoantibody secretion as set forth in WO 2005/027841, or a combination thereof. Preferably, such second medicament is a TNF antagonist, an immunosuppressive agent, an antagonist that binds a B-cell surface marker such as an antibody, e.g., anti-CD20 or anti-CD22 antibody, a BAFF antagonist, a chemotherapeutic agent, a DMARD, a cytotoxic agent, an integrin antagonist, a NSAID, a cytokine antagonist, or a hormone.

In treating the autoimmune disorders described above, the patient can be treated with the antibodies herein along with a B-cell depleting agent such as CD20-binding antibodies such as rituximab or a humanized 2H7 antibody in conjunction with a third therapeutic agent, such as an immunosuppressive agent, such as in a multi drug regimen. The antibodies herein can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

For the treatment of RA, for example, the patient can be treated with an antibody of the invention in conjunction with any one or more of the following drugs: DMARDs (e.g., MTX), NSAI or NSAID (non-steroidal anti-inflammatory drugs), HUMIRA™ (adalimumab; Abbott Laboratories), ARAVA® (leflunomide), REMICADE® (infliximab; Centocor Inc., of Malvern, Pa.), ENBREL (etanercept; Immunex, WA), COX-2 inhibitors. DMARDs commonly used in RA include hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNFα. Infliximab is a chimeric monoclonal antibody that binds to TNFα. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., American College of Rheumatology Subcommittee on Rheumatoid Arthritis Guidelines, Arthritis & Rheumatism, 46(2): 328-346 (2002). In a specific embodiment, the RA patient is treated with an IGF-1R antibody of the invention in conjunction with MTX. An exemplary dosage of MTX is about 7.5-25 mg/kg/wk. MTX can be administered orally and subcutaneously.

For the treatment of ankylosing spondylitis, psoriatic arthritis, and Crohn's disease, the patient can be treated with an antibody of the invention in conjunction with, for example, REMICADE® (infliximab; from Centocor Inc., of Malvern, Pa.), ENBREL® (etanercept; Immunex, WA).

Treatments for SLE include high-dose corticosteroids and/or cyclophosphamide (HDCC) in conjunction with the antibodies herein.

For the treatment of psoriasis, patients can be administered a IGF-1R-binding antibody in conjunction with topical treatments, such as topical steroids, anthralin, calcipotriene, clobetasol, and tazarotene, or with MTX, retinoids, cyclosporine, or PUVA and UVB therapies. In one embodiment, the psoriasis patient is treated with the IGF-1R-binding antibody sequentially or concurrently with cyclosporine.

The antibodies herein can be administered concurrently, sequentially, or alternating with the second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as the first medicaments, or from about 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

### Articles of Manufacture

In another embodiment of the invention, articles of manufacture containing materials useful for the treatment of the disorders described above are provided. In one aspect, the article of manufacture comprises (a) a container comprising the antibodies herein (preferably the container comprises the antibody and a pharmaceutically acceptable carrier or diluent within the container); and (b) a package insert with instructions for treating the disorder in a patient.

In a preferred embodiment, the article of manufacture herein further comprises a container comprising a second medicament, wherein the antibody is a first medicament. This article further comprises instructions on the package insert for treating the patient with the second medicament, in an effective amount.

The second medicament may be any of those set forth above, with an exemplary second medicament for cancer being another antibody, chemotherapeutic agent (including cocktails of chemotherapeutic agents), cytotoxic agent, anti-angiogenic agent, immunosuppressive agent, prodrug, cytokine, cytokine antagonist, cytotoxic radiotherapy, corticosteroid, anti-emetic, cancer vaccine, analgesic, anti-vascular agent, and/or growth-inhibitory agent. An exemplary second medicament for autoimmune disorders is an antagonist binding to a B-cell surface marker (e.g., a CD20 antibody), a BAFF antagonist, a TNF antagonist, a chemotherapeutic agent, an immunosuppressive agent, a cytotoxic agent, an integrin antagonist, a cytokine antagonist, or a hormone.

The preferred second medicaments for treating autoimmune diseases are those preferred as set forth above, including a steroid or an immunosuppressive agent or both.

Exemplary second medicaments for treating aging include a statin, bisphosphonate, cholesterol-lowering agent, hypertension-treating agent, interleukin-6 inhibitor, interleukin-6 receptor inhibitor, interleukin-6 anti-sense oligonucleotide, gp130 protein inhibitor, growth hormone, growth-hormone-releasing hormone, growth-hormone secregatogue, or insulin-resistance treating agent.

In this aspect, the package insert is on or associated with the container. Suitable containers include, e.g., bottles, vials, syringes, etc. The containers may be formed from a variety of materials such as glass or plastic. The container holds or contains a composition that is effective for treating the disorder in question and may have a sterile access port (e.g., the container may be an intravenous solution bag or a vial having a stopper pierceable by a hypodermic injection needle). At least one active agent in the composition is the antibody herein. The label or package insert indicates that the composition is used for treating the particular disorder in a patient or subject eligible for treatment with specific guidance regarding administration of the compositions to the patients, including dosing amounts and intervals of antibody and any other medicament being provided. Package insert refers to instructions customarily included in commercial packages of therapeutic products that contain information about the indications, usage, dosage, administration, contra-indications, and/or warnings concerning the use of such therapeutic products.

The article of manufacture may further comprise an additional container comprising a pharmaceutically acceptable diluent buffer, such as bacteriostatic water for injection (BWFI), phosphate-buffered saline (PBS), Ringer's solution, and/or dextrose solution. The article of manufacture may further include other materials desirable from a commercial and user standpoint, including other buffers, diluents, filters, needles, and syringes.

In another aspect, the invention provides a method for packaging or manufacturing an antibody herein or a pharmaceutical composition thereof comprising combining in a package the antibody or pharmaceutical composition and a label stating that the antibody or pharmaceutical composition is indicated for treating patients with a disorder such as cancer, aging, or an autoimmune disease such as RA, MS, lupus, or IBD.

### Methods of Advertising

The invention herein also encompasses a method for advertising an antibody herein or a pharmaceutically acceptable composition thereof comprising promoting, to a target audience, the use of the antibody or pharmaceutical composition thereof for treating a patient or patient population having a disorder such as cancer, aging, or an autoimmune disease such as RA, MS, lupus, or IBD.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the treatment methods herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed, generally in public places. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments. The advertising may be individualized or customized based on user characterizations defined by service interaction and/or other data such as user demographics and geographical location.

### Biomarkers

Also provided herein are methods for assessing the activity of an IGF-1R antibody. In this method, any anti-IGF-1R antibody can be used, but most preferably it is one of those disclosed herein.

One of these methods is a method for assessing the activity of an antibody in tumor tissue comprising subjecting tissue from tumors (such as breast cancer or neuroblastoma, but including all tumor types) treated with the antibody to FDG-PET imaging and then determining if the antibody inhibits FDG uptake into the tissue. Inhibition of FEG uptake correlates with delayed tumor growth in this method. Methods for carrying out the imaging and determining if FDG uptake is inhibited are known in the art, and include those described in the Examples below.

The following are non-limiting examples of the methods and compositions of the invention. In the Examples, the term “HVR” is used as defined herein, and is inclusive of “CDR.” It is understood that various other embodiments may be practiced, given the general description provided above. The disclosures of all citations in the specification are expressly incorporated herein by reference.

### EXAMPLE 1

### Generating Hybridoma and Phage Antibodies Against IGF-1R

### I. Hybridoma Production

The human IGF-1R was generated and purified as secreted recombinant protein containing the extracellular domain (ECD, amino acids 1-902) tagged with 10 or 12 histidine residues at the carboxyl terminus using standard procedures (Ullrich et al., EMBO, supra). Five Balb/c mice (Charles River Laboratories, Hollister, Calif.) were hyperimmunized with human IGF-1R-ECD (amino acids 1-902) in RIBI™ adjuvant (Ribi Immunochem Research, Inc.; Hamilton, Mo.). B-cells from these mice, all of which demonstrated high anti-IGF-1R antibody titers by direct ELISA and specific binding to IGF-1R expressed on MCF7 breast cancer cells by fluorescence activated cell sorting (FACS), were fused with mouse myeloma cells (X63.Ag8.653; ATCC, Manassas, Va.) using a modified protocol analogous to one previously described (Kohler and Milstein., supra; Hongo et al., supra). After 10-12 days, the supernatants were harvested and screened for antibody production by direct ELISA and FACS. Fourteen positive clones, selected based on their strong binding to the purified IGF-1R-ECD and cell-surface IGF-1R, as well as their blocking activity that prevents ligand/receptor interaction (see Example 2-II), were expanded and cultured for further characterization. The supernatants harvested from each hybridoma lineage were purified by affinity chromatography (PHARMACIA™ fast-protein liquid chromatography (FPLC); Pharmacia, Uppsala, Sweden) using a modified protocol analogous to one previously described (Hongo et al., supra). The purified antibody preparations were then sterile filtered (0.2-μm pore size; Nalgene, Rochester, N.Y.) and stored at 4° C. in PBS. Only antibodies with a level of endotoxin lower than 1.5 EU/mg were used for in vitro and in vivo work.

Hybridomas 1C2, 2A7, 2B4, 2B7, 3B9, 4D3, 4D7, 5E3, 6D2, 6F10, 9F2, 9A11, and 10H5 were submitted to the ATCC, 10801 University Boulevard, Manassas, Va. 20110 on Sep. 20, 2005 with the following deposit numbers:

| Hybridoma | Deposit No. |
|---|---|
| 10H5.3.4 | PTA-7007 |
| 1C2.8.1 | PTA-7008 |
| 2B4.2.8 | PTA-7009 |
| 2A7.5.1 | PTA-7010 |
| 2B7.4.1 | PTA-7011 |
| 3B9.4.1 | PTA-7012 |
| 4D3.6.2 | PTA-7013 |
| 6F10.1.1 | PTA-7014 |
| 5E3.1.1 | PTA-7015 |
| 6D2.6.1 | PTA-7016 |
| 4D7.1.4 | PTA-7017 |
| 9F2.6.2 | PTA-7018 |
| 9A11.3.1 | PTA-7019 |

### II. Preparation and Testing of Humanized Antibodies

### A. Materials and Methods

Residue numbers are according to Kabat (Kabat et al., supra). Single-letter amino-acid abbreviations are used. DNA degeneracies are represented using the IUB code (N=A/C/G/T, D=A/G/T, V=A/C/G, B=C/G/T, H=A/C/T, K=G/T, M=A/C, R=A/G, S=G/C, W=A/T, Y=C/T).

The preferred antibodies herein are the humanized antibodies 2B4 (rhuMAb 2B4; 2B4.vX), 9F2 (rhuMAb 9F2; 9F2.vX), and 10H5 (rhuMAb 10H5; 10H5.vX), as well as a mutant humanized antibody 10H5, which has a D265A mutation based on Kabat numbering, resulting in loss of binding to Fc gamma receptor (rhuMAb 10H5m; 10H5.vXm). Most preferred is 10H5.vX and its affinity-matured clones as described herein.

(1) Direct HVR Grafts onto the Acceptor Human Consensus Framework

The phagemid used for this work is a monovalent Fab-g3 display vector (pV0350-2B) having two open reading frames under control of the phoA promoter, essentially as described in Lee et al., J. Mol. Biol., supra (2004). The first open reading frame consists of the STII signal sequence fused to the VL and CH1 domains acceptor light chain and the second consists of the STII signal sequence fused to the VH and CH1 domains of the acceptor heavy chain followed by a truncated minor phage coat protein P3. See Lee et al., J. Mol. Biol., supra (2004).

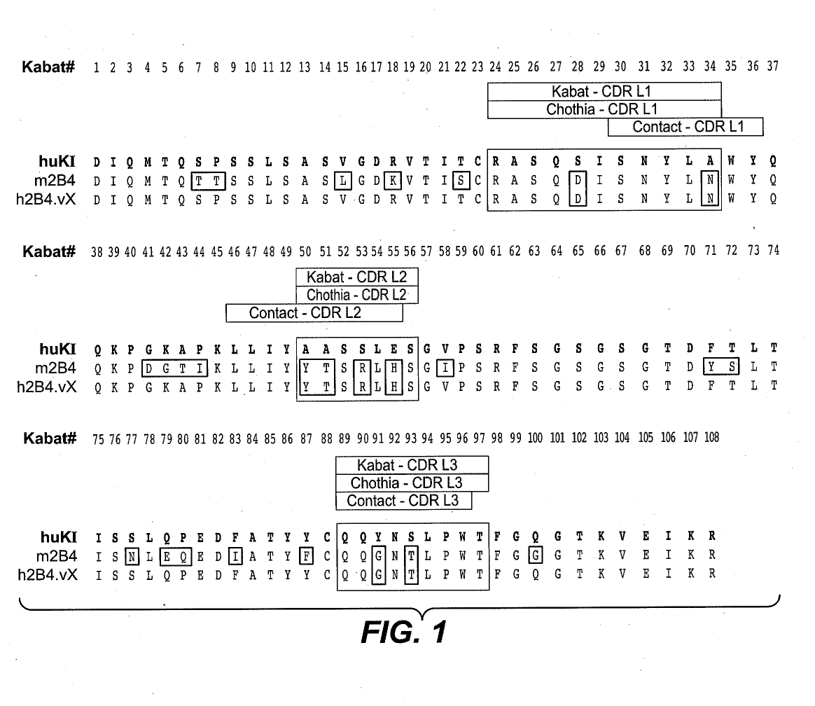

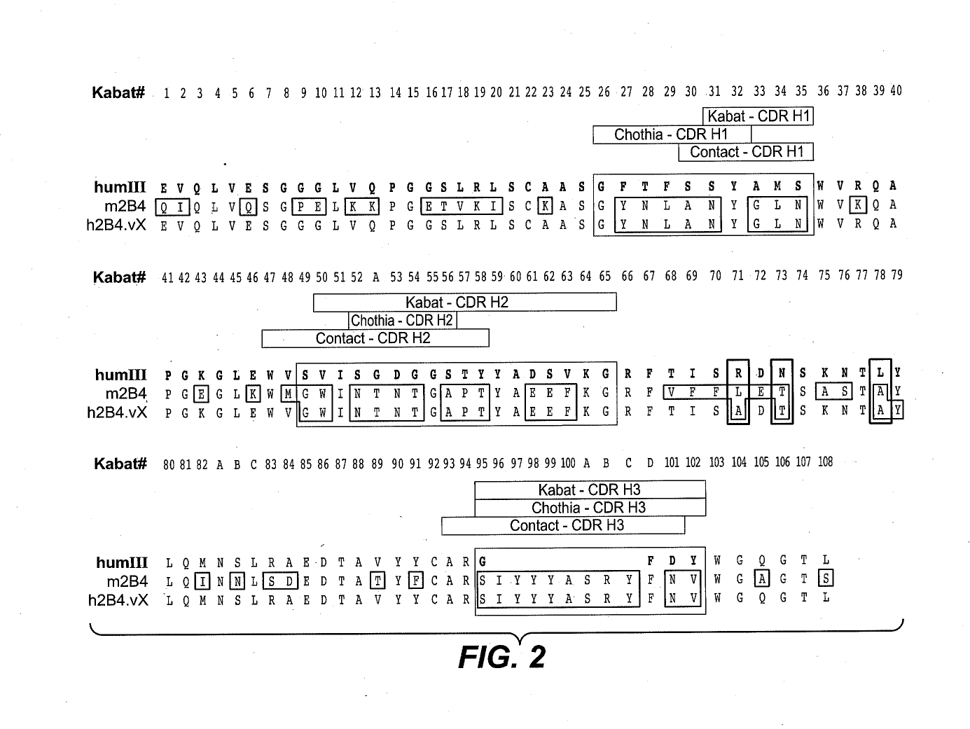

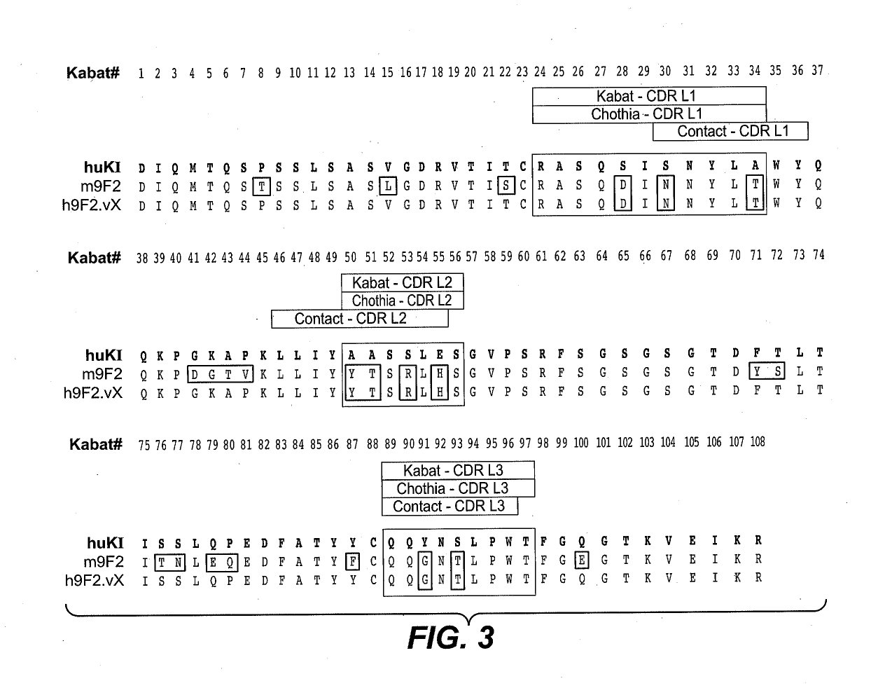

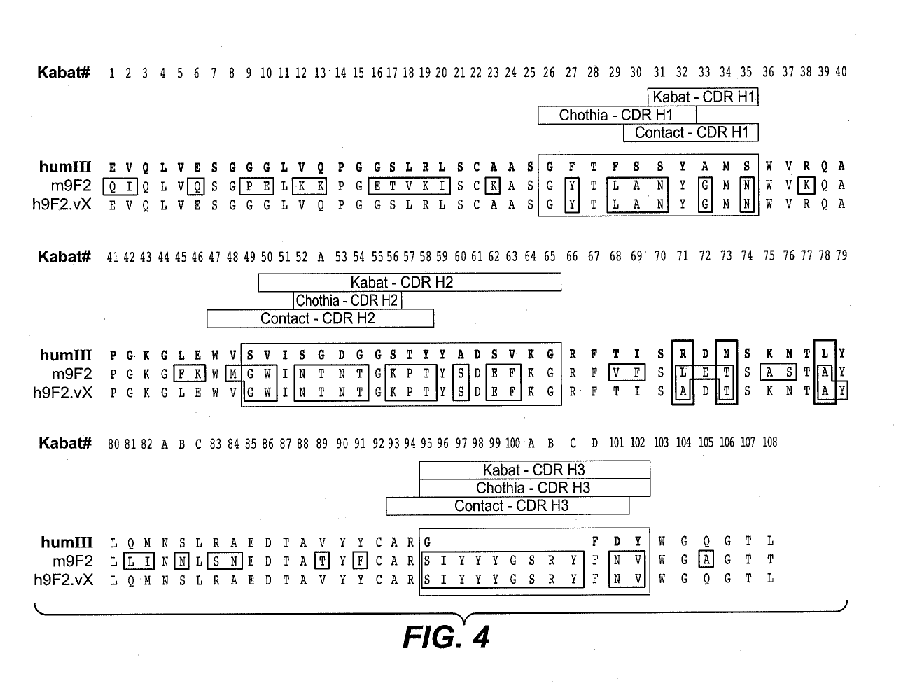

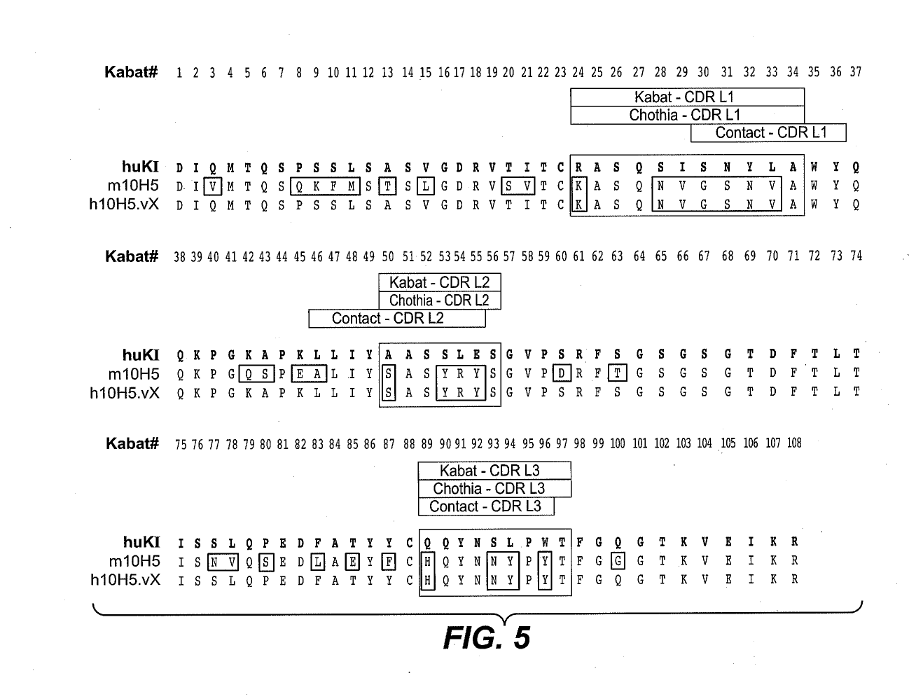

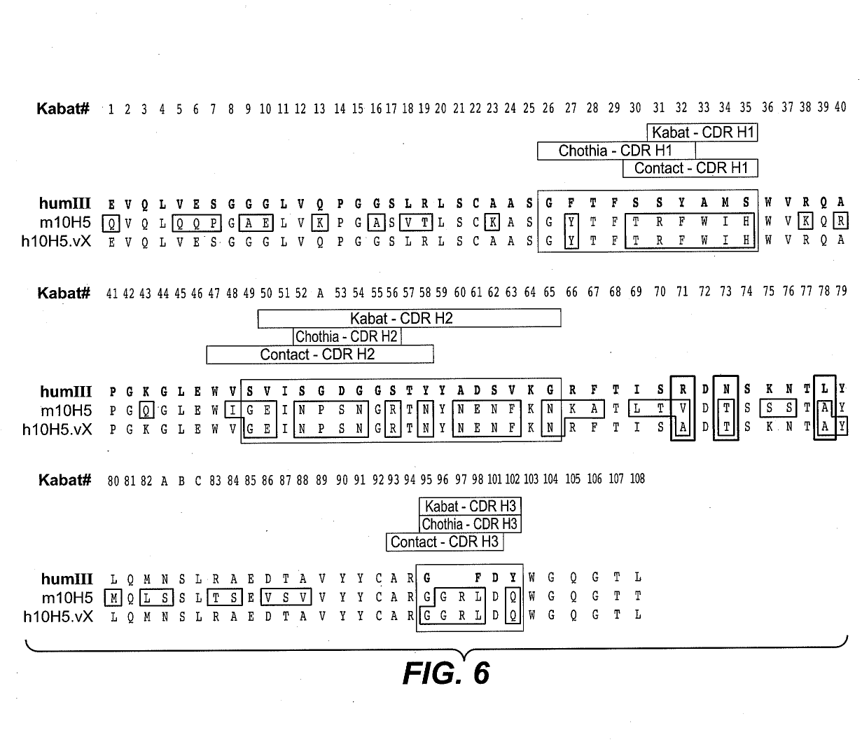

The VL and VH domains from murine 9F2 (see hybridoma 9F2, ATCC Deposit No. PTA-7018), murine 2B4 (see hybridoma 2B4, ATCC Deposit No. PTA-7009), and murine 10H5 (see hybridoma 10H5.3.4, ATCC Deposit No. PTA-7007) were aligned with the human consensus kappa I (huKI) and human subgroup III consensus VH (huIII) domains. To make the HVR graft, the acceptor VH framework, which differs from the human subgroup III consensus VH domain at 3 positions: R71A, N73T, and L78A (Carter et al., Proc. Natl. Acad. Sci. USA, supra (1992)), was used. HVRs from the murine antibodies above were engineered into the acceptor human consensus framework to generate a direct HVR-graft of 2B4, 9F2, and 10H5 (h2B4.vX, h9F2.vX, and h10H5.vX, respectively). In the VL domain, the following regions were grafted to the human consensus acceptor: positions 24-34 (L1), 50-56 (L2), and 89-97 (L3). In the VH domain, positions 26-35 (H1), 49-65 (H2), and 95-102 (H3) were grafted (see the sequence alignments for all three antibodies in FIGS. 1-6).

The direct-graft variants were generated by Kunkel mutagenesis using a separate oligonucleotide for each HVR. Correct clones were assessed by DNA sequencing.

### (2) Soft Randomization of the HVRs

Sequence diversity was introduced into each HVR using a soft randomization strategy that maintains a bias towards the murine HVR sequence. This was accomplished using a poisoned oligonucleotide synthesis strategy as described by Gallop et al., J. Med. Chem., 37:1233-1251 (1994). For a given position within a HVR to be mutated, the codon encoding the wild-type amino acid was poisoned with a 70-10-10-10 mixture of nucleotides, resulting in an average 50 percent mutation rate at each position.

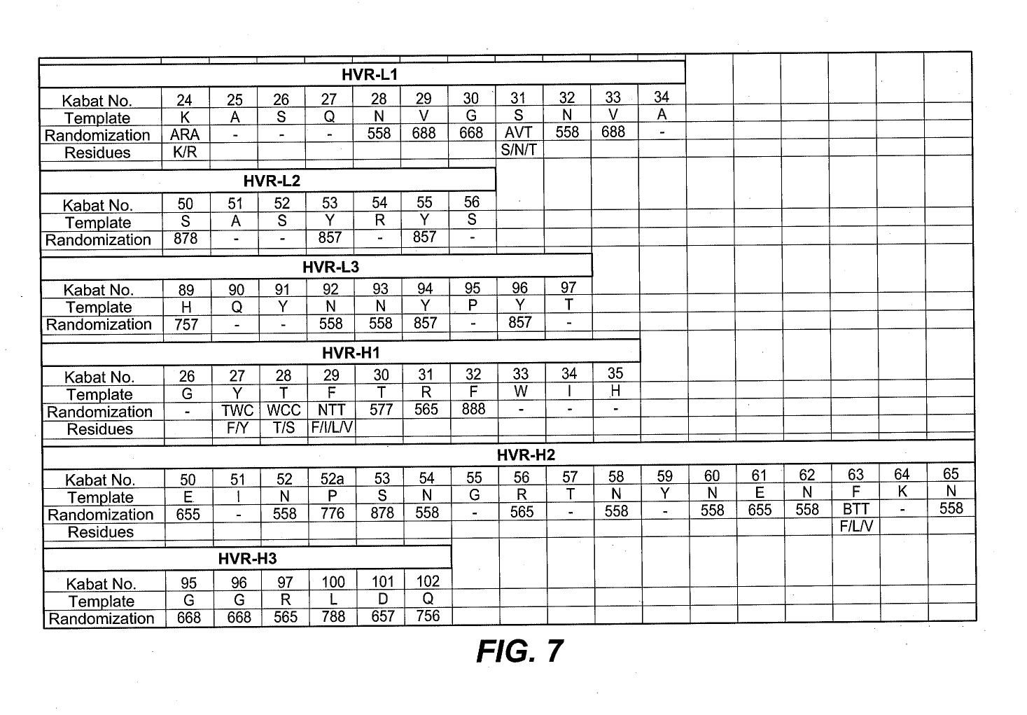

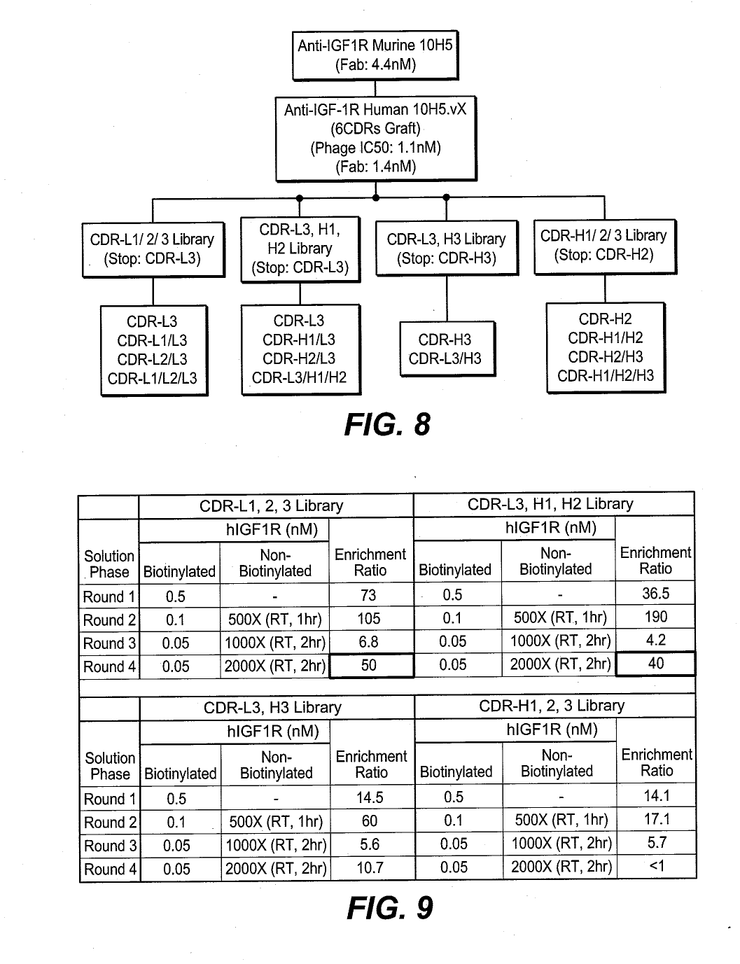

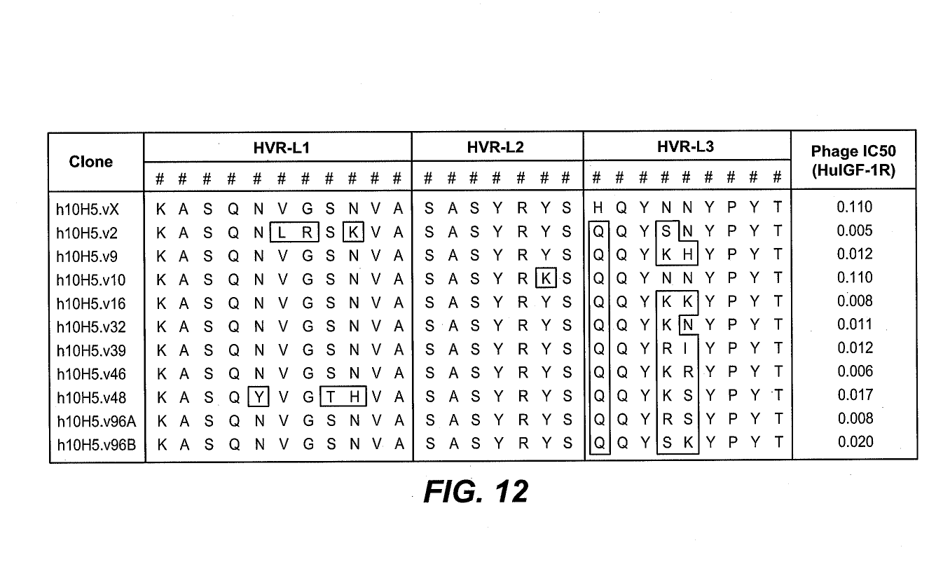

In the oligonucleotide design for h10H5.vX affinity maturation, certain HVR residues of this clone at positions 25-27 in HVR-L1, positions 51, 52, 54, and 56 in HVR-L2, positions 90, 91, 95, and 97 in HVR-L3, positions 26, 33, 34, and 35 in HVR-H1, and positions 51, 55, 57, 59, and 64 in HVR-H2 were reserved and not chosen for randomization because they were identical to the human consensus residues. Several residues were limited in sequence diversity, for example, position 24 in HVR-L1 for K or R by using codon ARA, position 31 in HVR-L1 for S, N, or T by using codon AVT, position 27 in HVR-H1 for F or Y by using codon TWC, position 28 in HVR-H1 for T or S by using codon WCC, position 29 in HVR-H1 for F, I, L, or V by using codon NTT, and position 63 in HVR-H2 for F, L, or V by using codon BTT. The rest of the HVR residues were randomized with soft randomization codons as described above. See FIG. 7. Also see FIGS. 8-11 for further details on affinity maturation, and FIG. 12 for the sequences of the final selected clones.

### (3) BIACORE™ Instrument Experiments

Binding affinities of antibodies to IGF-1R were determined by surface-plasmon resonance measurements on a BIACORE™ 3000 instrument (BIAcore, Inc.). Human IGF-1R-ECD was immobilized at a density of about 500 RU on the flow cells of a PIONEER™ CM5 sensor chip. Immobilization was achieved by random coupling through amino groups using a protocol provided by the manufacturer. Sensorgrams were recorded for binding of anti-IGF-1R Fab or IgG to these surfaces by injection of a series of solutions ranging from 500 nM to 3.1 nM and 250 nM to 0.78 nM in 2-fold increments, respectively. The signal from the reference cell was subtracted from the observed sensorgram. Kinetic constants were calculated by nonlinear regression analysis of the data according to a 1:1 Languir binding model using software supplied by the manufacturer.

### (4) Phage ELISA

MAXISORP™ microtiter plates were coated with human IGF-1R-ECD at 5 μg/ml in PBS overnight and then blocked with CASEIN BLOCKER™ reagent (Pierce Biotechnology, Inc.). Phage from culture supernatants were incubated with serially diluted IGF-1R-ECD in PBS with 0.05% TWEEN 20™ surfactant (PBST) containing 1% bovine serum albumin (BSA) in a tissue-culture microtiter plate for 1 hour, after which 80 μl of the mixture was transferred to the target-coated wells for 15 minutes to capture unbound phage. The plate was washed with PBST, and HRP-conjugated anti-M13 (Amersham Pharmacia Biotech) was added (1:5000 in PBST containing 1% BSA) for 40 minutes. The plate was washed with PBST and developed by adding tetramethylbenzidine (TMB) substrate (Kirkegaard and Perry Laboratories, Gaithersburg, Md.). The absorbance at 450 nm was plotted as a function of target concentration in solution to determine an IC₅₀. This was used as an affinity estimate for the Fab clone displayed on the surface of the phage. For the humanized hybridoma-derived clones, 2B4, 9F2, and 10H5, phage-Fab was used.

### (5) IgG and Fab Production and Affinity Determination for Hybridoma-Derived Antibodies

Clones of interest (chimeric 9F2, h9F2.vX, chimeric 10H5, h10H5.vX, and h10H5.vX-based affinity matured clones) were reformatted into a human IgG1 pRK vector (Carter et al., Proc. Natl. Acad. Sci. USA, supra (1992)), transiently expressed in 293 cells, and purified with Protein A affinity chromatography. Murine 2B4, 9F2, and 10H5Fab were generated using papain digestion at 37° C. overnight and purified through a sizing column.

Affinity determinations were performed by surface-plasmon resonance using a BIACORE™-2000 biosensor. IGF-1R-ECD was immobilized (about 500 RU) on a CM5 chip, and 2-fold serial diluted concentrations of Fab (3.1 to 500 nM) or IgG (0.78 to 250 nM) in PBST were injected. After each injection, the chip was regenerated using 20 mM HCl. Binding response was corrected by subtracting the RU from a blank flow cell. A 1:1 Languir model of simultaneous fitting of kₒₙ and k_{off} was used for kinetics analysis.

### (6) Affinity Maturation of Anti-IGF-1R Antibody

To generate the library template for affinity maturation of a selected anti-IGF-1R antibody clone, the GCN4 leucine zipper of parental phagemid was first removed using Kunkel mutagenesis to provide a monovalent display Fab format. Next, a stop codon was incorporated into all HVR-L3 positions targeted for randomization. Sequence diversity was introduced into each HVR using a soft randomization strategy that maintains a bias towards the murine HVR sequence. This was accomplished using a poisoned oligonucleotide synthesis strategy as described by Gallop et al., supra. For a given position within a HVR to be mutated, the codon encoding the wild-type amino acid is poisoned with a 70-10-10-10 mixture of nucleotides resulting in an average 50 percent mutation rate at each position.

Three different libraries with combinations of HVR loops, L1/L2/L3, L3/H1/H2, and L3/H3 randomization, were generated through soft-randomizing selected residues at positions 28-32 of HVR-L1; 50 and 53-55 of HVR-L2; 91, 92, 93, 94 and 96 of HVR-L3; 28-35 of HVR-H1; 50-58 of HVR-H2; and 95-100 of HVR-H3.

For affinity maturation, phage libraries were sorted using a solution-sorting method. IGF-1R-ECD was biotinylated by mixing 500 μl of 3.6 mg/ml IGF-1R-ECD in PBS, and 10 μl of 1 M potassium phosphate, pH 8 with 20 μl 4 mM SULFO-NHS-LC-BIOTIN™ reagent (Pierce Biotechnology, Inc.). Biotinylated IGF-1R-ECD was purified using a NAP5™ column (Amersham Biosciences) in PBS. Microtiter wells were coated with 10 μg/ml neutravidin in PBS overnight at 4° C. and then blocked for one hour using SUPERBLOCKER™ solution (Pierce Biotechnology, Inc.).

In the first round of panning, 200 μl phage suspended in SUPERBLOCKER® solution (Pierce Biotechnology, Inc.) containing 0.05% TWEEN™ 20 surfactant were mixed with 0.5 nM biotinylated IGF-1R-ECD for one hour. Phage bound to biotinylated IGF-1R-ECD were captured on neutravidin-coated wells for 15 minutes and unbound phage were washed away with PBST. Phage were eluted using 20 mM HCl, 500 mM KCl for 45 minutes, neutralized, and propagated in XL1 blue cells (Stratagene) in the presence of K07 helper phage (New England Biolabs). Subsequent rounds of sorting were performed similarly with the following exceptions: in round 2 the final biotinylated IGF-1R-ECD concentration was 0.1 nM and 50 nM non-biotinylated IGF-1R-ECD competitor (500×) was added to the mixture for one hour at 25° C. prior to capture on neutravidin, in round 3 the final biotinylated IGF-1R-ECD concentration was 0.05 nM and 50 nM non-biotinylated IGF-1R-ECD competitor (1000×) was added to the mixture for two hours at 25° C. prior to capture on neutravidin, and in round 4 the final biotinylated IGF-1R-ECD concentration was 0.05 nM and 100 nM non-biotinylated IGF-1R-ECD competitor (2000×) was added to the mixture for two hours at 25° C. prior to capture on neutravidin.

### (7) High-Throughput, Affinity-Screening Phage ELISA (Single-Spot Competition)

Colonies were picked from the fourth-round screens of two significantly enriched libraries, HVR-L1, -L2, L3 and HVR-L3, -H1, -H2, and grown overnight at 37° C. in 150 μL/well of 2YT media with 50 μg/ml carbenicillin and 1e10/ml M13/KO7 helper phage (New England Biolabs) in a 96-well plate (Falcon). From the same plate, a 1×1¹⁰ colony of XL1 BLUE™ cells (Stratagene) infected with parental phage (h10H5.vX) was picked as control. Ninety-six-well NUNC™ MAXISORP™ plates were coated with 100 μL/well of IGF-1R-ECD (2 μg/ml) in PBS at 4° C. overnight or at room temperature for two hours. The plates were blocked with 65 μL of 1% BSA for 30 minutes and 40 μL of 1% TWEEN™ 20 surfactant for another 30 minutes.

The phage supernatant was diluted 1:10 in ELISA buffer (PBS with 0.5% BSA, 0.05% TWEEN™ 20 surfactant) with or without 1 nM IGF-1R-ECD in 100 μL total volume and incubated at least one hour at room temperature in a NUNC™ F plate (Nalge Nunc International, Rochester, N.Y.). Seventy-five μL of mixture were transferred without or with IGF-1R-ECD side by side to the IGF-1R-ECD-coated plates. The plate was gently shaken for 15 minutes to allow the capture of unbound phage to the IGF-1R-ECD-coated plate. The plate was washed at least five times with PBS-0.05% TWEEN™ 20 surfactant. The binding was quantified by adding HRP-conjugated anti-M13 antibody (Amersham Biosciences) in ELISA buffer (1:5000) and incubated for 30 minutes at room temperature. The plates were washed with PBS-0.05% TWEEN™ 20 surfactant at least five times. Next, 100 μL/well of a 1:1 ratio of 3,3′,5,5′-TMB peroxidase substrate, and Peroxidase Solution B™ (H₂O₂) (Kirkegaard-Perry Laboratories (Gaithersburg, Md.)) was added to the well and incubated for five minutes at room temperature. The reaction was stopped by adding 100 μL 1M phosphoric acid (H₃PO₄) to each well and allowed to incubate for five minutes at room temperature. The OD of the yellow color in each well was determined using a standard ELISA plate reader at 450 nm. The OD reduction (%) was calculated by the following equation:

OD₄₅₀ₙₘ reduction(%)=[(OD₄₅₀ₙₘ of wells with competitor)/(OD₄₅₀ₙₘ of well with no competitor)]*100.

In comparison to the OD₄₅₀ₙₘ reduction (%) of the well of parental phage (75%), clones that had the OD₄₅₀ₙₘ reduction (%) lower than 20% were interesting and picked for analyzing sequence. Those chosen were selected for phage preparation to determine binding affinity (phage IC50) using phage-competition ELISA as described above by comparison with parental clones.

### B. Results and Discussion

### (1) Humanization of 2B4, 9F2, and 10H5

The human acceptor framework used for the humanization of 2B4, 9F2, and 10H5 comprises the consensus human kappa I VL domain and a variant of the human subgroup III consensus VH domain. The variant VH domain has three changes from the human consensus: R71A, N73T, and L78A. The VL and VH domains of murine 2B4, 9F2, and 10H5 were aligned with the human kappa I and subgroup II domains; each HVR was identified and then grafted into the human acceptor framework to generate a 2B4, 9F2, and 10H5 graft, called h2B4.vX, h9F2.vX, and h10H5.vX, respectively, that could be displayed as a Fab on phage.

The full-length sequence of h10H5.vX for the heavy chain, including signal peptide, is:

(SEQ ID NO: 90)

MGWSCIILFLVATATGVHSEVQLVESGGGLVQPGGSLRLSCAASGYTFTR
EWIHWVRQAPGKGLEWVGEINPSNGRTNYNENFKNRFTISADTSKNTAYL
QMNSLRAFDTAVYYCARGGRLDQWGQGTLVTVSSASTKGPSVFPLAPSSK
STSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALH
NHYTQKSLSLSPGK.

The full-length sequence of h10H5.vX for the light chain, including signal peptide, is:

(SEQ ID NO: 91)

MGWSCIILFLVATATGVHSDIQMTQSPSSLSASVGDRVTITCKASQNVGS
NVAWYQQKPGKAPKLLIYSASYRYSGVPSRFSGSGSGTDFTLTISSLQPE
DFATYYCHQYNNYPYTFGQGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC.

### (2) Phage-Competition ELISA Against Human IGF-1R

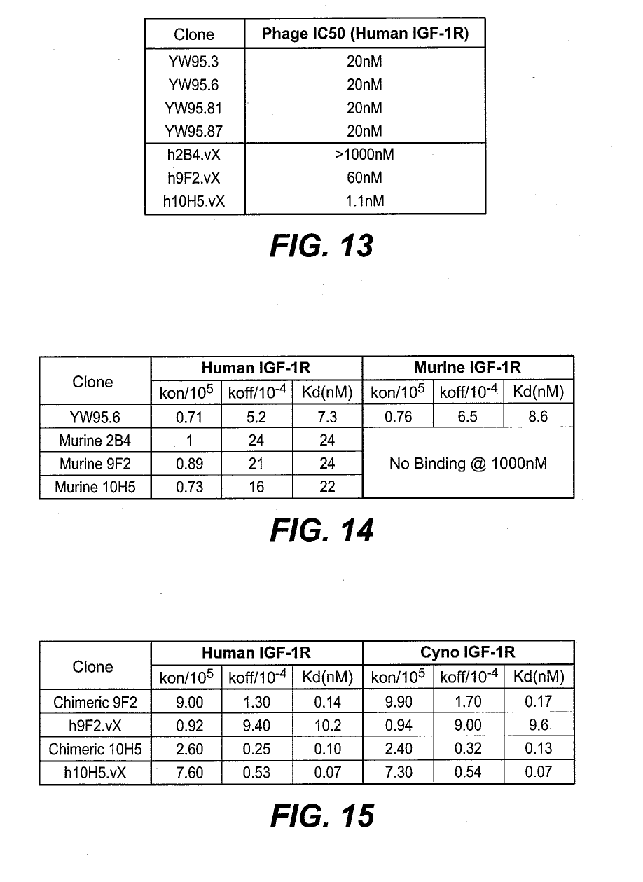

When the humanized hybridoma-derived clones h2B4.vX, h9F2.vX, and h10H5.vX were phage displayed against IGF-1R-ECD to assess binding affinity (phage IC50), h2B4.vX exhibited a micromolar binding affinity, while h9F2.vX and h10H5.vX had binding affinities of 60 nM and 1.1 nM, respectively. See FIG. 13.

### (3) BIACORE™ Instrument Analysis of Anti-IGF-1R Fab

Before HVR grafting, murine 2B4, 9F2, and 10H5Fab binding affinities were evaluated against human and murine IGF-1R-ECD using BIACORE™ instrument analysis. The procedure used was that for BIACORE™ instrument analyses generally, wherein the analyte was anti-IGF-1R Fab (500 nM-3.1 mM; two-fold serial dilution), the ligand was IGF-1R (about 500 RU)-coated CM5 sensor chip, and the temperature was 25° C. They all have similar affinity against human and murine IGF-1R ligands (FIG. 14).

After HVR grafting, only h9F2.vX and h10H5.vX were considered for reformatting into IgG, because the affinity of h2B4.vX decreased into the micromolar range. By comparison of BIACORE™ instrument analysis data of chimeric 9F2, h9F2.vX, chimeric 10H5, and h10H5.vX (using the analyte anti-IGF-1R IgG (250 nM about 0.78 nM; 2-fold serial dilution), the ligand IGF-1R (about 500 RU)-coated CM5 sensor chip, and a temperature of 25° C.), only clone h10H5.vX retained its binding affinity after HVR grafting. See FIG. 15. Therefore, the h10H5.vX clone was picked out for further affinity maturation.

(4) Affinity Maturation of h10H5.vX

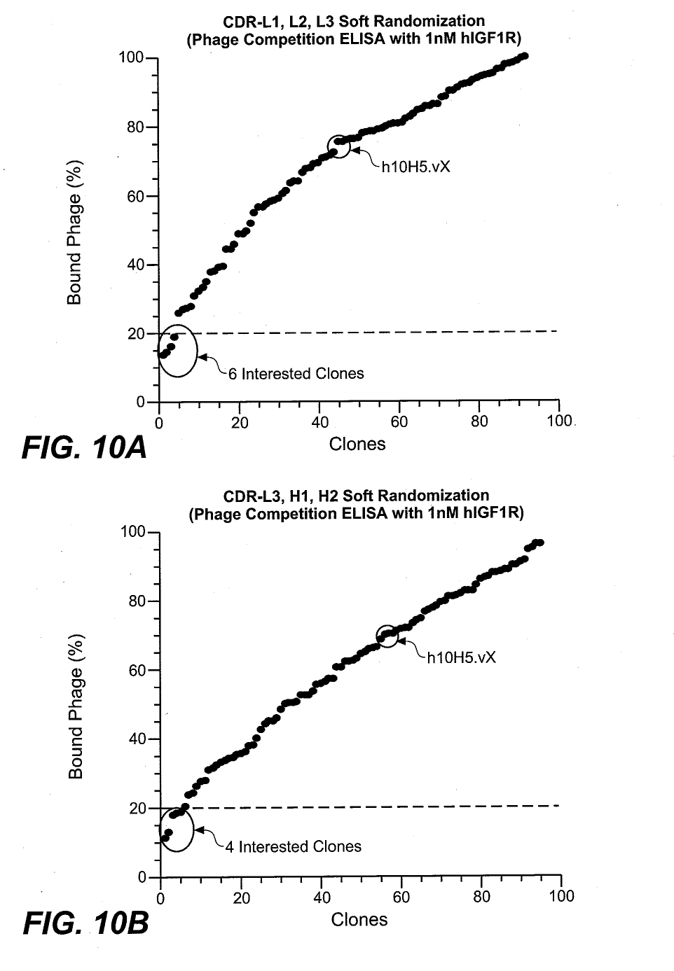

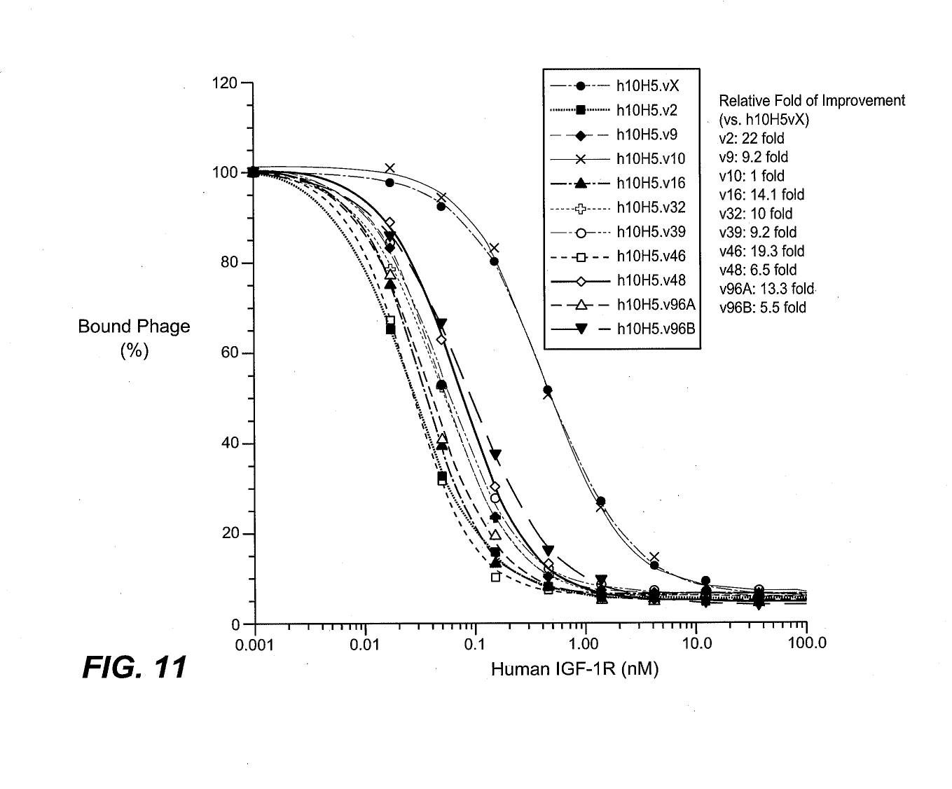

Four libraries were generated in which combinatorial HVR regions (HVR-L1, -L2, -L3, HVR-L3, -H1, -H2, HVR-L3, -H3, and HVR-H1, -H2, -H3) of h10H5.vX were soft randomized (FIG. 7). All four libraries were panned against immobilized IGF-1R-ECD for one round of selection and against solution-phase biotin-IGF-1R-ECD for another four rounds of selection (FIG. 8). Significant enrichment was observed in two libraries: HVR-L1, -L2, -L3 and HVR-L3, -H1, -H2 (FIG. 9). After the fourth round of panning, 96 clones that had the OD₄₅₀ₙₘ reduction (%) lower than 20% were screened using single-spot-competition phage ELISA with 1 mM IGF-1R-ECD (FIG. 10). Ten interesting and unique clones were finalized and selected for DNA sequence analysis (FIG. 11).

Six of the ten clones (h10H5.v2, v9, v10, v39, v48, v96A) were derived from the HVR-L1, -L2, -L3 library, and the remaining four clones (h10H5.v16, v32, v46, v96B) were derived from the HVR-L3, -H1, -H2 library. Most of the clones had residue changes at position 89, 92, and 93 in HVR-L3, and interestingly, clones derived from the HVR-L3, -H1, -H2 library still retained the parental clone heavy-chain HVR sequence, indicating that the heavy-chain HVRs were already optimized, with no room for further improvement. Analysis of those ten clones' binding affinity by phage-competition ELISA (IC50) by comparison with parental clones indicated the improvement was varied from five to 20 fold. See FIG. 11.

The sequences of these clones and the phage IC50 numbers against human IGF-1R, indicating how each antibody clone specifically bound to IGF-1R, are shown in FIG. 12.

### III. Phage Antibodies

Synthetic VH/VL antibody phage libraries, with the Fab-zip-pIII display format, were generated to produce antibodies. Pre-assembled trinucleotides were used to design mutagenic oligonucleotides, so as to precisely engineer the diversity of each selected position to attempt close mimicry of the diversity of natural compositions of antibody HVR regions and to avoid unnecessary stop codons in the template. As a result, a design of HVR libraries (especially the HVR-H3) that had higher quality than those that could be generated using conventional approaches was employed. Both VH and VH/VL libraries had the same scaffold; the key differences were the designed diversities in the HVRs. By selection against VH/VL libraries, antibodies were found that had a sufficiently high binding affinity or biological potency for therapeutic applications.

### A. Materials and Methods:

### (1) Materials

Enzymes and M13-KO7 helper phage were from New England Biolabs. E. coli XL-1 BLUE™ cells were from Stratagene (La Jolla, Calif.). Ninety-six-well MAXISORP™ immunoplates were from NUNC™ (Roskilde, Denmark). BSA, TWEEN™ 20 surfactant, and anti-human IgG-conjugated HRP were from Sigma-Aldrich (St. Louis, Mo.). Neutravidin, casein, streptavidin-conjugated HRP, and SUPERBLOCKER™ reagent were from Pierce Biotechnology, Inc. (Rockford, Ill.). Anti-M13-conjugated HRP was from Amersham Pharmacia (Piscataway, N.J.). TMB substrate was from Kirkegaard and Perry Laboratories (Gaithersburg, Md.). Carboxymethylated dextran biosensor chips (CM5), N-ethyl-N′-(3-dimethylaminopropyl)-carbodiimide hydrochloride (EDC), N-hydroxysuccinimide (NHS), and ethanolamine for BIACORE™ analysis were from BIAcore, Inc. (Piscataway, N.J.). Cell-dissociation buffer and PE-Fab′₂ goat anti-human IgG Fc-specific antibody for flow cytometry were from Gibco (Gaithersburg, Md.) and ImmunoResearch Laboratories (West Grove, Pa.), respectively. Equimolar DNA degeneracies are represented in the IUB code (B=C/G/T, D=A/G/T, M=A/C, N=A/C/G/T, R=A/G, S=G/C, W=A/T). The trimer phosphoramidite (trinucleotide codon) reagents were from Glen Research (Sterling, Va.). The native-sequence IGF-1R (Ullrich et al., EMBO, supra) and oligonucleotides were generated and provided by Genentech, Inc. (South San Francisco, Calif.) using standard techniques.

(2) VH/VL Naïve Library Construction

A VH/VL naïve library template comprising consensus HVR-L1, -L2, -L3, -H1 and -H2 sequences was generated using oligonucleotide-directed mutagenesis on phagemid pV0350-4 with stop codons in HVR-H3 and displaying bivalently on the surface of M13 bacteriophage particles (Lee et al., J. Mol. Biol., supra (2004)). Phage-displayed libraries were constructed using the Kunkel mutagenesis method as described by Kunkel et al., supra, with a mixture of mutagenic oligonucleotides designed to introduce mutations at the designed sites in HVR-L3, -H1, -H2 and -H3, and to repair template HVR-H3 stop codons. The mutagenesis reactions (˜10 μg DNA) were electroporated into E. coli SS320 cells (˜10¹¹ cells) as described by Sidhu et al., Methods Enzymol., 328: 333-363 (2000), and the cultures were grown overnight at 30° C. in 2YT broth with M13-KO7 helper phage and 50 μg/ml of carbenicillin and kanamycin.

Phage particles were harvested from the culture medium by precipitation with polyethylene glycol (PEG)/NaCl. Each library contained about 2×10⁹-8×10⁹ transformants. The functional display level of each library was evaluated by a phage ELISA with an anti-gD-tag antibody as a capture target, and approximately 40% and 20% of 48 randomly-picked clones from the different HVR-H3 length libraries, respectively, exhibited a positive ELISA signal.

### (3) Library Sorting and Screening to Identify Anti-IGF-1R Antibodies

IGF-1R-ECD (see hybridoma production above) was coated on MAXISORP™ microtiter plates (Nalge Nunc International, Rochester, N.Y.) at 5 μg/ml in PBS. Four libraries were selected separately. For the first round of selection, eight wells of target were used; a single well of target was used for successive rounds of selection. Wells were blocked for one hour using CASEIN BLOCKER™ reagent (Pierce Biotechnology, Inc.). Phage were harvested from the culture supernatant and suspended in PBS containing 1% BSA and 0.05% TWEEN™ 20 surfactant (PBSBT). After binding to the wells for two hours at 37° C., unbound phage were removed by extensive washing with PBST. Bound phage were eluted by incubating the wells with 50 mM HCl, 0.5 M KCl for 30 minutes. Phage were amplified using E. coli XL1 BLUE™ cells (Stratagene) and M13/KO7 helper phage (New England Biolabs) and grown overnight at 37° C. in 2YT, 50 μg/ml carbanecillin, and 50 μg/ml kanamycin. The titers of phage eluted from a target-coated well were compared to titers of phage recovered from a non-target-coated well to assess enrichment.

Randomly-picked 96 clones selected from the 4^{th} round were assayed using a high-throughput phage ELISA as described below to check binding to IGF-1R, an anti-gD antibody, and two non-relevant proteins (BSA and a commercially available anti-IgE antibody). Only clones with specific binding to IGF-1R and anti-gD antibody were subjected to DNA sequence analysis of the V_{L} and V_{H} region.

### (4) Fab Production and Affinity Determination for Phage-Derived Antibodies

To express Fab protein for affinity measurements of the phage-derived antibodies, a stop codon was introduced between the heavy chain and g3 in the phage-display vector. Clones were transformed into E. coli 34B8 cells and grown in AP5 media at 30° C. (Presta et al., Cancer Res., 57: 4593-4599 (1997)). Cells were harvested by centrifugation, suspended in 10 mM TRIS buffer, 1 mM EDTA pH 8, and broken open using a microfluidizer. Fab was purified with Protein-G-affinity chromatography.

### (5) High-Throughput, Affinity-Screening Phage ELISA (Single-Spot Competition)

Colonies were picked from the fourth-round screens of two significantly enriched libraries, HVR-L1, -L2, L3 and HVR-L3, -H1, -H2, and grown overnight at 37° C. in 150 μL/well of 2YT media with 50 μg/ml carbenicillin and 1×10¹⁰ PFU/ml M13/K07 helper phage (New England Biolabs) in a 96-well plate (Falcon). From the same plate, a colony of XL1 BLUE™ cells (Stratagene)-infected parental phage (h10H5.vX) was picked as control. Ninety-six-well NUNC™ MAXISORP™ plates were coated with 100 μL/well of IGF-1R-1-ECD (2 μg/ml) in PBS at 4° C. overnight or room temperature for two hours. The plates were blocked with 65 μL of 1% BSA for 30 minutes and 40 μL of 1% TWEEN™ 20 surfactant for another 30 minutes.

The phage supernatant was diluted 1:10 in ELISA buffer (PBS with 0.5% BSA, 0.05% TWEEN™ 20 surfactant) with or without 1 nM IGF-1R-ECD in 100 μL total volume and incubated at least one hour at room temperature in a NUNC™ F plate (Nalge Nunc International, Rochester, N.Y.). Seventy-five μL of mixture were transferred without or with IGF-1R-ECD side by side to the IGF-1R-ECD-coated plate. The plate was gently shaken for 15 minutes to allow the capture of unbound phage to the IGF-1R-ECD-coated plate. The plate was washed at least five times with PBS-0.05% TWEEN™ 20 surfactant. The binding was quantified by adding HRP-conjugated anti-M13 antibody (Amersham Biosciences) in ELISA buffer (1:5000) and incubated for 30 minutes at room temperature. The plates were washed with PBS-0.05% TWEEN™ 20 surfactant at least five times. Next, 100 μL/well of a 1:1 ratio of 3,3′,5,5′-TMB peroxidase substrate and Peroxidase Solution B™ (H₂O₂) (Kirkegaard-Perry Laboratories (Gaithersburg, Md.)) was added to the well and incubated for five minutes at room temperature. The reaction was stopped by adding 100 μL 1M phosphoric acid (H₃PO₄) to each well and allowed to incubate for five minutes at room temperature. The OD of the yellow color in each well was determined using a standard ELISA plate reader at 450 nm. The OD reduction (%) was calculated by the following equation:

OD₄₅₀ₙₘ reduction(%)=[(OD₄₅₀ₙₘ of wells with competitor)/(OD₄₅₀ₙₘ of well with no competitor)]*100.

In comparison to the OD₄₅₀ₙₘ reduction (%) of the well of parental phage (75%), clones that had the OD₄₅₀ₙₘ reduction (%) lower than 20% were interesting and picked for analyzing sequence. Those chosen were selected for phage preparation to determine binding affinity (phage IC50) using phage-competition ELISA as described below by comparison with parental clones.

### (6) Phage ELISA

This assay was conducted as described in Section 1-II-A-(4) of this Example. For the YW95-phage-derived clones from the VH/VL phage library, phage-Fab-Zip was used.

### (7) BIACORE™ Instrument Experiments

These experiments were performed as described previously in Section 1-II-A-(3) of this Example.

### B. Results:

### (1) Library Design and Construction

The VH/VL libraries described herein were generated by choosing the anti-ErbB2 antibody, humanized rhuMAb4D5-8, as the scaffold, since this antibody has been demonstrated to display well on bacteriophage, and to be expressed well in E. coli. Lee et al., J. Mol. Biol., supra (2004). In addition, the full-length IgG form of this antibody is expressed well in mammalian cells (Carter, supra (2001)). Bivalent Fabs (F(ab′)₂) were also displayed on phage, since bivalent binding would produce an avidity effect, which was expected to increase the apparent binding affinities for immobilized antigens. This avidity effect has been shown to improve the recovery of rare and low-affinity phage antibody clones. The HVR sequences of humanized rhuMAb4D5-8 in H1, H2, L1, L2, and L3 were replaced with human consensus sequences to avoid potential biases that may be inherited in the rhuMAb4D5-8 scaffold. The consensus sequences of most HVR positions were chosen based on the fact that they represent the amino acid that is most prevalent in that position in human antibodies. The boundaries of each HVR were defined based on the Kabat definition of HVRs (Kabat et al., supra).

As shown in Table 1, HVR-L1 (28-33) was SISSYL (SEQ ID NO:92), HVR-L2 (50-55) was GASSRA (SEQ ID NO:93), HVR-L3 (91-96) was YYSSPL (SEQ ID NO:94), HVR-H1 (27-35) was FTFSSYAMS (SEQ ID NO:95), and HVR-H2 (50-52, 52a, and 53-58) was RISPSGGSTY (SEQ ID NO:96). The prevalence of each residue at the corresponding positions in human antibodies is also shown in Table 1.

TABLE 1
Library Consensus HVRs Sequence

| | | | Prevalence in natural |
|---|---|---|---|
| HVRs | Positions | Residues | antibodies (%) |
| HVR-L1 | 28 | S | 33 |
| | 29 | I | 40 |
| | 30 | S | 55 |
| | 31 | S | 44 |
| | 32 | Y | 67 |
| | 33 | L | 94 |
| HVR-L2 | 50 | G | 25 |
| | 51 | A | 79 |
| | 52 | S | 95 |
| | 53 | S | 36 |
| | 54 | R | 60 |
| | 55 | A | 45 |
| HVR-L3 | 91 | Y | 54 |
| | 92 | Y | 23 |
| | 93 | S | 46 |
| | 94 | S | 24 |
| | 95 | P | 80 |
| | 96 | L | 22 |
| HVR-H1 | 27 | F | 45 |
| | 28 | T | 54 |
| | 29 | F | 73 |
| | 30 | S | 68 |
| | 31 | S | 50 |
| | 32 | Y | 64 |
| | 33 | A | 22 |
| | 34 | M | 46 |
| | 35 | S | 34 |
| HVR-H2 | 50 | R | 17 |
| | 51 | I | 84 |
| | 52 | S | 26 |
| | 52a | P | 29 |
| | 53 | S | 24 |
| | 54 | G | 37 |
| | 55 | G | 53 |
| | 56 | S | 28 |
| | 57 | T | 56 |
| | 58 | Y | 32 |

Since the third heavy-chain HVR (HVR-H3) plays a dominant role in antigen recognition (Xu and Davis, Immunity, 13 (1):37-45 (2000)), and the simplest synthetic antibody repertoires have relied on HVR-H3 libraries, several stop codons were placed in H3 to make sure functional antibody clones from the libraries were different from each other. Stop codons were put only in HVR-H3 to enable recovery of antibodies with diversity in H3 alone, diversity in all H1/H2/H3/L3, or combinations of L3/H3, H1/H2/H3, H1/H3/L3, H2/H3/L3, H1/H3, and H2/H3 (i.e., all diversity combinations invariably contain H3 diversity). This design of the phage libraries has the advantage of increasing the ratio of functional phage antibody clones in the context of a limited library size.

In phage-display technology, both the diversity design and the library size are crucial for the performance of a synthetic library. In the VH/VL library disclosed herein, a subset of HVR positions was chosen for diversification using criteria of high-solvent exposure and/or especially high variability among natural antibody sequences. Table 2 shows the positions in different HVRs that were chosen for mutagenesis of the VH/VL library. For example, in HVR-H1, positions 27, 28, 30, 31, 32, 33, and 34 were chosen to be diversified. Degenerate oligonucleotide codons or trinucleotides were used to introduce and generate desired sequence diversities. The criteria for designed sequence diversity were that most of the natural diversity would be included in each position selected for diversification, and the most dominant amino acids would also be well represented.

TABLE 2
Designed Diversity for VH/VL Library

| | | Design Diversity | | | | | Natural |
|---|---|---|---|---|---|---|---|
| | | Residues Encoded | | | | | diversity |
| | | | | | | Others | coverage |
| HVRs | Positions | Codon | Y | G | S | (−Cys) | (%) |
| HVR-L3 | Y91 | TAC | 100 | — | — | — | 77 |
| | | MGC | — | — | — | R/S (50) | |
| | Y92 | X5 | 19.2 | 19.2 | 19.2 | 2.5 | 100 |
| | S93 | X1 | 2.5 | 2.5 | 52.5 | 2.5 | 100 |
| | S94 | X6 | 20 | 3.3 | 20 | 3.3 | 100 |
| | L96 | NTC | — | — | — | F/I/L/V (45) | 45 |
| HVR- | F27 | TWC | 50 | — | — | F(50) | 65 |
| H1 | T28 | ASC | — | — | 50 | T(50) | 90 |
| | S30 | ASC | — | — | 50 | T(50) | 86 |
| | S31 | X1 | 2.5 | 2.5 | 52.5 | 2.5 | 100 |
| | Y32 | X2 | 52.5 | 2.5 | 2.5 | 2.5 | 100 |
| | A33 | X7 | 15.6 | 15.6 | 15.6 | 3.1 | 100 |
| | M34 | ATS | — | — | — | M/I(50) | 67 |
| HVR- | R50 | X3 | 5 | 5 | 5 | 5 | 100 |
| H2 | S52 | X6 | 20 | 3.3 | 20 | 3.3 | 100 |
| | P52a | CCT | — | | — | P(100) | 100 |
| | | X7 | 15.6 | 15.6 | 15.6 | 3.1 | |
| | S53 | X7 | 15.6 | 15.6 | 15.6 | 3.1 | 100 |
| | G54 | RRC | — | — | 25 | D/G/N(25) | 81 |
| | S56 | DMT | 16.6 | — | 16.6 | A/D/N/T | 81 |
| | Y58 | DAC | 33.3 | — | — | (16.6) | 70 |
| | | | | | | D/N(33.3) | |
| HVR- | 95 | X4 | 3.8 | 28.8 | 3.8 | 3.8 | 100 |
| H3 | 96 | X4 | 3.8 | 28.8 | 3.8 | 3.8 | 100 |
| | 97-100k | (X7)4- | 15.6 | 15.6 | 15.6 | 3.1 | >98 |
| | 100l | 15 | 15.6 | 15.6 | 15.6 | 3.1 | 100 |
| | | X7 | — | 33.3 | — | A/V(33.3) | |
| | 100m | GBT | — | — | — | F(100) | 89 |
| | | TTC | — | — | — | M(100) | |
| | 101 | ATG | — | — | — | D(100) | 92 |
| | 102 | GAT | 100 | — | — | — | 67 |
| | | TAC | — | — | — | V(100) | |
| | | GTC | | | | | |

In the VH/VL libraries, HVR-L3 was chosen for diversification, since HVR-H3 and HVR-L3 form the inner circle of the antigen-binding site, and therefore show the highest frequency of antigen contacts in structurally known antibody-antigen complexes. The purpose of the designed diversity in HVR-L3 was to obtain the highest degree of diversity in the most variable positions biased toward the known natural distribution of amino acids.

HVR-H3 has been shown to be far more variable than others in length, sequence, and structure. Since HVR-H3 length is a key component of the diversities in natural antibodies, 12 subset VH/VL libraries were constructed with different HVR-H3 lengths varying from 9 to 21 amino acids, which would cover approximately 90% of HVR-H3 length variation in natural antibodies (Kabat database). Since cysteines are rare in HVRs, all cysteines were excluded from the designed codons to avoid the potential of unpaired cystines and protein expression/manufacturing problems. In the Kabat immunoglobulin database, glycine, tyrosine, and serine are the most abundant residues in HVR-H3; accordingly, the oligonucleotides for VH/VL HVR-H3 positions 97 to 100k were designed to reflect this bias. A mixture of trinucleotide codons was used. For example, codon X7 (Table 2) represented 15.6% of each of serine, tyrosine, and glycine, and 3.1% for the remaining amino acids (except cysteine).

Table 3 shows the designed Xn codon to bias Y/G/S.

TABLE 3
Designed Xn codon to Bias Y/G/S

| | Trimer | | | | | | | | |
|---|---|---|---|---|---|---|---|---|---|
| Residue | Codon | X0 | X1 | X2 | X3 | X4 | X5 | X6 | X7 |
| A | GCT | 2.5 | 2.5 | 2.5 | 5.0 | 3.8 | 2.5 | 3.3 | 3.1 |
| D | GAC | 2.5 | 2.5 | 2.5 | 5.0 | 3.8 | 2.5 | 3.3 | 3.1 |
| E | GAA | 2.5 | 2.5 | 2.5 | 5.0 | 3.8 | 2.5 | 3.3 | 3.1 |
| F | TTC | 2.5 | 2.5 | 2.5 | 5.0 | 3.8 | 2.5 | 3.3 | 3.1 |
| G | GGT | 52.5 | 2.5 | 2.5 | 5.0 | 28.8 | 19.2 | 3.3 | 15.6 |
| H | CAT | 2.5 | 2.5 | 2.5 | 5.0 | 3.8 | 2.5 | 3.3 | 3.1 |
| I | ATC | 2.5 | 2.5 | 2.5 | 5.0 | 3.8 | 2.5 | 3.3 | 3.1 |
| K | AAA | 2.5 | 2.5 | 2.5 | 5.0 | 3.8 | 2.5 | 3.3 | 3.1 |
| L | CTG | 2.5 | 2.5 | 2.5 | 5.0 | 3.8 | 2.5 | 3.3 | 3.1 |
| M | ATG | 2.5 | 2.5 | 2.5 | 5.0 | 3.8 | 2.5 | 3.3 | 3.1 |
| N | AAC | 2.5 | 2.5 | 2.5 | 5.0 | 3.8 | 2.5 | 3.3 | 3.1 |
| P | CCG | 2.5 | 2.5 | 2.5 | 5.0 | 3.8 | 2.5 | 3.3 | 3.1 |
| Q | CAG | 2.5 | 2.5 | 2.5 | 5.0 | 3.8 | 2.5 | 3.3 | 3.1 |
| R | CGT | 2.5 | 2.5 | 2.5 | 5.0 | 3.8 | 2.5 | 3.3 | 3.1 |
| S | TCT | 2.5 | 52.5 | 2.5 | 5.0 | 3.8 | 19.5 | 20.0 | 15.6 |
| T | ACT | 2.5 | 2.5 | 2.5 | 5.0 | 3.8 | 2.5 | 3.3 | 3.1 |
| V | GTT | 2.5 | 2.5 | 2.5 | 5.0 | 3.8 | 2.5 | 3.3 | 3.1 |
| W | TGG | 2.5 | 2.5 | 2.5 | 5.0 | 3.8 | 2.5 | 3.3 | 3.1 |
| Y | TAC | 2.5 | 2.5 | 52.5 | 5.0 | 3.8 | 19.5 | 20.0 | 15.6 |

X0: All 19 amino acids (without Cys) + Gly (50:50)
X1: All 19 amino acids (without Cys) + Ser (50:50)
X2: All 19 amino acids (without Cys) + Tyr (50:50)
X3: All 19 amino acids (without Cys)
X4: (X0 + X3)/2
X5: (X0 + X1 + X2)/3
X6: (X1 + X2 + X3)/3
X7: (X0 + X1 + X2 + X3)/4

The Fab-Zip DNA was fused to the C-terminal domain of the M13 gene-3 minor coat protein, and the entire cassette inserted into a phagemid. Co-infection of E. coli with the display phagemid and a helper phage resulted in the display of bivalent Fab-Zip on the surfaces of M13 bacteriophage particles (Sidhu et al., 2000, supra). The display level of the library (library size about 10¹⁰) was tested by the specific capture of phage particles with immobilized anti-gD-tag antibody, and found comparable to that of previous libraries.

(2) Identifying Phage-Derived Antibodies that Bind to Human IGF-1R

The above-described VH/VL libraries were used for selection of antibody binders. CHO-cell-expressed human IGF-1R-ECD was used as the antigen for panning. All 12 phage libraries were incubated with plate-immobilized antigen in the first round of panning. The eluted phage from each library were amplified and combined for the second round of panning. A total of four rounds of pannings were performed on immobilized IGF-1R with varying protein concentrations and a number of washings after the initial binding process. Since human IGF-1R-ECD was used as the antigen, the phage libraries were pre-absorbed with excess irrelevant Fc fusion protein after a first round of panning to minimize the recovery of anti-Fc phage antibodies.

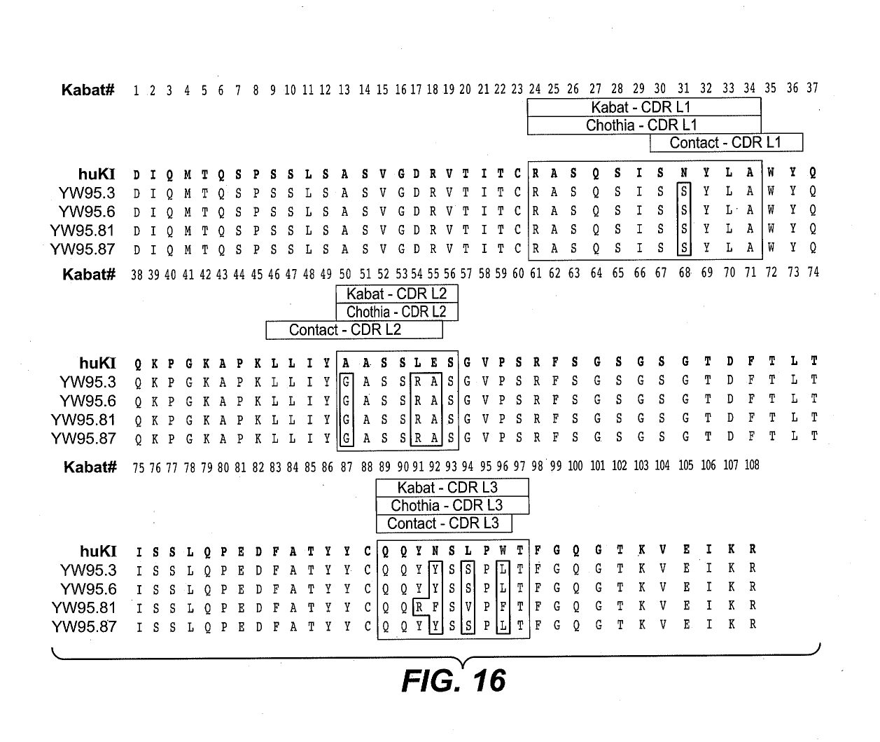

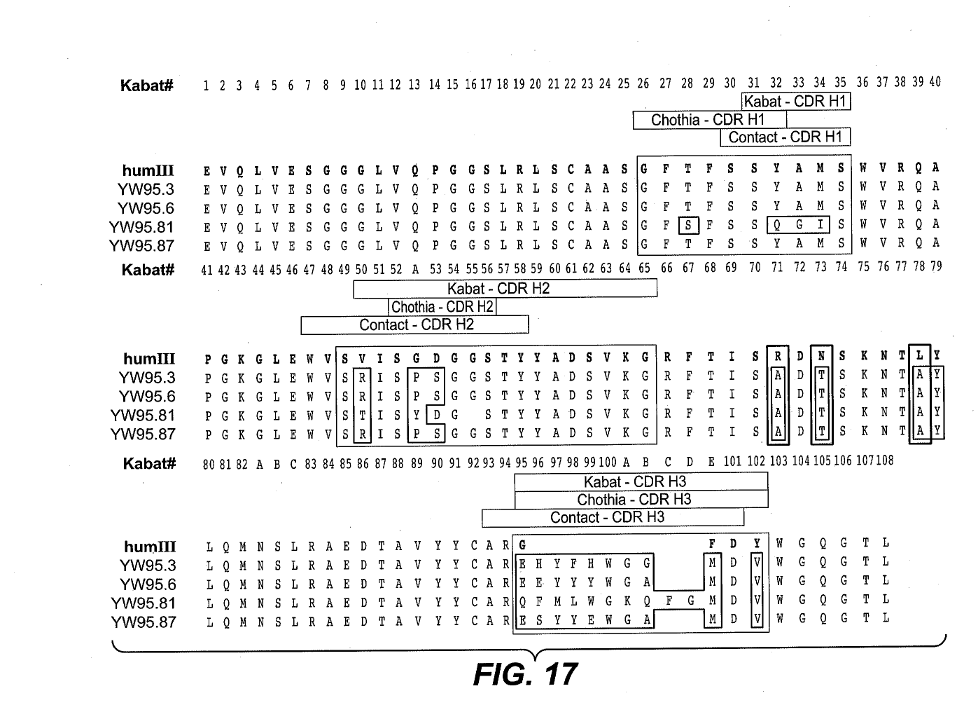

Functional clone sequences identified from the VH/VL library are shown in Table 4. FIGS. 16 and 17 show the sequences of the light- and heavy-chain variable regions, respectively, of four identified clones, YW95.3, YW95.6, YW95.81, and YW95.87.

TABLE 4

Functional Clone Sequence identified from VH/VL library

| | | | | |
|---|---|---|---|---|
| Clones | YW95.3 | YW95.6 | YW95.87 | YW95.81 |

| | | | | |
|---|---|---|---|---|
| HVR-L1 | SISSYL | SISSYL | SISSYL | SISSYL |
| 28-33 | (SEQ ID NO: 92) | (SEQ ID NO: 92) | (SEQ ID NO: 92) | (SEQ ID NO: 92) |
| HVR-L2 | GASSRA | GASSRA | GASSRA | GASSRA |
| 50-54 | (SEQ ID NO: 93) | (SEQ ID NO: 93) | (SEQ ID NO: 93) | (SEQ lD NO: 93) |
| HVR-L3 | YYSSPL | YYSSPL | YYSSPL | RFSVPF |
| 91-96 | (SEQ ID NO: 94) | (SEQ ID NO: 94) | (SEQ ID NO: 94) | (SEQ ID NO: 101) |
| HVR-H1 | FTFSSYAMS | FTFSSYAMS | FTFSSYAMS | FSFSSQGIS |
| 27-35 | (SEQ ID NO: 95) | (SEQ ID NO: 95) | (SEQ ID NO: 95) | (SEQ ID NO: 102) |
| HVR-H2 | SRISPSGGSTY | SRISPSGGSTY | SRISPSGGSTY | STISYDGSTY |
| 49-52 | (SEQ ID NO: 97) | (SEQ ID NO: 97) | (SEQ ID NO: 97) | (SEQ ID NO: 103) |
| 52a 53-58 | | | | |
| HVR-H3 | REHYFHWGGM | REEYYYWGAM | RESYYEWGAM | RQFMLWGKQFGM |
| 94-100 A-C | (SEQ ID NO: 98) | (SEQ ID NO: 99) | (SEQ ID NO: 100) | (SEQ ID NO: 104) |

The results of the phage-ELISA testing of the YW95 phage-derived clones (from the VH/VL phage library) (i.e., phage-Fab-Zip tested for binding affinity when displayed as a bivalent Fab on phage) are shown in FIG. 13. All four clones tested (YW95.3, YW95.6, YW95.81, and YW95.87) had a phage IC50 of 20 nM.

### (3) BIACORE™ Instrument Analysis

The binding affinity of the phage-derived clone YW95.6 was evaluated against IGF-1R-ECD using BIACORE™ instrument analysis. The procedure used was that for BIACORE™ instrument analyses generally, wherein the analyte was anti-IGF-1R Fab (500 nM-3.1 nM; two-fold serial dilution), the ligand was a IGF-1R-ECD (about 500 RU)-coated CM5 sensor chip, and the temperature was 25° C. See FIG. 14 for the binding affinity of clone YW95.6, as compared to the binding affinities of murine 2B4, murine 9F2, and murine 10H5Fab against human and murine IGF-1R ligands.

### (4) Crystal Structure Determination

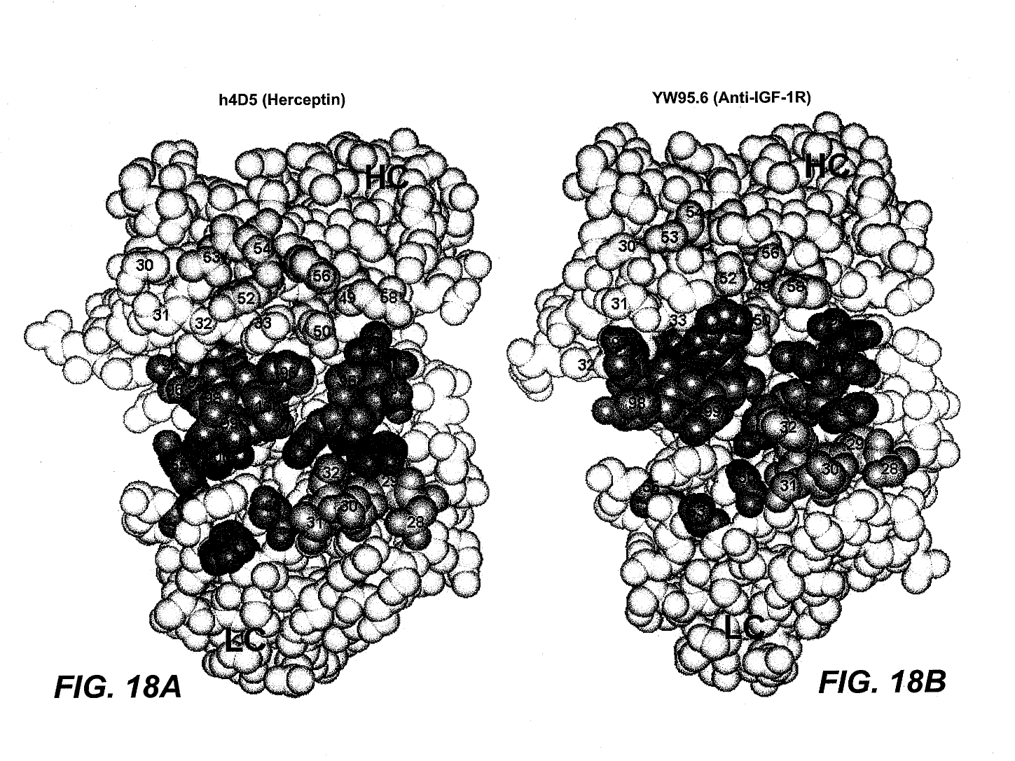

The crystal structure of YW95.6 Fab in unbound form with five consensus HVRs was solved and revealed expected canonical structure as h4D5 (HERCEPTIN® trastuzumab). See FIG. 18.

### C. Discussion:

Humanized synthetic phage antibody libraries, also referred to as VH/VL libraries, were generated by randomizing the HVRs of heavy-chain and L3 of light-chain using as a template the recombinant humanized antibody 4D5-8. This antibody is known to display well on phage surface, as well as being capable of being expressed as Fab (or other antibody fragment) or full-length antibody (e.g., IgG). In one embodiment, the template HVRs were substituted with consensus sequences to avoid biasing binding characteristics of polypeptides toward particular antigens (e.g., antigens that share similarity with the IGF-1R of 4D5-8). Accordingly, in the libraries described herein, the HERCEPTIN® trastuzumab (h4D5) framework as a template contains human consensus sequence in five HVRs (HVR-L1, -L2, -L3, -H1, and -H2) and a stop codon in HVR-H3, to improve the efficiency of mutagenesis (e.g., Kunkel mutagenesis) while minimizing background noise due to recovery of unmutagenized template sequences. Diversity at the libraries was introduced at high-solvent-exposed and/or highly variable positions in HVR-L3, -H1, -H2, and -H3 using tailored degenerate and pre-assembled trinucleotide codons that mimic natural human antibodies. The usage of trinucleotides for oligonucleotide synthesis enabled the increase of amino acid diversity without introducing cysteine, stop, and redundant codons. Functional clones targeting IGF-1R with binding affinities ranging from 1 to about 100 nM were identified from this library. Structurally, consensus HVRs were found to behave canonically as did HERCEPTIN® trastuzumab (h4D5).

The clone YW95.6 was further tested for its effect on blocking ligand/receptor interaction and ligand-mediated receptor signaling, and on the cell viability of the human breast tumor cell line MCF7, with results shown in Example 2.

### EXAMPLE 2

### Characterization of the Anti-IGF-1R Antibodies

### I. Anti-IGF-1R Antibodies Bind to Human and Cynomolgus-Monkey IGF-1R, But not to Murine IGF-1R or Insulin Receptor

Direct ELISA was performed to screen anti-IGF-1R monoclonal antibodies. Briefly, human IGF-1R-ECD was coated overnight at 4° C. at 1 μg/ml in a 96-well immunoplate (Nalge Nunc International, Rochester, N.Y., USA). The ELISA plate was washed with wash buffer (PBS, 0.05% TWEEN™ 20 surfactant, pH 7.4) and 150 μl/well of blocking buffer (PBS with 0.5% BSA) added for one hour at room temperature with gentle agitation. For direct ELISA, 100 μl/well of the supernatants of the tested anti-IGF-1R antibodies or medium alone was added and incubated at room temperature for 1 hour. The ELISA plate was washed three times and 100 μl/well of goat anti-murine-Fc antibody conjugated to HRP (Sigma, St. Louis, Mo., USA) was added at a dilution of 1:5000. After 45 minutes of incubation, the plate was washed and 100 μl/well of TMB (R&D Systems, Minneapolis, Minn., USA) was added for about ten minutes for signal revelation. When blue coloration appeared, 100 μl/well of phosphoric acid at 1 M was added to stop the revelation process. The optical density was read at 450 nm/620 using the MUTISCAN ASCENT™ system from Thermo Lab System (Milford, Mass., USA). In addition, hybridoma supernatants were further screened using FACS analysis of MCF7 cells. The hybridoma clones that were positive for both assays were selected for further analysis.

Binding affinities of selected antibodies to IGF-1R were determined by surface plasmon resonance measurements on a BIACORE™ 3000 instrument (BIAcore, Inc.). Human, murine, or cynomolgus-monkey IGF-1R was immobilized at a density of about 500 RU on the flow cells of a PIONEER™ CM5 sensor chip. Immobilization was achieved by random coupling through amino groups using a protocol provided by the manufacturer. Sensorgrams were recorded for binding of anti-IGF-1R Fab or IgG to these surfaces by injection of a series of solutions ranging from 500 nM to 3.1 nM and 250 nM to 0.78 nM in two-fold increments, respectively. The signal from the reference cell was subtracted from the observed sensorgram. Kinetic constants were calculated by non-linear regression analysis of the data according to a 1:1 Languir binding model using software supplied by the manufacturer.

The affinity measurements for 9F2 and 10H5 against human and murine IGF-1R are listed in FIG. 14. They exhibited strong binding to human IGF-1R, but not to mouse IGF-1R or human insulin receptor. The affinity measurements for chimeric 9F2-IgG, humanized 9F2.vX-IgG, chimeric 10H5-IgG, and humanized 10H5.vX-IgG against human and cynomolgus-monkey (cyno) IGF-1R are listed in FIG. 15. The binding was comparable for human and cyno IGF-1R.

### II. Blocking of IGF-I/II and IGF-1R Interaction

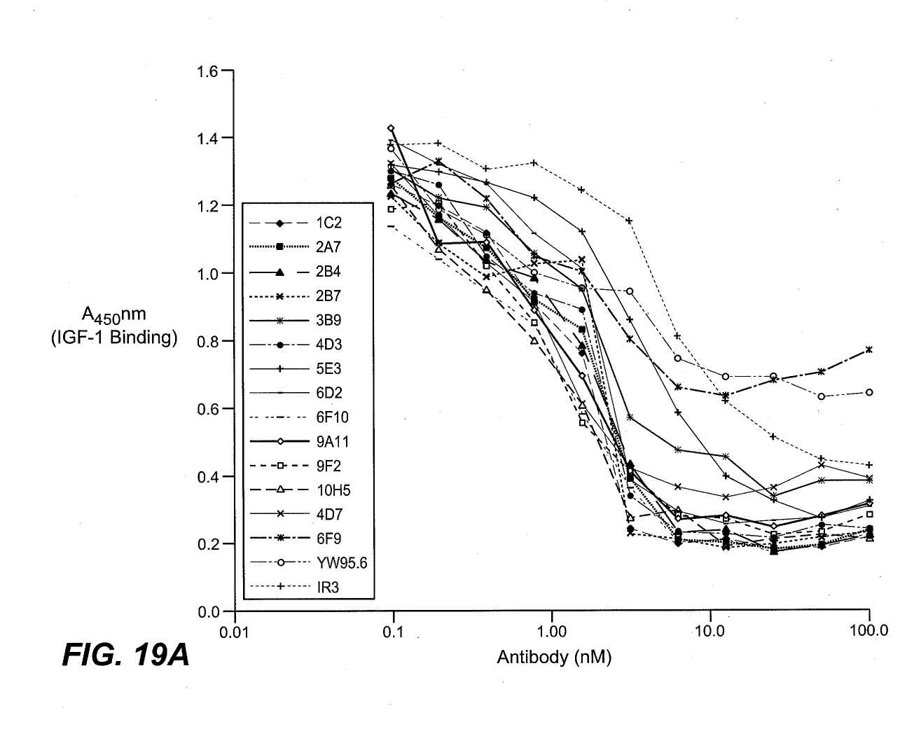

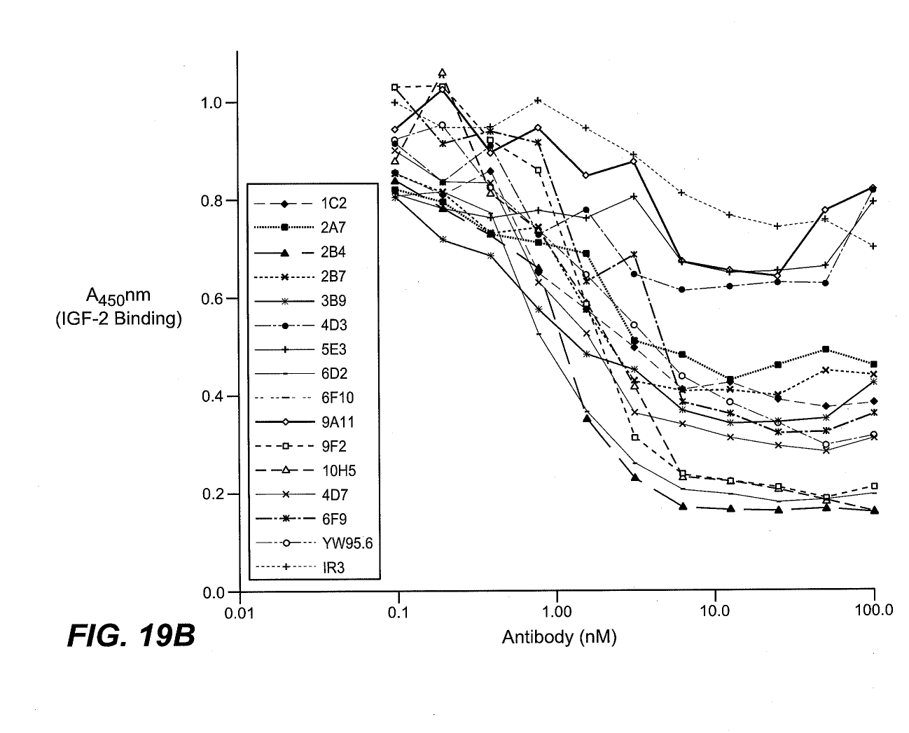

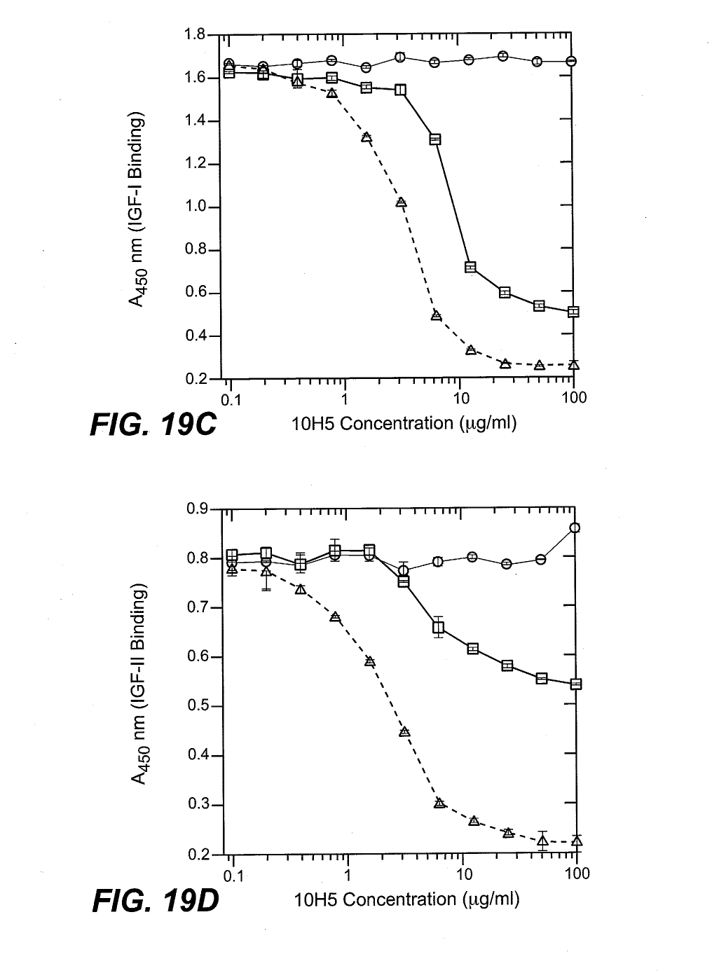

To determine whether the anti-IGF-1R antibodies could block ligand/receptor interaction, competitive ELISA was utilized. Human IGF-1R-ECD was coated overnight at 4° C. at 1 μg/ml in a 96-well immunoplate (Nalge Nunc International, Rochester, N.Y., USA). The ELISA plate was washed with wash buffer (PBS, 0.05% TWEEN™ 20 surfactant, pH 7.4), and 150 μl/well of blocking buffer (PBS with 0.5% BSA) was added for one hour at room temperature with gentle agitation. Purified anti-IGF-1R antibodies were first subjected to a two-fold dilution from 100 nM to 0.0976 nM in assay buffer (PBS, 0.5% BSA and 0.05% TWEEN™ 20 surfactant, pH 7.4) and added to the immunoplate to incubate at room temperature for one hour. Biothinyl-IGF-I or IGF-II at 60 ng/ml concentrations (Cell Science, Canton, Mass., USA) was then added and incubated for another 30 minutes. The ELISA plate was washed three times, and 100 μl/well of streptavidin-HRP (GE Healthcare, Piscataway, N.J., USA) at a dilution of 1:6,000 was added for 20 minutes. Color development was conducted as described in conjunction with the direct ELISA. The selected anti-IGF-1R antibodies inhibited IGF-I or IGF-II binding to IGF-1R to various degrees (FIGS. 19A and 19B for IGF-I and IGF-II, respectively). Antibodies 2B4, 6D2, 9F2, and 10H5 belonged to the most potent group and were significantly superior to IR3 (Electron Microscopy Sciences, Hatfield, Pa., USA), which is a known blocking antibody for IGF-1R.

### III. Inhibition of IGF-I/II-Dependent IGF-1R Phosphorylation and Downstream Signaling

Also examined was whether the blocking activity of these antibodies was sufficient to interfere with receptor activation, which is measured by IGF-1R phosphorylation. A KIRA assay (kinase receptor activation) was utilized to perform quantitative analysis of IGF-1R phosphorylation in MCF7 cells. Anti-IGF-1R antibodies in serial dilutions (0.19 to 200 nM in medium) were added to MCF7 cells in 96-well plates for one hour. IGF-I (10 ng/ml) or IGF-11 (50 ng/ml) was subsequently added and incubated for ten minutes. The cells were lysed, and the resulting lysates were transferred to an ELISA plate, which was coated with IGF-1R-capturing antibody 3B7 (250 ng/well). After incubation for two hours, the cell lysate was removed, and the plates were washed. Phosphotyrosine-specific antibody 4G10 (Upstate, Charlottesville, Va., USA) was added to detect IGF-1R phosphorylation through TMB peroxidase substrate.

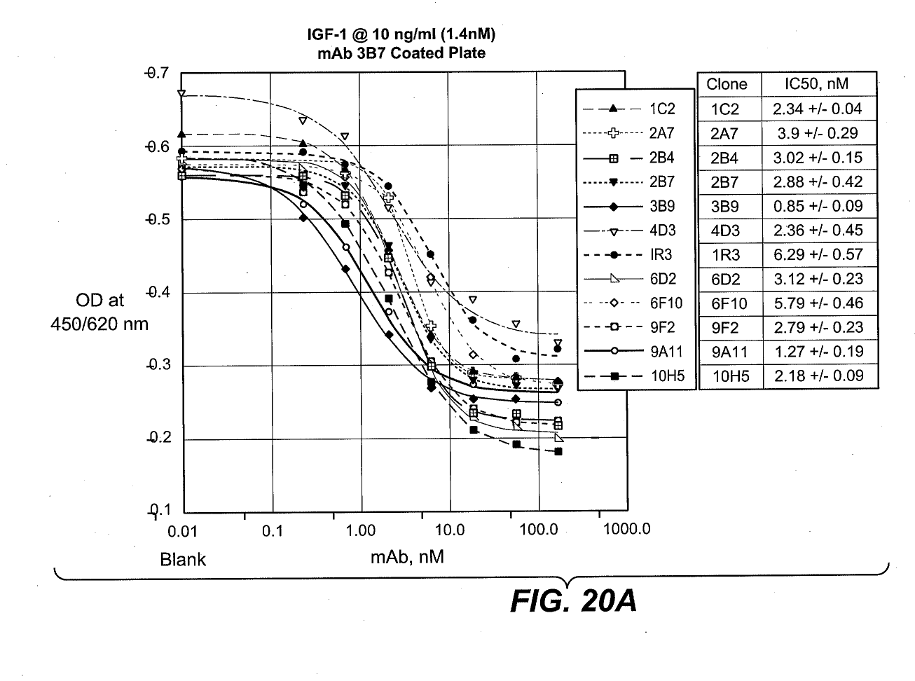

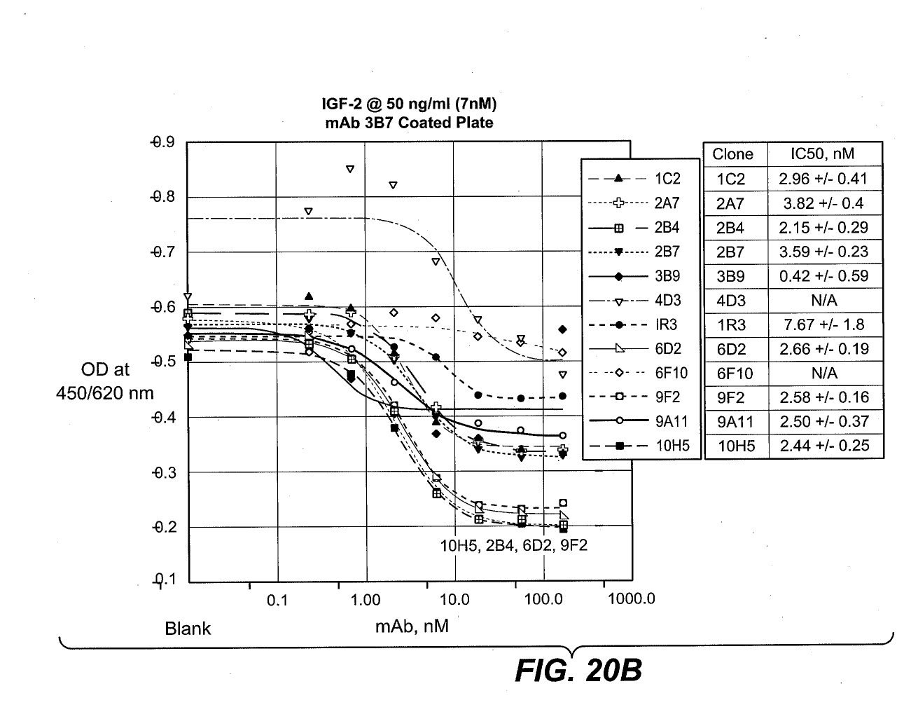

FIGS. 20A and 20B show the results from these experiments for IGF-I and IGF-II, respectively. Several antibodies of this invention were potent in inhibiting IGF-I/II-mediated IGF-1R phosphorylation. The relative potency of these antibodies could be easily stratified upon IGF-II stimulation, and 2B4, 6D2, 9F2, and 10H5 belonged to the most effective group.

Also examined was the effect of these antibodies on ligand-dependent IGF-1R signaling by directly analyzing IGF-1R phosphorylation as well as major downstream pathways, such as AKT and MAPK activation. In this assay, MCF7 cells were grown in 6-well plates and serum was withdrawn for 16 hours. The cells were first incubated with anti-IGF-1R antibodies for 20 minutes, and followed by stimulation with IGF-I (50 ng/ml) or IGF-II (100 ng/ml) for ten minutes. The cells were directly lysed in SDS sample buffer, and the lysate was analyzed by Western blotting using antibodies against IGF-1R, phospho-IGF-1R, AKT, phospho-AKT, ERK1/2, phospho-ERK1/2 (also called MAPK1/2 and phospho-MAPK1/2) (all from Cell Signaling Technology, Inc.; Beverly, Mass., USA).

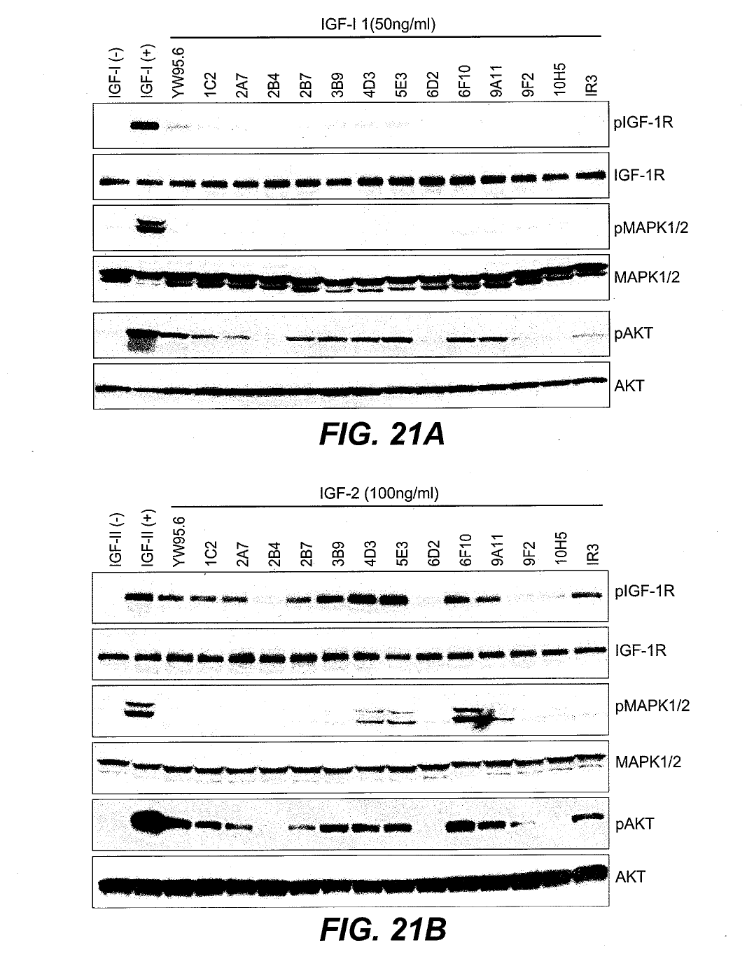

FIGS. 21A and 21B show the results of these experiments for IGF-I and IGF-II, respectively. While most of these antibodies were effective in inhibiting IGF-1-mediated IGF-1R phosphorylation and MAPK1/2 activation, only 2B4, 6D2, 9F2, and 10H5 also potently prevented AKT activation. In contrast, IGF-II-mediated signaling was less affected by several antibodies; however, 2B4, 6D2, 9F2, and 10H5 remained as strong inhibitors for IGF-II-mediated IGF-1R phosphorylation as well as for downstream AKT and MAPK1/2 activation.

Immunoprecipitation and Western Blotting

Cells were lysed in lysis buffer (20 mM TRIS-HCl (pH 7.5), 150 mM NaCl, 1 mM EDTA, 1 mM EGTA, 1% TRITON X-100™ buffer) with protease inhibitors and/or phosphatase inhibitor cocktail (Sigma, St. Louis, Mo.). Lysates were incubated on ice for 20 minutes and cleared by centrifugation at 14,000 rpm for 15 minutes. Equal amounts of cell lysates measured by BCA assay (Pierce Biotechnology, Rockford, Ill.) were used to immunoprecipitate IGF-1R with an anti-IGF-1R antibody 10F5 (generated through mouse hybridoma production as described for h10H5) or used for Western blotting using anti-IGF-1Rα or β subunit antibodies.

For the IGF-1R signaling pathway analysis, MCF7 cells were grown in six-well plates for 24 hours and then serum starved (serum-free and phenol-free RPMI medium supplemented with 1 mg/mL BSA) overnight. The cells were first incubated with h10H5 for 20 minutes, then stimulated with 50 ng/mL IGF-1 or 100 ng/mL IGF-11 for ten minutes. Cells were directly lysed in SDS sample buffer to preserve protein phosphorylation. After brief sonication, the lysates were separated by SDS-PAGE and transferred to nitrocellulose membranes (Invitrogen; Carlsbad, Calif.), which were probed with anti-phospho-IGF-1R, AKT, and ERK1/2 antibodies. The same blots were then stripped using Stripping Buffer™ (Pierce Biotechnology; Rockford, Ill.) and re-probed with antibodies against total IGF-1R β, AKT, and ERK1/2.

Results

To investigate the effects of h10H5 on IGF-1R signaling, human breast cancer MCF7 cells were pretreated with the antibody for 20 minutes, followed by IGF-I and IGF-II stimulation for ten minutes, and analyzed by Western blotting. IGF ligand treatment induced robust phosphorylation of IGF-1R as well as of downstream signaling components, such as AKT and ERK1/2, compared with controls. Exposure of cells to h10H5 resulted in a reduction in IGF-1R activation and downstream signaling. The inhibition was dose-dependent, being most effective at 1 or 10 μg/mL h10H5, with 0.1 μg/mL having only a minimal effect. In contrast, the control (anti-gp120) antibody failed to inhibit IGF-1- or IGF-II-mediated signaling even at 10 μg/mL.

Direct exposure of MCF7 cells to various concentrations of h10H5 (0.1-100 μg/mL) in the absence of ligand stimulation did not induce any observable phosphorylation of IGF-1R, AKT, and ERK1/2.

These data indicate that h10H5 is a potent antagonist of IGF-1R signaling induced by IGF-I and IGF-II.

### IV. Antibody-Induced IGF-1R Down-Regulation

Further exploration of the mechanism of action of anti-IGF-1R antibodies was carried out by testing the ability of the antibodies of the invention, particularly human chimeric antibodies of 2B4, 9F2, 10H5, as well as human phage antibody YW95.6, to induce IGF-1R down-regulation. MCF7 cells were treated with various antibodies (10 μg/ml) for 1, 4, 8, or 24 hours in the presence of 10% serum-containing medium. The cell lysates were harvested, separated by SDS-PAGE, and analyzed for IGF-1R levels by Western blotting using an anti-IGF-1R beta-chain antibody (Cell Signaling Technology, Inc.; Beverly, Mass., USA) or anti-beta-actin antibody.

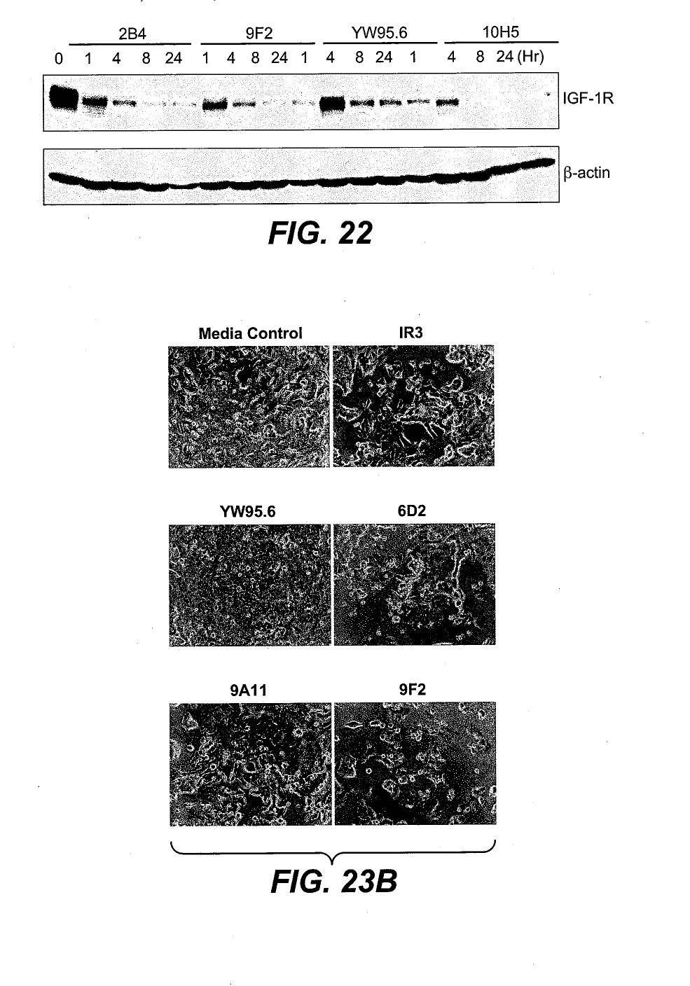

FIG. 22 shows the result of this experiment using human chimeric B4, 9F2, and 10H5 and YW95.6. Similar levels of beta-actin at various time points indicated that comparable amounts of cell lysates were loaded. IGF-1R was rapidly down-regulated and reached maximal reduction by eight hours, showing IGF-1R depletion was induced by antibody treatment.

In a separate experiment, MCF7 cells were treated with serially diluted h10H5, and the cell lysates were analyzed for IGF-1R and IR levels using anti-IGF-1R and anti-IR β-chain antibodies at 1, 2, 4, and 8 hours post-continuous treatment. Humanized 10H5.vX had a similar effect as chimeric 10H5.

### V. In Vitro Activity of Anti-IGF-1R Antibodies on Human Normal and Cancer Cell Lines

The in vitro activity of the different anti-IGF-1R antibodies generated was investigated using multiple normal and tumor human cell lines.

### A. Methods

### (1) Cell Lines

Human breast tumor cell lines MCF7, T47D, BT20, BT-474, and SW527, neuroblastoma cell line SK-N-AS, human colon tumor cell lines SW480, HT29, DLD1, HCT15, KM12, HCT116, and COLO205, human prostate tumor cell lines PC3 and DU145, human pancreatic cell lines MIA, PaCa2, and Capan1, human cervix tumor cell line HELA, human rhabdomyosarcoma cell line SJCRH30, and the human leukemia cell line HL60 were purchased from ATCC (Manassas, Va., USA). Human normal mammary epithelial cell line HMEC was purchased from Clonetics (Walkersville, Md., USA). All the cell lines were maintained in culture according to the conditions described by the manufacturer.

### (2) Cell-Based Assays

The different cell lines were cultured in their respective culture media at 37° C. in a humidified atmosphere of 5% CO₂. Subsequently, cells were detached from tissue-culture flasks using the cell-dissociation solution, TRIPLE SELECT™ solution (InVitrogen, Carlsbad, Calif., USA), and centrifuged at 1000 rpm for five minutes. Detached cells were washed once with PBS, counted, and seeded in 96-well plates (Corning, N.Y., USA) at 2000 cells per well in 100 μL of culture media. After an overnight incubation at 37° C. in a humidified atmosphere of 5% CO₂, the cell-culture medium was replaced with 50 μL of starvation medium consisting of serum-free RPMI 1640™ medium without phenol red. The cells were then starved for five hours in the incubator before addition of 25 μL of each anti-IGF-1R monoclonal antibody at various dilutions in serum-free RPMI 1640™ phenol red-free media. Later, 25 μL of phenol red-free RPMI 1640™ media supplemented with 4% of fetal bovine serum (FBS) (Sigma) with or without 4 ng/ml of human recombinant IGF-I were added on top of the 50 μL of antibody. The cells were then placed at 37° C. with 5% CO₂ for 5 to 6 days. At the end of the assay, cell viability was measured using the CELLTITER-GLO® kit according to the manufacturer's instructions (Promega, Madison, Wis., USA).

### B. Results

### (1) Initial Anti-IGF-1R Antibody Screen on Human Breast Tumor Cell Line MCF7

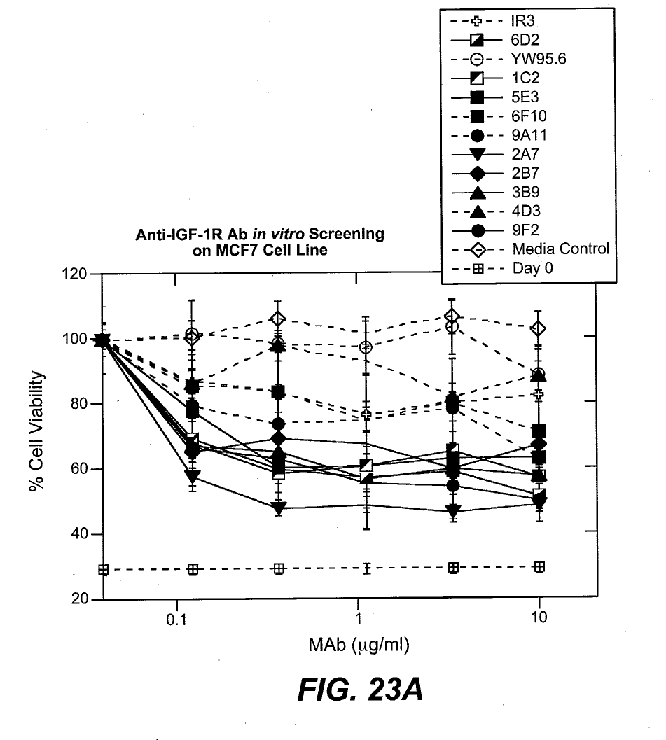

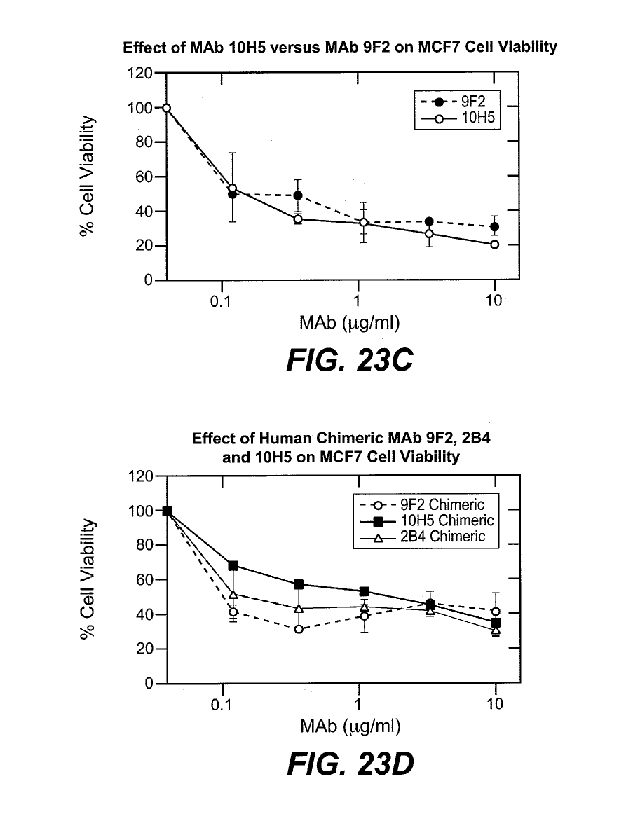

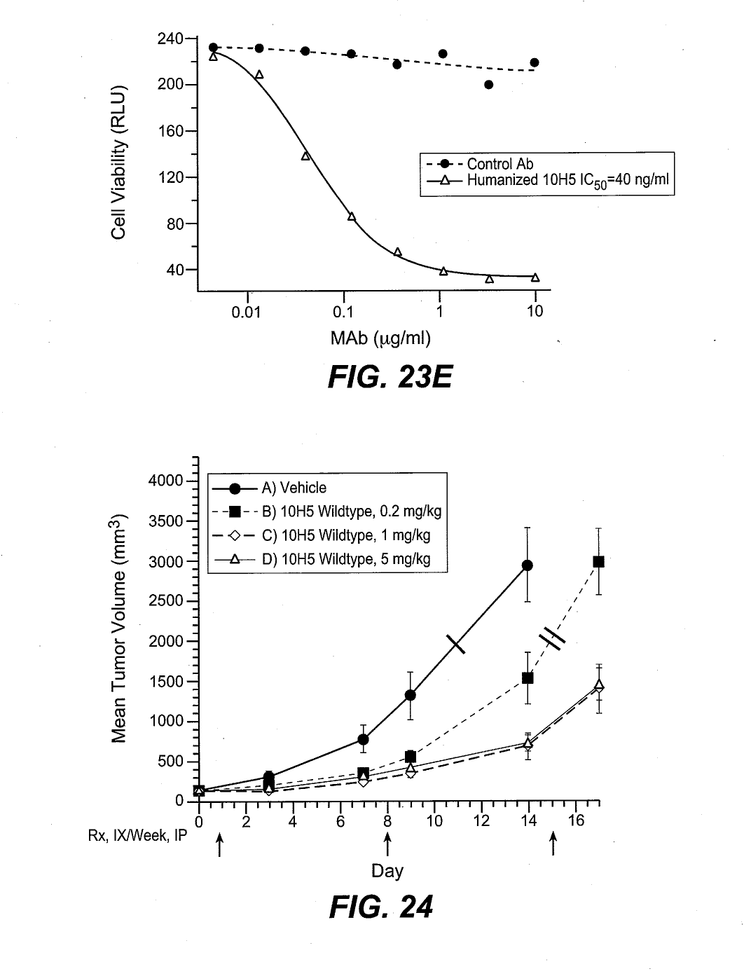

Using the optimized assay conditions described in the cell-based assay method section, ten mouse monoclonal antibodies (6D2, 1C2, 5E3, 6F10, 9A11, 2A7, 2B7, 3B9, 4D3, and 9F2 from the hybridomas deposited as set forth herein, PTA-7016, 7008, 7015, 7014, 7019, 7010, 7011, 7012, 7013, and 7018, respectively), one human phage antibody clone YW95.6, and one commercial antibody named IR3 used as a positive control were tested on the MCF7 cell line at concentrations ranging from 0.12 to 10 μg/ml (5×⅓ serial dilution point plus blank) (FIG. 23A).

Based on the cell-based assay data, the different anti-IGF-1R antibodies were ranked in three groups. The first group had no effect on the MCF7 cell viability compared to the media control. Only the human phage antibody YW95.6 fell into this category. The second group of antibodies (6F10, 4D3, and 9A11) had a similar effect (20% inhibition) on the MCF7 cell viability as the commercial antibody IR3. The third group of antibodies (1C2, 2B7, 3B9, 6D2, 5E3, 2A7, and 9F2) had a more pronounced effect with more than 20% inhibition of the MCF7 cell viability, which correlated with deceased cellular confluence by microscopic observations (FIG. 23B). Later, an additional antibody clone 10H5 (hybridoma deposited as set forth herein, PTA-7007) was purified and evaluated in the MCF7 cell-based assay in the presence of IGF-I in parallel with the antibody 9F2 (FIG. 23C).

### (2) Effect of the Selected Anti-IGF-1R Antibodies on Various Human Cell Lines

The monoclonal antibodies 6D2, 9F2, and 10H5 were chosen for further investigation on a large panel of human normal (HMEC) and tumor cell lines in addition to the breast-tumor cell line MCF7. The results are summarized in Table 5. Ultimately, this study would help the selection of mouse xenograft tumor models in which the anti-IGF-1R antibodies could be efficacious.

TABLE 5
Cell-line screening

| | | Effect of IR3, 6D2, 9F2, |
|---|---|---|
| | | and 10H5 |
| | | +: growth inhibition |
| Cell line | Cell type | −: no or weak effect |
| PC3 | Prostate Adenocarcinoma | − |
| DU145 | Prostate Adenocarcinoma | − |
| MCF7 | Breast Adenocarcinoma | + |
| T47D | Breast Ductal Carcinoma | − |
| HELA | Cervix Adenocarcinoma | + |
| KM12 | Colorectal Adenocarcinoma | − |
| SW480 | Colorectal Adenocarcinoma | + |
| HT29 | Colorectal Adenocarcinoma | + |
| DLD1 | Colorectal Adenocarcinoma | − |
| HCT15 | Colorectal Adenocarcinoma | − |
| HMEC | Human Mammary Epithelial Cell | − |
| SJCRH30 | Rhabdomyosarcoma | + |

Among the different cell lines tested, the breast-tumor cell line MCF7, the cervix-adenocarcinoma cell line HELA, the colorectal cell lines SW480 and HT29, and the rhabdomyosarcoma cell line SJCRH30 were significantly impacted by the treatment with anti-IGF-1R antibodies 6D2, 9F2, and 10H5 (>25% cell-viability reduction upon treatment).

(3) Effect of the Human Chimeric Version of 9F2, 2B4, and 10H5 and h10H5.vX on MCF7 Cell Viability

Based on the biochemical assay and the cell-based assay data, the antibodies 9F2, 2B4, 10H5, and h10H5.vX were selected for further investigation as to cell viability.

Cell Proliferation Method

For the cell viability assay, MCF7 cells were seeded onto 96-well plates at 2000 cells/well and incubated overnight at 37° C. in a humidified atmosphere of 5% CO₂/95% air in RPMI 1640™ medium supplemented with 10% FBS (Sigma, St. Louis, Mo.) and 1% GLUTAMAX™ reagent (Promega, Madison, Wis.). The next day cells were starved in serum-free RPMI 1640™ medium (phenol red-free) for five hours followed by addition of serially diluted human chimeric versions of 9F2, 2B4, and 10H5, as well as h10H5.vX, for one hour. Subsequently, phenol red-free PRMI 1640™ medium supplemented with FBS or IGF-I was added to reach a final concentration of 1% FBS and 1 ng/mL IGF-I. Cells were then incubated with continuous IGF-1R antibody exposure. After five days, the cell viability was then determined using a CELLTITER-GLO™ kit (Promega, Madison, Wis.) according to the manufacturer's instructions.

Results

The human chimeric versions of these antibodies were therefore generated and evaluated in the MCF7 cell-viability assay in the presence of IGF-I (FIG. 23D). The cell-based assay data indicated that the human chimeric versions of 9F2, 2B4, and 10H5 were equivalent at decreasing the MCF7 cell viability in vitro.

A five-day continuous h10H5 treatment of MCF-7 cells led to a marked reduction in cell viability, suggesting anti-proliferative activity, while a control antibody specific for B cells did not. The inhibition was dose-dependent with an IC₅₀ between 34 and 57 ng/mL. Confirming an effect on proliferation, h10H5 also exhibited a dose-dependent inhibition of [³H] thymidine incorporation in SK-N-AS neuroblastoma and SW527 breast cancer cells with IC₅₀ between 23 and 77 ng/mL. Because SK-N-AS and SW527 cells express endogenous IGF-II, serum-free or 0.1% serum-containing conditions were used to minimize the effect of other growth factors on the assay. The results suggest that h10H5 can inhibit DNA replication in certain cancer cells. See also FIG. 23E.

### VI. Anti-Tumor Activity in Xenograft Animal Models

### A. Material and Methods

Female nude (nu/nu) and C.B-17 SCID beige mice were obtained from Charles Rivers Laboratories, Inc. (Hollister, Calif.). Animals were about six to eight weeks old and weighed about 25 grams each. Mice were acclimated to study conditions for at least three days before tumor-cell inoculations. The animals were housed in standard rodent micro-isolator cages. Only animals that appeared to be healthy and that were free of obvious abnormalities were used for the study. Each mouse was given a subcutaneous injection of SK-N-AS cells (10 million), SW527 cells (5 million), Colo205 cells (5 million), or A549 cells (5 million) into the dorsal right flank in a volume of 0.2 mL. Data collected from each experimental group were expressed as mean tumor volume±the standard error of the mean (SEM). Tumors were monitored until they reached a mean volume of 130-260 mm3. Mice were then randomized into various treatment groups, including vehicle or control antibody groups, and antibody and/or chemotherapy treatments were started. Each group consisted of eight to ten mice, each of which was given an intraperitoneal injection of test material weekly for 2-4 weeks. For testing antibodies, the loading doses were twice as much as the subsequent ones. Tumors were measured twice weekly throughout the experiment starting on the first day of treatment. Tumor volumes were measured in two dimensions (length and width) using ULTRA CAL-IV™ calipers (Model 54-10-111, Fred V. Fowler Company, Inc.; Newton, Mass.), and calculated using the formula of:

Tumor Volume(mm³)=(length×width²)×0.5.

Tumor-inhibition and body-weight graphs were plotted using KALEIDAGRAPH™ 3.6 software (Synergy Software; Reading, Pa.). Percent tumor-growth inhibition (% TGI) derived from mean tumor volumes on a given day was calculated using the following formula, in which C=the mean tumor volume of the control group, and T=the mean volume from each group of mice given active treatment:

% TGI=100×[(C−T)/C]

Tumor incidence was determined by the number of measurable tumors in each group. Partial regression is defined as tumor regression of >50% but <100% of starting tumor volume at any day during the study. Complete regression is defined as tumor regression of 100% from initial starting tumor volume at any day during the study. Mean tumor volume and standard error of the mean (SEM) were calculated using JMP software, version 5.1.2 (SAS Institute; Cary, N.C.). Data analysis and generation of p-values were performed using the Dunnett's t-test for tumor volumes, or a log-rank test for doubling time, which is defined as the number of days for a tumor to double its size measured at the day of randomization, with JMP software, version 6.0 (SAS Institute; Cary, N.C.). This study was conducted in accordance with the Guide for the Care and Use of Laboratory Animals, published by the NIH (NIH Publications 85-23, revised 1985).

### B. Results and Discussion

(1) SK-N-AS Neuroblastoma Xenograft Model

Three different weekly dosings of h10H5.vX (0.2 mg/kg, 1 mg/kg, and 5 mg/kg) were tested using this model. Compared to the vehicle-treated control group, treatment with antibody h10H5.vX resulted in dose-dependent inhibition of tumor growth (FIG. 24). At 0.2 mg/kg, the response was moderate (48% TGI at day 14); however, increased dosing at 1 or 5 mg/kg led to stronger inhibition (76%-77% TGI at day 14).

There was a statistically significant difference (p=0.0002) using a log-rank test in the distribution of time to tumor doubling between the treated (1 or 5 mg/kg) group versus the controls. Although the delay in tumor growth was significant, these tumors eventually grew up to large sizes in later time points, suggesting, without being limited to any one theory, that 10H5 is a cytostatic agent. Ki-67 staining exhibited significantly decreased proliferative index in the treated samples.

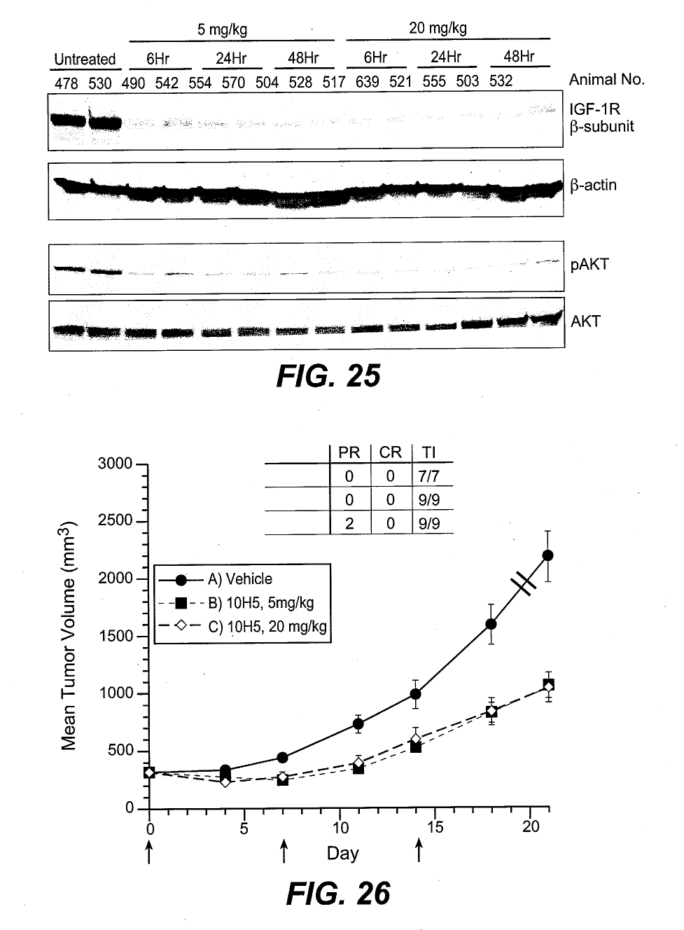

Whether the efficacy observed resulted from target inhibition was determined by performing pharmacodynamic analysis by treating pre-formed xenograft tumors, which were 400-600 mm³ in size, and the tumor samples were collected at before treatment, or 6, 24, and 48 hours post-treatment, and homogenized and analyzed by Western blotting. Compared to the untreated samples, 10H5 induced rapid IGF-1R down-regulation (FIG. 25), which was observed at 6 hours and lasted until 48 hours. More importantly, this target modulation resulted in decreased AKT phosphorylation, which is a well established marker for the downstream signaling. Since AKT can be activated by multiple upstream receptor tyrosine kinases, these data suggest, without being limited to any one theory, that IGF-1R is a major regulator of the PI3K-AKT pathway in SK-N-AS cells.

In addition, h10H5 displayed anti-tumor activity in vivo in association with decreased AKT activation and glucose uptake. To assess the effect of h10H5 on the growth of tumor xenografts, nu/nu mice bearing SC SK-N-AS tumors were given a single IV injection at doses ranging from 0.5 to 200 mg/kg. TGI was observed in all h10H5 dose groups as compared to control. Since several tumor-bearing mice in the control group (A) had to be euthanized at Day 9 due to excessive tumor burden, percentages of TGI were calculated for tumor volumes measured on this day. On Day 9, TGI was 51% (p=0.0003) at 0.5 mg/kg, and 66%-74% (p<0.0001) at 2-200 mg/kg, suggesting that maximal efficacy in this model could be reached at relatively low doses of h10H5.

Time to the first tumor doubling was significantly longer (p=0.03-0.0009) in all h10H5 groups with a range of 4.5-6.4 days compared with 2.7 days for the vehicle group.

These results demonstrate strong single-agent activity of h10H5 in this model.

In a separate xenograft experiment, it was examined whether h10H5 treatment in vivo affected tumor-associated IGF-1R signaling. SK-N-AS tumor samples treated at 5 or 20 mg/kg h10H5 were collected at 6, 24, and 48 hours post dosing and analyzed by Western blotting. IGF-1R down-regulation was observed as early as six hours and maintained at least up to 48 hours. Furthermore, phospho-AKT levels decreased significantly while total AKT levels remained unchanged in these tumors. Therefore, IGF-1R-mediated signaling in the SK-N-AS tumors was effectively inhibited by h10H5.

(2) SW527 Breast-Cancer Xenograft Model

Two different weekly dosings (5 mg/kg and 20 mg/kg) of h10H5.vX were tested using this SW527 breast-cancer cell line xenograft model in SCID beige mice (5 million cells/mouse). Compared to the vehicle control group, treatment with antibody 10H5 resulted in strong inhibition of tumor growth in either 5 mg/kg (52% TGI at day 21) or 20 mg/kg (53% TGI at day 21) groups (FIG. 26). Two partial responses were observed in the 20 mg/kg group. There was a statistically significant difference using a log-rank test in the distribution of time to tumor doubling between the 5 mg/kg (p=0.001) or the 20 mg/kg (p=0.002) group and the controls. These data provide evidence that h10H5.vX had a strong single-agent activity in the SW527 model.

(3) Colo-205 Colorectal-Cancer Model

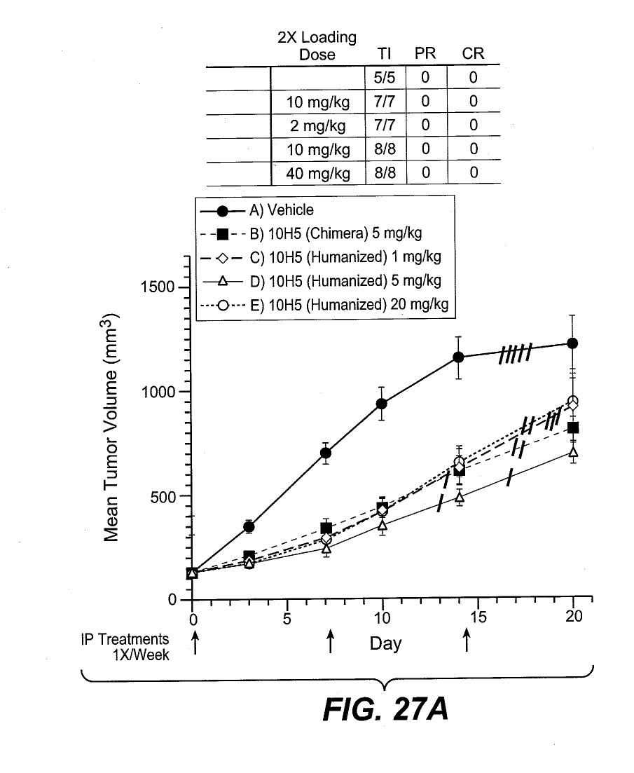

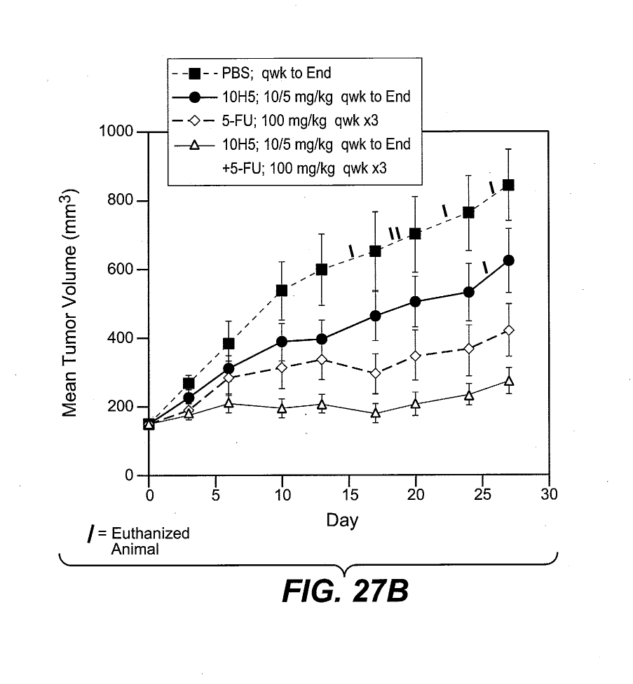

Humanized 10H5 (h10H5.vX) (1 mg/kg, 5 mg/kg, and 20 mg/kg) and chimeric 10H5 (5 mg/kg) were tested using a colorectal-cancer xenograft model involving Colo205 (5 million cells/mouse) tumors in athymic nude mice. Weekly dosings of humanized or chimeric 10H5 were delivered via intraperitoneal injection throughout the study. Compared to the vehicle control group, treatment with humanized and chimeric antibody 10H5 resulted in moderate inhibition of tumor growth (44-59% TGI at day 14, FIG. 27A). There was a statistically significant difference using a log-rank test in the distribution of time to tumor doubling between the various 10H5 groups (p=0.002 or below) and the controls. However, no significant differences were observed in either various dosing groups or between humanized and chimeric 10H5.

In a separate study, h10H5.vX was also tested in combination with 5-fluorouracil (5-FU) in a colorectal-cancer xenograft model involving Colo205-e215 xenograft tumors. Weekly dosings of humanized 10H5 were delivered via intraperitoneal injection throughout the study, while 5-FU (100 mg/kg) was given weekly for the first three weeks. 5-FU or h10H5.vX alone resulted in 53% and 25% TGI at day 27 (FIG. 27B), respectively. An additive inhibitory effect (64% TGI at day 27) was observed when 5-FU was used in combination with 10H5. These data provide evidence that h10H5.vX had single-agent activity and could enhance the inhibitory effect of 5-FU on tumor growth.

### (4) A549 Lung-Cancer Model

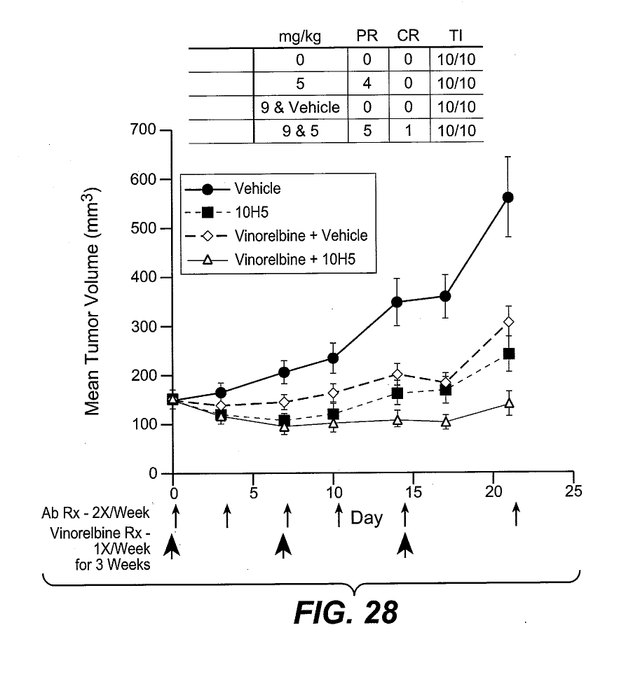

The effect of antibody h10H5.vX (5 mg/kg), vinorelbine (9 mg/kg), or the combination of both agents was tested using a lung-cancer model involving A549 (5 million cells/mouse) tumors in athymic nude mice. h10H5.vX was delivered via intraperitoneal injection two times per week throughout the study, while vinorelbine (9 mg/kg) was given weekly for the first three weeks. Compared to the vehicle control, either h10H5.vX or vinorelbine treatment resulted in 57% and 45% inhibition of tumor growth (TGI) at day 21, respectively (FIG. 28). There was a statistically significant difference using a log-rank test in the distribution of time to tumor doubling between the h10H5.vX (p=0.02) or vinorelbine (p=0.05) group versus the controls. Combination therapy led to a further inhibition of tumor growth (75%) and a delay in tumor doubling by an additional six days relative to the h10H5.vX group. These data provide evidence that h10H5.vX had single-agent activity and could enhance the inhibitory effect of vinorelbine on tumor growth.

It is expected that chimeric and humanized 10H5 and other IGF-1R antibodies herein would be effective in an IGF-II-responding model such as a colorectal cancer model that is predictive of the behavior of colorectal cancer in humans. It is expected that chimeric and humanized 10H5 and other IGF-1R antibodies herein would be clinically effective with arinotecan or with TARCEVA® (erlotinib), or another EGFR inhibitor, or with an anti-VEGF antibody such as AVASTIN® (bevacizumab), an Apo2L/TRAIL DR5 agonist (such as apomab, a DR-5-targeted dual proapoptotic receptor agonist), irinotecan, fulvestrant, or a chemotherapeutic agent such as FOLFOX (5-fluorouricil, leucovovin, and oxaliplatin), or ERBITUX™ (cetuximab), HERCEPTIN® (trastuzumab), OMNITARG™ (pertuzumab), and/or an aromatase inhibitor such as letrozole in treating such cancer types as colorectal, lung, or breast cancer.

It is expected that the patient treated with an antibody herein such as chimeric or humanized 10H5 using a clinical protocol based on the parameters described in this specification and as known to those skilled in this art will show clinical improvement in the signs or symptoms of breast, lung, or colorectal cancer as evaluated by any one or more of the primary or secondary efficacy endpoints known for treating these diseases. Moreover, the patient who is resistant or refractory to chemotherapy or another biological agent and who is treated, using a clinical protocol based on various parameters as described in this specification and as known to those skilled in the art, with the chimeric or humanized antibody 10H5 alone or in combination with a second medicament appropriate for the disease is expected to show greater improvement in any of the signs or symptoms of the cancer, compared to the patient who continues on with the medicament to which he or she is resistant or refractory, or compared to the patient who is treated with only the second medicament appropriate for the disease and not with the chimeric or humanized 10H5.

In the remaining Examples, the term “10H5” is used interchangeably with “rhuMAb 10H5, “h10H5,” and “10H5.vX” as identified above.

### EXAMPLE 3

### h10H5 Induces IGF-1R Internalization and Trafficking Through Transferrin-Containing Early Endosomes to Late Endosomes/Lysosomes

For the internalization assay, SK-N-AS and MCF7 cells grown on LABTEKII™ slides were incubated from five minutes to four hours with 5 μg/mL h10H5 at 37° C., 5% CO₂ in complete (10% FBS) growth medium containing lysosomal protease inhibitors (5 μM pepstatin A (Roche Applied Science; Indianapolis, Ind.), 10 μg/mL leupeptin (Roche Applied Science; Indianapolis, Ind.)), and, in some experiments, 10 μg/mL ALEXA488™-transferrin. Following incubation, cells were chilled, washed five times in cell media, fixed for 20 minutes with 3% paraformaldehyde, quenched for ten minutes with 50 mM NH₄Cl in PBS, and permeabilized with saponin buffer (0.4% w/v saponin, 2% FBS, 1% BSA). Internalized/uptaken rhuMAb 10H5 was detected with Cy3-anti-human detection reagent. Where indicated, cells were also stained with 1:1000 mouse anti-LAMP1 or 1:100 rabbit anti-IGF-1R 0 subunit followed by FITC-conjugated anti-mouse Fc (Jackson ImmunoResearch; West Grove, Pa.). Slides were coverslipped with DAPI-containing VECTASHIELD™ (Vector Labs) and imaged by epifluorescence microscopy using the 100× objective of a DELTAVISION™ deconvolution microscope (Applied Precision; Issaquah, Wash.) powered by SOFTWORX™ (version 3.4.4). Figures were compiled using ADOBE PHOTOSHOP CS™ software (San Jose, Calif.).

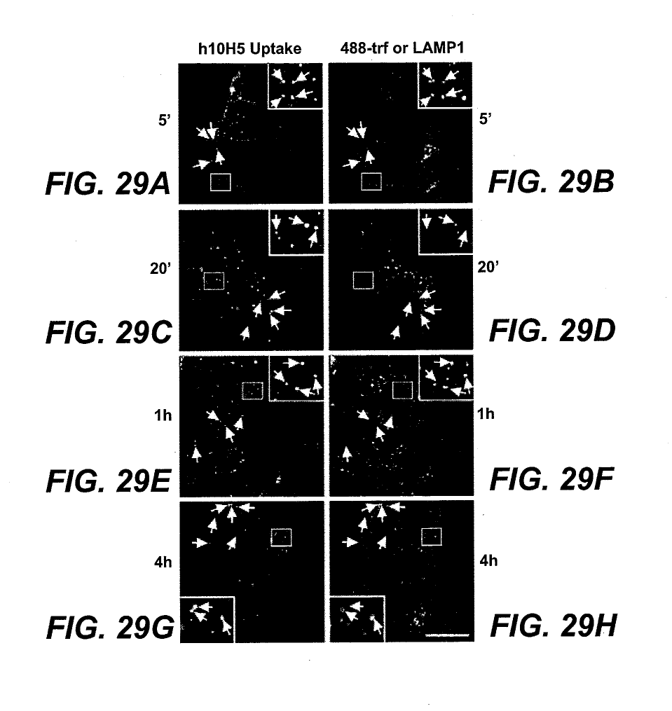

IGF-1R is normally localized at the plasma membrane of MCF7 cells. 10H5 was rapidly internalized within five minutes upon addition to cells, most likely via clathrin-coated vesicles, as demonstrated by co-localization with transferrin (see FIGS. 29A and 29B), which internalized by clathrin-mediated endocytosis (Watts and Marsh, J Cell Sci., 103:18 (1992)). Within 20 minutes, rhuMAb 10H5 had started to diverge from the transferrin recycling pathway (FIGS. 29C and 29D), and, after 60 minutes, it was detectable within a subset of late endosomes and lysosomes, as demonstrated by anti-LAMP1 staining (see FIGS. 29E and 29F). The total h10H5 signal was weaker at the 60-minute time point, indicating some degradation of the antibody, despite the presence of lysosomal protease inhibitors; after four hours, the signal was even weaker, but still detectable within the lysosomal lumen, surrounded by LAMP1 on the limiting membrane (see FIGS. 29G and 29H).

Since these data suggest that rhuMAb 10H5 can trigger the internalization and degradation of IGF-1R, IGF-1R levels were directly examined by Western blotting following h10H5 treatment. Indeed, IGF-1R was already partially downregulated at one hour, and reached maximal reduction at 4-8 hours. Prolonged incubation up to 24 hours did not lead to further reduction. Antibody h10H5 at 1 or 10 μg/mL had similar time-dependent effects on IGF-1R levels, but the downregulation at 0.1 μg/mL was slower at early time points and reached comparable reduction at 4-8 hours. In contrast, neither IGF-I nor anti-gp120 antibody treatment affected IGF-1R levels. Thus, although IGF-I stimulated IGF-1R phosphorylation, this did not lead to down-regulation, unlike h10H5 treatment. h10H5-mediated IGF-1R downregulation was specific because IR levels were unaffected by h10H5 treatment.

### EXAMPLE 4

### h10H5-Induced IGF-1R Down-Regulation is Mediated by Proteasome and Lysosome Pathways

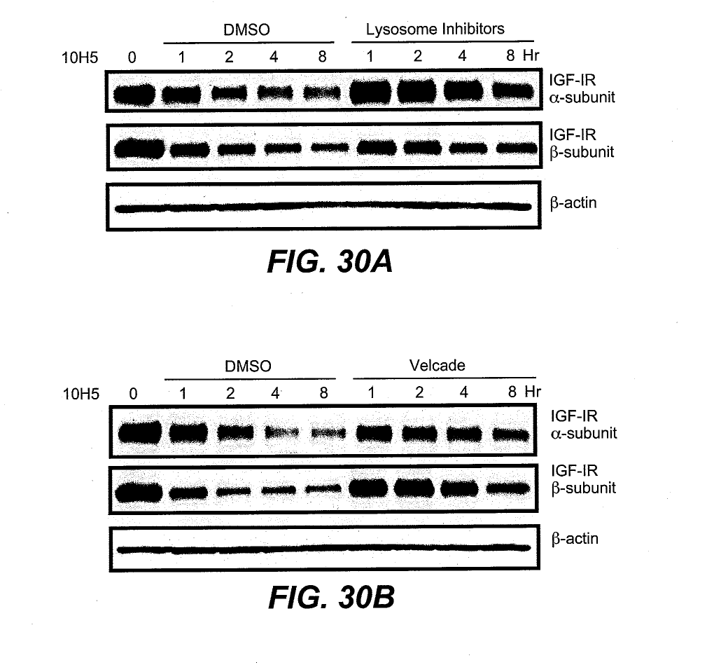

Both proteasome and lysosome pathways have been implicated in ligand-mediated IGF-1R degradation. To examine whether these pathways contribute to h10H5-induced IGF-1R downregulation, SK-N-AS cells were pretreated with either a combination of 5 μM of pepstatin A and 10 μg/ml of leupeptin or 30 μM of VELCADE® bortezomib for one hour, and subsequently exposed to h10H5 treatment for 1 to 8 hours in the presence of protease or protesome inhibitors. Cell lysates were analyzed for IGF-1R α-subunit and β-subunit by Western blotting. β-actin was used as a loading control. Because the extracellular (ECD) and intracellular (ICD) domains are exposed to different cellular environments during the internalization and trafficking processes, antibodies that specifically react to the α-subunit that is a major part of ECD or the C-terminus of the β-subunit, and therefore a part of ICD, were used for Western blot analysis. The α- and β-subunits of IGF-1R exhibited similar kinetics of 10H5-induced downregulation in SK-N-AS cells in the absence of proteasomal or lysosomal protease inhibitors (see FIGS. 30A and 30B).

Treatment with proteasome inhibitor VELCADE® bortezomib or lysosomal inhibitors leupeptin and pepstatin A resulted in delayed IGF-1R downregulation (see FIGS. 30A and 30B). However, VELCADE® bortezomib was more effective in preventing β-subunit downregulation, while leupeptin and pepstatin A preferentially inhibited α-subunit degradation. Comparison between α- and β-subunit downregulation was also performed using immunofluorescence in the presence of leupeptin and pepstatin A; only the α-subunit but not the β-subunit, was still detectable 4 hours post-10H5 treatment. Without being limited to any one theory, these data suggest that α- and β-subunits of IGF-1R are differentially regulated by proteasome and lysosomal pathways.

### EXAMPLE 5

### h10H5 Effectively Cooperates with Docetaxel and Anti-VEGF Antibody to Inhibit the Growth of SW527 Breast Cancer Xenograft Tumors

In additional to single-agent activity, 10H5 was examined as to whether it could be combined with docetaxel, a chemotherapeutic agent, or with anti-VEGF therapy in the SW527 breast-cancer xenograft model. SCID beige mice were given a subcutaneous injection of SW527 cells in a Hank's buffered salt solution/MATRIGEL™ suspension, and were randomized to six groups of ten mice per group when the tumors reached a volume of 109-345 mm3 (mean volume of 255 mm3). Athymic nude mice bearing SC SW527 tumors (n=10 per group) received one of the following six treatments: vehicle, h10H5 (loading dose of 10 mg/kg followed by weekly doses at 5 mg/kg, given intraperitoneally); docetaxel (12.5 mg/kg on Days 0, 4, and 8, given intravenously); cross-species anti-VEGF antibody B20-4.1 (weekly doses at 10 mg/kg, given intraperitoneally); rhuMAb IGFR (h10H5) in combination with docetaxel; or h10H5 in combination with B20-4.1.

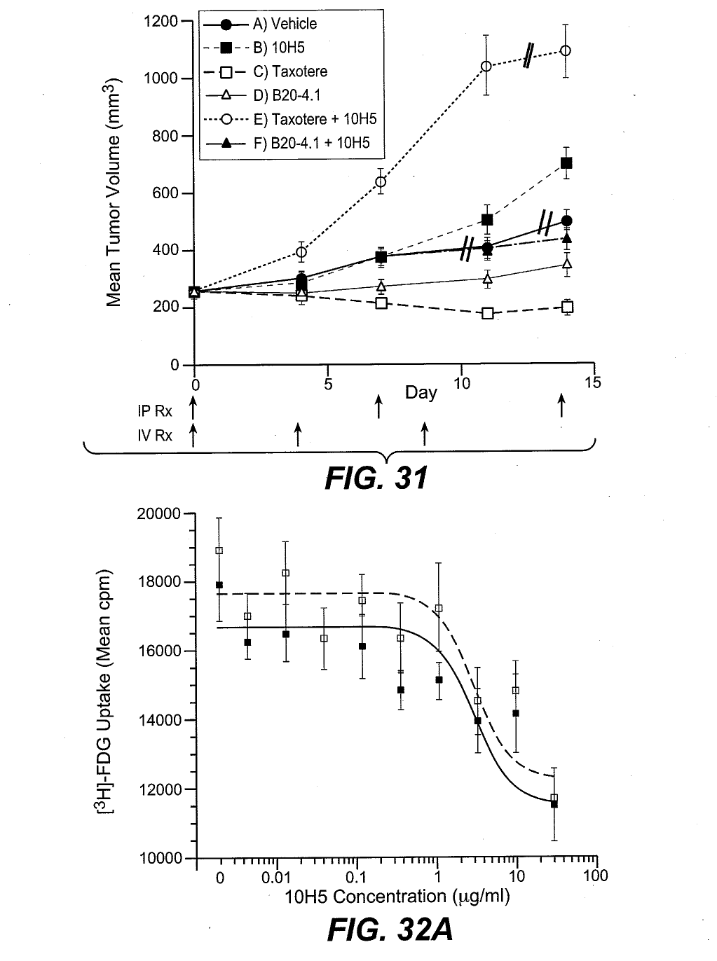

Administration of weekly IP doses of 5 mg/kg rhuMAb IGFR alone resulted in significant inhibition of SW527 tumor growth compared with vehicle treatment (p<0.01 by Dunnett t test) (See FIG. 31). TGI values on Days 11 and 14 were 52% and 36%, respectively. Treatment with rhuMAb IGFR also significantly slowed the time to first tumor doubling, from 5.6 days in the vehicle group to 10.2 days in the rhuMAb IGFR group (p<0.002 by log-rank test).

Since docetaxel is frequently used in the treatment of breast cancer, mice with SW527 human breast xenograft tumors were given docetaxel as a single agent or in combination with rhuMAb IGFR. In mice given 12.5 mg/kg docetaxel alone, tumor growth was significantly profoundly inhibited on Day 14 (54% TGI) compared with the group given vehicle (p<0.01 by Dunnett t test; see FIG. 31). The level of TGI when docetaxel was administered in combination with rhuMAb IGFR was increased on Day 14 (82% TGI) compared with the administration of docetaxel as a single agent (p<0.01 by Dunnett t test). Two mice treated with single-agent docetaxel were euthanized on Day 8, and another two mice in this group were euthanized on Day 12 because of body weight losses >20%. Time to the first tumor doubling was significantly prolonged when the combination group (docetaxel+rhuMAb IGFR) was compared with either the docetaxel or rhuMAb IGFR single-agent group (p<0.01 by log-rank test). The combination of rhuMAb IGFR and docetaxel was therefore significantly more effective in inhibiting tumor growth than either single agent.

The cross-species anti-VEGF antibody B20-4.1, which binds human and murine VEGF with affinity similar to that of bevacizumab with human VEGF (Liang et al., J. Biol. Chem., 281: 951-961 (2006)), was also tested in this breast tumor model. At doses of 10 mg/kg, B20-4.1 significantly inhibited tumor growth (60% TGI) compared with vehicle treatment on Day 14 (p<0.0001 by Dunnett t test; see FIG. 31). The combination of B20-4.1 and rhuMAb IGFR increased the level of TGI (68%) on Day 14 compared with the single-agent B20-4.1 group (p=0.0196 by the Dunnett t test). However, the difference in the time to the first tumor doubling in the combination group was not statistically significant when compared with the single-agent B20-4.1 group (p=0.133 by log-rank test); a similarly low tumor growth rate was observed in both of these groups.

These results demonstrate that while rhuMAb IGFR, docetaxel, and B20-4.1 delayed SW527 tumor growth as single agents, the combination of rhuMAb IGFR with either docetaxel or B20-4.1 increased the levels of tumor regression or tumor inhibition.

### EXAMPLE 6

### h10H5 Inhibits Glucose Uptake In Vitro and In Vivo

IGF-1R is closely related to insulin receptor. The possible effect of h10H5 on glucose uptake was examined.

### Glucose Uptake and Thymidine Incorporation Assays

SK-N-AS cells were plated in 96-well plates at 10,000 cells/well and allowed to adhere overnight. The following day, medium was removed and replaced with either glucose-free DMEM (for FDG uptake) or 50:50 Ham's F-12:DMEM (for thymidine incorporation) containing 0.1% or 0% FBS. Cells were incubated with a range of concentrations of h10H5 for a total of 48 hours. To determine FDG uptake, cells were labeled with 2 μCi/well [³H]-FDG (2-Fluoro-2-deoxy-D-glucose, [5,6-³H]; 0.74-2.22 TBq/mmol; 20-60 Ci/mmol; American Radiolabeled Chemicals, Inc., St. Louis, Mo.) for the last 24 hours.

To assess the effects of h10H5 on proliferation, cells were labeled with 1 μCi/well ³H-thymidine ([methyl-³H]thymidine; 1.5-2.2 TBq/mmol; 40-60 Ci/mmol; GE Healthcare/Amersham, Piscataway, N.J.) for the last six hours of the 48-hour incubation. Cells were then harvested onto UNIFILTER® GF/C™ filter plates using a FILTERMATE™ harvester (PerkinElmer, Shelton, Conn.). MICROSCINT™ 20 scintillation fluid was added to all wells and radioactivity incorporated per well (CPM) was determined using a Packard TOPCOUNT NXT™ microplate scintillation counter.

### Results

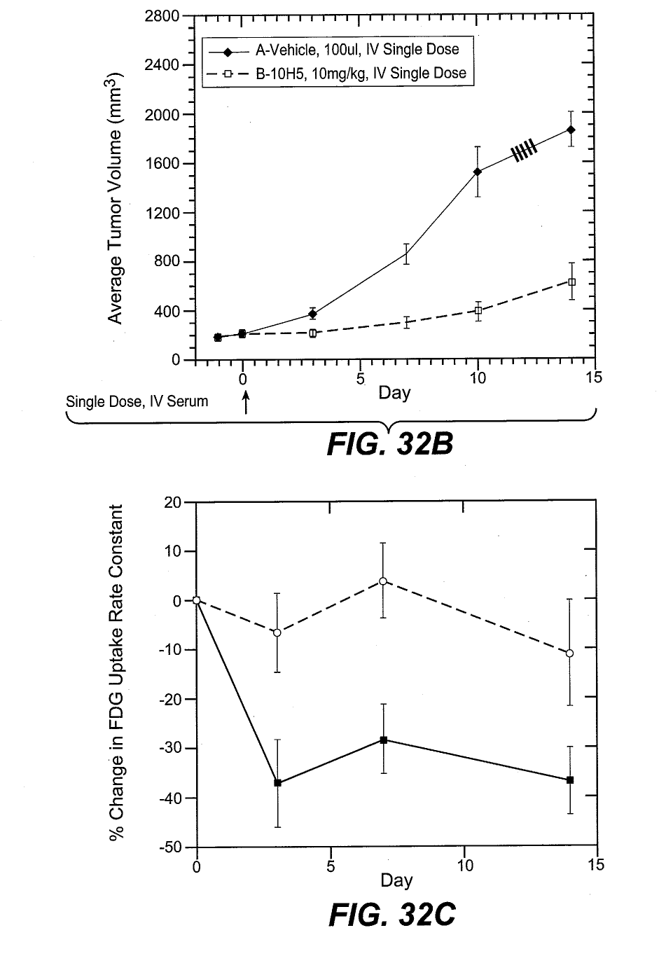

Although it is feasible to collect xenograft tumors to measure drug activity in preclinical studies, obtaining tumor biopsies from metastatic cancer patients poses a significant challenge. Therefore, noninvasive imaging techniques, such as measurement of glucose uptake in tumors as an indicator of viability by PDG-PET, is highly desirable for monitoring drug responses in clinical trials. Despite lack of cross-reactivity to IR, h10H5 inhibited FDG uptake with an IC₅₀ of 3 μg/ml (see FIG. 32A). These data, along with the fact that IGF-1R signaling modulates cell metabolic activity (LeRoith et al., Curr Opin Clin Nutr Metab Care, 5: 371-375 (2002)), and the desire to develop a non-invasive approach to monitor in vivo activity of the antibody, prompted the testing of FDG-PET imaging in monitoring changes in tumor FDG uptake rate as a pharmacodynamic marker of h10H5 activity. PDG-PET is a molecular imaging technique widely used in the diagnosis and staging of oncologic malignancies. Its use for monitoring treatment response is less widespread.

### FDG-PET Imaging Study

A separate SK-N-AS xenograft study was conducted to evaluate FDG-PET imaging as a measure of drug response. Athymic nude mice bearing SC SK-N-AS tumors were given a single IV injection of 10 mg/kg rhuMAb IGFR (h10H5). Dynamic PET scans were performed on tumors following caliper measurements on Day 0 prior to treatment, and again on Days 3, 7, 10, and 14. At the beginning of a 30-minute dynamic PET scan approximately 250 μCi of F18-FDG was injected into the lateral tail vein of each mouse. PET data were processed into a time series of images to allow quantification of the tumor relative to the blood pool, using the liver signal as a proxy for blood (Green et al., J. Nucl. Med., 39: 729-734 (1998)), and the region of interest analysis was performed using software provided with the microPET scanner by the vendor (Siemens Preclinical Solutions). Numerical integration and calculations for the conversion of the raw data into Patlak plots were performed with MICROSOFT EXCEL™ software. The slope of the linear portion of the Patlak plot is equal to Ki, the FDG uptake rate constant (units of per second). Treatment responses on a given post-treatment day were assessed as the percentage change in the FDG uptake rate constant Ki relative to the pre-treatment value. A negative change represents a decrease in FDG uptake. These difference data were used for the t-tests comparing vehicle and treatment groups at the imaging time points. Data collation and statistical analysis were performed with the MICROSOFT EXCEL™ program using the built-in Student t-test in a two-tailed comparison.

### Results

Several vehicle-treated tumor-bearing mice were removed from the study on Day 10 because of exceedingly large tumors. h10H5 treatment resulted in significant inhibition of tumor growth compared with the vehicle treatment (see FIG. 32B); TGI values for h10H5 at Days 3, 7, 10, and 14 were 42.2% (p=0.004), 63.2% (p<0.0001), 72.6% (p<0.0001), and 64.1% (p<0.0001), respectively (p-values derived from Dunnett t test). In addition, time to tumor doubling from Day 0 to Day 17 was significantly slower in the rhuMAb IGFR group (10.5 days) compared with the vehicle group (3.0 days; p<0.0001 by log-rank test).

FDG-PET imaging of the xenograft tumors was performed in parallel on Days 0, 3, 7, and 14. F18-FDG was injected intravenously, and PET data were processed into a time series of images to allow quantification of the tumor relative to the blood pool using the liver signal as a proxy for blood (Green et al., J Nucl Med, 39:729-734 (1998)). The region of interest analysis was performed using software provided with the microPET scanner by the vendor (Siemens Preclinical Solutions). Numerical integration and calculations for the conversion of the raw data into Patlak plots was done with MICROSOFT EXCEL® software. The slope of the linear portion of the Patlak plot is equal to Ki, the FDG uptake rate constant (units of per second). Treatment responses on a given post-treatment day were assessed as the percentage change in the FDG uptake rate constant Ki relative to the pre-treatment value. A negative change represents a decrease in FDG uptake. These difference data were used for the t-tests comparing vehicle and treatment groups at the imaging time points. Data collation and statistical analysis were performed using MICROSOFT EXCEL™ software using the built-in Student t test in a two-tailed comparison. h10H5 treatment resulted in decreases of 37%, 28%, and 37% in FDG uptake rate on Days 3, 7, and 14, respectively, relative to the baseline rate on Day 0 (p<0.05 by Student t test for all time points; see FIG. 32C).

These results confirm single-agent activity of rhuMAb IGFR in this model at 10 mg/kg, and show that FDG-PET changes accompanied treatment and correlated with the inhibition of tumor growth. The use of FDG-PET as a pharmacodynamic biomarker of rhuMAb IGFR activity in tumor tissue is supported by these results.

### EXAMPLE 7

### h10H5 Does not Mediate Significant ADCC

Monoclonal antibodies may achieve their therapeutic effect by recruiting cytotoxic cells through their cell-surface Fc receptors and kill tumor cells by ADCC. Therefore, the ability of h10H5 to mediate ADCC activity was examined.

### ADCC Methods

Blood from normal volunteers was drawn into heparinized syringes, mixed with an equal volume of Hanks' Balanced Salt Solution (HBSS), layered onto LSM™ lymphocyte separation medium (Mediatech, Inc.) and centrifuged at 400×g for 20 minutes. Peripheral blood mononuclear cells (PBMC) at the interface were harvested, washed in HBSS, and resuspended in RPMI 1640™ medium containing 0.1% BSA, 2 mM L-glutamine, 10 mM HEPES, and 50 mg/mL GENTAMICIN™ antibiotic.

SK-N-AS human neuroblastoma cells and BT474 human breast cancer cells were incubated with serially diluted 10H5 and HERCEPTIN® trastuzumab, respectively, in round-bottomed 96-well plates for 30 minutes at 37° C. PBMCs were subsequently added and the incubation was continued for four more hours at 37° C. After four hours, the plates were centrifuged and the supernatants were harvested. Cytotoxicity was measured by lactate dehydrogenase activity released by the supernatants, as determined according to the CYTOTOX-ONE HOMOGENEOUS MEMBRANE INTEGRITY ASSAY PROCEDURE™ technique (Promega). BT474 cells have amplified Her2 and allow the use of HERCEPTIN® trastuzumab as a positive control in this assay; they also have comparable levels of IGF-1R expression with those in SK-N-AS cells.

To determine the percentage of cell-mediated cytotoxicity, the average absorbance was calculated and the background was subtracted as follows:

$$Cytotoxicity ( \% ) = \frac{Experimental - Effector Spontaneous - Target Spontaneous}{Target Maximum - Target Spontaneous} \times 100$$

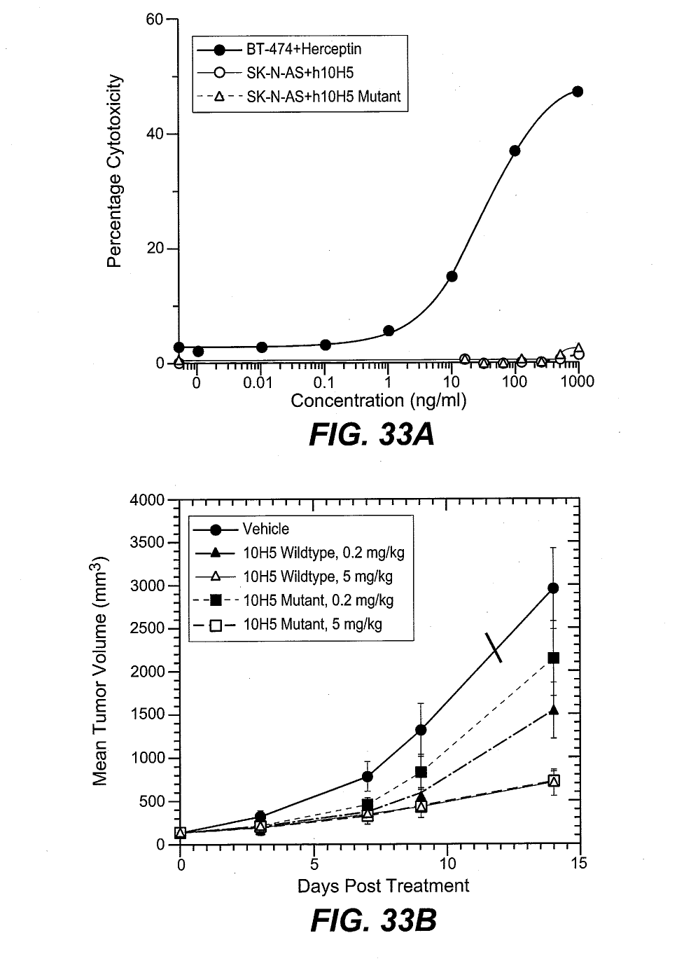

### Results

Whereas HERCEPTIN® trastuzumab showed detectable ADCC in BT-474 cells, no ADCC activity was observed with either wild-type (h10H5) or an Fcγ receptor binding-defective mutant h10H5 (D265A mutant 10H5) (Clynes et al., Nat. Med., 6: 443-446 (2000)), which lacks N-linked glycosylation at the Fc region and hence does not bind to Fcgamma (an interaction required for ADCC), in SK-N-AS cells (FIG. 33A) or BT-474 cells at concentrations up to 100 μg/mL. These data suggest, without being limited to any one theory, that h10H5 does not mediate detectable ADCC activity in vitro.

To confirm this, anti-tumor activity of the parental and D265A h10H5 antibodies was compared. Anti-tumor activities for the SK-N-AS model were identical with 5 mg/kg wild-type or mutant (D265A) h10H5 (FIG. 33B). At the suboptimal dose of 0.2 mg/kg, the TGIs of wild-type and D265A h10H5 were not significantly different (48% and 28%, respectively) (p−0.53). These data suggest, without being limited to any one theory, that tumor growth inhibition is likely a direct consequence of blockade of IGF-1R-mediated signaling.

### EXAMPLE 8

### h10H5 blocks IGF-I Binding to IGF-1R and Inhibits the Receptor-Mediated Signaling

### Solid Phase Binding Assay

Recombinant human IGF-1R ECD was coated onto 96-well plates at 200 ng/well, and incubated overnight at 4° C. The plates were washed with 0.05% TWEEN™ 20 surfactant in PBS. Then they were blocked at room temperature for one hour with 5% BSA in PBS. Serially diluted h10H5.vX, commercially available anti-IGF-1R antibody αIR3 (Calbiochem, San Diego, Calif.), and a control antibody anti-gp120 (herpes simplex virus glycoprotein D) were added to the plates, after which they were incubated at room temperature for one hour. Biotinylated IGF-I or IGF-II (80 ng/mL) was subsequently added and incubated at room temperature for one hour. After washing, wells were incubated with streptavidin-HRP (1:6,000) at ambient temperature for 20 minutes. The signals were developed with TMB substrate. When blue coloration appeared, 100 μL/well of phosphoric acid at 1 M was added to stop the revelation process. The optical density was read at A_{450nM}.

### Results

In the competitive solid-phase binding assays, h10H5 inhibited IGF-I and IGF-II binding to IGF-1R with an IC₅₀ of 3.4 nM (FIGS. 19C and 19D).

### EXAMPLE 9

### Rodent Models of Aging

A mouse model of the premature aging syndrome known as Hutchinson-Gilford Progeria Syndrome (HGPS), caused by defects in the A-type lamins, may be employed to test the antibodies herein for their ability to reverse effects of aging (Mounkes et al., Nature, 423 (6937):298-301 (2003)). Although normal at birth, children with progeria begin to develop growth retardation, thinning skin, and fragile bones as young as 18 months, and usually die of heart disease in their early teens.

Another mouse model for testing the antibodies herein for effect on aging is a prematurely aged mouse deficient in DNA repair and transcription (de Boer et al., Science, 296 (5571):1276-1279 (2002); see also Comment in Science, 296(5571): 1250-1251 (May 17, 2002)) Such mice have a mutation in XPD, a gene encoding a DNA helicase that functions in both repair and transcription and that is mutated in the human disorder, trichothiodystrophy (TTD). TTD mice were found to exhibit many symptoms of premature aging, including osteoporosis and kyphosis, osteosclerosis, early graying, cachexia, infertility, and reduced life-span. TTD mice carrying an additional mutation in XPA, which enhances the DNA-repair defect, showed a greatly accelerated aging phenotype, which correlated with an increased cellular sensitivity to oxidative DNA damage. Without being limited to any one theory, it is believed that aging in TTD mice is caused by unrepaired DNA damage that compromises transcription, resulting in functional inactivation of critical genes and enhanced apoptosis.

Additional inbred and hybrid rodent models suitable for screening the antibodies herein for anti-aging activity are discussed in the “Proceeding of the second international conference on animal models for aging research,” Experimental Gerontology, 32, 1/2: 1-242 (1997). For example, the SOD2 nullizygous mouse lives approximately seven days, and dies from a complex phenotype. The symptoms include neurodegeneration, dilated cardiomyopathy, anemia, hepatic lipid accumulation, ketosis, and a severe spongiform encephalopathy associated with disturbances in neurological function. The life span of these mice can be extended approximately three- to fourfold, and many of their pathological phenotypes alleviated, by treating them chronically with catalytic antioxidants.

The antibodies herein may also be tested for their effects on learning and memory impairment and functional measures in the senescence-accelerated mouse (Nishiyama et al., Experimental Gerontology, 32 (1/2): 149-160 (1997)).

Additionally, the effects of the antibodies on the probability of survival and mean body weight of C57BL/6N, DBA/2N, B6xD2F1, and B6xC3F1 mice, and of F344, Brown Norway, and F344xBN rats of both sexes can be tested See Sprott, Experimental Gerontology, 32 (1/2): 205-214 (1997).

It is expected that appropriate doses of h10H5 and other antibodies herein (for example, 100 mg, 200 mg, 300 mg, 400 mg, 500 mg, or 1000 mg/dose or 10-100 mg/kg once daily) will show improved function in such animal models versus a control, indicating efficacy in treating aging in mammals, including humans.

### EXAMPLE 10

### Clinical Aging Study in Military Recruits

Recruits are studied before and after 12 weeks of military training. They are randomized to receive a suitable dose of either h10H5 antibody or placebo, e.g., 100 mg, 200 mg, 300 mg, 400 mg, 500 mg, or 1000 mg/dose, or 10-100 mg/kg once daily. It is expected that the recruits receiving h 10H5 will show a consistent trend for improvement in VO₂ₘₐₓ and a reversal of muscle fatigue versus the placebo. Such measures would be indicative of an enhanced ability, especially for those of II genotype (and thus lower ACE activity), of the h10H5-treated recruits to perform higher workloads, before reaching anaerobic threshold. Thus, they would be expected to be less fatigued during moderate-to-intense exercise.

### EXAMPLE 11

### Clinical Aging Study in Cachectic Patients

A variety of cachectic conditions, for instance due to chronic heart failure, AIDS, liver cirrhosis, cancer, cardiac, idiopathic, and malnutrition have been studied. Activation of the renin angiotensin system (RAS) is found in patients with cachexia. One indication of RAS is an elevated plasma level of angiotensin II (AT II). The mean AT II plasma levels in patients with RAS are clearly above the upper limit of the normal range of 20 to 40 pg/ml. This correlation does not depend on any particular type of cachexia; elevated AT II plasma levels (ergo, RAS activity) are found in patients with idiopathic cachexia. Activation of the RAS is apparently limited to cachectic disorders; it is not observed in patients having a similar degree of weight loss due to malnutrition.

Experiments can be conducted to demonstrate if antibody h10H5 or other antibodies described herein would be of benefit for cachectic patients, even if they have been previously treated with an ACE inhibitor. One set of patients has cachexia due to congestive heart failure (CHF) (Group A). A second set of patients has CHF and a muscle myopathy suffering from idiopathic cachexia (Group B). Both groups of patients exhibit impaired exercise capacity and impaired left ventricular function (LVEF<40%). Each group is randomized to receive either h10H5 antibody or a placebo (10-100 mg/kg once daily of antibody or placebo, or doses of, e.g., 100 mg, 200 mg, 300 mg, 400 mg, 500 mg, or 1000 mg/dose). The clinical status and parameters of body composition, strength, and treadmill exercise capacity of all patients are studied at baseline and during follow-up.

Both groups are measured for plasma AT II levels. Blood samples are collected from each patient after supine rest of at least ten minutes. An antecubital polyethylene catheter is inserted and 10 ml of venous blood are drawn. After immediate centrifugation, aliquots (EDTA plasma sample) are stored at −70° C. until analysis. AT II is measured using a commercially available RIA (IBL, Hamburg, Germany, sensitivity 1.5 pg/ml). After extraction of the plasma samples, AT II is assayed by a competitive RIA. This RIA uses a rabbit anti-AT II antiserum and a radio-iodinated AT II tracer. Bound and free phases are separated by a second antibody bound to solid-phase particles, followed by a centrifugation step. The radioactivity in the bound fractions is measured and a typical standard curve can be generated. The test has a cross-reactivity with AT 1 of <0.1% and a within-and-between run reproducibility between 3.9 and 8.6%. Each patient is expected to have a higher AT II level than the normal range.

Both groups are analyzed for bioelectrical impedance, which is performed in the erect position using a body-fat analyzer (TANITA THF-305, Tanita Corporation, IL, USA). Lean and fat mass are automatically analyzed based on equations supplied and programmed into the machine by the manufacturer. These equations are based upon a comparison with measurements in a healthy population.

Whole-body dual-energy X-ray absorptiometry (DEXA) scans are performed using a LUNAR™ model DPXIQ total body scanner (Lunar Radiation Company, Madison, Wis., USA, Lunar system software version 4.3 c). At each time point, each patient is scanned rectilinearly from head to toe. A scan takes less than 20 minutes. The mean radiation dose per scan is reported to be about 0.75 μSv (Fuller et al., Clinical Physiology, 12:253-266 (1992)), about 1/50th of a normal chest X-ray. The DEXA method can be used to obtain fat and lean tissue mass. The technical details, performance, and segment demarcation of DEXA are described in Mazess et al., Calcif. Tissue Int., 44:228-232 (1989) and Mazess et al., Am. J. Clin. Nutr., 51:1106-1112 (1990)). The error of lean tissue measurements is >2% and of fat tissue measurements <5% (Ley et al., Am. J. Clin. Nutr., 55:950-954 (1992)).

The patients undergo symptom-limited treadmill exercise testing. A standard Bruce protocol with the addition of a “stage 0” consisting of three minutes at a speed of one mile per hour with a 5% gradient is used. The patients breathe through a one-way valve connected to a respiratory mass spectrometer (Amis 2000, Odense, Denmark). Minute ventilation, oxygen consumption, and carbon dioxide production are calculated on line every ten seconds using a standard inert gas dilution technique. Patients are encouraged to exercise to exhaustion. Exercise time and oxygen consumption at peak exercise adjusted for total body weight (peak VO₂ in ml/kg/min) are measured as an index of the exercise capacity.

In another test, the patients in both groups are seated in a rigid frame, with their legs hanging freely. An inelastic strap attaches the ankle to a pressure transducer. The recording (Multitrace 2, §, Jersey, Channel Islands) from the pressure transducer is used to assess strength and provide visual feedback to the patient. A plateau of maximum force production indicates that the contraction is maximal. The best of three voluntary contractions on each leg, with a rest period of at least one minute in-between, is taken to represent the maximal voluntary quadriceps muscle strength of the right and left leg, respectively.

Results include a follow-up of 120 days for Group A and 80 days for Group B. Both groups of patients are also studied at intermediate time points. The patients treated with h10H5 in each group are expected to improve during the study as compared to the placebo-treated patients in their exercise capacity (measured by exercise time in Groups A and B, and by peak VO₂ in Group B). Improvement over control is also expected in both Groups with respect to quadriceps muscle strength in both legs. These clinical benefits are expected to be achieved while the Group A patients experience a lean and fat tissue gain, and while the Group B patients experience no further weight loss and improvement in general clinical status and relative muscle performance. No side-effects of treatment are expected to be observed.

The SOLVD treatment study (Mazess et al., Calcif Tissue Int., supra) was a randomized, double-blind, and placebo-controlled trial investigating the effects of treatment with enalapril (an ACE inhibitor) on cachexia in patients with CHF. See also The SOLVD Investigators, N. Engl. J. Med., 325:293-302 (1991). That study demonstrated that significant weight loss, i.e. cardiac cachexia, is a frequent event in CHF patients. Spontaneous reversal of the weight loss is very rare. Cardiac cachexia is closely and independently linked to impaired survival of CHF patients. Treatment with an ACE inhibitor, enalapril, in addition to conventional therapy, reduced the frequency of the risk of death and of developing cardiac cachexia in CHF patients. Overall, enalapril therapy reduced the risk of developing cardiac cachexia by 19%. The same effects expected in Groups A and B as defined above would be expected to occur, versus the placebo control, if the patients in Group A and/or Group B, or other types of CHF patients, received an ACE inhibitor such as enalapril (2.5 to 20 mg per patient) along with an antibody herein such as h10H5 in a clinical study as conducted, for example, by the investigators in SOLVD, using a dosing of, e.g., 100 mg, 200 mg, 300 mg, 400 mg, 500 mg, or 1000 mg of antibody/dose.

### Deposit of Material

The following materials have been deposited with the ATCC, 10801 University Blvd., Manassas, Va. 20110-2209, USA:

| Material | ATCC Dep. No. | Deposit Date |
|---|---|---|
| murine hybridoma; Lymph | PTA-7007 | Sep. 20, 2005 |
| nodes: IGFIR: 4373 | | |
| (10H5.3.4) | | |
| murine hybridoma; Lymph | PTA-7008 | Sep. 20, 2005 |
| nodes: IGFIR: 4376 | | |
| (1C2.8.1) | | |
| murine hybridoma; Lymph | PTA-7009 | Sep. 20, 2005 |
| nodes: IGFIR: 4364 | | |
| (2B4.2.8) | | |
| murine hybridoma; Lymph | PTA-7010 | Sep. 20, 2005 |
| nodes: IGFIR: 4362 | | |
| (2A7.5.1) | | |
| murine hybridoma; Lymph | PTA-7011 | Sep. 20, 2005 |
| nodes: IGFIR: 4363 | | |
| (2B7.4.1) | | |
| murine hybridoma; Lymph | PTA-7012 | Sep. 20, 2005 |
| nodes: IGFIR: 4365 | | |
| (3B9.4.1) | | |
| murine hybridoma; Lymph | PTA-7013 | Sep. 20, 2005 |
| nodes: IGFIR: 4366 | | |
| (4D3.6.2) | | |
| murine hybridoma; Lymph | PTA-7014 | Sep. 20, 2005 |
| nodes: IGFIR: 4369 | | |
| (6F10.1.1) | | |
| murine hybridoma; Lymph | PTA-7015 | Sep. 20, 2005 |
| nodes: IGFIR: 4367 | | |
| (5E3.1.1) | | |
| murine hybridoma; Lymph | PTA-7016 | Sep. 20, 2005 |
| nodes: IGFIR: 4368 | | |
| (6D2.6.1) | | |
| murine hybridoma; Lymph | PTA-7017 | Sep. 20, 2005 |
| nodes: IGFIR: 4375 | | |
| (4D7.1.4) | | |
| murine hybridoma; Lymph | PTA-7018 | Sep. 20, 2005 |
| nodes: IGFIR: 4372 | | |
| (9F2.6.2) | | |
| murine hybridoma; Lymph | PTA-7019 | Sep. 20, 2005 |
| nodes: IGFIR: 4371 | | |
| (9A11.3.1) | | |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposits for 30 years from the date of deposits. The deposits will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the Director of the United States Patent and Trademark Office to be entitled thereto according to 35 USC § 122 and the Director's rules pursuant thereto (including 37 CFR § 1.14, with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written description is considered to be sufficient to enable one skilled in the art to practice the invention. The present invention is not to be limited in scope by the example presented herein. Indeed, various modifications of the invention in addition to those shown and described herein will become apparent to those skilled in the art from the foregoing description and fall within the scope of the appended claims.

What is claimed is:

1. An isolated anti-insulin-like growth factor-I receptor (IGF-1R) antibody comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid;
(b) a HVR-L2 sequence comprising amino acids B1-B7, wherein B1-B7 is SASYRYS (SEQ ID NO:7) or YTSRLHS (SEQ ID NO: 8) or SASYRKS (SEQ ID NO:9) or GASSRAS (SEQ ID NO:10);
(c) a HVR-L3 sequence comprising amino acids C₁-C₉, wherein C₁-C₉ is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid;
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid;
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32).

2. The antibody of claim 1 wherein SEQ ID NO:13 is QQYSNYPYT (SEQ ID NO:33), QQYKHYPYT (SEQ ID NO:34), QQYKKYPYT (SEQ ID NO:35), QQYKNYPYT (SEQ ID NO:36), QQYRIYPYT (SEQ ID NO:37), QQYKRYPYT (SEQ ID NO:38), QQYKSYPYT (SEQ ID NO:39), QQYRSYPYT (SEQ ID NO:40), or QQYSKYPYT (SEQ ID NO:41).

3. The antibody of claim 1 comprising all of SEQ ID NOS:1, 7, and 11.

4. The antibody of claim 1 comprising all of SEQ ID NOS:16, 21, and 26.

5. The antibody of claim 1 comprising all of SEQ ID NOS:1, 7, and 11 and all of SEQ ID NOS:16, 21, and 26.

6. The antibody of claim 1 wherein at least a portion of its framework sequence is a human consensus framework sequence.

7. The antibody of claim 1 that specifically binds to human IGF-1R and blocks the interaction of an insulin-like growth factor (IGF) with IGF-1R, wherein said antibody is an antagonist of human IGF-1R and has an Fc region.

8. The antibody of claim 7 wherein the IGF is IGF-I.

9. The antibody of claim 7 wherein the Fc region is a wild-type Fc region.

10. The antibody of claim 1 that does not bind specifically to the human insulin receptor.

11. The antibody of claim 1 wherein the sequence of its light-chain variable region has about 1-10 amino acid insertions, deletions, or substitutions from SEQ ID NO:53.

12. The antibody of claim 11 wherein the sequence of its light-chain variable region comprises no more than about eight amino acid changes from SEQ ID NO:53.

13. The antibody of claim 1 wherein the sequence of its heavy-chain variable region has about 1-10 amino acid insertions, deletions, or substitutions from SEQ ID NO:55.

14. The antibody of claim 13 wherein the sequence of its heavy-chain variable region comprises no more than about eight amino acid changes from SEQ ID NO:55.

15. The antibody of claim 1 that is humanized.

16. The antibody of claim 1 that binds IGF-1R with an affinity of at least about 10⁻¹² M.

17. The antibody of claim 1 that is of the IgG1 or IgG2a isotype.

18. The antibody of claim 1 having a monovalent affinity to human IGF-1R that is about the same as or greater than the monovalent affinity to human IGF-1R of a murine antibody produced by a hybridoma cell line deposited on Sep. 20, 2005 under American Type Culture Collection Accession Number PTA-7007, PTA-7008, PTA-7009, PTA-7010, PTA-7011, PTA-7012, PTA-7013, PTA-7014, PTA-7015, PTA-7016, PTA-7017, PTA-7018, or PTA-7019.

19. The antibody of claim 1 comprising the light-chain variable domain in SEQ ID NO:44, 49, 53, 57, 64, 65, 66, 67, 68, 69, 70, 71, 72, or 73, or the heavy-chain variable domain in SEQ ID NO:47, 51, 55, or 61, or comprising both SEQ ID NOS:44 and 47, or both SEQ ID NOS:49 and 51, or both SEQ ID NOS:53 and 55, or both SEQ ID NOS:57 and 61, or both SEQ ID NOS: 64, 65, 66, 67, 68, 69, 70, 71, 72, or 73 and 55.

20. The antibody of claim 1 comprising the variable light amino acid sequence in SEQ ID NO:53 or the variable heavy amino acid sequence in SEQ ID NO:55 or comprising both sequences.

21. The antibody of claim 1 comprising the full-length heavy-chain sequence in SEQ ID NO:90 and the full-length light-chain sequence in SEQ ID NO:91.

22. A method of producing an antibody comprising:
(i) culturing a host cell comprising nucleic acid encoding an anti-insulin-like growth factor-I receptor (IGF-1R) antibody comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid;
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10);
(c) a HVR-L3 sequence comprising amino acids C1-C9, wherein C1-C9 is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid;
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid;
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32), under conditions to produce the antibody; and
(ii) recovering the antibody.

23. A method of inhibiting insulin-like growth factor-I receptor (IGF-1R)-activated cell proliferation, said method comprising contacting a cell or tissue with an effective amount of an anti-insulin-like growth factor-I receptor (IGF-1R) antibody comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid;
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10);
(c) a HVR-L3 sequence comprising amino acids C₁-C₉, wherein C₁-C₉ is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid;
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid;
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:28) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32).

24. A method of treating a cancer in a subject comprising administering to the subject an effective amount of an anti-insulin-like growth factor-I receptor (IGF-1R) antibody comprising at least one hypervariable region (HVR) sequence selected from the group consisting of:
(a) a HVR-L1 sequence comprising amino acids A1-A11, wherein A1-A11 is KASQNVGSNVA (SEQ ID NO:1) or RASQDINNYLT (SEQ ID NO:2) or RASQDISNYLN (SEQ ID NO:3) or KASQNLRSKVA (SEQ ID NO:4) or KASQYVGTHVA (SEQ ID NO:5) or RASQSISSYLA (SEQ ID NO:6), where N is any amino acid;
(b) a HVR-L2 sequence comprising amino acids B1-B7, wherein B1-B7 is SASYRYS (SEQ ID NO:7) or YTSRLHS (SEQ ID NO:8) or SASYRKS (SEQ ID NO:9) or GASSRAS (SEQ ID NO:10);
(c) a HVR-L3 sequence comprising amino acids C₁-C₉, wherein C₁-C₉ is HQYNNYPYT (SEQ ID NO:11) or QQGNTLPWT (SEQ ID NO:12) or QQYNNYPYT (SEQ ID NO:13) or QQRFSVPFT (SEQ ID NO:14) or QQYYSSPLT (SEQ ID NO:15), where N is any amino acid;
(d) a HVR-H1 sequence comprising amino acids D1-D10, wherein D1-D10 is GYTFTRFWIH (SEQ ID NO:16) or GYTLANYGMN (SEQ ID NO:17) or GYNLANYGLN (SEQ ID NO:18) or GFSFSSQGIS (SEQ ID NO:19), or GFTFSSYAMS (SEQ ID NO:20), where N is any amino acid;
(e) a HVR-H2 sequence comprising amino acids E1-E18, wherein E1-E18 is GEINPSNGRTNYNENFKN (SEQ ID NO:21) or GWINTNTGKPTYSDEFKG (SEQ ID NO:22) or GWINTNTGAPTYAEEFKG (SEQ ID NO:23), or SRISPSGGSTYYADSVKG (SEQ ID NO:24), where N is any amino acid, or comprising amino acids E1-E17, wherein E1-E17 is STISYDGSTYYADSVKG (SEQ ID NO:25); and
(f) a HVR-H3 sequence comprising amino acids F1-F6, wherein F1-F6 is GGRLDQ (SEQ ID NO:26) or comprising amino acids F1-F12, wherein F1-F12 is SIYYYGSRYFNV (SEQ ID NO:27) or SIYYYASRYFNV (SEQ ID NO:228) or ESSYYEWGAMDV (SEQ ID NO:29), where N is any amino acid, or comprising amino acids F1-F11, wherein F1-F11 is EHYFHWGGMDV (SEQ ID NO:30) or EEYYYWGAMDV (SEQ ID NO:31), or comprising amino acids F1-F13, wherein F1-F13 is QFMLWGKQFGMDV (SEQ ID NO:32).

25. A method for assessing activity of an anti-insulin-like growth factor-1 receptor (IGF-1R) antibody in tumor tissue comprising subjecting tissue from tumors treated with the antibody to positron emission tomography with 2-fluoro-2-deoxy-D-glucose (FDG-PET) imaging and determining if the antibody inhibits FDG uptake into the tissue, with inhibition of FDG uptake correlating with delayed tumor growth.
